(19) United States
(12) Patent Application Publication
(10) Pub. No.: US 2026/0007750 A1
(43) Pub. Date: Jan. 8, 2026

(54) CELLS AND COMPOSITIONS FOR TREATING CANCER

(71) Applicants: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(72) Inventors: Michel SADELAIN, New York, NY (US); Karlo PERICA, New York, NY (US)

(73) Assignees: MEMORIAL SLOAN-KETTERING CANCER CENTER, New York, NY (US); SLOAN-KETTERING INSTITUTE FOR CANCER RESEARCH, New York, NY (US); MEMORIAL HOSPITAL FOR CANCER AND ALLIED DISEASES, New York, NY (US)

(21) Appl. No.: 19/328,825

(22) Filed: Sep. 15, 2025

Related U.S. Application Data
(63) Continuation of application No. PCT/US24/20615, filed on Mar. 20, 2024.
(60) Provisional application No. 63/491,146, filed on Mar. 20, 2023.

(51) Int. Cl. A61K 40/46; A61K 40/11; A61K 40/15; A61K 40/31; A61K 40/50; C07K 14/005; C07K 14/725

(52) U.S. Cl. CPC A61K 40/46; A61K 40/11; A61K 40/15; A61K 40/31; A61K 40/50; C07K 14/005; C07K 14/7051; C12N 2740/16022

(57) ABSTRACT

The presently disclosed subject matter provides compositions and methods for targeting immune responses toward tumor antigen-bearing cells. It relates to cells, e.g., modified immunoresponsive cells, comprising an antigen-recognizing receptor (e.g., a chimeric antigen receptor (CAR)) and an immunoevasins (e.g., a NEF polypeptide).

### CROSS-REFERENCE TO RELATED APPLICATIONS

The instant application is a continuation application of International Patent Application No. PCT/US2024/020615, filed Mar. 20, 2024, which claims priority to U.S. Provisional Patent Application No. 63/491,146, filed Mar. 20, 2023, and to U.S. Provisional Patent Application No. 63/536,258, filed Sep. 1, 2023, the content of each of which is incorporated in its entirety, and to each of which priority is claimed.

### SEQUENCE LISTING

The instant application contains a Sequence Listing which has been submitted electronically in XML format and is hereby incorporated by reference in its entirety. Said XML copy, created on Sep. 15, 2025, is named 0727341810.xml, and is 146,853 bytes in size.

### INTRODUCTION

The presently disclosed subject matter provides compositions and methods for targeting immune responses toward tumor antigen-bearing cells. It relates to cells, e.g., modified immunoresponsive cells, comprising an antigen-recognizing receptor (e.g., a chimeric antigen receptor (CAR)) and an immunoevasins (e.g., a NEF polypeptide).

### BACKGROUND OF THE INVENTION

Chimeric Antigen Receptor (CAR) T cells are a gene-edited cell therapy that is custom-manufactured from each patient's cells (autologous). CAR T cells have proven effective and even curative for some B cell cancer patients. However, custom manufacturing is costly and complex, leading to delays in treatment and limited patient access. In contrast, an allogeneic cell therapy would be immediately available for infusion “off-the-shelf,” which could be manufactured in bulk at a reduced cost, could be repeatedly infused, and would be of higher quality. One of the barriers to allogeneic therapies is rejection by the host immune system. Thus, novel approaches to protect allogeneic CAR T cells from rejection are needed.

### SUMMARY OF THE INVENTION

The presently disclosed subject matter provides compositions and methods for targeting immune responses toward tumor antigen-bearing cells. It relates to cells, e.g., modified immunoresponsive cells, comprising an antigen-recognizing receptor (e.g., a chimeric antigen receptor (CAR)) and an immunoevasin (e.g., a NEF polypeptide).

The presently disclosed subject matter provides cells comprising an immunoevasin and an antigen recognizing receptor that targets an antigen.

In certain embodiments, the immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91. In certain embodiments, the immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91. In certain embodiments, the immunoevasin comprises the amino acid sequence set forth in SEQ ID NO: 1.

In certain embodiments, the cell further comprises a second immunoevasin. In certain embodiments, the second immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91. In certain embodiments, the second immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91. In certain embodiments, the second immunoevasin comprises the amino acid sequence set forth in SEQ ID NO: 5. In certain embodiments, the cell further comprises a gene disruption of a NLRC5 locus, a RFX5 locus, or a combination thereof.

The presently disclosed subject matter also provides cells comprising an antigen recognizing receptor that targets an antigen and a gene disruption of a NLRC5 locus, a RFX5 locus, or a combination thereof.

In certain embodiments, the antigen-recognizing receptor is a T cell receptor (TCR), a chimeric antigen receptor (CAR), or a TCR like fusion molecule. In certain embodiments, the antigen-recognizing receptor is a chimeric antigen receptor (CAR).

In certain embodiments, the CAR comprises an extracellular antigen-binding domain that binds to the antigen, and an intracellular signaling domain that is capable of delivering an activation signal to the cell. In certain embodiments, the intracellular signaling domain of the CAR comprises a CD3ζ polypeptide. In certain embodiments, the CD3ζ polypeptide is a native CD3ζ polypeptide or a modified CD3ζ polypeptide. In certain embodiments, the modified CD3ζ polypeptide comprises a native ITAM1, an ITAM2 variant consisting of two loss-of-function mutations, and an ITAM3 variant consisting of two loss-of-function mutations. In certain embodiments, the modified CD3ζ polypeptide comprises the amino acid sequence set forth in SEQ ID NO: 52.

In certain embodiments, the intracellular signaling domain of the CAR further comprises at least one costimulatory signaling region. In certain embodiments, the at least one costimulatory signaling region comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D. In certain embodiments, the CAR comprises a transmembrane domain.

In certain embodiments, the antigen-recognizing receptor is a TCR-like fusion molecule comprising i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the antigen, wherein the TCR-like fusion molecule binds to the antigen in an HLA-independent manner. In certain embodiments, at least one of the TRAC polypeptide and the TRBC polypeptide is endogenous. In certain embodiments, the first and the second antigen-binding chains bind to the antigen with a dissociation constant (KD) of about 1×10⁻⁸ M or less. In certain embodiments, the first and the second antigen-binding chains bind to the antigen with a dissociation constant (KD) of about 5×10⁻⁹ M or less. In certain embodiments, the first antigen-binding chain comprises an antigen-binding fragment of a VH of an antibody and a TRBC polypeptide, and the second antigen-binding chain comprises an antigen-binding fragment of a VL of the antibody and a TRAC polypeptide. In certain embodiments, the first antigen-binding chain comprises an antigen-binding fragment of a VH of an antibody and a TRAC polypeptide, and the second antigen-binding chain comprises an antigen-binding fragment of a VL of the antibody and a TRBC polypeptide. In certain embodiments, the first and second antigen binding chains are capable of associating with a CD3ζ polypeptide. In certain embodiments, the first and second antigen binding chains, upon binding to the antigen, are capable of activating the CD3ζ polypeptide. In certain embodiments, the activation of the CD3ζ polypeptide is capable of activating the cell.

In certain embodiments, the cell further comprises a gene disruption of a TCR locus. In certain embodiments, the TCR locus is a TRAC locus.

In certain embodiments, the cell is a cell of the lymphoid lineage or a cell of the myeloid lineage. In certain embodiments, the cell of the lymphoid lineage is selected from the group consisting of a T cell, a B cell, a Natural Killer (NK) cell, and a dendritic cell. In certain embodiments, the cell is a T cell. In certain embodiments, the T cell is derived from an induced pluripotent stem cell. In certain embodiments, the T cell is a CD8+ T cell. In certain embodiments, the CD8+ T cell is CD4 independent. In certain embodiments, the T cell is selected from the group consisting of a cytotoxic T lymphocyte (CTL), a γδ T cell, a tumor-infiltrating lymphocyte (TIL), a regulatory T cell, and a Natural Killer T (NKT) cell. In certain embodiments, the T cell is CD62L+, CD45RA+, or CD45RA+ and CD62L+.

In certain embodiments, the immunoevasin is encoded by a polynucleotide integrated at a locus within the genome of the T cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the locus is a TRAC locus. In certain embodiments, the polynucleotide comprises an EF1 promoter. In certain embodiments, the EF1 promoter comprises or consists of the nucleotide sequence set forth in SEQ ID NO: 90.

In certain embodiments, the antigen recognizing receptor is encoded by a polynucleotide integrated at a locus within the genome of the T cell. In certain embodiments, the locus is selected from the group consisting of a TRAC locus, a TRBC locus, a TRDC locus, and a TRGC locus. In certain embodiments, the locus is a TRAC locus or a TRBC locus. In certain embodiments, the locus is a TRAC locus.

In certain embodiments, the antigen is a tumor antigen or a pathogen antigen. In certain embodiments, the tumor antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLDN18.2, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GPRC5d, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TROP2, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the cell further comprises a chimeric co-stimulating receptor (CCR). In certain embodiments, the CCR comprises an extracellular antigen-binding domain that binds to a second antigen and an intracellular domain that is capable of delivering a costimulatory signal to the cell but does not alone deliver an activation signal to the cell. In certain embodiments, the intracellular domain of the CCR comprises at least an intracellular domain of a co-stimulatory molecule or a portion thereof. In certain embodiments, the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the cell further comprises at least one exogenous costimulatory ligand. In certain embodiments, the at least one exogenous co-stimulatory ligand is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, FasL, GITRL, TNF-related apoptosis-inducing ligand (TRAIL), CD30L, LIGHT (TNFSF14), CD40L. In certain embodiments, the Ig superfamily member is selected from the group consisting of CD80, CD86, ICOSLG, and combinations thereof. In certain embodiments, the at least one exogenous costimulatory ligand comprises CD80. In certain embodiments, the at least one exogenous a costimulatory ligand comprises 4-1BBL. In certain embodiments, the cell comprises two exogenous costimulatory ligands. In certain embodiments, the at least two exogenous costimulatory ligands comprise CD80 and 4-1BBL.

In certain embodiments, the cell further comprises a fusion polypeptide comprising a) an extracellular domain and a transmembrane domain of a co-stimulatory ligand, and b) an intracellular domain of a first co-stimulatory molecule. In certain embodiments, the co-stimulatory ligand is selected from the group consisting of a tumor necrosis factor (TNF) family member, an immunoglobulin (Ig) superfamily member, and combinations thereof. In certain embodiments, the TNF family member is selected from the group consisting of 4-1BBL, OX40L, CD70, GITRL, CD40L, and combinations thereof. In certain embodiments, the Ig superfamily member is selected from the group consisting of CD80, CD86, ICOSLG, and combinations thereof. In certain embodiments, the co-stimulatory ligand is CD80. In certain embodiments, the first co-stimulatory molecule is selected from the group consisting of CD28, 4-1BB, OX40, ICOS, DAP-10, CD27, CD40, NKG2D, CD2, and combinations thereof. In certain embodiments, the first co-stimulatory molecule is 4-1BB. In certain embodiments, the co-stimulatory ligand is CD80 and the first co-stimulatory molecule is 4-1BB. In certain embodiments, the fusion polypeptide further comprises an intracellular domain of a second co-stimulatory molecule. In certain embodiments, the second co-stimulatory molecule is selected from the group consisting of CD28, 4-1BB, OX40, ICOS, DAP-10, CD27, CD40, NKG2D, CD2, and combinations thereof. In certain embodiments, the second co-stimulatory molecule is CD28. In certain embodiments, the co-stimulatory ligand is CD80, the first co-stimulatory molecule is 4-1BB, and the second co-stimulatory molecule is CD28.

In certain embodiments, the immunoevasin reduces the expression level of the major histocompatibility complex I (MHCI) from between about 60% to about 90% compared to a cell non expressing the immunoevasin. In certain embodiments, the cell is autologous. In certain embodiments, the cell is allogeneic.

The presently disclosed subject matter also provides a composition comprising the cells disclosed herein. In certain embodiments, the composition is a pharmaceutical composition further comprising a pharmaceutically acceptable excipient.

Further, the presently disclosed subject matter provides a nucleic acid comprising a first polynucleotide encoding an immunoevasin and a second polynucleotide encoding an antigen recognizing receptor that targets an antigen.

In certain embodiments, the immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91. In certain embodiments, the immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

In certain embodiments, the nucleic acid further comprises a third polynucleotide encoding a second immunoevasin. In certain embodiments, the second immunoevasin comprising or consisting of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91. In certain embodiments, the second immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

In certain embodiments, one or more of the first, second, and third polynucleotide is operably linked to a promoter element. In certain embodiments, the promoter element is an endogenous promoter or an exogenous promoter. In certain embodiments, the endogenous promoter is a TRAC promoter. In certain embodiments, the exogenous promoter is a EF1 promoter. In certain embodiments, the EF1 promoter comprises or consists of the nucleotide sequence set forth in SEQ ID NO: 90.

Additionally, the presently disclosed subject matter provides a nucleic acid composition comprising a first polynucleotide encoding an immunoevasin and a second polynucleotide encoding an antigen recognizing receptor that targets an antigen.

In certain embodiments, the immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91. In certain embodiments, the immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

In certain embodiments, the nucleic acid composition further comprises a third polynucleotide encoding a second immunoevasin. In certain embodiments, the second immunoevasin comprising or consisting of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91. In certain embodiments, the second immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

In certain embodiments, one or more of the first, second, and third polynucleotide is operably linked to a promoter element. In certain embodiments, the promoter element is a EF1 promoter. In certain embodiments, the EF1 promoter comprises or consists of the nucleotide sequence set forth in SEQ ID NO: 90.

The presently disclosed subject matter provides a vector comprising the nucleic acid of any or the nucleic acid composition disclosed herein. In certain embodiments, the vector is a lentiviral vector. In certain embodiments, the vector is a γ-retroviral vector.

Moreover, the presently disclosed subject matter provides a lipid nanoparticle comprising the nucleic acid or the nucleic acid composition disclosed herein.

Also provided by the presently disclosed subject matter is a composition comprising the nucleic acid, the vector, or the lipid nanoparticle disclosed herein. In certain embodiments, the composition is a pharmaceutical composition further comprising a pharmaceutically acceptable excipient.

The presently disclosed subject matter further provides a method for producing a cell disclosed herein, the method comprising introducing into the cell the nucleic acid, the nucleic acid composition, the vector, the lipid nanoparticle, or a composition thereof disclosed herein. In certain embodiments, the method further comprises generating a gene disruption of a TRAC locus, a NLRC5 locus, and a RFX5 locus, or a combination thereof. In certain embodiments, generating the gene disruption of comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

The presently disclosed subject matter provides also a cell produced by the method disclosed herein.

The presently disclosed subject matter provides methods of reducing tumor burden in a subject, preventing and/or treating a neoplasm or a tumor in the subject, preventing and/or treating a pathogen infection in a subject, preventing and/or treating an autoimmune disease in a subject, or preventing and/or treating an infectious disease in a subject, the method comprising administering to the subject an effective amount of the cells, or the compositions disclosed herein.

In certain embodiments, the method reduces the number of tumor cells, reduces tumor size, and/or eradicates the tumor in the subject. In certain embodiments, the neoplasm or tumor is cancer. In certain embodiments, the neoplasm or tumor is a solid tumor. In certain embodiments, the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the neoplasm or tumor is a blood cancer. In certain embodiments, the neoplasm or tumor is a myeloid disorder. In certain embodiments, the myeloid disorder is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera. In certain embodiments, the myeloid disorder is acute myeloid leukemia (AML).

In certain embodiments, the neoplasm or tumor is a B-cell malignancy. In certain embodiments, the B-cell malignancy is selected from the group consisting of B cell non-Hodgkin lymphoma (NHL), B cell Hodgkin's lymphoma, B cell acute lymphocytic leukemia (ALL), B cell chronic lymphocytic leukemia (CLL), multiple myeloma (MM), CLL with Richter's transformation, and CNS lymphoma.

In certain embodiments, the neoplasm or tumor is a leukemia. In certain embodiments, the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, B cell prolymphocytic leukemia, B-cell precursor acute lymphoblastic leukemia, and T-cell precursor acute lymphoblastic leukemia.

In certain embodiments, the neoplasm or tumor is a lymphoma. In certain embodiments, the lymphoma is selected from the group consisting of Hodgkin's lymphoma, non-Hodgkin's lymphoma, B-cell non-Hodgkin's lymphoma, T-cell non-Hodgkin's lymphoma, and T-cell precursor acute lymphoblastic lymphoma.

In addition, the presently disclosed subject matter provides the cells or the compositions disclosed herein for use in reducing tumor burden, treating and/or preventing a neoplasm or a tumor, preventing and/or treating a pathogen infection, preventing and/or treating an autoimmune disease, and/or preventing and/or treating an infectious disease, in a subject. Additionally, the presently disclosed subject matter provides the cells or the compositions disclosed herein for reducing tumor burden, treating and/or preventing a neoplasm or a tumor, preventing and/or treating a pathogen infection, preventing and/or treating an autoimmune disease, and/or preventing and/or treating an infectious disease, in a subject. Alternatively, the presently disclosed subject matter provides use of the cells or the compositions disclosed herein for the manufacture of a medicament for reducing tumor burden, treating and/or preventing a neoplasm or a tumor, preventing and/or treating a pathogen infection, preventing and/or treating an autoimmune disease, and/or preventing and/or treating an infectious disease, in a subject.

Finally, the presently disclosed subject matter provides kits comprising the cells or the compositions disclosed herein. In certain embodiments, the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

### BRIEF DESCRIPTION OF THE FIGURES

The following Detailed Description, given by way of example, but not intended to limit the presently disclosed subject matter to specific embodiments described, may be understood in conjunction with the accompanying drawings.

FIGS. 1A-1F depict that allogeneic CAR T cells are sensitive to immune rejection. FIG. 1A shows challenges to allogeneic cell therapy include GVHD and host-versus-graft (HVG) rejection of immune cells. FIG. 1B shows schematic of allogeneic CAR T cell generation. In order to eliminate GVHD, T cell receptor knockout CAR T cells can be generated by CRISPR-Cas9 targeting of TRAC locus. CAR insertion can occur via either semi-random gamma-retrovirus mediated transduction, or homology-directed repair driven insertion of CAR into the TRAC locus using adeno-associated virus (AAV). FIG. 1C illustrates CARs edited with Cas9 and a TRAC-directed guide RNA showed loss of expression of the TCRα-associated membrane protein CD3ε. CARs generated with either γ-retrovirus or AAV showed robust CAR expression. FIG. 1D shows animal model of immune rejection. NALM6 leukemia cell line transduced with GFP and firefly luciferase for bioluminescent imaging are injected on Day 0. PBMCs from either the same donor (autologous) or a different donor (allogeneic) as CAR T cells are injected intravenously on Day 3, followed by CAR T cells the next day. Tumor growth is monitored by BLI. FIG. 1E shows CARs infused with autologous PBMCs had superior tumor control to either a PBMC-alone control or CARs infused with allogeneic PBMCs. FIG. 1F shows fewer CAR T cells were found in bone marrow ten days after CAR injection in mice carrying allogeneic compared to autologous PBMCs, consistent with immune rejection.

FIGS. 2A-2F depict B2m KO renders cells sensitive to NK cell rejection. FIG. 2A shows schematic of allogeneic host-versus-graft immunity against CAR T cells. CD8 T cells typically recognize antigen via MHC Class I. Expression of Class I can be eliminated by knockout of the structural component beta-2-macroglobulin via B2M gene. FIG. 2B shows CAR T cells with loss of TCR/CD3 expression by TRAC KO, MHC Class I expression by B2m editing. FIG. 2C shows CARs that lack MHC Class I via TRAC and B2m editing (TRAC+B2m) were protected from CD8 T cell killing (left) but sensitive to NK cell killing at a 2:1 effector:target ratio in an in vitro survival assay. *p<0.05 by t-test (paired). FIG. 2D shows that in an in vivo model, TRAC KO CAR T cells infused with autologous but not allogeneic PBMCs mediated tumor control, whereas CAR T cells with TRAC and B2m KO were unable to mediate tumor control in either condition, motivating the desire to find a more effective allogeneic CAR T cell strategy. FIG. 2E shows a similar in vivo model as in FIG. 2D, but using NSG-IL15 mice, which secreted the human cytokine IL15 and thus support human NK cell engraftment. TRAC+B2m cells had reduced activity in this allogeneic model. FIG. 2F shows that TRAC cell numbers in bone marrow were reduced when infused in mice bearing allogeneic cells (Allo). TRAC+B2m KO cells (red) recovered to normal in absence of NK cells (Allo-NK).

FIGS. 3A-3F illustrate experiments to achieve an intermediate level of MHC expression. FIG. 3A shows that an intermediate level of MHC expression can promote protection against CD8 T cell killing without triggering NK cell rejection. FIG. 3B shows proof of concept with transient knockdown of MHC using siRNA electroporation to generate CAR T cells with varying levels of MHC. FIG. 3C shows CAR T cells with <40% of endogenous levels were protected from CD8 T cell killing, but the most robust NK cell rejection was triggered at levels <10%, indicating an intermediate expression zone of 10-40%. FIGS. 3D and 3E show that expression of viral evasion proteins that modulate MHC expression via retroviral vectors led to stable, intermediate levels of MHC (FIG. 3D), with HIV-1 NEF and EBV BNLF2a leading to levels of 10-40% in the intermediate range (FIG. 3E). FIG. 3F shows that expression of NEF and BNLF2a in CAR T cells led to protection from allogeneic CD8 T cell rejection in vitro without triggering NK cell killing.

FIGS. 4A-4C illustrate the impact of immunoevasins on CAR T cell function studied in a two-component model, with only tumor and CAR T cells. There are no allogeneic immune cells in this model, and thus it evaluates intrinsic CAR T cell function. FIG. 4A shows that among viral immunoevasins, NEF enhanced CAR T cell tumor control. FIG. 4B shows a repeat of experiment of FIG. 4A with a different donor confirming benefit of NEF overexpression on tumor control. FIG. 4C shows that the combination of the 1928 wild type CAR construct with NEF functions similarly to a previously published enhanced CAR T cell construct termed 1928z1xx. Furthermore, NEF can be combined with 1928z1xx and led to robust tumor control.

FIGS. 5A-5D illustrate a variety of NEF mutants disclosed herein. FIG. 5A show that these variants can eliminate key functions of NEF. As a result, they serve as valuable tools to study the role of NEF in modulating T cell function, and can be used in place of wild type NEF in therapeutic constructs. For example, the NEFWP variant, which leads to maintained expression of CD4, can be utilized if CD4 expression is desired. The D123G variant can be used if maintained MHC expression is desired. FIG. 5B shows FACS analysis indicating expression profile of the NEF mutants disclosed herein. FIG. 5C shows that the NEFAXXA variant was functional in tumor killing in vitro. FIG. 5D shows that the NEFAXXA variant lost the enhanced function of NEF in vivo, and is a valuable tool for studying NEF function.

FIGS. 6A-6E illustrate the effects of NEF on CAR T cell signaling. FIG. 6A shows that both NEF and the 1xx modification, which enhanced CAR T cell function, but not NEFAXXA mutant, which did not enhance CAR function, reduced signaling at the CD3zeta ITAM3 locus. FIG. 6B shows that phosphoproteomic analysis of these constructs revealed that NEF and 1xx mediated different effects on early T cell signaling proteins. FIGS. 6C and 6D show that there was little in differentially phosphorylated proteins between NEF and 1928z1xx, motivating the possibility of combining these two constructs. FIG. 6E shows that despite downmodulation of early signaling events, later events such as ERK phosphorylation and calcium flux were maintained.

FIGS. 7A-7H illustrate that reduction in MHC Class I Expression by Editing of Transcription Factors and Expression of viral immunoevasins can protect against allogeneic CD8 T cell recognition. FIG. 7A shows that MHC Class I expression is under control of numerous transcription factors including NLRC5 (Kobayashi and van den Elsen, Nat Rev Immunol 12, 813-820 (2012)). FIG. 7B shows that Cas9 sgRNAs targeting key regulators of MHC expression were screened, identifying NLRC5 and RFX5 as potential candidates to reduce expression. FIG. 7C shows that editing of regulators of MHC expression can be combined with overexpression of viral immune evasions. FIG. 7D shows that editing of regulators of MHC expression can be achieved through a variety of means, encompassing Cas9 or base editor based strategies for knockout, combined with retroviral or site specific expression of (minimally) a CAR construct, and in some cases overexpression of a viral immunoevasins. FIG. 7E shows that a bicistronic retroviral vector expressing the HIV Clade B immunoevasins NEF shows that EGFRt+ cells had reduced expression of HLA-A in an A2+ donor by fluorescence cytometry. Combined CRISPR-Cas9 editing of NLRC5 and overexpression of NEF led to further decreased expression in both the EGFRt+ and EGFRt− population. FIG. 7F shows that combinations of NLRC5 editing and viral immunoevasins overexpression led to a broad range of expression in HLA-A, HLA-C, MHC Class I, and HLA-E. FIG. 7G shows that T cells edited with either NLRC5 or B2m had similar levels of survival when cultured for 24 hours in presence of MLR-stimulated allogeneic PBMCs, which was enhanced compared to unedited (Mock) cells. FIG. 7H shows that a CAR T cell expressing NEF combined with NLRC5 KO using sgRNA led to enhanced tumor control in an in vivo model of allogeneic tumor rejection.

FIGS. 8A-8E illustrate that evasion protein expression from site-specific insertion via AAV is more stable and robust. FIG. 8A shows that gene cassettes including a CAR coupled to an evasion protein can be introduced via semi-random insertion from retrovirus and driven from retroviral promoter (top), or via site-specific insertion using AAV. Viral evasion protein can also be expressed from TRAC promoter, or via an introduced promoter such as EF1α. FIG. 8B shows the transcriptional output of exogenous (EF1α, viral LTR, PGK) and endogenous (TRAC, B2m) promoters. Promoter choice influences expression level. FIG. 8C shows that the expression of NEF from EF1α in TRAC locus (red) led to stable and lower MHC levels compared to retroviral vector (blue). FIG. 8D shows that the expression from EF1α compared to PGK led to greater MHC reduction. FIG. 8E shows that greater reduction of MHC using EF1α NEF further enhanced protection from CD8 killing in an in vitro survival assay.

FIGS. 9A-9C illustrate in vivo efficacy of CARs designed with viral evasion proteins. FIG. 9A shows that NEF was expressed using a co-transduction strategy from a retroviral vector. In this model, NEF and NEFWP did not enhance CAR T cell activity compared to a control CAR. FIG. 9B shows NEF and BNLF2a expressed from EF1a promoter inserted into TRAC locus. NEF enhanced intrinsic CAR T cell activity (in absence of allogeneic PBMCs, left). NEF and BNLF2a enhanced CAR T cell activity in an allogeneic setting. FIG. 9C shows that in the NSG-IL15 model, which captures both CD8 T cell and NK cell rejection, NEF expression promoted tumor control better than B2m KO and a control CAR.

FIGS. 10A and 10B illustrate exemplary nucleic acids and vectors disclosed herein. FIG. 10A shows a construct expressing a 1XX-CAR and a NEF polypeptide. FIG. 10B shows alternative constructs expressing antigen-recognizing receptors and immunoevasins.

FIGS. 11A-11F illustrate differential signaling cascades of cells expressing a 1XX CAR (1928z1xx) and the immunoevasin Nef (1928z+Nef). FIG. 11A illustrates key functional domains of the Nef protein. FIG. 11B shows that a mutation in the PxxP domain (NefAXXA) but not the AP-1 interaction domain (NEFD123G) of Nef abrogates the ability of Nef to enhance tumor control by Nef-expressing T cells in vivo. FIG. 11C shows the effects of Nef and 1928z1XX on CD3 zeta ITAM3 phosphorylation. FIG. 11D illustrates the distinct signaling cascades associated with 1928z1xx and 1928z+Nef. FIG. 11E illustrates the distinct phosphorylation patterns associated with Nef and 1928z1xx. FIG. 11F depicts with a Venn diagram showing distinct phosphorylation changes.

FIGS. 12A-12C depict that the combination of Nef and 1928z1xx enhances CAR T function. FIG. 12A illustrates that both 1928z1xx and 1928z1xx-Nef have superior tumor control in comparison to 1928z-Nef and 1928z. FIG. 12B illustrates total amount of CAR T cells detected in the bone marrow (BM) at day 9 and 16 post-injection. FIG. 12C shows effects of 1928z1xx-Nef on tumor growth (flux) and survival in a model of allogeneic rejection.

FIG. 13 illustrates the combined effect of Nef with other CAR variants.

FIGS. 14A-14N depict viral evasin HLA-I reduction protects against allogeneic CD8 T cell killing. FIG. 14A shows schematic of immune rejection model with CAR T cells derived from a single donor injected one day after PBMC from the same (Auto) or different (Allo) donor. FIG. 14B shows site-specific insertion of 1928z CAR into TRAC locus (left) leads to loss of TCR/CD3 and expression of CAR. FIG. 14C shows tumor control (left) and survival (right) by CAR T cells in mice treated with autologous (solid) compared to allogeneic (dashed) PBMC. P-value for survival by log-rank test. FIG. 14D shows CAR+ T cells isolated from bone marrow in autologous (solid) compared to allogeneic (dashed) PBMC-bearing mice at indicated timepoints post-infusion. 5 mice/group/timepoint for a single donor pair. P-value by two-way ANOVA. FIG. 14E shows total CAR+ T cells isolated from mice bearing no PBMC, autologous (Auto) PBMC, allogeneic PBMC (Allo), or allogeneic PBMC depleted of indicated subsets. P-value by ANOVA with post-test compared to Auto PBMC group. FIG. 14F shows HLA-I expression by flow cytometry of scramble siRNA treated T cells (black), or T cells treated with increasing dose of B₂m-siRNA or B₂m Cas9 edited (bottom row). FIG. 14G shows cells with decreasing HLA-I level (x-axis) generated by siRNA or Cas9 treatment. CAR T cell survival after 18-hour co-culture with MLR-stimulated allogeneic CD8 T cells. Survival expressed as relative frequency of live cells in allogeneic CD8-treated vs. untreated well; median survival for each group at base. Connected lines represent matched donor pairs, results pooled from two experiments. *p<0.05 by T-test compared to scramble treated control. FIG. 14H shows bicistronic retroviral vectors expressing viral evasin and EGFR marker. Relative HLA-I calculated as median HLA MFI in EGFR+/EGFRneg cell fraction. 4-6 unique donors/group. FIG. 14I shows site-specific insertion of CAR and viral evasin into TRAC locus. CAR+ cells expressing Nef and BNLF2a display similar CAR expression and decreased HLA-I expression as control cells. FIG. 14J shows relative 18-hour CAR T cell survival as in G. Each line represents donor pair with median survival of each group at base. p value by ANOVA with post-test relative to LNGFR control. FIG. 14K shows in vivo tumor control (left) and survival (right), p-value for survival by log-rank test with pairwise comparisons with Bonferroni adjustment. FIG. 14L shows total hCD45+ CAR+ T cells in bone marrow ten days after infusion, p<0.01 by ANOVA with indicated post-test relative to LNGFR control. FIG. 14M shows tumor control (left) and survival (right) in a CD19+ GBM flank model treated with PBMC with allogeneic PBMC), p-value for survival by log-rank test with pairwise comparisons with Bonferroni adjustment. FIG. 14N shows ratio of hCD45+ CAR+ T cells/tumor cells in flank tumors isolated thirty days after CAR infusion. Unless otherwise indicated, Black—TRAC^{KO} 1928z-EF1α-LNGFR (control), Light Blue—TRAC^{KO} 1928z-EF1α-Nef, Purple—TRAC^{KO} 1928z-EF1α-BNLF2a, Red—TRAC^{KO}B2 m^{KO} 1928z-EF1α-LNGFR.

FIGS. 15A-15H depict low or absent HLA-I expression leads to NK cell rejection.

FIG. 15A shows schematic of optimal range of HLA-I expression, with high expression leading to CD8 rejection, and low expression leading to NK cell rejection. FIG. 15B shows cells with decreasing HLA-I expression (x-axis) generated by B₂M siRNA or Cas9+ B₂M gRNA (KO) treatment. Live (sytox low) CAR T cells quantified by flow cytometry after 18-hour co-culture with allogeneic NK cells. Survival expressed as relative frequency of live cells in allogeneic NK-exposed vs. unexposed control. Connected lines represent matched donor pairs, results pooled from two experiments. *p<0.05 by T-test with adjustment compared to scramble treated control. FIG. 15C shows CD107a degranulation measured by flow cytometry in allogeneic NK cells exposed to CAR T cells of varying HLA-I, as in 15B. Degranulation expressed as percent CD107a+ cells after eight hours in co-culture with or without (No Target control) CAR T cells. *p<0.05 by T-test with adjustment compared to scramble treated control. FIG. 15D shows survival of indicated CAR T cells after 18-hour co-culture with allogeneic NK cells. Lines represent matched donor pairs. P value by ANOVA with indicated post-test relative to LNGFR control. FIG. 15E shows total hCD45+ CAR+ T cells in bone marrow ten days after infusion in the presence of autologous PBMC, allogeneic PBMC, or allogeneic PBMC depleted of NK cells. p values between indicated samples by Mann-whitney U test. FIG. 15F shows tumor control (left) and survival (right) of NSG15 mice treated with indicated CAR T cells in presence of allogeneic PBMC, p value by log-rank. FIG. 15G shows tumor control (left) and survival (right) of NSG15 mice treated with indicated CAR T cells in presence of allogeneic PBMC, p value by log-rank. FIG. 15H shows total hCD45+ CAR+ T cells in bone marrow ten days after infusion in the presence of allogeneic PBMC or allogeneic PBMC depleted of NK cells. Mann-whitney U test between samples with same CAR. Unless otherwise indicated, Black—TRAC^{KO} 1928z-EF1α-LNGFR (control), Light Blue—TRAC^{KO} 1928z-EF1α-Nef, Purple—TRAC^{KO} 1928z-EF1α-BNLF2a, Red—TRAC^{KO} B2m^{KO} 1928z-EF1α-LNGFR.

FIGS. 16A-16G depict Nef enhances intrinsic CAR T cell function via SH3 domain. FIG. 16A shows tumor control in NSG mice without PBMC treated with control LNGFR (black) or Nef (light blue) CAR T cells. FIG. 16B shows hCD45+ CAR+ cells in mouse bone marrow 10 days after infusion. P value by Mann-whitney U test. FIG. 16C shows survival in NALM6-bearing mice treated with CAR T cells cotransduced with vector containing viral evasin (top) and 1928z CAR (bottom). P value by log-rank. FIG. 16D shows schematic of dual Nef indications, including SH3 domain interactions and AP-1 clathrin membrane internalization. FIG. 16E shows representative CAR and CD4 expression by flow cytometry of CAR T cells generated by cotransduction of vectors containing 1928z CR and indicated Nef variants. FIG. 16F shows survival in NALM6-bearing mice treated with CAR T cells cotransduced with 1928z and indicated Nef variant. p value by log-rank. FIG. 16G shows tumor growth (left) and survival (right) in mice bearing U251 GBM flank tumors treated with TRAC^{KO}CD70^{KO} CAR T cells specific for CD70 bearing a CD28 costimulatory domain (CD70-28z) and cotransduced with either Nef (light blue) or NefAxxA (dark blue). P value by log rank.

FIGS. 17A-17H depict Nef promotes anti-apoptotic signaling via Pak2. FIG. 17A shows differential protein phosphorylation (volcano plot) by quantitative phosproteomics in 1928z CAR T cells cotransduced with Nef (top) or NefAxxA (bottom) compared to cells transduced with 1928z alone. Top differentially expressed proteins are labeled. FIG. 17B shows Western blot showing phosphorylation of Pak2 at S192 and Pak2 expression in 1928z CAR T cells (−) or 1928z CAR T cells cotransduced with WT Nef (WT) or Nef variants and stimulated for 10 minutes with CD19-beads. FIG. 17C shows CAR T cell survival 18 hours after stimulation with 1:1 ratio of NALM6 targets:CAR T cells. Measured as fraction of live (sytox low) LNGFR+ CAR T cells to unstimulated control. p value by ANOVA with indicated post-test relative to 1928z control. FIG. 17D shows fold expansion of CAR T cells repeatedly stimulated every 24 hours with adherent 3T3-CD19+ target cells. FIG. 17E shows Western blot showing Pak2 phosphorylation at S192 and total Pak2 for either Pak2 KO or Mock-edited CAR T cells co-transduced with either Nef (WT) or NefAxxA (Ax). Cell lysate generated with resting cells (No restim) or cells restimulated for 12 minutes with CD19-beads. FIG. 17F shows CAR T cell survival 18 hours after stimulation with 1:1 ratio of NALM6 targets:CAR T cells. Pooled results from 3 separate donors. p-value by ANOVA with post-test as indicated relative to Mock 1928z+Nef. FIG. 17G shows Western blot of BCL2 family proteins for either Mock or Pak2 KO CARs cotransduced with either WT Nef (WT) or NefAxxA (Ax). FIG. 17H shows quantification of Bcl-2 and Bax expression relative to housekeeping gene from western blot. Median value at base. Five independent donors. p value by ANOVA with indicated post-test.

FIGS. 18A-18F depict Nef inhibits AICD independently of signaling strength. FIG. 18A shows Western blot showing protein and phosphoprotein expression for 1928z CAR T cells co-transduced with either Nef (WT) or NefAxxA (Ax). Cell lysate generated with resting cells (No restim) or cells restimulated for 12 minutes with CD19-beads. FIG. 18B shows phospho-flow cytometry for indicated cells stimulated for 12 minutes with NALM6 tumor cells. FIG. 18C shows schematic of 1928z1XX (1XX) CAR with mutations in 2^{nd} and 3^{rd} ITAM of CD3ζ. FIG. 18D shows quantitative phosphoproteomics in 1928z CAR T cells (black), cells cotransduced with Nef (light blue) or NefAxxA (dark blue), or 1928z1XX CAR T cells. Indicated overall p-values for ANOVA. *p<0.05, **p<0.01, ***p<0.001 by post-test compared to 1928z control. FIG. 18E shows Western blot of 1928z and 1928z1XX CAR T cells cotransduced with Nef (WT) or NefAxxA (Ax) and stimulated as in B. FIG. 18F shows CAR T cell survival 18 hours after stimulation with 1:1 ratio of NALM6 targets:CAR T cells. Pooled results from 3 separate donors. p-value <0.001 by ANOVA with post-test as indicated relative to Nef-transduced construct.

FIG. 19A-19F depict combination of 1XX and Nef leads to durable and effective CAR T cells. FIG. 19A shows expression of CAR (left) and HLA-I (right) for indicated constructs by flow cytometry. FIG. 19 B shows total hCD45+ CAR+ T cells by flow cytometry in bone marrow at day 8 (left) and day 16 (right) post-infusion. P-value by Mann-Whitney U test with indicated post-test. FIG. 19C shows tumor control (left) and survival (right) of NALM6 bearing mice (without PBMC) treated with indicated CAR T cells. p value by log-rank. FIG. 19D shows total hCD45+ CAR+ T cells in bone marrow at indicated timepoints, 5 mice/group. P-value by Mann-Whitney U test. FIG. 19E shows tumor control in setting of allogeneic PBMC for single donor pair. FIG. 19F shows survival curves in setting of allogeneic PBMC for mice treated with indicated constructs. Pooled results of 4 experiments with different donor pairs, 10-20 mice per treatment group. Black—TRAC^{KO} 1928z-EF1α-LNGFR (1982z-LNGFR), Light Blue—TRAC^{KO} 1928z-EF1α-Nef (1928z-Nef), Red—TRAC^{KO} 1928z1xx-EF1α-LNGFR (1XX-LNGFR), Purple—TRAC^{KO} 1928z1xx-EF1α-Nef (1XX-Nef).

FIGS. 20A and 20B show in vivo effects of the presently disclosed subject matter. FIG. 20A shows in vivo tumor control (left) for mice treated with autologous PBMC and 1XX-LNGFR (Red) and 1XX-Nef (Purple) CAR T cells. Survival curves (right) for same (dashed lines) as well as CAR T cells in presence of allogeneic PBMC (solid lines), p<0.01 for survival by log-rank test. FIG. 20B shows total hCD45+ CAR+ T cells in bone marrow sixteen days after infusion in the presence of autologous (Auto) or Allogeneic (Allo) PBMC for 1XX-LNGFR (Red) and 1XX-Nef (Purple) CAR T cells, p<0.01 by ANOVA with indicated post-test relative to Auto/1XX-LNGFR control.

FIGS. 21A-21C show tumor control in setting of allogeneic PBMC for mice treated with three additional donor pairs. TRAC^{KO} 1928z-EF1α-LNGFR (1982z-LNGFR), TRAC^{KO} 1928z-EF1α-Nef (1928z-Nef), TRAC^{KO} 1928z1xx-EF1α-LNGFR (1XX-LNGFR), TRAC^{KO} 1928z1xx-EF1α-Nef (1XX-Nef).

FIG. 22 shows schematic of allogeneic CAR T cell survival and evasion. Allogeneic CAR T cells have impaired tumor rejection capacity due to immune rejection limiting survival (left). Immune evasion strategies can enhance CAR T cell persistence and survival (Top Left). However, even if cells are ultimately eliminated, tumor control can be greatly enhanced if CAR T cell proliferation and survival is enhanced during the peak effector period (bottom right).

### DETAILED DESCRIPTION OF THE INVENTION

The presently disclosed subject matter provides compositions, e.g., modified immune cells, useful for immunotherapy (e.g., T cell immunotherapy). The presently disclosed subject matter provides, inter alia, an off-the-shelf allogeneic Chimeric Antigen Receptor (CAR) T cell that is resistant to immune rejection. The presently disclosed subject matter is based, in part, on the observation that CRISPR-Cas9 mediated gene editing and insertion of a CAR paired with a viral evasion protein reduces expression of the immune protein MHC-I.

Non-limiting embodiments of the presently disclosed subject matter are described by the present specification and Examples.

For purposes of clarity of disclosure and not by way of limitation, the detailed description is divided into the following subsections:
1. Definitions;
2. Immunoevasins
3. Antigen-Recognizing Receptors;
4. Nucleic Acids and Vectors;
5. Cells
6. Formulations and Administration;
7. Methods of Treatment;
8. Kits;
9. Exemplary Embodiments.

### 1. Definitions

Unless defined otherwise, all technical and scientific terms used herein have the meaning commonly understood by a person skilled in the art. The following references provide one of skill with a general definition of many of the terms used in the presently disclosed subject matter: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold or within 2-fold, of a value.

As used herein, a “co-stimulatory molecule” refer to a cell surface molecule other than an antigen receptor or its ligand that can provide an efficient response of lymphocytes to an antigen. In certain embodiments, a co-stimulatory molecule can provide optimal lymphocyte activation.

As used herein, a “co-stimulatory ligand” refers to a molecule that upon binding to its receptor (e.g., a co-stimulatory molecule) produces a co-stimulatory response, e.g., an intracellular response that effects the stimulation provided when an antigen-recognizing receptor (e.g., a chimeric antigen receptor (CAR)) binds to its target antigen.

By “immunoresponsive cell” is meant a cell that functions in an immune response or a progenitor, or progeny thereof. In certain embodiments, the immunoresponsive cell is a cell of lymphoid lineage. Non-limiting examples of cells of lymphoid lineage include T cells, Natural Killer (NK) cells, B cells, and stem cells from which lymphoid cells may be differentiated. In certain embodiments, the immunoresponsive cell is a cell of myeloid lineage.

By “activates an immunoresponsive cell” is meant induction of signal transduction or changes in protein expression in the cell resulting in initiation of an immune response. For example, when CD3 Chains cluster in response to ligand binding and immunoreceptor tyrosine-based inhibition motifs (ITAMs) a signal transduction cascade is produced. In certain embodiments, when an endogenous TCR or an exogenous CAR binds to an antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated. This phosphorylation in turn initiates a T cell activation pathway ultimately activating transcription factors, such as NF-κB and AP-1. These transcription factors induce global gene expression of the T cell to increase IL-2 production for proliferation and expression of master regulator T cell proteins in order to initiate a T cell mediated immune response.

By “stimulates an immunoresponsive cell” is meant a signal that results in a robust and sustained immune response. In various embodiments, this occurs after immune cell (e.g., T-cell) activation or concomitantly mediated through receptors including, but not limited to, CD28, CD137 (4-1BB), OX40, CD40, ICOS, DAP-10, CD27, NKG2D, CD2, CD150, CD226. Receiving multiple stimulatory signals can be important to mount a robust and long-term T cell mediated immune response. T cells can quickly become inhibited and unresponsive to antigen. While the effects of these co-stimulatory signals may vary, they generally result in increased gene expression in order to generate long lived, proliferative, and anti-apoptotic T cells that robustly respond to antigen for complete and sustained eradication.

The term “antigen-recognizing receptor” as used herein refers to a receptor that is capable of activating an immune or immunoresponsive cell (e.g., a T-cell) in response to its binding to an antigen.

As used herein, the term “antibody” means not only intact antibody molecules, but also fragments of antibody molecules that retain immunogen-binding ability. Such fragments are also well known in the art and are regularly employed both in vitro and in vivo. Accordingly, as used herein, the term “antibody” means not only intact immunoglobulin molecules but also the well-known active fragments F(ab′)₂, and Fab. F(ab′)₂, and Fab fragments that lack the Fe fragment of intact antibody, clear more rapidly from the circulation, and may have less non-specific tissue binding of an intact antibody (Wahl et al., J. Nucl. Med. 24:316-325 (1983). As used herein, antibodies include whole native antibodies, bispecific antibodies; chimeric antibodies; Fab, Fab′, single chain variable fragment (scFv), fusion polypeptides, and unconventional antibodies. In certain embodiments, an antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant (C_{H}) region. The heavy chain constant region is comprised of three domains, CH1, CH2 and CH3. Each light chain is comprised of a light chain variable region (abbreviated herein as V_{L}) and a light chain constant C_{L} region. The light chain constant region is comprised of one domain, C_{L}. The V_{H} and V_{L} regions can be further sub-divided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1 q) of the classical complement system.

As used herein, “CDRs” are defined as the complementarity determining region amino acid sequences of an antibody which are the hypervariable regions of immunoglobulin heavy and light chains. See, e.g., Kabat et al., Sequences of Proteins of Immunological Interest, 4th U.S. Department of Health and Human Services, National Institutes of Health (1987). Generally, antibodies comprise three heavy chain and three light chain CDRs or CDR regions in the variable region. CDRs provide the majority of contact residues for the binding of the antibody to the antigen or epitope. In certain embodiments, the CDRs regions are delineated using the Kabat system (Kabat, E. A., et al. (1991) Sequences of Proteins of Immunological Interest, Fifth Edition, U.S. Department of Health and Human Services, NIH Publication No. 91-3242). In certain embodiments, the CDRs regions are delineated using the PyIgClassify system (Adolf-Bryfogle et al., Nucleic acids research 43.D1 (2015): D432-D438).

As used herein, the term “Linker” shall mean a functional group (e.g., chemical or polypeptide) that covalently attaches two or more polypeptides or nucleic acids so that they are connected to one another. As used herein, a “peptide linker” refers to one or more amino acids used to couple two proteins together (e.g., to couple V_{H} and V_{L} domains). In certain embodiments, the linker is a G4S linker. In certain embodiments, the linker comprises or consists of the amino acid sequence set forth in SEQ ID NO: 6, which is provided below:

[SEQ ID NO: 6]
GGGGSGGGGSGGGGS

In certain embodiments, the linker comprises or consists of the amino acid sequence set forth in SEQ ID NO: 7, which is provided below:

[SEQ ID NO: 7]
GGGGSGGGGSGGGSGGGGS

In certain embodiments, the linker comprises or consists of the amino acid sequence set forth in SEQ ID NO: 8, which is provided below:

[SEQ ID NO: 8]
GGGGSGGGGSGGGGSGGGSGGGGS

In certain embodiments, the linker comprises or consists of the amino acid sequence set forth in SEQ ID NO: 9, which is provided below:

[SEQ ID NO: 9]
GGGGSGGGGSGGGGSGGGGSGGGSGGGGS

In certain embodiments, the linker comprises or consists of the amino acid sequence set forth in SEQ ID NO: 10, which is provided below:

[SEQ ID NO: 10]
GGGGS

In certain embodiments, the linker comprises or consists of the amino acid sequence set forth in SEQ ID NO: 11, which is provided below:

[SEQ ID NO: 11]
GGGGSGGGGS

By “signal sequence” or “leader sequence” is meant a peptide sequence (e.g., 5, 10, 15, 20, 25 or 30 amino acids) present at the N-terminus of newly synthesized proteins that directs their entry to the secretory pathway. Exemplary leader sequences include, but is not limited to, a human IL-2 signal sequence (e.g., a human IL-2 signal sequence comprising or consisting of the amino acid sequence set forth in SEQ ID NO: 12), a mouse IL-2 signal sequence (e.g., a mouse IL-2 signal sequence comprising or consisting of the amino acid sequence set forth in SEQ ID NO: 13); a human kappa leader sequence (e.g., a human kappa leader sequence comprising or consisting of the amino acid sequence set forth in SEQ ID NO: 14), a mouse kappa leader sequence (e.g., a mouse kappa leader sequence comprising or consisting of the amino acid sequence set forth in SEQ ID NO: 15); a human CD8 leader sequence (e.g., a human CD8 leader sequence comprising or consisting of the amino acid sequence set forth in SEQ ID NO: 16); a truncated human CD8 signal peptide (e.g., a truncated human CD8 signal peptide comprising or consisting of the amino acid sequence set forth in SEQ ID NO: 17); a human albumin signal sequence (e.g., a human albumin signal sequence comprising or consisting of the amino acid sequence set forth in SEQ ID NO: 18); and a human prolactin signal sequence (e.g., a human prolactin signal sequence comprising or consisting of the amino acid sequence set forth in SEQ ID NO: 19). SEQ ID NO: 12-19 are provided below.

[SEQ ID NO: 12]
MYRMQLLSCIALSLALVTNS
[SEQ ID NO: 13]
MYSMQLASCVTLTLVLLVNS
[SEQ ID NO: 14]
METPAQLLFLLLLWLPDTTG
[SEQ ID NO: 15]
METDTLLLWVLLLWVPGSTG
[SEQ ID NO: 16]
MALPVTALLLPLALLLHAARP
[SEQ ID NO: 17]
MALPVTALLLPLALLLHA
[SEQ ID NO: 18]
MKWVTFISLLFSSAYS
[SEQ ID NO: 19]
MDSKGSSQKGSRLLLLLVVSNLLLCQGVVS

By “soluble” is meant a polypeptide that is freely diffusible in an aqueous environment (e.g., not membrane bound).

As used herein, the term “single-chain variable fragment” or “scFv” is a fusion protein of the variable regions of the heavy (V_{H}) and light chains (V_{L}) of an immunoglobulin covalently linked to form a V_{H}::V_{L} heterodimer. The V_{H} and V_{L} are either joined directly or joined by a peptide-encoding linker (e.g., 10, 15, 20, 25 amino acids), which connects the N-terminus of the V_{H} with the C-terminus of the V_{L}, or the C-terminus of the V_{H} with the N-terminus of the V_{L}. The linker is usually rich in glycine for flexibility, as well as serine or threonine for solubility. Despite removal of the constant regions and the introduction of a linker, scFv proteins retain the specificity of the original immunoglobulin. Single chain Fv polypeptide antibodies can be expressed from a nucleic acid including V_{H}- and V_{L}-encoding sequences as described by Huston, et al. (Proc. Nat. Acad. Sci. USA, 85:5879-5883, 1988). See, also, U.S. Pat. Nos. 5,091,513, 5,132,405 and 4,956,778; and U.S. Patent Publication Nos. 20050196754 and 20050196754. Antagonistic scFvs having inhibitory activity have been described (see, e.g., Zhao et al., Hyrbidoma (Larchmt) 2008 27(6):455-51; Peter et al., J Cachexia Sarcopenia Muscle 2012 Aug. 12; Shieh et al., J Imunol 2009 183(4):2277-85; Giomarelli et al., Thromb Haemost 2007 97(6):955-63; Fife et a., J Clin Invst 2006 116(8):2252-61; Brocks et al., Immunotechnology 1997 3(3):173-84; Moosmayer et al., Ther Immunol 1995 2(10:31-40). Agonistic scFvs having stimulatory activity have been described (see, e.g., Peter et al., J Biol Chem 2003 25278(38):36740-7; Xie et al., Nat Biotech 1997 15(8):768-71; Ledbetter et al., Crit Rev Immunol 997 17(5-6):427-55; Ho et al., BioChim Biophys Acta 2003 1638(3):257-66).

As used herein, the term “affinity” is meant a measure of binding strength. Affinity can depend on the closeness of stereochemical fit between antibody combining sites and antigen determinants, on the size of the area of contact between them, and/or on the distribution of charged and hydrophobic groups. As used herein, the term “affinity” also includes “avidity”, which refers to the strength of the antigen-antibody bond after formation of reversible complexes. Methods for calculating the affinity of an antibody for an antigen are known in the art, including, but not limited to, various antigen-binding experiments, e.g., functional assays (e.g., flow cytometry assay).

The term “chimeric antigen receptor” or “CAR” as used herein refers to a molecule comprising an extracellular antigen-binding domain that is fused to an intracellular signaling domain that is capable of activating or stimulating an immune or immunoresponsive cell, and a transmembrane domain. In certain embodiments, the extracellular antigen-binding domain of a CAR comprises an scFv. The scFv can be derived from fusing the variable heavy and light regions of an antibody. Alternatively or additionally, the scFv may be derived from Fab's (instead of from an antibody, e.g., obtained from Fab libraries). In certain embodiments, the scFv is fused to the transmembrane domain and then to the intracellular signaling domain. In certain embodiments, the CAR is selected to have high binding affinity or avidity for the antigen.

As used herein, the term “substantially identical” or “substantially homologous” refers to a polypeptide or a nucleic acid molecule exhibiting at least about 50% identical or homologous to a reference amino acid sequence (for example, any of the amino acid sequences described herein) or a reference nucleic acid sequence (for example, any of the nucleic acid sequences described herein). In certain embodiments, such a sequence is at least about 60%, at least about 65%, at least about 70%, at least about 75%, at least about 80%, at least about 85%, at least about 90%, at least about 95%, at least about 99%, or at least about 100% identical or homologous to the amino acid sequence or the nucleic acid sequence used for comparison.

Sequence identity can be measured by using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e-3 and e-100 indicating a closely related sequence.

The percent homology between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent homology between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. Additionally or alternatively, the amino acids sequences of the presently disclosed subject matter can further be used as a “query sequence” to perform a search against public databases to, for example, identify related sequences. Such searches can be performed using the XBLAST program (version 2.0) of Altschul, et al. (1990) J. Mol. Biol. 215:403-10. BLAST protein searches can be performed with the XBLAST program, score=50, wordlength=3 to obtain amino acid sequences homologous to the specified sequences (e.g., heavy and light chain variable region sequences) disclosed herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used.

As used herein, the term “a conservative sequence modification” refers to an amino acid modification that does not significantly affect or alter the binding characteristics of the presently disclosed antigen recognizing receptors (e.g., the extracellular antigen-binding domain of the CAR) comprising the amino acid sequence. Conservative modifications can include amino acid substitutions, additions and deletions. Modifications can be introduced into the extracellular antigen-binding domain of the presently disclosed CAR by standard techniques known in the art, such as site-directed mutagenesis and PCR-mediated mutagenesis. Amino acids can be classified into groups according to their physicochemical properties such as charge and polarity. Conservative amino acid substitutions are ones in which the amino acid residue is replaced with an amino acid within the same group. For example, amino acids can be classified by charge: positively-charged amino acids include lysine, arginine, histidine, negatively-charged amino acids include aspartic acid, glutamic acid, neutral charge amino acids include alanine, asparagine, cysteine, glutamine, glycine, isoleucine, leucine, methionine, phenylalanine, proline, serine, threonine, tryptophan, tyrosine, and valine. In addition, amino acids can be classified by polarity: polar amino acids include arginine (basic polar), asparagine, aspartic acid (acidic polar), glutamic acid (acidic polar), glutamine, histidine (basic polar), lysine (basic polar), serine, threonine, and tyrosine; non-polar amino acids include alanine, cysteine, glycine, isoleucine, leucine, methionine, phenylalanine, proline, tryptophan, and valine. Thus, one or more amino acid residues within a CDR region can be replaced with other amino acid residues from the same group and the altered antibody can be tested for retained function (i.e., the functions set forth in (c) through (l) above) using the functional assays described herein. In certain embodiments, no more than one, no more than two, no more than three, no more than four, no more than five residues within a specified sequence or a CDR region are altered.

By “disease” is meant any condition, disease or disorder that damages or interferes with the normal function of a cell, tissue, or organ, e.g., neoplasm, and pathogen infection of cell.

By “effective amount” is meant an amount sufficient to have a therapeutic effect. In certain embodiments, an “effective amount” is an amount sufficient to arrest, ameliorate, or inhibit the continued proliferation, growth, or metastasis (e.g., invasion, or migration) of a neoplasm.

By “endogenous” is meant a nucleic acid molecule or polypeptide that is normally expressed in a cell or tissue.

By “exogenous” is meant a nucleic acid molecule or polypeptide that is not endogenously present in a cell. The term “exogenous” would therefore encompass any recombinant nucleic acid molecule or polypeptide expressed in a cell, such as foreign, heterologous, and over-expressed nucleic acid molecules and polypeptides. By “exogenous” nucleic acid is meant a nucleic acid not present in a native wild-type cell; for example, an exogenous nucleic acid may vary from an endogenous counterpart by sequence, by position/location, or both. For clarity, an exogenous nucleic acid may have the same or different sequence relative to its native endogenous counterpart; it may be introduced by genetic engineering into the cell itself or a progenitor thereof, and may optionally be linked to alternative control sequences, such as a non-native promoter or secretory sequence.

By “increase” is meant to alter positively by at least about 5%. An alteration may be by about 5%, about 10%, about 25%, about 30%, about 50%, about 75%, about 100% or more.

By “reduce” is meant to alter negatively by at least about 5%. An alteration may be by about 5%, about 10%, about 25%, about 30%, about 50%, about 75%, or even by about 100%.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated cell” is meant a cell that is separated from the molecular and/or cellular components that naturally accompany the cell.

The term “antigen-binding domain” as used herein refers to a domain capable of specifically binding a particular antigenic determinant or set of antigenic determinants present on a cell.

By “neoplasm” or “malignancy” is meant a disease characterized by the pathological proliferation of a cell or tissue and its subsequent migration to or invasion of other tissues or organs. Neoplasm growth is typically uncontrolled and progressive, and occurs under conditions that would not elicit, or would cause cessation of, multiplication of normal cells. Neoplasm can affect a variety of cell types, tissues, or organs, including but not limited to an organ selected from bladder, bone, brain, breast, cartilage, glia, esophagus, fallopian tube, gallbladder, heart, intestines, kidney, liver, lung, lymph node, nervous tissue, ovaries, pancreas, prostate, skeletal muscle, skin, spinal cord, spleen, stomach, testes, thymus, thyroid, trachea, urogenital tract, ureter, urethra, uterus, and vagina, or a tissue or cell type thereof. Neoplasms include cancers, such as sarcomas, carcinomas, or plasmacytomas (malignant tumor of the plasma cells). In certain embodiments, the neoplasm is cancer.

By “specifically binds” is meant a polypeptide or a fragment thereof that recognizes and binds to a biological molecule of interest (e.g., a polypeptide), but which does not substantially recognize and bind other molecules in a sample, for example, a biological sample, which naturally includes a presently disclosed polypeptide.

The term “tumor antigen” as used herein refers to an antigen (e.g., a polypeptide) that is uniquely or differentially expressed on a tumor cell compared to a normal or non-neoplastic cell. In certain embodiments, a tumor antigen includes any polypeptide expressed by a tumor that is capable of activating or inducing an immune response via an antigen recognizing receptor or capable of suppressing an immune response via receptor-ligand binding.

The terms “comprises”, “comprising”, and are intended to have the broad meaning ascribed to them in U.S. Patent Law and can mean “includes”, “including” and the like.

As used herein, “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys. The term “immunocompromised” as used herein refers to a subject who has an immunodeficiency. The subject is very vulnerable to opportunistic infections, infections caused by organisms that usually do not cause disease in a person with a healthy immune system, but can affect people with a poorly functioning or suppressed immune system.

As used herein, “a functional fragment” of a molecule or polypeptide includes a fragment of the molecule or polypeptide that retains at least about 80%, at least about 85%, at least about 90%, at least about 95%, or at least about 100% of the primary function of the molecule or polypeptide.

Other aspects of the presently disclosed subject matter are described in the following disclosure and are within the ambit of the presently disclosed subject matter.

### 2. Immunoevasins

The presently disclosed subject matter provides cells comprising an immunoevasin. As used herein, the term “immunoevasin” refers to proteins and polypeptides that are expressed by certain viruses and that can evade immune recognition by interfering with the antigen presentation process and with the major histocompatibility complex (e.g., MHCI or MHCII). Immunoevasins block the recognition of viral fragments by cytotoxic immune cells (e.g., CD8⁺ cytototoxic T cells).

In certain non-limiting embodiments, the immunoevasin is selected from the group consisting of NEF, BNLF2a, US2, US6, US10, US11, U21, ORF37, K3, K5, ICP47, ORF66, VPU, GP42, E9, UL49.5, E3-19K (from adenovirus), CPXV012 and CPXV203 from cowpoxvirus, mK3 from MHV-68, EBNA1 and BGLF5 from EBV, and UL41 from HSV. Additional information on immunoevasins encompassed by the presently disclosed subject matter can be found in van de Weijer et al., Seminars in immunology. Vol. 27. No. 2. Academic Press, 2015, the content of which is incorporated herein in its entirety.

In certain embodiments, the immunoevasin is a NEF polypeptide. NEF, also known as Protein Nef, 3′ORF, or Negative factor (F-protein), is an HIV1-derived immunoevasin involved in the replication cycle of HIV-1. NEF modifies several T cell functions and down-regulates immunity surface molecules in order to evade host defense and increase viral infectivity. Any NEF polypeptide (e.g., derived from different HIV-1 group or subtype) can be used as immunoevasin of the presently disclosed subject matter. In certain embodiments, the NEF polypeptide can be encoded by any NEF allele (or clade). In certain embodiments, the NEF allele can be A1 allele, A2 allele, B allele, C allele, F1 allele, F2 allele, G allele, H allele, J allele, or K allele. In certain embodiments, the NEF polypeptide is encoded by a B allele. In certain embodiments, the NEF polypeptide comprises or consists of a consensus sequence based upon comparison of NEF polypeptides of different alleles (e.g., A1 allele, A2 allele, B allele, C allele, F1 allele, F2 allele, G allele, H allele, J allele, or K allele) derived from different HIV-1 clades (e.g., clade A1, clade A2, clade B, clade C, clade F1, clade F2, clade G, clade H, clade J, or clade K). Additional information on the consensus of NEF polypeptides can be found in Jubier-Maurin et al., AIDS research and human retroviruses 15.1 (1999): 23-32, and Kavanagh et al., Blood 107.5 (2006): 1963-1969, the content of each of which is incorporated by reference in its entirety.

In certain embodiments, the NEF polypeptide comprises or consists of the amino acid sequence of NCBI Reference No.: AAX86040.1 (SEQ ID NO: 1) or a fragment thereof. SEQ ID NO: 1 is provided below.

MGGKWSKRSVVGWPAVRERMRRAEPAADGVGAVSRDLEKHGAITSS
NTAANNADCAWLEAQEEEEVGFPVRPQVPLRPMTYKAAVDLSHFLK
EKGGLEGLIYSQKRQDILDLWVYHTQGYFPDWQNYTPGPGIRYPLT
FGWCFKLVPVEPEKVEEANEGENNSLLHPMSLHGMDDPEKEVLVWK
FDSRLAFHHMARELHPEYYKDC NO: 1]

In certain embodiments, the NEF polypeptide comprises or consists of an amino acid sequence that is at least about 80%, at least about 85%, at least about 90%, at least about 95%, at least about 96%, at least about 97%, at least about 98%, at least about 99%, or at least about 100% identical to the sequence set forth in SEQ ID NO: 1 or a fragment thereof. In certain embodiments, the NEF polypeptide comprises or consists of an amino acid sequence that is a consecutive portion of SEQ ID NO: 1, which is at least about 20, or at least about 30, or at least about 40, or at least about 50, at least about 60, at least about 70, at least about 80, at least about 90, or at least about 100, and up to about 206 amino acids in length. In certain embodiments, the NEF polypeptide comprises or consists of an amino acid sequence of amino acids 1 to 206, 1 to 20, 1 to 40, 1 to 60, 1 to 80, 1 to 100, 1 to 150, 1 to 200, 1 to 50, 50 to 100, 50 to 150, 50 to 206, 100 to 150, 100 to 206, or 150 to 206 of SEQ ID NO: 1. In certain embodiments, the NEF polypeptide comprises or consists of an amino acid sequence of amino acids 1 to 206 of SEQ ID NO: 1.

In certain embodiments, the NEF polypeptide comprises or consists of an amino acid sequence that is at least about 80%, at least about 85%, at least about 90%, at least about 95%, at least about 96%, at least about 97%, at least about 98%, at least about 99%, or at least about 100% identical to the sequence set forth in SEQ ID NO: 2 or a fragment thereof. In certain embodiments, the NEF polypeptide comprises or consists of an amino acid sequence that is a consecutive portion of SEQ ID NO: 2, which is at least about 20, or at least about 30, or at least about 40, or at least about 50, at least about 60, at least about 70, at least about 80, and up to about 85 amino acids in length. SEQ ID NO: 2 is provided below:

[SEQ ID NO: 2]
MGGKWLKSSMVRWPAVREKMKQAEPAAEGVGAISRDLGKHGAIPSS
NTTTNNANCAWLEAQEEEEVGFPVKPQVPLRPMTYKATF

In certain embodiments, the NEF polypeptide comprises a deletion of amino acids 1 to 5 of SEQ ID NO: 1. In certain embodiments, the NEF polypeptide comprises or consists of an amino acid sequence that is at least about 80%, at least about 85%, at least about 90%, at least about 95%, at least about 96%, at least about 97%, at least about 98%, at least about 99%, or at least about 100% identical to the sequence set forth in SEQ ID NO: 3 or a fragment thereof. In certain embodiments, the NEF polypeptide comprises or consists of an amino acid sequence that is a consecutive portion of SEQ ID NO: 3, which is at least about 20, or at least about 30, or at least about 40, or at least about 50, at least about 60, at least about 70, at least about 80, at least about 90, or at least about 100, and up to about 252 amino acids in length. SEQ ID NO: 3 is provided below:

[SEQ ID NO: 3]
SKKLSKHSRGLRERLLRARGDGYGKQRDASGGEYSQFQEESGREQN
SPSCEGQQYQQGEYMNSPWRNPATERQKDLYRQQNMDDVDSDDDDL
IGVPVTPRVPRREMTYKLAIDMSHFIKEKGGLQGMFYSRRRHRILD
IYLEKEEGIIPDWQNYTHGPGVRYPMYFGWLWKLVSVELSQEAEED
EANCLVHPAQTSRHDDEHGETLVWQFDSMLAYNYKAFTLYPEEFGH
KSGLPEKEWKAKLKARGIPYSE

In certain embodiments, the NEF polypeptide comprises a deletion of amino acids 12 to 39 of SEQ ID NO: 1. In certain embodiments, the NEF polypeptide comprises or consists of an amino acid sequence that is at least about 80%, at least about 85%, at least about 90%, at least about 95%, at least about 96%, at least about 97%, at least about 98%, at least about 99%, or at least about 100% identical to the sequence set forth in SEQ ID NO: 4 or a fragment thereof. In certain embodiments, the NEF polypeptide comprises or consists of an amino acid sequence that is a consecutive portion of SEQ ID NO: 4, which is at least about 20, or at least about 30, or at least about 40, or at least about 50, at least about 60, at least about 70, at least about 80, at least about 90, or at least about 100, and up to about 229 amino acids in length. SEQ ID NO: 4 is provided below:

[SEQ ID NO: 4]
MGASGSKKLSKSQFQEESGREQNSPSCEGQQYQQGEYMNSPWRNPA
TERQKDLYRQQNMDDVDSDDDDLIGVPVTPRVPRREMTYKLAIDMS
HFIKEKGGLQGMFYSRRRHRILDIYLEKEEGIIPDWQNYTHGPGVR
YPMYFGWLWKLVSVELSQEAEEDEANCLVHPAQTSRHDDEHGETLV
WQFDSMLAYNYKAFTLYPEEFGHKSGLPEKEWKAKLKARGIPYSE

In certain embodiments, the NEF polypeptide comprises at least one amino acid substitution. These amino acid substitutions can modify the ability to bind to certain intracellular proteins (e.g., CD4, CD8, CD28, etc.) or regulate certain cellular processes (e.g., actin remodeling and Lck recruitment). Additional information on the NEF polypeptide comprising at least one amino acid substitution encompassed by the presently disclosed subject matter can be found in Buffalo et al., Journal of virology 93.24 (2019): e01322-19, and in Haller et al., PloS one 2.11 (2007): e1212.

In certain embodiments, the NEF polypeptide comprises or consists of the amino acid sequence of UniProt Reference No.: P18092 (SEQ ID NO: 91) or a fragment thereof. SEQ ID NO: 91 is provided below. In certain embodiments, the disordered domain comprises or consists of amino acids 1 to 72 of SEQ ID NO: 91. In certain embodiments, the acidic domain comprises or consists of amino acids 88 to 96 of SEQ ID NO: 91. In certain embodiments, the PxxP domain comprises or consists of amino acids 104 to 107 of SEQ ID NO: 91. In certain embodiments, the dimerization domain comprises or consists of amino acids 140 to 156 of SEQ ID NO: 91. In certain embodiments, the NEF polypeptide comprises or consists of an amino acid sequence that is at least about 80%, at least about 85%, at least about 90%, at least about 95%, at least about 96%, at least about 97%, at least about 98%, at least about 99%, or at least about 100% identical to the sequence set forth in SEQ ID NO: 91 or a fragment thereof. In certain embodiments, the NEF polypeptide comprises or consists of an amino acid sequence that is a consecutive portion of SEQ ID NO: 91, which is at least about 20, or at least about 30, or at least about 40, or at least about 50, at least about 60, at least about 70, at least about 80, at least about 90, or at least about 100, and up to about 257 amino acids in length. In certain embodiments, the NEF polypeptide comprises or consists of an amino acid sequence of amino acids 1 to 257, 1 to 72, 88 to 96, 1 to 96, 1 to 107, 1 to 156, 104 to 107, 140 to 156, 155 to 257, 108 to 257, 97 to 257, or 73 to 257 of SEQ ID NO: 91. In certain embodiments, the NEF polypeptide comprises or consists of an amino acid sequence of amino acids 1 to 257 of SEQ ID NO: 91.

[SEQ ID NO: 91]
MGASGSKKLSKHSRGLRERLLRARGDGYGKQRDASGGEYSQFQEES
GREQNSPSCEGQQYQQGEYMNSPWRNPATERQKDLYRQQNMDDVDS
DDDDLIGVPVTPRVPRREMTYKLAIDMSHFIKEKGGLQGMFYSRRR
HRILDIYLEKEEGIIPDWQNYTHGPGVRYPMYFGWLWKLVSVELSQ
EAEEDEANCLVHPAQTSRHDDEHGETLVWQFDSMLAYNYKAFTLYP
EEFGHKSGLPEKEWKAKLKARGIPYSE

In certain embodiments, the immunoevasin is a BNLF2a polypeptide. BNLF2a is an Epstein Barr Virus-derived immunoevasin involved in the replication cycle of EBV. BNLF2a regulates viral evasion from HLA class I-restricted T-cell immunity and interacts with TAP1 and TAP2 to prevent TAP-mediated peptide transport and subsequent loading.

In certain embodiments, the BNLF2a polypeptide comprises or consists of the amino acid sequence of UniProt Reference No.: P0C739 (SEQ ID NO: 5) or a fragment thereof. SEQ ID NO: 5 is provided below. In certain embodiments, the disordered domain comprises or consists of amino acids 1 to 60 of SEQ ID NO: 5.

[SEQ ID NO: 5]
MVHVLERALLEQQSSACGLPGSSTETRPSHPCPEDPDVSRLRLLLV
VLCVLFGLLCLLLI

In certain embodiments, the BNLF2a polypeptide comprises or consists of an amino acid sequence that is at least about 80%, at least about 85%, at least about 90%, at least about 95%, at least about 96%, at least about 97%, at least about 98%, at least about 99%, or at least about 100% identical to the sequence set forth in SEQ ID NO: 5 or a fragment thereof. In certain embodiments, the BNLF2a polypeptide comprises or consists of an amino acid sequence that is a consecutive portion of SEQ ID NO: 5, which is at least about 20, or at least about 30, or at least about 40, or at least about 50, and up to about 60 amino acids in length. In certain embodiments, the BNLF2a polypeptide comprises or consists of an amino acid sequence of amino acids 1 to 60, 1 to 50, 1 to 40, 1 to 30, 1 to 20, 1 to 10, 10 to 60, 10 to 50, 10 to 40, 10 to 30, 10 to 20, 20 to 30, 20 to 40, 20 to 50, 20 to 60, 30 to 40, 30 to 50, 30 to 60, 40 to 50, 40 to 60, or 50 to 60 of SEQ ID NO: 5. In certain embodiments, the BNLF2a polypeptide comprises or consists of an amino acid sequence of amino acids 1 to 60 of SEQ ID NO: 5.

### 2.1. Delivery of the Immunoevasins

In certain embodiments, the immunoevasin is delivered to a cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the immunoevasin is delivered to the cell by a non-viral method. Any targeted genome editing methods can also be used to deliver the immunoevasin to a cell. In certain embodiments, the immunoevasin is delivered to a cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

### 3. Antigen-Recognizing Receptors

In certain embodiments, the presently disclosed cells further comprise an antigen-recognizing receptor that binds to an antigen. The subject matter of the instant application, e.g., cells comprising an immunoevasin and expressing an antigen-recognizing receptor, finds use irrespective of the particular antigen-recognizing receptor. In certain embodiments, the antigen-recognizing receptor is a chimeric antigen receptor (CAR). In certain embodiments, the antigen-recognizing receptor is a T-cell receptor (TCR). In certain embodiments, the antigen-recognizing receptor is a TCR like fusion molecule. The antigen-recognizing receptor can bind to a tumor antigen or a pathogen antigen. In certain embodiments, the antigen-recognizing receptor binds to a tumor antigen. In certain embodiments, the tumor antigen is a tumor-specific antigen or a tumor-associated antigen.

### 3.1. Antigens

In certain embodiments, the antigen-recognizing receptor binds to a tumor antigen. Any tumor antigen (antigenic peptide) can be used in the tumor-related embodiments described herein. Sources of antigen include, but are not limited to, cancer proteins. The antigen can be expressed as a peptide or as an intact protein or portion thereof. The intact protein or a portion thereof can be native or mutagenized. In certain embodiments, the tumor antigen is a tumor specific antigen (TSA). In certain embodiment, the tumor antigen is a tumor-associated antigen (TAA).

Non-limiting examples of tumor antigens include CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD276, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLDN18.2, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-a, FOLR2, FRMD5, GABRB2, GAS2, GD2, GD3, GDPD3, GNA14, GNAZ, GPR153, GPR56, GPRC5d, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TROP2, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

In certain embodiments, the antigen-recognizing receptor binds to a pathogen antigen, e.g., for use in treating and/or preventing a pathogen infection. Non-limiting examples of pathogens include viruses, bacteria, fungi, parasites, and protozoans capable of causing disease.

Non-limiting examples of pathogenic viruses include, Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Naira viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses), human papilloma virus (i.e. HPV), JC virus, Epstein Bar Virus, Merkel cell polyoma virus.

Non-limiting examples of pathogenic bacteria include Pasteurella, Staphylococci, Streptococcus, Escherichia coli, Pseudomonas species, and Salmonella species. Specific examples of infectious bacteria include but are not limited to, Helicobacter pyloris, Borrelia burgdorferi, Legionella pneumophilia, Mycobacteria sps (e.g. M. tuberculosis, M. avium, M. intracellulare, M. kansasii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, Corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, Clostridium difficile, and Actinomyces israelli.

In certain embodiments, the pathogen antigen is a viral antigen present in Cytomegalovirus (CMV), a viral antigen present in Epstein Barr Virus (EBV), a viral antigen present in Human Immunodeficiency Virus (HIV), or a viral antigen present in influenza virus.

### 3.2. T-Cell Receptor (TCR)

In certain embodiments, the antigen-recognizing receptor is a TCR. A TCR is a disulfide-linked heterodimeric protein comprising two variable chains expressed as part of a complex with the invariant CD3 chain molecules. A TCR is found on the surface of T cells, and is responsible for recognizing antigens as peptides bound to major histocompatibility complex (MHC) molecules. In certain embodiments, a TCR comprises an alpha chain and a beta chain (encoded by TRA and TRB, respectively). In certain embodiments, a TCR comprises a gamma chain and a delta chain (encoded by TRG and TRD, respectively).

Each chain of a TCR is composed of two extracellular domains: Variable (V) region and a Constant (C) region. The Constant region is proximal to the cell membrane, followed by a transmembrane region and a short cytoplasmic tail. The variable region binds to the peptide/MHC complex. The variable domain of both chains each has three complementarity determining regions (CDRs).

In certain embodiments, a TCR can form a receptor complex with three dimeric signaling modules CD3δ/ε, CD3γ/ε and CD247ζ/ζ or ζ/η. When a TCR complex engages with its antigen and MHC (peptide/MHC), the T cell expressing the TCR complex is activated.

In certain embodiments, the TCR is an endogenous TCR. In certain embodiments, the TCR is naturally occurring TCR.

In certain embodiments, the TCR is an exogenous TCR. In certain embodiments, the TCR is a recombinant TCR. In certain embodiments, the TCR is a non-naturally occurring TCR. In certain embodiments, the non-naturally occurring TCR differs from any naturally occurring TCR by at least one amino acid residue. In certain embodiments, the non-naturally occurring TCR differs from any naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues. In certain embodiments, the non-naturally occurring TCR is modified from a naturally occurring TCR by at least one amino acid residue. In certain embodiments, the non-naturally occurring TCR is modified from a naturally occurring TCR by at least about 2, about 3, about 4, about 5, about 6, about 7, about 8, about 9, about 10, about 11, about 12, about 13, about 14, about 15, about 20, about 25, about 30, about 40, about 50, about 60, about 70, about 80, about 90, about 100 or more amino acid residues.

In certain embodiments, the TCR recognizes a viral antigen. In certain embodiments, the TCR is expressed in a virus-specific T cell. In certain embodiments, the virus-specific T cell is derived from an individual immune to a viral infection, e.g., BK virus, human herpesvirus 6, Epstein-Barr virus (EBV), cytomegalovirus or adenovirus. In certain embodiments, the virus-specific T cell is a T cell disclosed in Leen et al., Blood, Vol. 121, No. 26, 2013; Barker et al., Blood, Vol. 116, No. 23, 2010; Tzannou et al., Journal of Clinical Oncology, Vol. 35, No. 31, 2017; or Bollard et al., Blood, Vol. 32, No. 8, 2014, each of which is incorporated by reference in its entirety. In certain embodiments, the TCR recognizes a tumor antigen (including a TAA or TSA). In certain embodiments, the TCR is expressed in a tumor-specific T cell. In certain embodiments, the tumor-specific T cell is a tumor-infiltrating T cell generated by culturing T cells with explants of a tumor, e.g., melanoma or an epithelial cancer. In certain embodiments, the tumor-specific T cell is a T cell disclosed in Stevanovic et al, Science, 356, 200-205, 2017; Dudley et al. Journal of Immunotherapy, 26(4): 332-342, 2003; or Goff et al, Journal of Clinical Oncology, Vol. 34, No. 20, 2016, each of which is incorporated by reference in its entirety.

### 3.3. Chimeric Antigen Receptor (CAR)

In certain embodiments, the antigen-recognizing receptor is a CAR. CARs are engineered receptors, which graft or confer a specificity of interest onto an immune effector cell. CARs can be used to graft the specificity of a monoclonal antibody onto a T cell; with transfer of their coding sequence facilitated by retroviral vectors.

There are three generations of CARs. “First generation” CARs are typically composed of an extracellular antigen-binding domain (e.g., an scFv), which is fused to a transmembrane domain, which is fused to cytoplasmic/intracellular signaling domain. “First generation” CARs can provide de novo antigen recognition and cause activation of both CD4⁺ and CD8⁺ T cells through their CD3ζ chain signaling domain in a single fusion molecule, independent of HLA-mediated antigen presentation. “Second generation” CARs add intracellular signaling domains from various co-stimulatory molecules (e.g., CD28, 4-1BB, ICOS, OX40) to the cytoplasmic tail of the CAR to provide additional signals to the T cell. “Second generation” CARs comprise those that provide both co-stimulation (e.g., CD28 or 4-1BB) and activation (CD3ζ). “Third generation” CARs comprise those that provide multiple co-stimulation (e.g., CD28 and 4-1BB) and activation (CD3ζ). In certain embodiments, the antigen-recognizing receptor is a first-generation CAR. In certain embodiments, the antigen-recognizing receptor is a CAR that does not comprise an intracellular signaling domain of a co-stimulatory molecule or a fragment thereof. In certain embodiments, the antigen-recognizing receptor is a second-generation CAR.

In accordance with the presently disclosed subject matter, a CAR comprises an extracellular antigen-binding domain that binds to the antigen, and an intracellular signaling domain. In certain embodiments, the CAR further comprises a transmembrane domain. In certain embodiments, the CAR further comprises a hinger/spacer region.

### 3.3.1. Extracellular Antigen-Binding Domain

In certain embodiments, the extracellular antigen-binding domain of the CAR (for example, an scFv) binds to the antigen with a dissociation constant (K_{D}) of about 5×10⁻⁷ M or less, about 1×10⁻⁷ M or less, about 5×10⁻⁸ M or less, about 1×10⁻⁸ M or less, about 5×10⁻⁹ M or less, or about 1×10⁻⁹ M or less, or about 1×10⁻¹⁰ M or less. In certain embodiments, the extracellular antigen-binding domain of the CAR (for example, an scFv) binds to the antigen with a K_{D} of about 1×10⁻⁸ M or less.

Binding of the extracellular antigen-binding domain (for example, in an scFv) can be confirmed by, for example, enzyme-linked immunosorbent assay (ELISA), radioimmunoassay (RIA), FACS analysis, bioassay (e.g., growth inhibition), or Western Blot assay. Each of these assays generally detect the presence of protein-antibody complexes of particular interest by employing a labeled reagent (e.g., an antibody, or an scFv) specific for the complex of interest. For example, the scFv can be radioactively labeled and used in a radioimmunoassay (RIA) (see, for example, Weintraub, B., Principles of Radioimmunoassays, Seventh Training Course on Radioligand Assay Techniques, The Endocrine Society, March, 1986, which is incorporated by reference herein). The radioactive isotope can be detected by such means as the use of a γ counter or a scintillation counter orby autoradiography. In certain embodiments, the extracellular antigen-binding domain of the CAR is labeled with a fluorescent marker. Non-limiting examples of fluorescent markers include green fluorescent protein (GFP), blue fluorescent protein (e.g., EBFP, EBFP2, Azurite, and mKalama1), cyan fluorescent protein (e.g., ECFP, Cerulean, and CyPet), and yellow fluorescent protein (e.g., YFP, Citrine, Venus, and YPet).

The extracellular antigen-binding domain can comprise or be an scFv, a Fab (which is optionally crosslinked), or a F(ab)₂. In certain embodiments, any of the foregoing molecules may be comprised in a fusion protein with a heterologous sequence to form the extracellular antigen-binding domain. In certain embodiments, the extracellular antigen-binding domain comprises or is an scFv. In certain embodiments, the scFv is a human scFv. In certain embodiments, the scFv is a humanized scFv. In certain embodiments, the scFv is a murine scFv.

### 3.3.1.1. Exemplary Extracellular Antigen-Binding Domains

In certain embodiments, the antigen-recognizing receptor is a CAR comprising an extracellular antigen-binding domain that binds to CD19. In certain embodiments, the extracellular antigen-binding domain of the CAR comprises or consists of the amino acid sequence set forth in SEQ ID NO: 30 and specifically binds to CD19, e.g., a human CD19 polypeptide. SEQ ID NO: 30 is provided in Table 1 below.

In certain embodiments, the extracellular antigen-binding domain of the CAR comprises a heavy chain variable region (V_{H}) comprising a CDR1 comprising the amino acid sequence set forth in SEQ ID NO: 20 or a conservative modification thereof, a CDR2 comprising the amino acid sequence set forth in SEQ ID NO: 21 or a conservative modification thereof, and a CDR3 comprising the amino acid sequence set forth in SEQ ID NO: 22 or a conservative modification thereof. In certain embodiments, the extracellular antigen-binding domain of the CAR comprises a V_{H} comprising a CDR1 comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22. SEQ ID NOs: 20-22 are provided in Table 1 below.

In certain embodiments, the extracellular antigen-binding domain of the CAR comprises a light chain variable region (V_{L}) comprising a CDR1 comprising the amino acid sequence set forth in SEQ ID NO: 23 or a conservative modification thereof, a CDR2 comprising the amino acid sequence set forth in SEQ ID NO: 24 or a conservative modification thereof, and a CDR3 comprising the amino acid sequence set forth in SEQ ID NO: 25 or a conservative modification thereof. In certain embodiments, the extracellular antigen-binding domain of the CAR comprises a V_{L} comprising a CDR1 comprising the amino acid sequence set forth in SEQ ID NO: 23, a CDR2 comprising the amino acid sequence set forth in SEQ ID NO: 24, and a CDR3 comprising the amino acid sequence set forth in SEQ ID NO: 25. SEQ ID NOs: 23-25 are provided in Table 1 below.

In certain embodiments, the extracellular antigen-binding domain of the CAR comprises a V_{H} comprising a CDR1 comprising the amino acid sequence set forth in SEQ ID NO: 20 or a conservative modification thereof, a CDR2 comprising the amino acid sequence set forth in SEQ ID NO: 21 or a conservative modification thereof, and a CDR3 comprising the amino acid sequence set forth in SEQ ID NO: 22, a conservative modification thereof; a V_{L} comprising a CDR1 comprising the amino acid sequence set forth in SEQ ID NO: 23 or a conservative modification thereof, a CDR2 comprising the amino acid sequence set forth in SEQ ID NO: 24 or a conservative modification thereof, and a CDR3 comprising the amino acid sequence set forth in SEQ ID NO: 25 or a conservative modification thereof. In certain embodiments, the extracellular antigen-binding domain of the CAR comprises a V_{H} comprising a CDR1 comprising amino acids having the sequence set forth in SEQ ID NO: 20, a CDR2 comprising the amino acid sequence set forth in SEQ ID NO: 21, and a CDR3 comprising the amino acid sequence set forth in SEQ ID NO: 22; and a V_{L} comprising a CDR1 comprising the amino acid sequence set forth in SEQ ID NO: 23, a CDR2 comprising the amino acid sequence set forth in SEQ ID NO: 24, and a CDR3 comprising the amino acid sequence set forth in SEQ ID NO: 25.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 26 or SEQ ID NO: 27. For example, the extracellular antigen-binding domain of the antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 26 or SEQ ID NO: 27. In certain embodiments, the extracellular antigen-binding domain of the CAR comprises a V_{H} comprising the amino acid sequence set forth in SEQ ID NO: 26 or SEQ ID NO: 27. SEQ ID NO: 26 and SEQ ID NO: 27 are provided in Table 1 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 28 or SEQ ID NO: 29. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 28 or SEQ ID NO: 29. In certain embodiments, the extracellular antigen-binding domain of the CAR comprises a V_{L} comprising the amino acid sequence set forth in SEQ ID NO: 28 or SEQ ID NO: 29. SEQ ID NO: 28 or SEQ ID NO: 29 is provided in Table 1 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 26 or SEQ ID NO: 27, and a V_{L} comprising an amino acid sequence that is at least about 80% (e.g., at least about 85%, at least about 90%, or at least about 95%) homologous or identical to the amino acid sequence set forth in SEQ ID NO: 28 or SEQ ID NO: 29.

In certain embodiments, the extracellular antigen-binding domain of the CAR comprises a V_{H} comprising the amino acid sequence set forth in SEQ ID NO: 26 or SEQ ID NO: 27. In certain embodiments, the extracellular antigen-binding domain of the CAR comprises a V_{L} comprising the amino acid sequence set forth in SEQ ID NO: 28 or SEQ ID NO: 29. In certain embodiments, the extracellular antigen-binding domain of the CAR comprises V_{H} comprising the amino acid sequence set forth in SEQ ID NO: 26 or SEQ ID NO: 27 and a V_{L} comprising the amino acid sequence set forth in SEQ ID NO: 28 or SEQ ID NO: 29.

In certain embodiments, the extracellular antigen-binding domain of the CAR is an scFv that comprises or consists of the amino acid sequence set forth in SEQ ID NO: 30. SEQ ID NOs: 20-30 are provided in the following Table 1. In certain embodiments, the V_{H} and V_{L} are linked via a linker. In certain embodiments, the linker comprises the amino acid sequence set forth in SEQ ID NO: 6, SEQ ID NO: 7, SEQ ID NO: 8, SEQ ID NO: 9, SEQ ID NO: 10, or SEQ ID NO: 11.

In certain embodiments, the CDRs regions/sequences disclosed herein are delineated using the Kabat system (Swindells et al., J Mol Biol. 2017 Feb. 3; 429(3):356-364).

TABLE 1

| | anti-human CD19 scFv (SJ25C1) | | |
|---|---|---|---|
| CDRs | 1 | 2 | 3 |
| V_{H} | GYAFSS | YPGDGD | KTISSVVDF |
| | [SEQ ID NO: 20] | [SEQ ID NO: 21] | [SEQ ID NO: 22] |
| V_{L} | KASQNVGTNVA | SATYRN | QQYNRYPYT |
| | [SEQ ID NO: 23] | [SEQ ID NO: 24] | [SEQ ID NO: 25] |

| | |
|---|---|
| Full V_{H} | EVKLQQSGAELVRPGSSVKISCKASGYAFSSYWMNWVKQRPGQGLEWIGQIYPGDGDTNY |
| | NGKFKGQATLTADKSSSTAYMQLSGLTSEDSAVYFCARKTISSVVDFYFDYWGQGTTVTV |
| | SS [SEQ ID NO: 26] |
| Full V_{H} v2 | EVKLQQSGAELVRPGSSVKISCKASGYAFSSYWMNWVKQRPGQGLEWIGQIYPGDGDTNY |
| | NGKFKGQATLTADKSSSTAYMQLSGLTSEDSAVYFCARKTISSVVDFYFDYWGQGTTVTV |
| | [SEQ ID NO: 27] |
| Full V_{L} | DIELTQSPKFMSTSVGDRVSVTCKASQNVGTNVAWYQQKPGQSPKPLIYSATYRNSGVPD |
| | RFTGSGSGTDFTLTITNVQSKDLADYFCQQYNRYPYTSGGGTKLEIKR [SEQ ID NO: 28] |
| Full V_{L} v2 | DIELTQSPKFMSTSVGDRVSVTCKASQNVGTNVAWYQQKPGQSPKPLIYSATYRNSGVPD |
| | RFTGSGSGTDFTLTITNVQSKDLADYFCQQYNRYPYTSGGGTKLEI [SEQ ID NO: 29] |
| scFv | MALPVTALLLPLALLLHAEVKLQQSGAELVRPGSSVKISCKASGYAFSSYWMNWVKQRPG |
| | QGLEWIGQIYPGDGDTNYNGKFKGQATLTADKSSSTAYMQLSGLTSEDSAVYFCARKTIS |
| | SVVDFYFDYWGQGTTVTVSSGGGGSGGGGSGGGGSDIELTQSPKFMSTSVGDRVSVTCKA |
| | SQNVGTNVAWYQQKPGQSPKPLIYSATYRNSGVPDRFTGSGSGTDFTLTITNVQSKDLAD |
| | YFCQQYNRYPYTSGGGTKLEIKR [SEQ ID NO: 30] |

In certain embodiments, the antigen-recognizing receptor is a CAR comprising an extracellular antigen-binding domain that binds to CD70.

In certain embodiments, the extracellular antigen-binding domain of the CAR comprises a heavy chain variable region (V_{H}) comprising a CDR1 comprising the amino acid sequence set forth in SEQ ID NO: 104 or a conservative modification thereof, a CDR2 comprising the amino acid sequence set forth in SEQ ID NO: 105 or a conservative modification thereof, and a CDR3 comprising the amino acid sequence set forth in SEQ ID NO: 106 or a conservative modification thereof. In certain embodiments, the extracellular antigen-binding domain of the CAR comprises a V_{H} comprising a CDR1 comprising the amino acid sequence set forth in SEQ ID NO: 104, a CDR2 comprising the amino acid sequence set forth in SEQ ID NO: 105, and a CDR3 comprising the amino acid sequence set forth in SEQ ID NO: 106. SEQ ID NOs: 104-106 are provided in Table 2 below.

In certain embodiments, the extracellular antigen-binding domain of the CAR comprises a light chain variable region (V_{L}) comprising a CDR1 comprising the amino acid sequence set forth in SEQ ID NO: 107 or a conservative modification thereof, a CDR2 comprising the amino acid sequence set forth in SEQ ID NO: 108 or a conservative modification thereof, and a CDR3 comprising the amino acid sequence set forth in SEQ ID NO: 109 or a conservative modification thereof. In certain embodiments, the extracellular antigen-binding domain of the CAR comprises a V_{L} comprising a CDR1 comprising the amino acid sequence set forth in SEQ ID NO: 107, a CDR2 comprising the amino acid sequence set forth in SEQ ID NO: 108, and a CDR3 comprising the amino acid sequence set forth in SEQ ID NO: 109. SEQ ID NOs: 107-109 are provided in Table 2 below.

In certain embodiments, the extracellular antigen-binding domain of the CAR comprises a V_{H} comprising a CDR1 comprising the amino acid sequence set forth in SEQ ID NO: 104 or a conservative modification thereof, a CDR2 comprising the amino acid sequence set forth in SEQ ID NO: 105 or a conservative modification thereof, and a CDR3 comprising the amino acid sequence set forth in SEQ ID NO: 106, a conservative modification thereof; a V_{L} Comprising a CDR1 comprising the amino acid sequence set forth in SEQ ID NO: 107 or a conservative modification thereof, a CDR2 comprising the amino acid sequence set forth in SEQ ID NO: 108 or a conservative modification thereof, and a CDR3 comprising the amino acid sequence set forth in SEQ ID NO: 109 or a conservative modification thereof. In certain embodiments, the extracellular antigen-binding domain of the CAR comprises a V_{H} comprising a CDR1 comprising amino acids having the sequence set forth in SEQ ID NO: 104, a CDR2 comprising the amino acid sequence set forth in SEQ ID NO: 105, and a CDR3 comprising the amino acid sequence set forth in SEQ ID NO: 106; and a V_{L} comprising a CDR1 comprising the amino acid sequence set forth in SEQ ID NO: 107, a CDR2 comprising the amino acid sequence set forth in SEQ ID NO: 108, and a CDR3 comprising the amino acid sequence set forth in SEQ ID NO: 109.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 110. For example, the extracellular antigen-binding domain of the antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 110. In certain embodiments, the extracellular antigen-binding domain of the CAR comprises a V_{H} comprising the amino acid sequence set forth in SEQ ID NO: 110. SEQ ID NO: 110 is provided in Table 2 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 111. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 111. In certain embodiments, the extracellular antigen-binding domain of the CAR comprises a V_{L} comprising the amino acid sequence set forth in SEQ ID NO: 111. SEQ ID NO: 111 is provided in Table 2 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 110, and a V_{L} comprising an amino acid sequence that is at least about 80% (e.g., at least about 85%, at least about 90%, or at least about 95%) homologous or identical to the amino acid sequence set forth in SEQ ID NO: 111.

In certain embodiments, the extracellular antigen-binding domain of the CAR comprises a V_{H} comprising the amino acid sequence set forth in SEQ ID NO: 110. In certain embodiments, the extracellular antigen-binding domain of the CAR comprises a V_{L} comprising the amino acid sequence set forth in SEQ ID NO: 111. In certain embodiments, the extracellular antigen-binding domain of the CAR comprises V_{H} comprising the amino acid sequence set forth in SEQ ID NO: 110 and a V_{L} comprising the amino acid sequence set forth in SEQ ID NO: 111.

In certain embodiments, the V_{H} and V_{L} are linked via a linker. In certain embodiments, the linker comprises the amino acid sequence set forth in SEQ ID NO: 6, SEQ ID NO: 7, SEQ ID NO: 8, SEQ ID NO: 9, SEQ ID NO: 10, or SEQ ID NO: 11.

In certain embodiments, the CDRs regions/sequences disclosed herein are delineated using the Kabat system (Swindells et al., J Mol Biol. 2017 Feb. 3; 429(3):356-364).

TABLE 2

| CDRs | 1 | 2 | 3 |
|---|---|---|---|
| V_{H} | SYIMH | VISYDGRNKYYADSVKG | DTDGYDFDY |
| | [SEQ ID NO: 104] | [SEQ ID NO: 105] | [SEQ ID NO: 106] |
| V_{L} | RASQSVSSYLA | DASNRAT | QQRTNWPLT |
| | [SEQ ID NO: 107] | [SEQ ID NO: 108] | [SEQ ID NO: 109] |

| | |
|---|---|
| Full V_{H} | MELGLSWIFLLAILKGVQCQVQLVESGGGVVQPGRSLRLSCAASGFTFSSYIMHWVRQAP |
| | GKGLEWVAVISYDGRNKYYADSVKGRFTISRDNSKNTLYLQMNSLRAEDTAVYYCARDTD |
| | GYDFDYWGQGTLVTV [SEQ ID NO: 110] |
| Full V_{L} | EIVLTQSPATLSLSPGERATLSCRASQSVSSYLAWYQQKPGQAPRLLIYDASNRATGI |
| | PARFSGSGSGTDFTLTISSLEPEDFAVYYCQQRTNWPLTFGGGTKVEIK [SEQ ID |
| | NO: 111] |

In certain embodiments, the V_{H} comprises a CDR1, a CDR2, and a CDR3 of a V_{H} sequence of an anti-CD70 antibody disclosed in International Patent Publication No. WO 2007/038637, which is incorporated by reference in its entirety. In certain embodiments, the V_{H} Comprises a CDR1, a CDR2, and a CDR3 of a V_{H} sequence of an anti-CD70 antibody 2H5 disclosed in International Patent Publication No. WO 2007/038637. In certain embodiments, the V_{L} comprises a CDR1, a CDR2, and a CDR3 of a V_{H} sequence of an anti-CD70 antibody disclosed in International Patent Publication No. WO 2007/038637. In certain embodiments, the V_{L} comprises a CDR1, a CDR2, and a CDR3 of a V_{H} sequence of an anti-CD70 antibody 2H5 disclosed in International Patent Publication No. WO 2007/038637.

In certain embodiments, the extracellular antigen-binding domain of the CAR comprises a heavy chain variable region (V_{H}) comprising a CDR1 comprising the amino acid sequence set forth in SEQ ID NO: 112 or a conservative modification thereof, a CDR2 comprising the amino acid sequence set forth in SEQ ID NO: 113 or a conservative modification thereof, and a CDR3 comprising the amino acid sequence set forth in SEQ ID NO: 114 or a conservative modification thereof. In certain embodiments, the extracellular antigen-binding domain of the CAR comprises a V_{H} comprising a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114. SEQ ID NOs: 112-114 are provided in Table 3 below.

In certain embodiments, the extracellular antigen-binding domain of the CAR comprises a light chain variable region (V_{L}) comprising a CDR1 comprising the amino acid sequence set forth in SEQ ID NO: 115 or a conservative modification thereof, a CDR2 comprising the amino acid sequence set forth in SEQ ID NO: 116 or a conservative modification thereof, and a CDR3 comprising the amino acid sequence set forth in SEQ ID NO: 117 or a conservative modification thereof. In certain embodiments, the extracellular antigen-binding domain of the CAR comprises a V_{L} comprising a CDR1 comprising the amino acid sequence set forth in SEQ ID NO: 115, a CDR2 comprising the amino acid sequence set forth in SEQ ID NO: 116, and a CDR3 comprising the amino acid sequence set forth in SEQ ID NO: 117. SEQ ID NOs: 115-117 are provided in Table 3 below.

In certain embodiments, the extracellular antigen-binding domain of the CAR comprises a V_{H} comprising a CDR1 comprising the amino acid sequence set forth in SEQ ID NO: 112 or a conservative modification thereof, a CDR2 comprising the amino acid sequence set forth in SEQ ID NO: 113 or a conservative modification thereof, and a CDR3 comprising the amino acid sequence set forth in SEQ ID NO: 114, a conservative modification thereof; a V_{L} Comprising a CDR1 comprising the amino acid sequence set forth in SEQ ID NO: 115 or a conservative modification thereof, a CDR2 comprising the amino acid sequence set forth in SEQ ID NO: 116 or a conservative modification thereof, and a CDR3 comprising the amino acid sequence set forth in SEQ ID NO: 117 or a conservative modification thereof. In certain embodiments, the extracellular antigen-binding domain of the CAR comprises a V_{H} comprising a CDR1 comprising amino acids having the sequence set forth in SEQ ID NO: 112, a CDR2 comprising the amino acid sequence set forth in SEQ ID NO: 113, and a CDR3 comprising the amino acid sequence set forth in SEQ ID NO: 114; and a V_{L} comprising a CDR1 comprising the amino acid sequence set forth in SEQ ID NO: 115, a CDR2 comprising the amino acid sequence set forth in SEQ ID NO: 116, and a CDR3 comprising the amino acid sequence set forth in SEQ ID NO: 117.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 118. For example, the extracellular antigen-binding domain of the antigen-recognizing receptor comprises a V_{H} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 118. In certain embodiments, the extracellular antigen-binding domain of the CAR comprises a V_{H} comprising the amino acid sequence set forth in SEQ ID NO: 118. SEQ ID NO: 118 is provided in Table 3 below.

In certain embodiments, the extracellular antigen-binding domain of the CAR comprises a V_{L} comprising an amino acid sequence that is at least about 80% (e.g., at least about 85%, at least about 90%, or at least about 95%) homologous or identical to the amino acid sequence set forth in SEQ ID NO: 119. For example, the extracellular antigen-binding domain of the CAR comprises a V_{L} comprising an amino acid sequence that is about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99% homologous or identical to the amino acid sequence set forth in SEQ ID NO: 119. In certain embodiments, the extracellular antigen-binding domain of the CAR comprises a V_{L} comprising the amino acid sequence set forth in SEQ ID NO: 119. SEQ ID NO: 119 is provided in Table 3 below.

In certain embodiments, the extracellular antigen-binding domain of the CAR comprises a V_{H} comprising an amino acid sequence that is at least about 80% (e.g., at least about 85%, at least about 90%, or at least about 95%) homologous or identical to the amino acid sequence set forth in SEQ ID NO: 118, and a V_{L} comprising an amino acid sequence that is at least about 80% (e.g., at least about 85%, at least about 90%, or at least about 95%) homologous or identical to the amino acid sequence set forth in SEQ ID NO: 119.

In certain embodiments, the extracellular antigen-binding domain of the CAR comprises a V_{H} comprising the amino acid sequence set forth in SEQ ID NO: 118. In certain embodiments, the extracellular antigen-binding domain of the CAR comprises a V_{L} comprising the amino acid sequence set forth in SEQ ID NO: 119. In certain embodiments, the extracellular antigen-binding domain of the CAR comprises V_{H} comprising the amino acid sequence set forth in SEQ ID NO: 118 and a V_{L} comprising the amino acid sequence set forth in SEQ ID NO: 119.

In certain embodiments, the V_{H} and V_{L} are linked via a linker. In certain embodiments, the linker comprises the amino acid sequence set forth in SEQ ID NO: 6, SEQ ID NO: 7, SEQ ID NO: 8, SEQ ID NO: 9, SEQ ID NO: 10, or SEQ ID NO: 11.

In certain embodiments, the CDRs regions/sequences disclosed herein are delineated using the Kabat system (Swindells et al., J Mol Biol. 2017 Feb. 3; 429(3):356-364).

TABLE 3

| CDRs | 1 | 2 | 3 |
|---|---|---|---|
| V_{H} | NYLMN | RIDPYDGDIDYNQNFKD | GYGTAYGVDY |
| | [SEQ ID NO: 112] | [SEQ ID NO: 113] | [SEQ ID NO: 114] |
| V_{L} | RASESVDNYGISEMN | AASRQGS | QQSKEVPWT |
| | [SEQ ID NO: 115] | [SEQ ID NO: 116] | [SEQ ID NO: 117] |

| | |
|---|---|
| Full V_{H} | QVQLQQPGAELVRPGASVKLSCKASGYTFSNYLMNWVKQRPEQDLDWIGRIDPYDGDI |
| | DYNQNFKDKAILTVDKSSSTAYMQLSSLTSEDSAVYYCARGYGTAYGVDYWGQGTSVT |
| | VSSAAA [SEQ ID NO: 118] |
| Full V_{L} | DIVLTQSPASLAVSLGQRATISCRASESVDNYGISEMNWFQQKPGQPPKLLIYAASRQ |
| | GSGVPARFSGSGSGTDFSLNIHPMEEDDTAMYFCQQSKEVPWTFGGGTKLEIK |
| | [SEQ ID NO: 119] |
| Full V_{H} | CAGGTCCAACTGCAGCAGCCTGGGGCTGAGCTGGTGAGGCCTGGGGCTTCAGTGAAGC |
| DNA | TGTCCTGCAAGGCTTCTGGCTACACGTTCTCCAACTACTTGATGAACTGGGTTAAGCA |
| | GAGGCCTGAGCAAGACCTTGACTGGATTGGAAGGATTGATCCTTACGATGGTGACATT |
| | GACTACAATCAAAACTTCAAGGACAAGGCCATATTGACTGTAGACAAATCCTCCAGCA |
| | CAGCCTACATGCAACTCAGCAGCCTGACATCTGAGGACTCTGCGGTCTATTACTGTGC |
| | AAGAGGTTATGGCACGGCCTATGGTGTGGACTACTGGGGTCAAGGAACCTCAGTCACC |
| | GTCTCCTCAGCGGCCGCA [SEQ ID NO: 103] |
| Full V_{L} | CAGGTCCAACTGCAGCAGCCTGGGGCTGAGCTGGTGAGGCCTGGGGCTTCAGTGAAGC |
| DNA | TGTCCTGCAAGGCTTCTGGCTACACGTTCTCCAACTACTTGATGAACTGGGTTAAGCA |
| | GAGGCCTGAGCAAGACCTTGACTGGATTGGAAGGATTGATCCTTACGATGGTGACATT |
| | GACTACAATCAAAACTTCAAGGACAAGGCCATATTGACTGTAGACAAATCCTCCAGCA |
| | CAGCCTACATGCAACTCAGCAGCCTGACATCTGAGGACTCTGCGGTCTATTACTGTGC |
| | AAGAGGTTATGGCACGGCCTATGGTGTGGACTACTGGGGTCAAGGAACCTCAGTCACC |
| | GTCTCCTCAGCGGCCGCA [SEQ ID NO: 120] |

The V_{H} and/or V_{L} amino acid sequences having at least about 80%, at least about 80%, at least about 85%, at least about 90%, or at least about 95% (e.g., about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, or about 99%) homology or identity to a specific sequence (e.g., SEQ ID NOs: 26, 27, 28, 29, 110, 111, 118, and 119) may contain substitutions (e.g., conservative substitutions), insertions, or deletions relative to the specified sequence(s), but retain the ability to bind to a target antigen (e.g., CD19, CD70, IL13). In certain embodiments, a total of 1 to 10 amino acids are substituted, inserted and/or deleted in a specific sequence (e.g., SEQ ID NOs: 26, 27, 28, 29, 110, 111, 118, and 119). In certain embodiments, substitutions, insertions, or deletions occur in regions outside the CDRs (e.g., in the FRs) of the extracellular antigen-binding domain. In certain embodiments, the extracellular antigen-binding domain of the CAR comprises V_{H} and/or V_{L} sequence selected from SEQ ID NOs: 26, 27, 28, 29, 110, 111, 118, and 119 including post-translational modifications of that sequence (SEQ ID NO: 26, 27, 28, 29, 110, 111, 118, and 119).

In certain the antigen-recognizing receptor is a CAR comprising an extracellular antigen-binding domain that binds to BCMA. Non-limiting examples of extracellular antigen-binding domains that bind to BCMA can be found in International Patent Publication No. WO 2016/090320, the contents of which are incorporated by reference in their entirety.

In certain the antigen-recognizing receptor is a CAR comprising an extracellular antigen-binding domain that binds to Fc Receptor-like 5 (FcRL5). Non-limiting examples of extracellular antigen-binding domains that bind to FcRL5 can be found in International Patent Publication No. WO 2016/090337, the contents of which are incorporated by reference in their entirety.

In certain the antigen-recognizing receptor is a CAR comprising an extracellular antigen-binding domain that binds to G-protein coupled receptor family C group 5 member D (GPRC5D). Non-limiting examples of extracellular antigen-binding domains that bind to GPRC5D can be found in International Patent Publication No. WO 2016/090312, the contents of which are incorporated by reference in their entirety.

In addition, the extracellular antigen-binding domain of the CAR can comprise a leader or a signal peptide that directs the nascent protein into the endoplasmic reticulum. Signal peptide or leader can be essential if the CAR is to be glycosylated and anchored in the cell membrane. The signal sequence or leader can be a peptide sequence (about 5, about 10, about 15, about 20, about 25, or about 30 amino acids long) present at the N-terminus of newly synthesized proteins that directs their entry to the secretory pathway. In certain embodiments, the signal peptide is covalently joined to the 5′ terminus (N-terminus) of the extracellular antigen-binding domain of the CAR.

In certain embodiments, the signal peptide comprises a CD8 polypeptide, e.g., the CAR comprises a truncated CD8 signal peptide. In certain embodiments, the signal peptide comprises or consists of the amino acid sequence set forth in SEQ ID NO: 17.

### 3.3.2. Transmembrane Domain of a CAR

In certain embodiments, the CAR comprises a transmembrane domain. In certain embodiments, the transmembrane domain of the CAR comprises a hydrophobic alpha helix that spans at least a portion of the membrane. Different transmembrane domains result in different receptor stability. After antigen recognition, receptors cluster and a signal are transmitted to the cell. In accordance with the presently disclosed subject matter, the transmembrane domain of the CAR can comprise a native or modified transmembrane domain of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof.

In certain embodiments, the transmembrane domain of the CAR comprises a CD8 polypeptide (e.g., a transmembrane domain of CD8 or a portion thereof).

In certain embodiments, the transmembrane domain of the CAR comprises a CD28 polypeptide (e.g., a transmembrane domain of CD28 or a portion thereof). In certain embodiments, the transmembrane domain comprises a transmembrane domain of human CD28 or a portion thereof. In certain embodiments, the CD28 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the sequence with a NCBI Reference No: NP_006130 (SEQ ID NO: 31) or a fragment thereof, and/or may optionally comprise up to one or up to two or up to three conservative amino acid substitutions. In certain embodiments, the CD28 polypeptide comprises or consists of an amino acid sequence that is a consecutive portion of SEQ ID NO: 31, which is at least about 20, at least about 25, or at least about 30, or at least about 40, or at least about 50, and up to about 220 amino acids in length. In certain embodiments, the CD28 polypeptide comprises or consists of an amino acid sequence of amino acids 1 to 220, 1 to 50, 50 to 100, 100 to 150, 114 to 220, 153 to 179, 150 to 200, or 200 to 220 of SEQ ID NO: 31. In certain embodiments, the transmembrane domain of the CAR comprises a CD28 polypeptide comprising or consisting of amino acids 153 to 179 of SEQ ID NO: 31). SEQ ID NO: 31 is provided below.

[SEQ ID NO: 31]
MLRLLLALNLFPSIQVTGNKILVKQSPMLVAYDNAVNLSCKYSYNLFSREFRASLHKGLDSAVE
VCVVYGNYSQQLQVYSKTGFNCDGKLGNESVTFYLQNLYVNQTDIYFCKIEVMYPPPYLDNEKS
NGTIIHVKGKHLCPSPLFPGPSKPFWVLVVVGGVLACYSLLVTVAFIIFWVRSKRSRLLHSDYM
NMTPRRPGPTRKHYQPYAPPRDFAAYRS

An exemplary nucleic acid sequence encoding amino acids 153 to 179 of SEQ ID NO: 31 is set forth in SEQ ID NO: 32, which is provided below.

[SEQ ID NO: 32]
ttttgggtgctggtggtggttggtggagtcctggcttgctatagcttgc
tagtaacagtggcctttattattttctgggtg

In certain embodiments, the transmembrane domain of the CAR comprises a CD8 polypeptide (e.g., a transmembrane domain of CD8 or a portion thereof). In certain embodiments, the transmembrane domain comprises a transmembrane domain of human CD8 or a portion thereof. In certain embodiments, the CD8 polypeptide comprises or consists of an amino acid sequence that is at least about 85%, about 90%, about 95%, about 96%, about 97%, about 98%, about 99% or about 100% homologous or identical to the amino acid sequence having a NCBI Reference No: NP_001139345.1 (SEQ ID NO: 33) or a fragment thereof, and/or may optionally comprise up to one or up to two or up to three conservative amino acid substitutions. In certain embodiments, the CD8 polypeptide comprises or consists of an amino acid sequence that is a consecutive portion of SEQ ID NO: 33, which is at least 20, or at least 30, or at least 40, or at least 50, and up to 235 amino acids in length. In certain embodiments, the CD8 polypeptide comprises or consists of an amino acid sequence of amino acids 1 to 235, 1 to 50, 50 to 100, 100 to 150, 150 to 200, or 200 to 235 of SEQ ID NO: 33. In certain embodiments, the transmembrane domain of the CAR comprises or consists of a CD8 polypeptide comprising or consisting of amino acids 137 to 209 of SEQ ID NO: 33.

[SEQ ID NO: 33]
MALPVTALLLPLALLLHAARPSQFRVSPLDRTWNLGETVELKCQVLLSNPTSGCSWLFQPRGAA
ASPTFLLYLSQNKPKAAEGLDTQRFSGKRLGDTFVLTLSDFRRENEGYYFCSALSNSIMYFSHF
VPVFLPAKPTTTPAPRPPTPAPTIASQPLSLRPEACRPAAGGAVHTRGLDFACDIYIWAPLAGT
CGVLLLSLVITLYCNHRNRRRVCKCPRPVVKSGDKPSLSARYV

### 3.3.3. Hinge/Spacer Region of the CAR

In certain embodiments, the CAR comprises a hinge/spacer region that links the extracellular antigen-binding domain to the transmembrane domain. The hinge/spacer region can be flexible enough to allow the antigen binding domain to orient in different directions to facilitate antigen recognition. In certain embodiments, the hinge/spacer region is positioned between the extracellular antigen-binding domain and the transmembrane domain. In certain embodiments, the hinge/spacer region of the CAR can comprise a native or modified hinge region of a CD8 polypeptide, a CD28 polypeptide, a CD3ζ polypeptide, a CD40 polypeptide, a 4-1BB polypeptide, an OX40 polypeptide, a CD84 polypeptide, a CD166 polypeptide, a CD8a polypeptide, a CD8b polypeptide, an ICOS polypeptide, an ICAM-1 polypeptide, a CTLA-4 polypeptide, a CD27 polypeptide, a CD40/My88 peptide, a NKGD2 peptide, a synthetic polypeptide (not based on a protein associated with the immune response), or a combination thereof. The hinge/spacer region can be the hinge region from IgG1, or the CH₂CH₃ region of immunoglobulin and portions of CD3, a portion of a CD28 polypeptide (e.g., a portion of SEQ ID NO: 31), a portion of a CD8 polypeptide, a variation of any of the foregoing which is at least about 80%, at least about 85%, at least about 90%, at least about 95%, or at least about 100% homologous or identical thereto, or a synthetic spacer sequence.

In certain embodiments, the hinge/spacer region of the CAR comprises a native or modified hinge region of a CD28 polypeptide or a portion thereof, as described herein. In certain embodiments, the hinge/spacer region of the CAR comprises a CD28 polypeptide comprising or consisting of amino acids 114 to 152 of SEQ ID NO: 31. In certain embodiments, the hinge/spacer region of the CAR comprises a CD28 polypeptide comprising or consisting of amino acids 117 to 152 of SEQ ID NO: 31.

An exemplary nucleotide sequence encoding amino acids 114 to 152 of SEQ ID NO: 31 is set forth in SEQ ID NO: 34, which is provided below.

[SEQ ID NO: 34]
attgaagttatgtatcctcctccttacctagacaatgagaagagcaatg
gaaccattatccatgtgaaagggaaacacctttgtccaagtcccctatt
tcccggaccttctaagccc

In certain embodiments, the transmembrane domain and the hinge/spacer region are derived from the same molecule. In certain embodiments, the transmembrane domain and the hinge/spacer region are derived from different molecules. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD28 polypeptide and the transmembrane domain of the CAR comprises a CD28 polypeptide. In certain embodiments, the hinge/spacer region of the CAR comprises a CD84 polypeptide and the transmembrane domain of the CAR comprises a CD84 polypeptide. In certain embodiments, the hinge/spacer region of the CAR comprises a CD166 polypeptide and the transmembrane domain of the CAR comprises a CD166 polypeptide. In certain embodiments, the hinge/spacer region of the CAR comprises a CD8a polypeptide and the transmembrane domain of the CAR comprises a CD8a polypeptide. In certain embodiments, the hinge/spacer region of the CAR comprises a CD8b polypeptide and the transmembrane domain of the CAR comprises a CD8b polypeptide. In certain embodiments, the hinge/spacer region of the CAR comprises a CD28 polypeptide and the transmembrane domain of the CAR comprises an ICOS polypeptide.

### 3.3.4. Intracellular Signaling Domain of a CAR

In certain embodiments, the CAR comprises an intracellular signaling domain. In certain embodiments, the intracellular signaling domain of the CAR comprises a CD3ζ polypeptide, which can activate or stimulate a cell (e.g., a cell of the lymphoid lineage, e.g., a T cell). Wild type (“native”) CD3ζ comprises three immunoreceptor tyrosine-based activation motifs (“ITAMs”) (e.g., ITAM1, ITAM2 and ITAM3), three basic-rich stretch (BRS) regions (BRS1, BRS2 and BRS3), and transmits an activation signal to the cell (e.g., a cell of the lymphoid lineage, e.g., a T cell) after antigen is bound. The intracellular signaling domain of the native CD3ζ-chain is the primary transmitter of signals from endogenous TCRs.

In certain embodiments, the intracellular signaling domain of the CAR comprises a native CD3ζ polypeptide. In certain embodiments, the native CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the sequence with a NCBI Reference No: NP_932170 (SEQ ID NO: 35) or a fragment thereof. In certain embodiments, the native CD3ζ polypeptide comprises or consists of an amino acid sequence that is a consecutive portion of SEQ ID NO: 35, which is at least about 20, or at least about 30, or at least about 40, or at least about 50, or at least about 100, or at least about 110, and up to about 164 amino acids in length. In certain embodiments, the native CD3ζ polypeptide comprises or consists of an amino acid sequence of amino acids 1 to 50, 50 to 100, 100 to 150, 50 to 164, 52 to 164, or 150 to 164 of SEQ ID NO: 35. In certain embodiments, the native CD3ζ polypeptide comprises or consists of an amino acid sequence of amino acids 52 to 164 of SEQ ID NO: 35. SEQ ID NO: 35 is provided below:

[SEQ ID NO: 35]
MKWKALFTAAILQAQLPITEAQSFGLLDPKLCYLLDGILFIYGVILTALFLRVKFSRSADAPAY
QQGQNQLYNELNLGRREEYDVLDKRRGRDPEMGGKPQRRKNPQEGLYNELQKDKMAEAYSEIGM
KGERRRGKGHDGLYQGLSTATKDTYDALHMQALPPR

An exemplary nucleotide sequence encoding amino acids 52 to 164 of SEQ ID NO: 35 is set forth in SEQ ID NO: 36, which is provided below.

[SEQ ID NO: 36]
AGAGTGAAGTTCAGCAGGAGCGCAGACGCCCCCGCGTACCAGCAGGGCCAGAACCAGCTCTATA
ACGAGCTCAATCTAGGACGAAGAGAGGAGTACGATGTTTTGGACAAGAGACGTGGCCGGGACCC
TGAGATGGGGGGAAAGCCGAGAAGGAAGAACCCTCAGGAAGGCCTGTACAATGAACTGCAGAAA
GATAAGATGGCGGAGGCCTACAGTGAGATTGGGATGAAAGGCGAGCGCCGGAGGGGCAAGGGGC
ACGATGGCCTTTACCAGGGTCTCAGTACAGCCACCAAGGACACCTACGACGCCCTTCACATGCA
GGCCCTGCCCCCTCGCTAA

In certain embodiments, the native CD3ζ polypeptide comprises or consists of an amino acid sequence that is at least about 85%, about 90%, about 95%, about 96%, about 97%, about 98%, about 99% or about 100% identical or homologous to the amino acid sequence set forth in SEQ ID NO: 37. SEQ ID NO: 37 is provided below:

[SEQ ID NO: 37]
RVKFSRSADAPAYQQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

In certain embodiments, the intracellular signaling domain of the CAR comprises a modified CD3ζ polypeptide. In certain embodiments, the intracellular signaling domain of the CAR comprises a modified human CD3ζ polypeptide. In certain embodiments, the modified CD3ζ polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 38 or a fragment thereof, and/or may optionally comprise up to one or up to two or up to three conservative amino acid substitutions. SEQ ID NO: 38 is provided below:

[SEQ ID NO: 38]
RVKFSRSADAPAYQQGQNQLYNELNLGRREEYDVLDKRRGRDPEMGGKP
RRKNPQEGLFNELQKDKMAEAFSEIGMKGERRRGKGHDGLFQGLSTATK
DTFDALHMQALPPR

An exemplary nucleotide sequence encoding the amino acid sequence of SEQ ID NO: 38 is set forth in SEQ ID NO: 39, which is provided below.

[SEQ ID NO: 39]
agagtgaagttcagcaggagcgcagacgcccccgcgtaccagcagggccagaaccagctctata
acgagctcaatctaggacgaagagaggagtacgatgttttggacaagagacgtggccgggaccc
tgagatggggggaaagccgagaaggaagaaccctcaggaaggcctgtTcaatgaactgcagaaa
gataagatggcggaggcctTcagtgagattgggatgaaaggcgagcgccggaggggcaaggggc
acgatggcctttTccaggggctcagtacagccaccaaggacacctTcgacgcccttcacatgca
ggccctgccccctcgc

In certain embodiments, the modified CD3ζ polypeptide comprises one, two or three ITAMs. In certain embodiments, the modified CD3ζ polypeptide comprises a native ITAM1. In certain embodiments, the native ITAM1 comprises or consists of the amino acid sequence set forth in SEQ ID NO: 40.

[SEQ ID NO: 40]
QNQLYNELNLGRREEYDVLDKR

An exemplary nucleic acid sequence encoding the amino acid sequence of SEQ ID NO: 40 is set forth in SEQ ID NO: 41, which is provided below.

[SEQ ID NO: 41]
CAGAACCAGCTCTATAACGAGCTCAATCTAGGACGAAGAGAGGAGTACG
ATGTTTTGGACAAGAGA

In certain embodiments, the modified CD3ζ polypeptide comprises an ITAM1 variant comprising one or more loss-of-function mutations. In certain embodiments, the ITAM1 variant comprises or consists of two loss-of-function mutations. In certain embodiments, each of the one or more (e.g., two) loss of function mutations comprises a mutation of a tyrosine residue in ITAM1. In certain embodiments, the ITAM1 variant consists of two loss-of-function mutations. In certain embodiments, the ITAM1 variant comprises or consists of the amino acid sequence set forth in SEQ ID NO: 42, which is provided below.

[SEQ ID NO: 42]
QNQLENELNLGRREEFDVLDKR

An exemplary nucleic acid sequence encoding the amino acid sequence of SEQ ID NO: 42 is set forth in SEQ ID NO: 43, which is provided below.

[SEQ ID NO: 43]
CAGAACCAGCTCTTTAACGAGCTCAATCTAGGACGAAGAGAGGAGTTCG
ATGTTTTGGACAAGAGA

In certain embodiments, the modified CD3ζ polypeptide comprises a native ITAM2. In certain embodiments, the native ITAM2 comprises or consists of the amino acid sequence set forth in SEQ ID NO: 44, which is provided below.

[SEQ ID NO: 44]
QEGLYNELQKDKMAEAYSEIGMK

An exemplary nucleic acid sequence encoding the amino acid sequence of SEQ ID NO: 44 is set forth in SEQ ID NO: 45, which is provided below.

[SEQ ID NO: 45]
CAGGAAGGCCTGTACAATGAACTGCAGAAAGATAAGATGGCGGAGGCCT
ACAGTGAGATTGGGATGAAA

In certain embodiments, the modified CD3ζ polypeptide comprises an ITAM2 variant. In certain embodiments, the ITAM2 variant comprises or consists of one or more loss-of-function mutations. In certain embodiments, the ITAM2 variant comprises or consists of two loss-of-function mutations. In certain embodiments, each of the one or more (e.g., two) the loss of function mutations comprises a mutation of a tyrosine residue in ITAM2. In certain embodiments, the ITAM1 variant consists of two loss-of-function mutations. In certain embodiments, the ITAM2 variant comprises or consists of the amino acid sequence set forth in SEQ ID NO: 46, which is provided below.

[SEQ ID NO: 46]
QEGLFNELQKDKMAEAFSEIGMK

An exemplary nucleic acid sequence encoding the amino acid sequence of SEQ ID NO: 46 is set forth in SEQ ID NO: 47, which is provided below.

[SEQ ID NO: 47]
CAGGAAGGCCTGTTCAATGAACTGCAGAAAGATAAGATGGCGGAGGCCT
TCAGTGAGATTGGGATGAAA

In certain embodiments, the modified CD3ζ polypeptide comprises a native ITAM3. In certain embodiments, the native ITAM3 comprises or consists of the amino acid sequence set forth in SEQ ID NO: 48, which is provided below.

[SEQ ID NO: 48]
HDGLYQGLSTATKDTYDALHMQ

An exemplary nucleic acid sequence encoding the amino acid sequence of SEQ ID NO: 48 is set forth in SEQ ID NO: 49, which is provided below.

[SEQ ID NO: 49]
CACGATGGCCTTTACCAGGGTCTCAGTACAGCCACCAAGGACACCTACG
ACGCCCTTCACATGCAG

In certain embodiments, the modified CD3ζ polypeptide comprises an ITAM3 variant. In certain embodiments, the ITAM3 variant comprises or consists of two loss-of-function mutations. In certain embodiments, each of the one or more (e.g., two) the loss of function mutations comprises a mutation of a tyrosine residue in ITAM3. In certain embodiments, the ITAM3 variant comprises or consists of two loss-of-function mutations. In certain embodiments, the ITAM3 variant comprises or consists of the amino acid sequence set forth in SEQ ID NO: 50, which is provided below.

[SEQ ID NO: 50]
HDGLFQGLSTATKDTEDALHMQ

An exemplary nucleic acid sequence encoding the amino acid sequence of SEQ ID NO: 50 is set forth in SEQ ID NO: 51, which is provided below.

[SEQ ID NO: 51]
CACGATGGCCTTTTCCAGGGGCTCAGTACAGCCACCAAGGACACCTTCG
ACGCCCTTCACATGCAG

Additional modified CD3ζ polypeptides and CARs comprising modified CD3ζ polypeptides are disclosed in International Patent Application Publication No. WO2019/133969, which is incorporated by reference hereby in its entirety.

In certain embodiments, the intracellular signaling domain of the CAR comprises a modified CD3ζ polypeptide comprising a native ITAM1, an ITAM2 variant comprising or consisting of one or more (e.g., two) loss-of-function mutations, and an ITAM3 variant comprising or consisting of one or more (e.g., two) loss-of-function mutations. In certain embodiments, the intracellular signaling domain of the CAR comprises a modified CD3ζ polypeptide comprising a native ITAM1, an ITAM2 variant consisting of two loss-of-function mutations, and an ITAM3 variant consisting of two loss-of-function mutations. In certain embodiments, the intracellular signaling domain of the CAR comprises a modified CD3ζ polypeptide comprising a native ITAM1 consisting of the amino acid sequence set forth in SEQ ID NO: 40, an ITAM2 variant consisting of the amino acid sequence set forth in SEQ ID NO: 46, and an ITAM3 variant consisting of the amino acid sequence set forth in SEQ ID NO: 50. In certain embodiments, the modified CD3ζ polypeptide is designated as “1XX”. In certain embodiments, the modified CD3ζ polypeptide comprises or consists of the amino acid sequence set forth in SEQ ID NO: 52. SEQ ID NO: 52 is provided below:

[SEQ ID NO: 52]
RVKFSRSADAPAYQQGQNQLYNELNLGRREEYDVLDKRRGRDPEMGGKP
RRKNPQEGLFNELQKDKMAEAFSEIGMKGERRRGKGHDGLFQGLSTATK
DTFDALHMQALPPR

In certain embodiments, the intracellular signaling domain of the CAR comprises a modified CD3ζ polypeptide comprising or consisting of an amino acid sequence that is at least about 80%, at least about 85%, at least about 90%, at least about 95%, at least about 96%, at least about 97%, at least about 98%, or at least about 99%, at least about 100% identical to SEQ ID NO: 52 or a fragment thereof, and/or may optionally comprise up to one or up to two or up to three conservative amino acid substitutions.

An exemplary nucleic acid sequence encoding the amino acid sequence of SEQ ID NO: 52 is set forth in SEQ ID NO: 53, which is provided below.

[SEQ ID NO: 53]
AGAGTGAAGTTCAGCAGGAGCGCAGACGCCCCCGCGTACCAGCAGGGCCAGAACCAGCTCTATA
ACGAGCTCAATCTAGGACGAAGAGAGGAGTACGATGTTTTGGACAAGAGACGTGGCCGGGACCC
TGAGATGGGGGGAAAGCCGAGAAGGAAGAACCCTCAGGAAGGCCTGTTCAATGAACTGCAGAAA
GATAAGATGGCGGAGGCCTTCAGTGAGATTGGGATGAAAGGCGAGCGCCGGAGGGGCAAGGGGC
ACGATGGCCTTTTCCAGGGGCTCAGTACAGCCACCAAGGACACCTTCGACGCCCTTCACATGCA
GGCCCTGCCCCCTCGC

In certain embodiments, the intracellular signaling domain of the CAR further comprises at least a co-stimulatory signaling region. In certain embodiments, the co-stimulatory signaling region comprises at least a portion of a co-stimulatory molecule, which can provide optimal lymphocyte activation.

As used herein, “co-stimulatory molecules” refer to cell surface molecules other than antigen receptors or their ligands that are required for an efficient response of lymphocytes to antigen. Non-limiting examples of co-stimulatory molecules include CD28, 4-1BB, OX40, ICOS, DAP-10, CD27, CD40, and NKGD2. The co-stimulatory molecule can bind to a co-stimulatory ligand, which is a protein expressed on cell surface that upon binding to its receptor produces a co-stimulatory response, i.e., an intracellular response that effects the stimulation provided when an antigen binds to its CAR molecule. Co-stimulatory ligands include, but are not limited to CD80, CD86, CD70, OX40L, and 4-1BBL. As one example, a 4-1BB ligand (i.e., 4-1BBL) may bind to 4-1BB (also known as “CD137”) for providing an intracellular signal that in combination with a CAR signal induces an effector cell function of the CAR⁺ T cell. CARs comprising an intracellular signaling domain that comprises a co-stimulatory signaling region comprising 4-1BB, ICOS or DAP-10 are disclosed in U.S. Pat. No. 7,446,190, which is herein incorporated by reference in its entirety.

In certain embodiments, the intracellular signaling domain of the CAR comprises a co-stimulatory signaling region that comprises a CD28 polypeptide (e.g., an intracellular domain of CD28 or a portion thereof). In certain embodiments, the co-stimulatory signaling region comprises an intracellular domain of human CD28 or a portion thereof. In certain embodiments, the co-stimulatory signaling region comprises a CD28 polypeptide comprising or consisting of amino acids 180 to 220 of SEQ ID NO: 31.

An exemplary nucleotide sequence encoding amino acids 180 to 220 of SEQ ID NO: 31 is set forth in SEQ ID NO: 54, which is provided below.

[SEQ ID NO: 54]
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCC

In certain embodiments, the co-stimulatory signaling region comprises a portion of a first co-stimulatory molecule and a portion of a second co-stimulatory molecule, e.g., an intracellular domain of CD28 and an intracellular domain of 4-1BB or an intracellular domain of CD28 and an intracellular domain of OX40.

In certain embodiments, the co-stimulatory signaling region comprises a 4-1BB polypeptide (e.g., an intracellular domain of 4-1BB or a portion thereof). In certain embodiments, the co-stimulatory signaling region comprises an intracellular domain of human 4-1BB or a portion thereof. 4-1BB can act as a tumor necrosis factor (TNF) ligand and have stimulatory activity. In certain embodiments, the 4-1BB polypeptide comprises or consists of an amino acid sequence that is at least about 80%, at least about 85%, at least about 90%, at least about 95%, at least about 96%, at least about 97%, at least about 98%, or at least about 99%, at least about 100% homologous or identical to the sequence with a NCBI Reference No: NP_001552.2 (SEQ ID NO: 55) or a fragment thereof, and/or may optionally comprise up to one or up to two or up to three conservative amino acid substitutions. In certain embodiments, the 4-1BB polypeptide comprises or consists of an amino acid sequence that is a consecutive portion of SEQ ID NO: 55, which is at least about 20, at least about 25, or at least about 30, or at least about 40, or at least about 50, and up to about 255 amino acids in length. In certain embodiments, the 4-1BB polypeptide comprises or consists of an amino acid sequence of amino acids 1 to 255, 1 to 50, 50 to 100, 100 to 150, 150 to 200, 214 to 255, or 200 to 255 of SEQ ID NO: 55. In certain embodiments, the co-stimulatory signaling region comprises a 4-1BB polypeptide comprising or consisting amino acids 214 to 255 of SEQ ID NO: 55. SEQ ID NO: 55 is provided below:

[SEQ ID NO: 55]
MGNSCYNIVATLLLVLNFERTRSLQDPCSNCPAGTFCDNNRNQICSPCPPNSFSSAGGQRTCDI
CRQCKGVFRTRKECSSTSNAECDCTPGFHCLGAGCSMCEQDCKQGQELTKKGCKDCCFGTQNDQ
KRGICRPWTNCSLDGKSVLVNGTKERDVVCGPSPADLSPGASSVTPPAPAREPGHSPQIISFFL
ALTSTALLFLLFFLTLRFSVVKRGRKKLLYIFKQPFMRPVQTTQEEDGCSCRFPEEEEGGCEL

An exemplary nucleotide sequence encoding amino acids 214 to 255 of SEQ ID NO: 55 is set forth in SEQ ID NO: 56, which is provided below.

[SEQ ID NO: 56]
AAACGGGGCAGAAAGAAACTCCTGTATATATTCAAACAACCATTTATGA
GACCAGTACAAACTACTCAAGAGGAAGATGGCTGTAGCTGCCGATTTCC
AGAAGAAGAAGAAGGAGGATGTGAACTG

In certain embodiments, a presently disclosed mesothelin-targeted CAR further comprises an inducible promoter, for expressing nucleic acid sequences in human cells. Promoters for use in expressing CAR genes can be a constitutive promoter, such as ubiquitin C (UbiC) promoter.

### 3.3.4.1. Exemplified CARs

In certain embodiments, the CAR can target any of the antigens disclosed in Section 3.1. In certain embodiments, the CAR is designated as “28z1XX”. In certain embodiments, the CAR comprises:
(a) an scFv of an antibody binding to the desired target;
(b) a transmembrane domain comprising a CD28 polypeptide (e.g., a transmembrane domain of human CD28 or a portion thereof);
(c) a CD28 hinge/spacer region (e.g., a hinge/spacer region of human CD28 or a portion thereof, e.g., amino acids 114 to 152 of SEQ ID NO: 31); and
(d) an intracellular signaling domain comprising (i) a modified CD3ζ polypeptide, and (ii) a co-stimulatory signaling region comprising a CD28 polypeptide (e.g., a human CD28 polypeptide, e.g., an intracellular domain of a human CD28 or a portion thereof).

In certain embodiments, the transmembrane domain comprises a CD28 polypeptide consisting of amino acids 153 to 179 of SEQ ID NO: 31. In certain embodiments, the CD28 hinge/spacer region consists of amino acids 114 to 152 of SEQ ID NO: 31. In certain embodiments, the modified CD3ζ polypeptide comprising a native ITAM1 consisting of the amino acid sequence set forth in SEQ ID NO: 40, an ITAM2 variant consisting of the amino acid sequence set forth in SEQ ID NO: 46, and an ITAM3 variant consisting of the amino acid sequence set forth in SEQ ID NO: 50. In certain embodiments, the modified CD3ζ polypeptide comprises the amino acid sequence set forth in SEQ ID NO: 52. In certain embodiments, the co-stimulatory signaling region comprises a CD28 polypeptide consisting of amino acids 180 to 220 of SEQ ID NO: 31.

In certain embodiments, the CAR is a CD19-targeted CAR. In certain embodiments, the CAR is designated as “1928z”. In certain embodiments, the CD19-targeted CAR comprises:
(a) an extracellular antigen-binding domain comprising a V_{H} comprising a CDR1 comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22; and a V_{L} comprising a CDR1 comprising the amino acid sequence set forth in SEQ ID NO: 23, a CDR2 comprising the amino acid sequence set forth in SEQ ID NO: 24, and a CDR3 comprising the amino acid sequence set forth in SEQ ID NO: 25;
(b) a transmembrane domain comprising a CD28 polypeptide (e.g., a transmembrane domain of human CD28 or a portion thereof);
(c) a CD28 hinge/spacer region (e.g., a hinge/spacer region of human CD28 or a portion thereof, e.g., amino acids 114 to 152 of SEQ ID NO: 31); and
(d) an intracellular signaling domain comprising (i) a CD3ζ polypeptide, and (ii) a co-stimulatory signaling region comprising a CD28 polypeptide (e.g., a human CD28 polypeptide, e.g., an intracellular domain of a human CD28 or a portion thereof).

In certain embodiments, the transmembrane domain comprises a CD28 polypeptide consisting of amino acids 153 to 179 of SEQ ID NO: 31.

In certain embodiments, the CD28 hinge/spacer region consists of amino acids 114 to 152 of SEQ ID NO: 31. In certain embodiments, the CD3ζ polypeptide consists of the amino acids 52 to 164 of SEQ ID NO: 35.

In certain embodiments, the co-stimulatory signaling region comprises a CD28 polypeptide consisting of amino acids 180 to 220 of SEQ ID NO: 31.

In certain embodiments, the CAR is a CD19-targeted CAR. In certain embodiments, the CAR is designated as “1928z1XX”. In certain embodiments, the CD19-targeted CAR comprises:
(a) an extracellular antigen-binding domain comprising a V_{H} comprising a CDR1 comprising the amino acid sequence set forth in SEQ ID NO: 20, a CDR2 comprising the amino acid sequence set forth in SEQ ID NO: 21, and a CDR3 comprising the amino acid sequence set forth in SEQ ID NO: 22; and a V_{L} comprising a CDR1 comprising the amino acid sequence set forth in SEQ ID NO: 23, a CDR2 comprising the amino acid sequence set forth in SEQ ID NO: 24, and a CDR3 comprising the amino acid sequence set forth in SEQ ID NO: 25;
(b) a transmembrane domain comprising a CD28 polypeptide (e.g., a transmembrane domain of human CD28 or a portion thereof);
(c) a CD28 hinge/spacer region (e.g., a hinge/spacer region of human CD28 or a portion thereof, e.g., amino acids 114 to 152 of SEQ ID NO: 31); and
(d) an intracellular signaling domain comprising (i) a modified CD3ζ polypeptide, and (ii) a co-stimulatory signaling region comprising a CD28 polypeptide (e.g., a human CD28 polypeptide, e.g., an intracellular domain of a human CD28 or a portion thereof).

In certain embodiments, the transmembrane domain comprises a CD28 polypeptide consisting of amino acids 153 to 179 of SEQ ID NO: 31.

In certain embodiments, the CD28 hinge/spacer region consists of amino acids 114 to 152 of SEQ ID NO: 31. In certain embodiments, the modified CD3ζ polypeptide comprising a native ITAM1 consisting of the amino acid sequence set forth in SEQ ID NO: 40, an ITAM2 variant consisting of the amino acid sequence set forth in SEQ ID NO: 46, and an ITAM3 variant consisting of the amino acid sequence set forth in SEQ ID NO: 50. In certain embodiments, the modified CD3ζ polypeptide comprises the amino acid sequence set forth in SEQ ID NO: 52.

In certain embodiments, the co-stimulatory signaling region comprises a CD28 polypeptide consisting of amino acids 180 to 220 of SEQ ID NO: 31.

In certain embodiments, the CAR is a CD70-targeted CAR. In certain embodiments, the CAR is designated as “70-28z1XX”. In certain embodiments, the CD70-targeted CAR comprises:
(a) an extracellular antigen-binding domain comprising a V_{H} comprising a CDR1 comprising the amino acid sequence set forth in SEQ ID NO: 104, a CDR2 comprising the amino acid sequence set forth in SEQ ID NO: 105, and a CDR3 comprising the amino acid sequence set forth in SEQ ID NO: 106; and a V_{L} comprising a CDR1 comprising the amino acid sequence set forth in SEQ ID NO: 107, a CDR2 comprising the amino acid sequence set forth in SEQ ID NO: 108, and a CDR3 comprising the amino acid sequence set forth in SEQ ID NO: 109;
(b) a transmembrane domain comprising a CD28 polypeptide (e.g., a transmembrane domain of human CD28 or a portion thereof);
(c) a CD28 hinge/spacer region (e.g., a hinge/spacer region of human CD28 or a portion thereof, e.g., amino acids 114 to 152 of SEQ ID NO: 31); and
(d) an intracellular signaling domain comprising (i) a modified CD3ζ polypeptide, and (ii) a co-stimulatory signaling region comprising a CD28 polypeptide (e.g., a human CD28 polypeptide, e.g., an intracellular domain of a human CD28 or a portion thereof).

In certain embodiments, the transmembrane domain comprises a CD28 polypeptide consisting of amino acids 153 to 179 of SEQ ID NO: 31.

In certain embodiments, the CD28 hinge/spacer region consists of amino acids 114 to 152 of SEQ ID NO: 31. In certain embodiments, the modified CD3ζ polypeptide comprising a native ITAM1 consisting of the amino acid sequence set forth in SEQ ID NO: 40, an ITAM2 variant consisting of the amino acid sequence set forth in SEQ ID NO: 46, and an ITAM3 variant consisting of the amino acid sequence set forth in SEQ ID NO: 50. In certain embodiments, the modified CD3ζ polypeptide comprises the amino acid sequence set forth in SEQ ID NO: 52.

In certain embodiments, the co-stimulatory signaling region comprises a CD28 polypeptide consisting of amino acids 180 to 220 of SEQ ID NO: 31.

In certain embodiments, the CAR is a IL13R-targeted CAR. In certain embodiments, the CAR is designated as “IL13R-28z1XX”. In certain embodiments, the IL13R-targeted CAR comprises:
(a) an extracellular antigen-binding domain comprising a VH comprising a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114; and a VL comprising a CDR1 comprising the amino acid sequence set forth in SEQ ID NO: 115, a CDR2 comprising the amino acid sequence set forth in SEQ ID NO: 116, and a CDR3 comprising the amino acid sequence set forth in SEQ ID NO: 117;
(b) a transmembrane domain comprising a CD28 polypeptide (e.g., a transmembrane domain of human CD28 or a portion thereof);
(c) a CD28 hinge/spacer region (e.g., a hinge/spacer region of human CD28 or a portion thereof, e.g., amino acids 114 to 152 of SEQ ID NO: 31); and
(d) an intracellular signaling domain comprising (i) a modified CD3ζ polypeptide, and (ii) a co-stimulatory signaling region comprising a CD28 polypeptide (e.g., a human CD28 polypeptide, e.g., an intracellular domain of a human CD28 or a portion thereof).

In certain embodiments, the transmembrane domain comprises a CD28 polypeptide consisting of amino acids 153 to 179 of SEQ ID NO: 31.

In certain embodiments, the CD28 hinge/spacer region consists of amino acids 114 to 152 of SEQ ID NO: 31. In certain embodiments, the modified CD3ζ polypeptide comprising a native ITAM1 consisting of the amino acid sequence set forth in SEQ ID NO: 40, an ITAM2 variant consisting of the amino acid sequence set forth in SEQ ID NO: 46, and an ITAM3 variant consisting of the amino acid sequence set forth in SEQ ID NO: 50. In certain embodiments, the modified CD3ζ polypeptide comprises the amino acid sequence set forth in SEQ ID NO: 52.

In certain embodiments, the co-stimulatory signaling region comprises a CD28 polypeptide consisting of amino acids 180 to 220 of SEQ ID NO: 31.

### 3.4. Chimeric Ligand Receptors

In certain embodiments, the antigen-recognizing receptor is a chimeric ligand receptor that comprises a ligand or a portion thereof that binds to the first antigen. In certain embodiments, the chimeric ligand receptor further comprises a transmembrane domain and an intracellular signaling domain.

In certain embodiments, the transmembrane domain is fused to the ligand or portion thereof. In certain embodiments, the transmembrane domain is fused to the intracellular signaling domain. In certain embodiments, the transmembrane domain is positioned between the ligand or portion thereof and the intracellular signaling domain. In certain embodiments the transmembrane domain of the chimeric ligand receptor is a transmembrane domain disclosed in Section 3.3.2. In certain embodiments, the intracellular signaling domain of the chimeric ligand receptor comprises a CD3ζ polypeptide (e.g., as disclosed in Section 3.3.4).

Additional information on the presently disclosed chimeric ligand receptor can be found in Sauer et al., Blood (2021) 138 (4): 318-330, the content of which is incorporated by reference in its entirety.

### 3.5. TCR-Like Fusion Molecules

In certain embodiments, the antigen-recognizing receptor is a TCR-like fusion molecule. Non-limiting examples of TCR fusion molecules include HLA-Independent TCR-based Chimeric Antigen Receptor (also known as “HIT”, e.g., those disclosed in International Patent Application No. PCT/US19/017525, which is incorporated by reference in its entirety), and T cell receptor fusion constructs (TRuCs) (e.g., those disclosed in Baeuerle et al., “Synthetic TRuC receptors engaging the complete T cell receptor for potent anti-tumor response,” Nature Communications volume 10, Article number: 2087 (2019), which is incorporated by reference in its entirety).

In certain embodiments, the TCR-like fusion molecule is a recombinant T cell receptor (TCR). In certain embodiments, the recombinant TCR comprises at least one antigen-binding chain. In certain embodiments, the antigen-binding domain of the recombinant TCR comprises a ligand for a cell-surface receptor, a receptor for a cell surface ligand, an antigen binding portion of an antibody or a fragment thereof, or an antigen binding portion of a TCR. In certain embodiments, the recombinant TCR comprises two antigen binding chains, i.e., a first antigen binding chain and a second antigen binding chain. In certain embodiments, the first and second antigen-binding chains each comprises a constant domain. In certain embodiments, the recombinant TCR binds to an antigen (e.g., a tumor antigen) in an HLA-independent manner. Thus, in certain embodiments, the recombinant TCR is an HLA-independent (or non-HLA restricted) TCR (referred to as “HIT”).

In certain embodiments, the first antigen-binding chain comprises an antigen-binding fragment of a heavy chain variable region (V_{H}) of an antibody. In certain embodiments, the second antigen-binding chain comprises an antigen-binding fragment of a light chain variable region (V_{L}) of an antibody. In certain embodiments, the first antigen-binding chain comprises an antigen-binding fragment of a V_{H} of an antibody, and the second antigen-binding chain comprises an antigen-binding fragment of a V_{L} of the antibody.

In certain embodiments, the constant domain comprises a TCR constant region selected from the group consisting of a native or modified TRAC polypeptide, a native or modified TRBC polypeptide, a native or modified TRDC polypeptide, a native or modified TRGC polypeptide and any variants or functional fragments thereof. In certain embodiments, the constant domain comprises a native or modified TRAC polypeptide. In certain embodiments, the constant domain comprises a native or modified TRBC polypeptide. In certain embodiments, the first antigen-binding chain comprises a TRAC polypeptide, and the second antigen-binding chain comprises a TRBC polypeptide. In certain embodiments, the first antigen-binding chain comprises a TRBC polypeptide, and the second antigen-binding chain comprises a TRAC polypeptide.

In certain embodiments, the first antigen-binding chain comprises a V_{H} of an antibody and a TRAC polypeptide, and the second antigen-binding chain comprises a V_{L} of an antibody and a TRBC polypeptide.

In certain embodiments, the first antigen-binding chain comprises a V_{H} of an antibody and a TRBC polypeptide, and the second antigen-binding chain comprises a V_{L} of an antibody and a TRAC polypeptide.

In certain embodiments, at least one of the TRAC polypeptide and the TRBC polypeptide is endogenous. In certain embodiments, the TRAC polypeptide is endogenous. In certain embodiments, the TRBC polypeptide is endogenous. In certain embodiments, both the TRAC polypeptide and the TRBC polypeptide are endogenous.

In certain embodiments, the antigen binding chain is capable of associating with a CD3ζ polypeptide. In certain embodiments, the antigen binding chain, upon binding to an antigen, is capable of activating the CD3ζ polypeptide associated to the antigen binding chain. In certain embodiments, the activation of the CD3ζ polypeptide is capable of activating an immunoresponsive cell. In certain embodiments, the TCR-like fusion molecule is capable of integrating with a CD3 complex and providing HLA-independent antigen recognition. In certain embodiments, the TCR-like fusion molecule replaces an endogenous TCR in a CD3/TCR complex.

In certain embodiments, the first and second antigen binding chains bind to an antigen with a dissociation constant (K_{D}) of about 2×10⁻⁷ M or less. In certain embodiments, the first and second antigen binding chains bind to an antigen with a high binding affinity. In certain embodiments, the K_{D} is about 2×10⁻⁷ M or less, about 1×10⁻⁷ M or less, about 9×10⁻⁸ M or less, about 1×10⁻⁸ M or less, about 9×10⁻⁹ M or less, about 5×10⁻⁹ M or less, about 4×10⁻⁹ M or less, about 3×10⁻⁹ or less, about 2×10⁻⁹ M or less, or about 1×10⁻⁹ M or less. In certain embodiments, the K_{D} is about 1×10⁻⁸ M or less. In certain embodiments, the K_{D} is about 3×10⁻⁹ M or less. In certain embodiments, the K_{D} is about 5×10⁻⁹ M or less. In certain embodiments, the K_{D} is from about 1×10⁻⁹ M to about 1×10⁻⁸ M. In certain embodiments, the KD is from about 1.5×10⁻⁹ M to about 1×10⁻⁸ M. In certain embodiments, the K_{D} is from about 5×10⁻⁹ M to about 1×10⁻⁸ M.

In certain embodiments, the constant domain comprises a TCR constant region, e.g., T cell receptor alpha constant region (TRAC), T cell receptor beta constant region (TRBC, e.g., TRBC1 or TRBC2), T cell receptor gamma constant region (TRGC, e.g., TRGC1 or TRGC2), T cell receptor delta constant region (TRDC) or any variants or functional fragments thereof.

In certain embodiments, the first antigen binding chain or the second antigen binding chain comprises a constant domain that comprises a native or modified TRAC polypeptide. In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 57 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 57. SEQ ID NO: 57 is provided below.

[SEQ ID NO: 57]
IQNPDPAVYQLRDSKSSDKSVCLFTDFDSQTNVSQSKDSDVYITDKTVL
DMRSMDFKSNSAVAWSNKSDFACANAENNSIIPEDTFFPSPESSCDVKL
VEKSFETDTNLNFQNLSVIGFRILLLKVAGFNLLMTLRLWSS

An exemplary nucleotide sequence encoding the amino acid sequence of SEQ ID NO: 57 is set forth in SEQ ID NO: 58, which is provided below.

[SEQ ID NO: 58]
ATCCAGAACCCTGACCCTGCCGTGTACCAGCTGAGAGACTCTAAATCCAGTGACAAGTCTGTCTGCCTAT
TCACCGATTTTGATTCTCAAACAAATGTGTCACAAAGTAAGGATTCTGATGTGTATATCACAGACAAAAC
TGTGCTAGACATGAGGTCTATGGACTTCAAGAGCAACAGTGCTGTGGCCTGGAGCAACAAATCTGACTTT
GCATGTGCAAACGCCTTCAACAACAGCATTATTCCAGAAGACACCTTCTTCCCCAGCCCAGAAAGTTCCT
GTGATGTCAAGCTGGTCGAGAAAAGCTTTGAAACAGATACGAACCTAAACTTTCAAAACCTGTCAGTGAT
TGGGTTCCGAATCCTCCTCCTGAAAGTGGCCGGGTTTAATCTGCTCATGACGCTGCGGCTGTGGTCCAGC

In certain embodiments, the TRAC polypeptide comprises an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 59 or a fragment thereof, and/or may optionally comprise up to one or up to two or up to three conservative amino acid substitutions. In certain embodiments, the TRAC polypeptide comprises or consists of the amino acid sequence set forth in SEQ ID NO: 59. SEQ ID NO: 59 is provided below.

[SEQ ID NO: 59]
IPNIQNPDPAVYQLRDSKSSDKSVCLFTDFDSQTNVSQSKDSDVYITDKTVLDMRSMDFKSNSAVAWSNK
SDFACANAFNNSIIPEDTFFPSPESSCDVKLVEKSFETDTNLNFQNLSVIGFRILLLKVAGFNLLMTLRL
WSS

An exemplary nucleotide sequence encoding the amino acid sequence of SEQ ID NO: 59 is set forth in SEQ ID NO: 60, which is provided below.

[SEQ ID NO: 60]
ATTCCCAATATCCAGAACCCTGACCCTGCCGTGTACCAGCTGAGAGACTCTAAATCCAGTGACAAGTCTG
TCTGCCTATTCACCGATTTTGATTCTCAAACAAATGTGTCACAAAGTAAGGATTCTGATGTGTATATCAC
AGACAAAACTGTGCTAGACATGAGGTCTATGGACTTCAAGAGCAACAGTGCTGTGGCCTGGAGCAACAAA
TCTGACTTTGCATGTGCAAACGCCTTCAACAACAGCATTATTCCAGAAGACACCTTCTTCCCCAGCCCAG
AAAGTTCCTGTGATGTCAAGCTGGTCGAGAAAAGCTTTGAAACAGATACGAACCTAAACTTTCAAAACCT
GTCAGTGATTGGGTTCCGAATCCTCCTCCTGAAAGTGGCCGGGTTTAATCTGCTCATGACGCTGCGGCTG
TGGTCCAGC

In certain embodiments, the TRAC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by the gene of NCBI Genbank ID: 28755, NG_001332.3, range 925603 to 930229 (SEQ ID NO: 61) or a fragment thereof, and/or may optionally comprise up to one or up to two or up to three conservative amino acid substitutions. In certain embodiments, the TRAC polypeptide comprises or consists of the amino acid sequence encoded by the nucleotide sequence of SEQ ID NO: 61. SEQ ID NO: 61 is provided below.

[SEQ ID NO: 61]
ATATCCAGAACCCTGACCCTGCCGTGTACCAGCTGAGAGACTCTAAATCCAGTGACAAGTCTGTCTGCCT
ATTCACCGATTTTGATTCTCAAACAAATGTGTCACAAAGTAAGGATTCTGATGTGTATATCACAGACAAA
ACTGTGCTAGACATGAGGTCTATGGACTTCAAGAGCAACAGTGCTGTGGCCTGGAGCAACAAATCTGACT
TTGCATGTGCAAACGCCTTCAACAACAGCATTATTCCAGAAGACACCTTCTTCCCCAGCCCAGGTAAGGG
CAGCTTTGGTGCCTTCGCAGGCTGTTTCCTTGCTTCAGGAATGGCCAGGTTCTGCCCAGAGCTCTGGTCA
ATGATGTCTAAAACTCCTCTGATTGGTGGTCTCGGCCTTATCCATTGCCACCAAAACCCTCTTTTTACTA
AGAAACAGTGAGCCTTGTTCTGGCAGTCCAGAGAATGACACGGGAAAAAAGCAGATGAAGAGAAGGTGGC
AGGAGAGGGCACGTGGCCCAGCCTCAGTCTCTCCAACTGAGTTCCTGCCTGCCTGCCTTTGCTCAGACTG
TTTGCCCCTTACTGCTCTTCTAGGCCTCATTCTAAGCCCCTTCTCCAAGTTGCCTCTCCTTATTTCTCCC
TGTCTGCCAAAAAATCTTTCCCAGCTCACTAAGTCAGTCTCACGCAGTCACTCATTAACCCACCAATCAC
TGATTGTGCCGGCACATGAATGCACCAGGTGTTGAAGTGGAGGAATTAAAAAGTCAGATGAGGGGTGTGC
CCAGAGGAAGCACCATTCTAGTTGGGGGAGCCCATCTGTCAGCTGGGAAAAGTCCAAATAACTTCAGATT
GGAATGTGTTTTAACTCAGGGTTGAGAAAACAGCTACCTTCAGGACAAAAGTCAGGGAAGGGCTCTCTGA
AGAAATGCTACTTGAAGATACCAGCCCTACCAAGGGCAGGGAGAGGACCCTATAGAGGCCTGGGACAGGA
GCTCAATGAGAAAGGAGAAGAGCAGCAGGCATGAGTTGAATGAAGGAGGCAGGGCCGGGTCACAGGGCCT
TCTAGGCCATGAGAGGGTAGACAGTATTCTAAGGACGCCAGAAAGCTGTTGATCGGCTTCAAGCAGGGGA
GGGACACCTAATTTGCTTTTCTTTTTTTTTTTTTTTTTTTTTTTTTTTTTTGAGATGGAGTTTTGCTCTT
GTTGCCCAGGCTGGAGTGCAATGGTGCATCTTGGCTCACTGCAACCTCCGCCTCCCAGGTTCAAGTGATT
CTCCTGCCTCAGCCTCCCGAGTAGCTGAGATTACAGGCACCCGCCACCATGCCTGGCTAATTTTTTGTAT
TTTTAGTAGAGACAGGGTTTCACTATGTTGGCCAGGCTGGTCTCGAACTCCTGACCTCAGGTGATCCACC
CGCTTCAGCCTCCCAAAGTGCTGGGATTACAGGCGTGAGCCACCACACCCGGCCTGCTTTTCTTAAAGAT
CAATCTGAGTGCTGTACGGAGAGTGGGTTGTAAGCCAAGAGTAGAAGCAGAAAGGGAGCAGTTGCAGCAG
AGAGATGATGGAGGCCTGGGCAGGGTGGTGGCAGGGAGGTAACCAACACCATTCAGGTTTCAAAGGTAGA
ACCATGCAGGGATGAGAAAGCAAAGAGGGGATCAAGGAAGGCAGCTGGATTTTGGCCTGAGCAGCTGAGT
CAATGATAGTGCCGTTTACTAAGAAGAAACCAAGGAAAAAATTTGGGGTGCAGGGATCAAAACTTTTTGG
AACATATGAAAGTACGTGTTTATACTCTTTATGGCCCTTGTCACTATGTATGCCTCGCTGCCTCCATTGG
ACTCTAGAATGAAGCCAGGCAAGAGCAGGGTCTATGTGTGATGGCACATGTGGCCAGGGTCATGCAACAT
GTACTTTGTACAAACAGTGTATATTGAGTAAATAGAAATGGTGTCCAGGAGCCGAGGTATCGGTCCTGCC
AGGGCCAGGGGCTCTCCCTAGCAGGTGCTCATATGCTGTAAGTTCCCTCCAGATCTCTCCACAAGGAGGC
ATGGAAAGGCTGTAGTTGTTCACCTGCCCAAGAACTAGGAGGTCTGGGGTGGGAGAGTCAGCCTGCTCTG
GATGCTGAAAGAATGTCTGTTTTTCCTTTTAGAAAGTTCCTGTGATGTCAAGCTGGTCGAGAAAAGCTTT
GAAACAGGTAAGACAGGGGTCTAGCCTGGGTTTGCACAGGATTGCGGAAGTGATGAACCCGCAATAACCC
TGCCTGGATGAGGGAGTGGGAAGAAATTAGTAGATGTGGGAATGAATGATGAGGAATGGAAACAGCGGTT
CAAGACCTGCCCAGAGCTGGGTGGGGTCTCTCCTGAATCCCTCTCACCATCTCTGACTTTCCATTCTAAG
CACTTTGAGGATGAGTTTCTAGCTTCAATAGACCAAGGACTCTCTCCTAGGCCTCTGTATTCCTTTCAAC
AGCTCCACTGTCAAGAGAGCCAGAGAGAGCTTCTGGGTGGCCCAGCTGTGAAATTTCTGAGTCCCTTAGG
GATAGCCCTAAACGAACCAGATCATCCTGAGGACAGCCAAGAGGTTTTGCCTTCTTTCAAGACAAGCAAC
AGTACTCACATAGGCTGTGGGCAATGGTCCTGTCTCTCAAGAATCCCCTGCCACTCCTCACACCCACCCT
GGGCCCATATTCATTTCCATTTGAGTTGTTCTTATTGAGTCATCCTTCCTGTGGTAGCGGAACTCACTAA
GGGGCCCATCTGGACCCGAGGTATTGTGATGATAAATTCTGAGCACCTACCCCATCCCCAGAAGGGCTCA
GAAATAAAATAAGAGCCAAGTCTAGTCGGTGTTTCCTGTCTTGAAACACAATACTGTTGGCCCTGGAAGA
ATGCACAGAATCTGTTTGTAAGGGGATATGCACAGAAGCTGCAAGGGACAGGAGGTGCAGGAGCTGCAGG
CCTCCCCCACCCAGCCTGCTCTGCCTTGGGGAAAACCGTGGGTGTGTCCTGCAGGCCATGCAGGCCTGGG
ACATGCAAGCCCATAACCGCTGTGGCCTCTTGGTTTTACAGATACGAACCTAAACTTTCAAAACCTGTCA
GTGATTGGGTTCCGAATCCTCCTCCTGAAAGTGGCCGGGTTTAATCTGCTCATGACGCTGCGGCTGTGGT
CCAGCTGAGGTGAGGGGCCTTGAAGCTGGGAGTGGGGTTTAGGGACGCGGGTCTCTGGGTGCATCCTAAG
CTCTGAGAGCAAACCTCCCTGCAGGGTCTTGCTTTTAAGTCCAAAGCCTGAGCCCACCAAACTCTCCTAC
TTCTTCCTGTTACAAATTCCTCTTGTGCAATAATAATGGCCTGAAACGCTGTAAAATATCCTCATTTCAG
CCGCCTCAGTTGCACTTCTCCCCTATGAGGTAGGAAGAACAGTTGTTTAGAAACGAAGAAACTGAGGCCC
CACAGCTAATGAGTGGAGGAAGAGAGACACTTGTGTACACCACATGCCTTGTGTTGTACTTCTCTCACCG
TGTAACCTCCTCATGTCCTCTCTCCCCAGTACGGCTCTCTTAGCTCAGTAGAAAGAAGACATTACACTCA
TATTACACCCCAATCCTGGCTAGAGTCTCCGCACCCTCCTCCCCCAGGGTCCCCAGTCGTCTTGCTGACA
ACTGCATCCTGTTCCATCACCATCAAAAAAAAACTCCAGGCTGGGTGCGGGGGCTCACACCTGTAATCCC
AGCACTTTGGGAGGCAGAGGCAGGAGGAGCACAGGAGCTGGAGACCAGCCTGGGCAACACAGGGAGACCC
CGCCTCTACAAAAAGTGAAAAAATTAACCAGGTGTGGTGCTGCACACCTGTAGTCCCAGCTACTTAAGAG
GCTGAGATGGGAGGATCGCTTGAGCCCTGGAATGTTGAGGCTACAATGAGCTGTGATTGCGTCACTGCAC
TCCAGCCTGGAAGACAAAGCAAGATCCTGTCTCAAATAATAAAAAAAATAAGAACTCCAGGGTACATTTG
CTCCTAGAACTCTACCACATAGCCCCAAACAGAGCCATCACCATCACATCCCTAACAGTCCTGGGTCTTC
CTCAGTGTCCAGCCTGACTTCTGTTCTTCCTCATTCCAGATCTGCAAGATTGTAAGACAGCCTGTGCTCC
CTCGCTCCTTCCTCTGCATTGCCCCTCTTCTCCCTCTCCAAACAGAGGGAACTCTCCTACCCCCAAGGAG
GTGAAAGCTGCTACCACCTCTGTGCCCCCCCGGCAATGCCACCAACTGGATCCTACCCGAATTTATGATT
AAGATTGCTGAAGAGCTGCCAAACACTGCTGCCACCCCCTCTGTTCCCTTATTGCTGCTTGTCACTGCCT
GACATTCACGGCAGAGGCAAGGCTGCTGCAGCCTCCCCTGGCTGTGCACATTCCCTCCTGCTCCCCAGAG
ACTGCCTCCGCCATCCCACAGATGATGGATCTTCAGTGGGTTCTCTTGGGCTCTAGGTCCTGCAGAATGT
TGTGAGGGGTTTATTTTTTTTTAATAGTGTTCATAAAGAAATACATAGTATTCTTCTTCTCAAGACGTGG
GGGGAAATTATCTCATTATCGAGGCCCTGCTATGCTGTGTATCTGGGCGTGTTGTATGTCCTGCTGCCGA
TGCCTTC

In certain embodiments, the first antigen binding chain or the second antigen binding chain comprises a constant domain comprising a native or modified TRBC polypeptide. In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 62 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 62. SEQ ID NO: 62 is provided below.

[SEQ ID NO: 62]
DLKNVFPPEVAVFEPSEAEISHTQKATLVCLATGFYPDHVELSWWVNGKEVHSGVSTDPQPLKEQPALND
SRYCLSSRLRVSATFWQNPRNHERCQVQFYGLSENDEWTQDRAKPVTQIVSAEAWGRADCGFTSESYQQG
VLSATILYEILLGKATLYAVLVSALVLMAMVKRKDSRG

An exemplary nucleotide sequence encoding the amino acid sequence of SEQ ID NO: 62 is set forth in SEQ ID NO: 63, which is provided below.

[SEQ ID NO: 63]
GATCTGAAAAACGTGTTCCCTCCTGAAGTGGCTGTCTTTGAACCATCCGAGGCCGAGATTTCCCATACCC
AGAAAGCAACTCTGGTCTGTCTGGCCACTGGATTCTACCCCGATCACGTGGAACTGTCTTGGTGGGTGAA
CGGCAAGGAAGTCCATTCCGGAGTCTCTACCGACCCTCAGCCCCTCAAGGAGCAGCCTGCTCTCAACGAT
TCTCGGTACTGCCTGTCATCTCGACTGAGAGTGTCTGCCACCTTCTGGCAGAACCCTAGAAACCACTTTC
GGTGTCAGGTCCAGTTTTACGGCCTGAGCGAGAACGATGAGTGGACACAGGATAGAGCCAAACCTGTGAC
ACAGATTGTGAGCGCCGAGGCTTGGGGACGAGCCGATTGTGGCTTCACATCCGAGTCTTACCAGCAGGGA
GTGCTGTCTGCTACAATCCTCTACGAAATTCTCCTGGGGAAGGCCACCCTGTACGCTGTCCTCGTGTCTG
CTCTGGTGCTCATGGCTATGGTCAAACGAAAGGACTCTAGAGGC

In certain embodiments, the TRBC polypeptide is a TRBC2 polypeptide. In certain embodiments, the TRBC2 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 64 or a fragment thereof, and/or may optionally comprise up to one or up to two or up to three conservative amino acid substitutions. In certain embodiments, the TRBC2 polypeptide comprises or consists of the amino acid sequence set forth in SEQ ID NO: 64. SEQ ID NO: 64 is provided below.

[SEQ ID NO: 64]
LEDLKNVFPPEVAVFEPSEAEISHTQKATLVCLATGFYPDHVELSWWVNGKEVHSGVSTDPQPLKEQPAL
NDSRYCLSSRLRVSATFWQNPRNHERCQVQFYGLSENDEWTQDRAKPVTQIVSAEAWGRADCGFTSESYQ
QGVLSATILYEILLGKATLYAVLVSALVLMAMVKRKDSRG

An exemplary nucleotide sequence encoding the amino acid sequence of SEQ ID NO: 64 is set forth in SEQ ID NO: 65, which is provided below.

[SEQ ID NO: 65]
CTGGAGGATCTGAAAAACGTGTTCCCTCCTGAAGTGGCTGTCTTTGAACCATCCGAGGCCGAGATTTCCC
ATACCCAGAAAGCAACTCTGGTCTGTCTGGCCACTGGATTCTACCCCGATCACGTGGAACTGTCTTGGTG
GGTGAACGGCAAGGAAGTCCATTCCGGAGTCTCTACCGACCCTCAGCCCCTCAAGGAGCAGCCTGCTCTC
AACGATTCTCGGTACTGCCTGTCATCTCGACTGAGAGTGTCTGCCACCTTCTGGCAGAACCCTAGAAACC
ACTTTCGGTGTCAGGTCCAGTTTTACGGCCTGAGCGAGAACGATGAGTGGACACAGGATAGAGCCAAACC
TGTGACACAGATTGTGAGCGCCGAGGCTTGGGGACGAGCCGATTGTGGCTTCACATCCGAGTCTTACCAG
CAGGGAGTGCTGTCTGCTACAATCCTCTACGAAATTCTCCTGGGGAAGGCCACCCTGTACGCTGTCCTCG
TGTCTGCTCTGGTGCTCATGGCTATGGTCAAACGAAAGGACTCTAGAGGC

In certain embodiments, the TRBC polypeptide is a TRBC1 polypeptide. In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 66 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 66. SEQ ID NO: 66 is provided below.

[SEQ ID NO: 66]
LNKVEPPEVAVFEPSEAEISHTQKATLVCLATGFFPDHVELSWWVNGKEVHSGVSTDPQPLKEQPALNDS
RYCLSSRLRVSATFWQNPRNHFRCQVQFYGLSENDEWTQDRAKPVTQIVSAEAWGRADCGFTSVSYQQGV
LSATILYEILLGKATLYAVLVSALVLMAMVKRKDF

In certain embodiments, the TRBC1 polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence set forth in SEQ ID NO: 67 or a fragment thereof, and/or may optionally comprise up to one or up to two or up to three conservative amino acid substitutions. In certain embodiments, the TRBC1 polypeptide comprises or consists of the amino acid sequence set forth in SEQ ID NO: 67. SEQ ID NO: 67 is provided below.

[SEQ ID NO: 67]
DLNKVFPPEVAVFEPSEAEISHTQKATLVCLATGFFPDHVELSWWVNGKEVHSGVSTDPQPLKEQPALND
SRYCLSSRLRVSATFWQNPRNHFRCQVQFYGLSENDEWTQDRAKPVTQIVSAEAWGRADCGFTSVSYQQG
VLSATILYEILLGKATLYAVLVSALVLMAMVKRKDF

An exemplary nucleotide sequence encoding the amino acid sequence of SEQ ID NO: 67 is set forth in SEQ ID NO: 68, which is provided below.

[SEQ ID NO: 68]
GACCTGAACAAGGTGTTCCCACCCGAGGTCGCTGTGTTTGAGCCATCAGAAGCAGAGATCTCCCACACCC
AAAAGGCCACACTGGTGTGCCTGGCCACAGGCTTCTTCCCCGACCACGTGGAGCTGAGCTGGTGGGTGAA
TGGGAAGGAGGTGCACAGTGGGGTCAGCACAGACCCGCAGCCCCTCAAGGAGCAGCCCGCCCTCAATGAC
TCCAGATACTGCCTGAGCAGCCGCCTGAGGGTCTCGGCCACCTTCTGGCAGAACCCCCGCAACCACTTCC
GCTGTCAAGTCCAGTTCTACGGGCTCTCGGAGAATGACGAGTGGACCCAGGATAGGGCCAAACCCGTCAC
CCAGATCGTCAGCGCCGAGGCCTGGGGTAGAGCAGACTGTGGCTTTACCTCGGTGTCCTACCAGCAAGGG
GTCCTGTCTGCCACCATCCTCTATGAGATCCTGCTAGGGAAGGCCACCCTGTATGCTGTGCTGGTCAGCG
CCCTTGTGTTGATGGCCATGGTCAAGAGAAAGGATTTC

In certain embodiments, the TRBC polypeptide comprises or consists of an amino acid sequence that is at least about 80%, at least about 85%, at least about 90%, at least about 95%, at least about 96%, at least about 97%, at least about 98%, at least about 99% or at least about 100% homologous or identical to the amino acid sequence encoded by a transcript expressed by a gene of NCBI Genbank ID: 28639, NG_001333.2, range 645749 to 647196 (TRBC1, SEQ ID NO: 69), NCBI Genbank ID: 28638, NG_001333.2 range 655095 to 656583 (TRBC2, SEQ ID NO: 70) or a fragment thereof, and/or may optionally comprise up to one or up to two or up to three conservative amino acid substitutions. In certain embodiments, the TRBC polypeptide comprises or consists of the amino acid sequence encoded by the nucleotide sequence of SEQ ID NO: 69. In certain embodiments, the TRBC polypeptide comprises or consists of the amino acid sequence encoded by the nucleotide sequence of SEQ ID NO: 70. SEQ ID NO: 69 and 70 are provided below.

[SEQ ID NO: 69]
AGGACCTGAACAAGGTGTTCCCACCCGAGGTCGCTGTGTTTGAGCCATCAGAAGCAGAGATCTCCCACAC
CCAAAAGGCCACACTGGTGTGCCTGGCCACAGGCTTCTTCCCCGACCACGTGGAGCTGAGCTGGTGGGTG
AATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTT
CCGCTGTCAAGTCCAGTTCTACGGGCTCTCGGAGAATGACGAGTGGACCCAGGATAGGGCCAAACCCGTC
ACCCAGATCGTCAGCGCCGAGGCCTGGGGTAGAGCAGGTGAGTGGGGCCTGGGGAGATGCCTGGAGGAGA
TTAGGTGAGACCAGCTACCAGGGAAAATGGAAAGATCCAGGTAGCAGACAAGACTAGATCCAAAAAGAAA
GGAACCAGCGCACACCATGAAGGAGAATTGGGCACCTGTGGTTCATTCTTCTCCCAGATTCTCAGCCCAA
CAGAGCCAAGCAGCTGGGTCCCCTTTCTATGTGGCCTGTGTAACTCTCATCTGGGTGGTGCCCCCCATCC
CCCTCAGTGCTGCCACATGCCATGGATTGCAAGGACAATGTGGCTGACATCTGCATGGCAGAAGAAAGGA
GGTGCTGGGCTGTCAGAGGAAGCTGGTCTGGGCCTGGGAGTCTGTGCCAACTGCAAATCTGACTTTACTT
TTAATTGCCTATGAAAATAAGGTCTCTCATTTATTTTCCTCTCCCTGCTTTCTTTCAGACTGTGGCTTTA
CCTCGGGTAAGTAAGCCCTTCCTTTTCCTCTCCCTCTCTCATGGTTCTTGACCTAGAACCAAGGCATGAA
GAACTCACAGACACTGGAGGGTGGAGGGTGGGAGAGACCAGAGCTACCTGTGCACAGGTACCCACCTGTC
CTTCCTCCGTGCCAACAGTGTCCTACCAGCAAGGGGTCCTGTCTGCCACCATCCTCTATGAGATCCTGCT
AGGGAAGGCCACCCTGTATGCTGTGCTGGTCAGCGCCCTTGTGTTGATGGCCATGGTAAGCAGGAGGGCA
GGATGGGGCCAGCAGGCTGGAGGTGACACACTGACACCAAGCACCCAGAAGTATAGAGTCCCTGCCAGGA
TTGGAGCTGGGCAGTAGGGAGGGAAGAGATTTCATTCAGGTGCCTCAGAAGATAACTTGCACCTCTGTAG
GATCACAGTGGAAGGGTCATGCTGGGAAGGAGAAGCTGGAGTCACCAGAAAACCCAATGGATGTTGTGAT
GAGCCTTACTATTTGTGTGGTCAATGGGCCCTACTACTTTCTCTCAATCCTCACAACTCCTGGCTCTTAA
TAACCCCCAAAACTTTCTCTTCTGCAGGTCAAGAGAAAGGATTTCTGA
[SEQ ID NO: 70]
AGGACCTGAAAAACGTGTTCCCACCCGAGGTCGCTGTGTTTGAGCCATCAGAAGCAGAGATCTCCCACAC
CCAAAAGGCCACACTGGTATGCCTGGCCACAGGCTTCTACCCCGACCACGTGGAGCTGAGCTGGTGGGTG
AATGGGAAGGAGGTGCACAGTGGGGTCAGCACAGACCCGCAGCCCCTCAAGGAGCAGCCCGCCCTCAATG
ACTCCAGATACTGCCTGAGCAGCCGCCTGAGGGTCTCGGCCACCTTCTGGCAGAACCCCCGCAACCACTT
CCGCTGTCAAGTCCAGTTCTACGGGCTCTCGGAGAATGACGAGTGGACCCAGGATAGGGCCAAACCCGTC
ACCCAGATCGTCAGCGCCGAGGCCTGGGGTAGAGCAGGTGAGTGGGGCCTGGGGAGATGCCTGGAGGAGA
TTAGGTGAGACCAGCTACCAGGGAAAATGGAAAGATCCAGGTAGCGGACAAGACTAGATCCAGAAGAAAG
CCAGAGTGGACAAGGTGGGATGATCAAGGTTCACAGGGTCAGCAAAGCACGGTGTGCACTTCCCCCACCA
AGAAGCATAGAGGCTGAATGGAGCACCTCAAGCTCATTCTTCCTTCAGATCCTGACACCTTAGAGCTAAG
CTTTCAAGTCTCCCTGAGGACCAGCCATACAGCTCAGCATCTGAGTGGTGTGCATCCCATTCTCTTCTGG
GGTCCTGGTTTCCTAAGATCATAGTGACCACTTCGCTGGCACTGGAGCAGCATGAGGGAGACAGAACCAG
GGCTATCAAAGGAGGCTGACTTTGTACTATCTGATATGCATGTGTTTGTGGCCTGTGAGTCTGTGATGTA
AGGCTCAATGTCCTTACAAAGCAGCATTCTCTCATCCATTTTTCTTCCCCTGTTTTCTTTCAGACTGTGG
CTTCACCTCCGGTAAGTGAGTCTCTCCTTTTTCTCTCTATCTTTCGCCGTCTCTGCTCTCGAACCAGGGC
ATGGAGAATCCACGGACACAGGGGCGTGAGGGAGGCCAGAGCCACCTGTGCACAGGTGCCTACATGCTCT
GTTCTTGTCAACAGAGTCTTACCAGCAAGGGGTCCTGTCTGCCACCATCCTCTATGAGATCTTGCTAGGG
AAGGCCACCTTGTATGCCGTGCTGGTCAGTGCCCTCGTGCTGATGGCCATGGTAAGGAGGAGGGTGGGAT
AGGGCAGATGATGGGGGCAGGGGATGGAACATCACACATGGGCATAAAGGAATCTCAGAGCCAGAGCACA
GCCTAATATATCCTATCACCTCAATGAAACCATAATGAAGCCAGACTGGGGAGAAAATGCAGGGAATATC
ACAGAATGCATCATGGGAGGATGGAGACAACCAGCGAGCCCTACTCAAATTAGGCCTCAGAGCCCGCCTC
CCCTGCCCTACTCCTGCTGTGCCATAGCCCCTGAAACCCTGAAAATGTTCTCTCTTCCACAGGTCAAGAG
AAAGGATTCCAGAGGCTAG

In certain embodiments, the first antigen binding chain or the second antigen binding chain comprises a constant domain comprising a native or modified TRGC polypeptide. In certain embodiments, the TRGC polypeptide is a native or modified TRGC1 polypeptide. In certain embodiments, the TRGC1 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 71, which is provided below. In certain embodiments, the TRGC1 polypeptide comprises or consists of the amino acid sequence set forth in SEQ ID NO: 71.

[SEQ ID NO: 71]
DKQLDADVSPKPTIFLPSIAETKLQKAGTYLCLLEKFFPDVIKIHWQEKKSNTILGSQEGNTMKINDTYM
KFSWLTVPEKSLDKEHRCIVRHENNKNGVDQEIIFPPIKTDVITMDPKDNCSKDANDTLLLQLTNTSAYY
MYLLLLLKSVVYFAIITCCLLRRTAFCCNGEKS

In certain embodiments, the TRGC polypeptide is a native or modified TRGC2 polypeptide. In certain embodiments, the TRGC2 polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 72, which is provided below. In certain embodiments, the TRGC2 polypeptide comprises or consists of the amino acid sequence set forth in SEQ ID NO: 72.

[SEQ ID NO: 72]
DKQLDADVSPKPTIFLPSIAETKLQKAGTYLCLLEKFFPDIIKIHWQEKKSNTILGSQEGNTMKINDTYM
KFSWLTVPEESLDKEHRCIVRHENNKNGIDQEIIFPPIKTDVTTVDPKYNYSKDANDVITMDPKDNWSKD
ANDTLLLQLTNTSAYYTYLLLLLKSVVYFAIITCCLLRRTAFCCNGEKS

In certain embodiments, the TRGC polypeptide comprises or consists of an amino acid sequence that is at least about 85%, about 90%, about 95%, about 96%, about 97%, about 98%, about 99% or 100% homologous or identical to the amino acid sequence encoded by a transcript expressed by a gene of NCBI Genbank ID: 6966, NG_001336.2, range 108270 to 113860 (TRGC1, SEQ ID NO: 73), NCBI Genbank ID: 6967, NG_001336.2, range 124376 to 133924 (TRGC2, SEQ ID NO: 74) or a fragment thereof, and/or may optionally comprise up to one or up to two or up to three conservative amino acid substitutions. In certain embodiments, the TRGC polypeptide comprises or consists of the amino acid sequence encoded by the nucleotide sequence of SEQ ID NO: 73. In certain embodiments, the TRGC polypeptide comprises or consists of the amino acid sequence encoded by the nucleotide sequence of SEQ ID NO: 74. SEQ ID NO: 73 and 74 are provided below.

[SEQ ID NO: 73]
ATAAACAACTTGATGCAGATGTTTCCCCCAAGCCCACTATTTTTCTTCCTTCAATTGCTGAAACAAAGCT
CCAGAAGGCTGGAACATACCTTTGTCTTCTTGAGAAATTTTTCCCTGATGTTATTAAGATACATTGGCAA
GAAAAGAAGAGCAACACGATTCTGGGATCCCAGGAGGGGAACACCATGAAGACTAACGACACATACATGA
AATTTAGCTGGTTAACGGTGCCAGAAAAGTCACTGGACAAAGAACACAGATGTATCGTCAGACATGAGAA
TAATAAAAACGGAGTTGATCAAGAAATTATCTTTCCTCCAATAAAGACAGGTATGTGTTTACGCATATCA
TCTGTCAGAACACTTCTTTGAAAGTGAATGCTGCATTTTTTCCTTTCAGTATTAATGAAAAACAAACATA
AATCTTTCTTAAATATTGTTACATTTAATGGTAGCATAAATGCCCTGCTACTTTTCTATAGAATTAAAAT
GGTATAGGTTTTGGAGAAAACAAAATTGAAAAAGTTACTGAAGGTTTGTCAGCCTCAGCTCCATTATCCA
AAATAAGAAAGTCACGTGCTGGTTTTTAGGGTTGTTAGATGGATTAAAGAAACAACATACACAGAAGCAT
CTAGCAACGTGACACGTGGTAAACGCTCAAAAAGTGTTCTCCCTTCTTTTGATGACTTTACTTGATCAGG
AAATAACATATATATGTCTTTCAGGAATGTTCTGCCCAAGCAGGAGAGTCACTCACCTCAATCTTGCTAC
CCACAAAGTTTAACCTAAAAACAACGGGTTCATTGTTGACAAAATGATGTTTATCTGTTGTTGACAGAAT
GATGTTTATCTAAAAACAGTTCCAATTTTCTATTTCCTTTGCTGAGACACAAAGGGGAGGCAAATGTGCA
AAGCTTGAGGGTAGTCTTACCACTGTGCTTAAGTGTTCTGATTTTTCTAGTGATCAGGGCAAAATAAAAA
GTATAGTAAGTTCCAAGGCAGTGAATATTATACAGGAGAGAAGTTACAGTTTTATAATGTGTTTTCCTTT
ACACTAAATTCTAAAAGTAAAAAGTCTTTTTTTTTTTTTGACAGAGTTTCACTCTTGTTGCCCAAGCAGG
TGTGCTATGGTATGATCTCAGCTCACTGCAACCTCCACCTCCCGGGTTCAAGTGATTCTCTTACTTCAGC
CTCCCGACAGGCTGGGATTGCAGGCGCCTGCCACCACACCTGGCTAATTTTTGTGTTTTTAGTAGAGATG
GGGTTTCACCATGTTGGCCAGGCTGGTCTCAAATTCCTGACCTCAAGTGATCCATCCACCTCGGCCTCCA
AGTGCTGGGATTATGGGCGTCAGCCACTGTGCCCAGCCTAAAAGTAAAATGTCTTTCATGAGCTTCCCAA
GGCAGCTACGTTAAGGAGGACACTTCTCTTAATGTCATTCTACAGTAGATTTCTAATGCTCTTTCTTGGA
AGTTTGTTTTTCTGAGAAAAGCTAAAAATATAACATGGAAGTGATCATATTATATAATCAATGAAGTGCT
TTTCAAGGAGATAAAACTAATCTGGTCCACACTTGCAACCAACCTTGATTGAGAGAGAGAGAGAACTCAG
GATACACTTGAAGATTTTATTATGGGGAACAGTTACTTTATTCTTTTTACCTCAATCAATGCATGGAAAT
AAGTGATAGTCATTTTCATTTATCTTTTAATAAATGAAGTCACCATGAGGAAAATAAAAAGACATTGAAA
ACCCATTAAAGTCAGCCCTTAAAGATATTTGGACATGCAGACTTGATAACTAACGTTTGCATTCTTGAGA
CTTACCCAAAACCCATACCTCAAGTCCAAGTTTTTAGAATTCATGAAATAAAGATCTCAGTGAGTGCATA
AAATTGCGCACCAGAATCATATCCGTATAGACAAGAACACATCTACTAGAAAAATAATAAACCAACACAC
CAATGCAACTGTGTTTTCTTCTGTTTTAAAGTATGTTGTCTTTGTATGCATGTTTGCTTCTTCCTTTTTT
TTTTTAACATCACAGATAAATTCAACTCTCACCTCAGGTTTTATTGAGAGAACTGTCAATGTGACTTGGC
CTCTGTCTTTCTAGTCCCAGAAAGAATTGCACTGAAATCTGAGCTCCTGTAATAAAAACAACCATTTGCT
GAGAGTAATTAACATACTGAAAGAGATTTTCTTAGAGTACACAATGGTGACATTATATTGCCTCTTTATA
AATAACTTTCTATCTATTTCTGTGGATTATTCCTACAAAGTACTTTTCATATGTCCAATTTCTTTTCTTC
CCCTACAACTACTGTCTGAATACTGGCTCTGCTATTTGCTGATATGATTCTCGGCAAGTTGCCTGCACTT
TTTAAACTTTATTTCCTCATTCAGAACATGGGGCCATACATAATACAACTCACTTCAGTGTTATTGGGGA
ATTAAACAAAAAATGCATGGGAAGCATTTAACATAGTGCCTGACACAATAATGAGTACTCAGTAGATGTT
AGCTTTTATTAATATTGTTGTTGTTATGTCCAGAAACACTATACCTCCAGAAAATCATGGGTACTTGCTG
GGGACATTGGGGATATGCATGATTTGGAAAAGAATGACTGCTTTTTTTGCTTAGATGAGAAATTTTTCTA
AGCCAGACTCCTTCAAATATGTAAGATTCTGTTGTGGATTCAAGGACTGAAAGAATTCTTGGCCGAGTGT
GGTGGCTTATCCCTGTAATCCCAGCATTTTGTGAGGACAAGGCAGGAAGATTGCTTGAGTCCAGGAGTTT
GAAACCAGCCTGCGCAACATGGCGAAACCCTGTCTCTACAAAAAATACAAACATTAGCTCGGAGTGAGTG
CTGACATGTGCCTGTACTCCCAGCTACTCAGAAGGCTGAGATGGGAGGATCTCATGAGCCTGGGGAGTTT
GAGGCTTCAGTGAGCCGTGATGACACCGTACTATACTCCACTCCAGCCTGGGTGACAGTGAGACCCTGCC
TCAAAAAACAAACAAACAAACAAACAAAACAAAATTAATCTTTTTGCTGATGTCATGTCAGCAGTGTGTG
TTGAAGGCTGTAAAGCAGCCATTTGTTCAGTTTATTTTTCCATTGAACAAGTATTTATCAAAAACATACT
TTGTGGCAGTCACTATGCTAGGAGCTATGAATACAGAAGGAAAAGTAAATGCTCTTGGATACTACACTCC
AGTIGTGATAAAAAAGAAAAAATGTATTCTTCACCAACTTCAACATCTTGATGTGCAAAAACATAATACA
TGAATTAGATCTACCTAATTACACAGAATTAGACCAATTGTTTCTGGAATTGTGGGCTCATATTTTTAAT
AACTGTCCTCCTGCCTCTCTGTCGACAGGTTTTATAAATATTCATTTAATTACACACACACACACGAACA
ATTGACTAGTACTTGCTCTCATTCTTCTAGATGTCATCACAATGGATCCCAAAGACAATTGTTCAAAAGA
TGCAAATGGTAAGCTTTTGTGTTTTTCCCTTCCTCCTGATCATTTTGTTTTGAACTTCTCTGGCTTGAAA
AATCAGGGAATGGATTTTGCTAGGTTGGATGCTGCAGAATGGACCTAGTGATATTTTAAATTAGTCCCTC
ATTTTCTAGGAGTTGTATTAACAAACCTAACTACTGCTTTGGGGTATGAGATGACTGTAAATTAGAGAGG
GTACAGTGGTATAGTGATATGCTTTTAATTATTTCAAAAAAAAGATTTTATTCATTCATGTGTCTTTTTT
CTTTTTCTTTTCTTTTTTTTTTTTTTTTGGACAGAGTCTTGCTCTGTCACCCAGGCTGGAGTGCGGTGGC
AGTATCTCAGCTCACCACAACCTCCGCCTCCCGGCTTCAAGTGATTCTCCTGCCTCAGCTTCTCGAGTAG
CTGGGACTACAGGCGCGTGCCACCATGCCCGGCTAATTTTTGTATTTTTAGTAGAGTTGGGGTTTCACCA
TGTTGGCCAGGATGGCCTCGAATTTGTGACCTCGTGATCTGCCCCCTCGCCCTCCCGAACTGTTGGGATT
ACAGGCGTGAGTCACTGTGCCCGGCCTCCTGTCCTGTCTTTTGTTTAATGACTGGGAAAAACATGATACC
ATGTTGCTTCTCGAGTTGTTTTGTTTTAGTCTTTGGTCTTTGCTAGTAGCTAATAACACGAACTAGTGTT
TATCAAGTGCTTTTTACACAGAAGGGCTTGGGCTGTGTTCTGCATTTTCTTGTTTAACCCTCTTAAAACT
CCTATAAAATGGTACATATTTTTCTCCCAATTTACAGTCCCTTTAAAGCAAATAATTATAAAAATCCCTA
TACATGTCACACAGCTAGATCTGGGATTTCAAATCAGGCC
ATCAAACAAAGAGTTTATGTACTTAGTAAGTTTTCTGTTCTTTTTCTACAATAGAGTCAGATAGCAAGAA
ATTACCAAGCCAGGAACCTGAAACAAAACGGACATCATGTGGGGCTGGGTGGGTGCATGGGCTTTGCAGA
CTGGACTTTCACTCCAGCTCTTTTAATGATTAGGTGTAAGTGACCTACATTTTGTGAGCAACAGTTTTCT
CATCAGCCAACAAAGAATAATTACACCAGATTCACAGTTATTGAAGAGATAAAGGCATGAATGTGAGATG
TCTGGCATAGGGCATCTCATTTAGCAGACACAGAATGAGTACTTGTTTCTGGCTTTTTCTCTCTACATAT
GCACAAAGAATGCGACTAGAAGCATGGGCTCTAGCCCTGCTCAACTTTCCTCTATTTCCAATACCAAGGG
GCTCTGACTTAGGCTGCCACACCAGGCAAGGAGGGCAGTACCACCTCACTTGACCAAGGGCAGGGAGTCA
CGGACACATCACTTCTTGAGATCCTTTTCCACACCAAGGACTGATGTTTCTGGAATTCTCACTTTATGAA
GACAAAACATATAAATGGAAATTTTCTCAGGTAGAGACTCACTCTTGTAGCTCATTGAGTAGGCACTAGT
GGTCCACCCCCACTGTCTTTACTTATTCCTTGACATCACATATCTCTTGCAAAACCTCAAATAATATTAA
ATGCAATCACCCAATAATAGCATAGCCATAATTAGAGGCATTTAGGAAAGACAGGTGAGTGTGCCACAAC
TACCTAACACATCAGCAAATCTGGATTAACCACTTTCTTTGATTTTCCACAATGCAACCTTACTTTTTAA
TAGTTGGGAATGTTCTAAGTGAATTTAGCAGAGGTTGTTAATCAACTTGAAAGCTGAATTCTGACTTGTC
TGACTCTTGGTGGTGCTGGTAGCAGTAGATGTTTACTTTTAGGTTTTGGTGGTGGTGGAATATCACTTCA
ACGTAAATCATCAGAAATAAGTATTTGTGAACCCCTCTCGCATTAATGTATCTTATTCTGTAAAAAGAAC
ATGTGCAATTTCTCTTAGATACACTACTGCTGCAGCTCACAAACACCTCTGCATATTACATGTACCTCCT
CCTGCTCCTCAAGAGTGTGGTCTATTTTGCCATCATCACCTGCTGTCTGCTTAGAAGAACGGCTTTCTGC
TGCAATGGAGAGAAATCATAA
[SEQ ID NO: 74]
ATAAACAACTTGATGCAGATGTTTCCCCCAAGCCCACTATTTTTCTTCCTTCGATTGCTGAAACAAAACT
CCAGAAGGCTGGAACATACCTTTGTCTTCTTGAGAAATTTTTCCCAGATATTATTAAGATACATTGGCAA
GAAAAGAAGAGCAACACGATTCTGGGATCCCAGGAGGGGAACACCATGAAGACTAACGACACATACATGA
AATTTAGCTGGTTAACGGTGCCAGAAGAGTCACTGGACAAAGAACACAGATGTATCGTCAGACATGAGAA
TAATAAAAACGGAATTGATCAAGAAATTATCTTTCCTCCAATAAAGACAGGTATGTGTTTACACATATCA
TCTGTCAGAACACTTCTTTGAAAGTGAATGCTGCATTTTTTCCTTTCAGTATTAATGAAAAACATAAATC
TTTCTTAAAAATTGTTACATTTAATGGTAGCGTAAATGCCCTGCTACTTTTCTATAGAATTAAAATGGTA
TAGGTTTTGGAGAAAACAAAATTGAAAAAGTTGCTGAAGGTTTGTCAGCCTCAGCTCCATTATCCAAAAT
AAGAAAGTCACGTGCTGGTTTTTAGGGTTGTTAGATGGATTAAAGAAACAACATACACAGAAGCATCTAG
CAACGTGACACGTGGTAAACGCTCAAAAAGTGTTCTCCCTTCTTTTGATGACTTTACTTGATCAGGAAAT
AACATATATATGTCTTTCAGGAATGTTCTGCCCAAGCAGGAGAGTCACTCACCTCAATCTTGCTACCCAC
AAAGTTTAACCTAAAAACAACGGGTTCATTGTTGACAAAATAATGTTTATCTGAAGATAACTGTAGATCA
TATTTATCTGTAGATAATGTTTATCTGTGGAGTGTGGCTCTACAAAACATAGAATAGTCTTGGTCACTGC
AGTTTTATAGAGGCCTTGGGTTTTTCAGAGTTTCATTTTATATATCACCATAAAGTAACATTTCATAATT
ACAGGTTGGTAAGGCTTACATGTACAAACATTCTTCCATTTTCCATAATAAATGCATTTCCTGCCATTGG
TGAATGCAGCTCAATAAACATTTATTGTACAATTATGACACGCCAGGCTTAGTGGAAATGTGGATGAACA
GACAAGGATGAGTTACTGTCCTAAGGATGATGCATGACAGTGCAGAGAATATACTCTCTTCCTGATCACT
CAGGGTCACTCATGATTCATGCGCGAGGTCCCAAAACAGTGCCTTTGATGCAGATTCTGTACATCTCTAG
ACGATTGGTCCAAGGGCTGAATGTGCTCTGGCCCAGTGGTCCAGTCTGTCACTATATGTCAACATCCTGA
ATATGAACATAACAGTCCAACATCTCAAGAGTGGGCATGAAAAGGACTCATTTTGTGCTTTTTCCTGTGG
TTAACAAGTCCTTTTTAGCCTGGGGGAACAAGCATTAACAAAATGTTTGAAGATCTTTGCCACGTACCAT
TCCAAATTTCTAGGGTAAGTCTTTAGCTTTTCAGATCCTGAGTTTCTGCAATGATCAAATGTGATTTGGA
CAGTTGCGTTGACTTTCTCCTGGGGCTATAATGGAGTGCAAAGGAAACAATGGCAGGGAAAATGCTTGCT
TTCAAAATGGTAGCATGGATGTGTTCATTCGTGTAGTTACTGTATTAGGTATAGCCTTTCCTGAAACTAA
CTGAAGTGGGGTTATAAAAACAGTCCCAATTTTCTATTTCCTTTGCTGAGACACAAAGAGGAGACAAAAG
AGCAAAGCTTGAGGGTAGTTTTACCACTGTGCTTAAGTGTTCTGATTTTTCCAGTGATCAGGGTGAAATA
AAAAGCATAGTAAGTTCCAGGGCAGTGAATACCATACAGGAGACAAGTTACAGTTTTATAATGTGTTTTA
CTTTACACTAAATTCTAAAAGTAAAATGTCTTTTTTTTTTTCCGAGACAGAGTTTCACTCTTGTAGCCCA
GGCAGGAGTGCTATGGTGTGATCTCGGCTCACAGCAACCTCCACCTCCCAGTTTCAAGCGATTCTTCTGC
CTCAGCCTCCCGAGAAGTTGAAATTACAGGTGCCTGGCACCATATCTCGCTAATTATTCTATTTTTAGTA
GAGATCGGGTTTTACCATGTTGGCCAGGCTGGTCTCGAACTCCTGACTTCAAGTGATCCACCCGCCTCAG
CCTCCCAAAGTGCTGGGATTACAGGTGTGAGTCACTGTGCCGGACCTAACAGTAAAATGTCTTTCATGTG
CTTCTCAAGGCAACTACATTAAGGAGGACACATCTCTTAATGTCATTCTACAGTAGATTTCTAATGCTCT
TTCTTGGAAGTTTGTTTTTCTGAGAAGAGCTAAAAATATAATAACATGGAAGTGATCATATTATATAATC
AATGAAGTGCTTTCAAAGGAGATAAAACTAACCTGGTCTGCATTTGCAACCAGCCTTGATTGAGAGAGAG
AGAACTCAGGATACACTTAGAGATTTTATTATGGGGAATAGTTACTTTATTCATTTTACCTCAATCAATG
CATGGAAATAAGTGACAGTCATTTTCATTTATCTTTTAATAAATAAAGTCACCATGAGGAAAATGAAAAC
CCATTAAAGTCAGTCCTTAAAGATATTTGGACATGCAGACATGATAACTAACATTTCCATTCGTGAGACT
TACCCAAAACCTATACCTCAAGTCCATTTCTTAGAATACATGAAATAAAGATCTCAGTGAGTGTATAAAA
CTGCACACCAGAATCATATCCGTATAGACAAGAATACATCTACTAGAAAAATATAAACCAAAACACCAAG
GTGACTCTGTTTTTTTCTGTTTTAAAATATGTTGTCTTTGTATGCATGTTTGCTTCTTCCTTTTTTTTTT
TAAACATCGCAGATAAATTCAACTCTCACCTCAGTTGAGAGAGAACTGTCAATGTGACTTGGCCTCTCTC
TTTCTAGTCCCAGAAAGAATTGCACTGAAATGCTGAGCTCCTGTAATAAAAATGACCATTTGCTGAGAGT
AATTAACATACTGAAAGAGATTTTCTTAGAATAGTGCACAATGGCCCAATGGTGACATTATATTGTCTCT
TTATAAATTATTTTCTATCTATTTCTGTGGATTATTTCTACAAAGCACTTTTCATATGTCCAATTCCTTT
TATTCCCCTACAAGTACTGACTGACTACTGGCTCTGCTGTTCACTGATATGACTTTCGGCAAGTTGCCTG
CACTTTTTAAACGTTATTTCCTCATTCAGAACATGGGGCCATACAAAATACAACTCACTTCAGTGTTATT
GGGGAATTAAACAAATAAATGCATGGGAAGCATTTAACATAGTGCCTGACACAATAATGAGCACTCAGTA
GATGTTAGCTTTTATTAATATTGTTGTTGCTATGTCCAGAAACACTATACCTCCAGAAAATCATGGGTAC
TTGCTGGGGACGTTGGGGATATGCATGATTTTGAAAGGAGTGACTGCTCTTTACTGCTCAGATGAGAAAT
TTTTCTAAGCCAGACTCCTTCAAACATGTAAGATTCTGTTGTGGATTCTAGGACTGAAAGAATTCTTGGC
CGAGTGTGGTGGCTTATCCTGGTAATCTCATCATTTGGGAGGACAAGGCAGGAAGATTGCTTGAGCCCAG
GAGTTGGAAACAAGCCTGGACAACATGGCGAAACCCTGTCTCTACAAAAAATACAAACATTAGCTGGTCA
TGGGAGTGAGTGCCTGTACTCCCAGCTACTCAGGAGGCTAAGATAGGAGGATCACCTGAGCCTGGGCAGT
TTGAGGTTTCAGTGAGCCGTGATGACACCATACTATACTCCACTCCAGCCTGGGTGACAGTGACATCCTG
CCTCAAAAAAACCCCCAAAATTATTCTTTTTGCTGATTTCATGTCAGCAGTGTGTGCTGAAGGCTGTAAA
GTAGCCACTTGTTCTGTTTATTTTTCCATTGAACAAGTATTTATCAAAAACGTACTTTGTGGAAGGCACT
GTGCTAGGAACTATGCATACAGAAGGAAAACCAAATGTTCTTGGATACTACACTCCAGTTGTGATAAAAA
AGAAAAAAGTATTCTTCACAAACTTCAACATTTTGATGTGCAAAAACATAATATATGAATTAGATCTACC
TAACTACACAGAATTAGACCAATTATTTCTGGGATTATGGGCTCATATTTTTAATAACTGTCCTCCTACC
TCTCTGTTGACAGGTTTTATAAATATTCATTTAATTACACACAGTCACAGACACACTCAGACACACACAC
ATACACACACACACACACCTTGACAAATAATGGGCATGAACAATTGACTGGTACTTGCTCTCATTCTTCT
AGATGTCACCACAGTGGATCCCAAATACAATTATTCAAAG
GATGCAAATGGTAAGTTTTTGTGTTTTTTATTTCCTCCTGATCATTTTAAGTTTTGAACTTCTCTGGCTT
GAAAAATCAGGGAATGGATTTTGCTAGGTTGGATGCTGCAGAATGGACCTAATCATATTTTAAATTAGTC
CCTCTTTTTCTAGGAGTTGTATTAACAAACCTAACTACTGCTTCATGTAAGAGATGACTGTAAATTGAAG
GGTACAGTGATATGCTTTCAGTTATTTCAAAAAACAGACTTTACTCATCCATGTGTCTTTTTTCTTTTCT
TTTTTTTCTTTTTTGAGACGGAGTCTCGCTCTGTTGAACAGGCTGGATTGCAGTGACGCGATCTCACCTC
ACTACAACCTCCGCCTCTGGAGTTCAAGCGATTCTCCAGCCTCAGCTTCTCAAGTAGCTGGGACTACAGG
CACATGCCACCATGTCCGGGTCATCTTTGTATTTTTAGCAGAGACCGGGTTTCACTATGTTGGCCAGGCT
GGTCTAGAATTCCTGACTTCGTGATCTGCCCCCTCAGCCCTCCGAAGTGCTGGGATTACAGACGTGAGTC
ACTGTGCCCGGCCTAACAGTAAAATGTCTTTCATGCGCTTCTCAAGGCAACTACGTTAAGGAGGACACTT
CTCTTAATGTCATTCTACAGTAGATTTCTAATGCTCTTTCTTGGAAGTTTGTTTTTCTGAGAAAAGCTAA
AAATATAACATGGAAGTGATCATATTGTATAATCAATGAAGTGCTTTTCAAGGAGATAAAACTAATCTGG
TCCACGTTTGCAACCAACCTTGATTGAGAGAGAGAGAGAACTCAGGATACACTTGGAGATTTTATTATGG
GGAATAGTTACTTTATTCTTTTTTCCTCAATCAATTCATGGAAATAAGTGATAGTCATATTCATTTATCT
TTTAATAAATGAAGTCACCATGAGGAAAATAAAAAGACATTGAAAACCCATTAAAGTTAGCCCTTAAAGA
TATTTGGACATGCAGACTTGATAACTAACGTTTGCATTCTTGAGACTTACCCAAAACCCATACCTCAAGT
CCATGTTTTTAGAATTCATGAAATAAAGATCTCAGTGAGTGCATAAAATTGCGCACCAGAATCATATCCG
TATAGACAAGAACACATCTACTAGAAAAATAATAAACCAACACACCAATGCAACTGTGTTTTCTTCTGTT
TTAAAATATGTTGTCTTTGTATGCATGTTTGCTTCTTCCTTTTTTTTTTTTAACATCACAGATAAATTCA
ACTCTCACCTCAGGTTTTATTGAGAGAACTGTCAATGTGACTTGGCCTCTGTCTTTCTAGTCCCAGAAAG
AATCGCACTGAAATGCTGAGCTCCTGTAATAAAAATGACCATTTGCTGAGAGTAATTAACATACTGAAAG
AGATTTTCTTAGAGTACACAATGGTGACATTATATTGTCTCTTTATAAATAACTTTCTATCTATTTCTGT
GGATTATTCCTACAAAGTACTTTTCATATGTCCAGTTTCTTTTCTTCCCCTACAACTACCGTCTGAATAC
TGGCTCTGCTATTTGCTGATATGATTCTCGGCAAGTTGCCTGCACTTTTTAAACTTTATTTCCTCATTCA
GAACATGGGGCCATGTAATACTCATGTACGTGAGTATTACGTAATAATGCTCACTTAAGTGTTACTGGGG
AATTAAACAAAAAAATGCATGGCAAGCATTTAACATAGTGCCTGACACAATAATGAGCACTCAGTAGATG
TTAGATTTTATTAATATTGTTGTTGTTATGTCCGGAAACACTATACCTCCAGAAAATCATGGGTACTTGC
TTGGGATGTTGGGGATATGCATGATTTGGAAAGGTATGACTGCTTTTTTCTGCTTAGATGAGAAATTTTT
CTAAGCCAGACTCCTTCAAATATGTAAGATTCTGTTGTGGATTCTAGGACGGAAAGAATTCTTGGTCAGG
TGTGGTTTCTTATCCCTGTAATCCCAGAATTTTGGGAGGACAAGGCAGGAAGATTGCTTGAGCCCAGGAG
TTTGAAACCAGCCTGGGCAACAAGACGAAACCCTGTCTCTACAAAAGTACATAAATTAGCTTGGCTTGGT
GGTGTGTGCCTGTATTACCAGCTATTCGGGAGACTGAGATGGGAGGATCTCCTGAACCTGTGAAGTTTGA
GGCTTCAGTGAGCCGTGATGACACCATACTATACTCGACTCCAGCCTGTGCGACAGTGAGACTCTGCGTC
AAAAAAAAAACCCCAAAATTATTGTTTTTGCTGATTTCAGGTCAGCAGTGTGTGCTGAAGGGTGTAAAGT
AGCCACTTGATCAGTTTATTTTTCCACTGAACAAGTATTTATCAAAAACATACTTTGTGGTCTGTTTTTG
ATAAATAAAAAGGCACTGTGCTAGGAGCCATGAATACAGAAGGAAAACCAAATGTTCTTGGATACTACAC
TCCAGTTGTGATAAAAAAGAAAAATGTATTCTTCACGAACTTCAACATTTTGATATGCAAAAACATAGTA
TATAAATTAGATCTACCTGATTACGTAGAATCAGACCAATTATTTCTGGAATTGAGGGCTCATATTTTTA
ATAACTGTCCTCCTGCCTCTCTGTTGACAGGTTTTATAAATATTCATTTAATTACACACACACACACACA
CACCTTGACAAATAATGGACATGAACAATTGACTAGTACTTGCTCTCATTCTTCTAGATGTCATCACAAT
GGATCCCAAAGACAATTGGTCAAAAGATGCAAATGGTAAGCTTTTGTGTTTTTCCTTTCCTCCTGATCAT
TTTAAGTTTTGAACTTCTCTGGCTTGAAAAATCAGGGAATGGGCCGGGTGCGGTGGCTCACGCCTGTAAT
CCCAGCACTTTGGGAGGCCGAGGCGGGCGGATCACGAGGTCAGGAGATCGAGACCATCCCGGCTAAAACG
GTGAAACCCCGTCTCTACTAAAAATACAAAAAATTAGCCGGGCTTAGTGGCGGGCGCCTGTAGTCCCAGC
TACTTGGGAGGCTGAGGCAGGAGAATGGCGTGAACCCGGGAGGCGGAGCTTGCAGTGAGCCGAGATTGCG
CCACTGCACTCCACTCCAGCCTGGGCGACAGAGCGAGACTCCGTCTCAAAAAAAAAAAAAAAAAAAAAAA
AAGAAAAATCAGGGAATGGATTTTGCTAGGTTGGATGCTGCAGAATGGACCTAGTGATATTTTAAATTAG
TCCCTCTTTTTCTAGGAGTTGTATTAACAAACCTAACTACTGCTTCGGGTATGAGATGACTGTAAATTAG
AGGGTACAGTGATATGCTTTCAGTTATTTCAAAAAACAGACTTTATTCATCCGTCTGTCTTTTTTTTTTT
TTTTTTTTTTTTTTTTTGAGACGGAGGAGTCTCACTCTATCACCCAGGCTGGAGTGCAGTGGCGCGATCT
CGGCTCACCATAACCTCCGCCTTACTGGTTCAAGCGATTCTCCAGCCTCAGCTTCTCAAGTAGCTGGGAC
TACAGGTGCACACCACCATACCTGGCTAATTTTTGTATTTTTAATAGAGATGGGGTTTCACCACGCTGGC
CAGGATGGTCTTGAATTCTTGACCTCGTGATCTGCCCCCTCGGGCTCCCAAACTTCTGGGATTATAGGCG
TGAGCCACTGTGCCCGGCCTTCTGTCTTTTGTTATAATGACTGGGGAAAACATGATACCATGTTGCTTCT
TGAGTTGTTTTGTTTTAGTCTTTGGTCTTTGCTAGTAGCTAATAACACGAACTAGTGTTTATCAAGTGCT
TTTTACACAGAAGGGCTTGTTCTGCATTTTCTAGTTTAATCATCTTAATACTCCTATAAAGTAGTACAAT
ATATTTTCTCCCATTTTACAGTCCCTTTAAAGTAAATAACTATAAAAATCCCTTATACATGTCACACAGC
TAGGTCTGGCATTTCAAATCAGGACATCAAACAAAGAATTCGTGCAGTTACTAAGTCCTCTATTTTTTCT
ACAATAGAAAAAATAGCAAGAATTACAGATAGCAAGACATTACAAGGCAGGAATCTGAAACGAAAGGGAC
ATAATGTGGGGCTGGGTGGGTGCATGAGCTTTGCAGACTAGACTTTCATTCCAGCTCTTTTAATGATTAG
GTGTAAGTGACCTACATTTTGTGAGTAACAGTTTTCTCATCAGCCAACTAAGAATAATTACACCAGATTC
ACAGTTATTGAAGAGATAAGGGCATGAATGTGAGATGTCTGGCGTAGGGTATCTCATTTAGCAGACACAG
AATGAATACTTGTTTCTGGCTTTTTCTCTCTACATATGCACAAAGAATGTGACTAGAAGCATTGGCTCTA
GCCCTGCTCAACTTTCCTCTATTTCCAATACCAAGGGGCT
CTGACTTAGGCTGCCACACCAGGCAAGGAGGGGCAGTACCACCTCACTTGACCAAGGGCAGGGAGTCACG
GACACATCACTTCCTGAGATCCTTTTCCACACCAAGGACTGATGTTTCTGGAATTCTCACTTTATGAAGA
CAAAACATATAAATGGAAATTTCTGCAGGAAGAGACTCACTCTTGTAGCTCATTGAGTAGGCACTAGTGG
TCCACCCCCACTGTCTTTACTTATTCCTTGACATCACATATCTCTTGTAAAACCTCAAATAATGTTAAAT
GCAATCACCCAATAATAGCATAGCCATAATTAGAGGCATTTAGGAAAGACAGGTGAGTGTGCCACAACTA
CCTAACACATCAGCAAATCTGGATTAACCACTTTCTTTGATTTTCCACAATGCAACCTTACTTTTTAATA
GTTGGGAATGTTCTAAGTGAATTTAGCAGAGGTTGTTAATCAACTTGAAAGCTGAATTCTGACTTGTCTG
ACTCTTGGTGGTGCTGGTAGCAGTAGATGTTTACTTTTAGGTTTTGGTGGTGGTGGAATATCACTTCAAC
GTAAATCATCAGAAATAAGTATTTGTGAACCCCTCTCGCATTAATATATCTTATTCTGTAAAAAGAACAT
GTGCAATTTCTCTTAGATACACTACTGCTGCAGCTCACAAACACCTCTGCATATTACACGTACCTCCTCC
TGCTCCTCAAGAGTGTGGTCTATTTTGCCATCATCACCTGCTGTCTGCTTAGAAGAACGGCTTTCTGCTG
CAATGGAGAGAAATCATAA

In certain embodiments, the first antigen binding chain or the second antigen binding chain comprises a constant domain comprising a native or modified TRDC polypeptide. In certain embodiments, the TRDC polypeptide comprises or consists of an amino acid sequence that is at least about 85%, about 90%, about 95%, about 96%, about 97%, about 98%, about 99% or 100% homologous or identical to the amino acid sequence set forth in SEQ ID NO: 75, which is provided below. In certain embodiments, the TRDC polypeptide comprises or consists of the amino acid sequence set forth in SEQ ID NO: 75.

[SEQ ID NO: 75]
SQPHTKPSVFVMKNGTNVACLVKEFYPKDIRINLVSSKKITEFDPAIVI
SPSGKYNAVKLGKYEDSNSVTCSVQHDNKTVHSTDFEVKTDSTDHVKPK
ETENTKQPSKSCHKPKAIVHTEKVNMMSLTVLGLRMLFAKTVAVNELLT
AKLFFL

In certain embodiments, the TCR-like fusion molecule comprises a hinge/spacer region that links the first antigen binding chain to the constant domain. In certain embodiments, the TCR-like fusion molecule comprises a hinge/spacer region that links the second antigen binding chain to the constant domain. The hinge/spacer region can be flexible enough to allow the antigen binding chain to orient in different directions to facilitate antigen recognition. In certain embodiments, the hinge/spacer region can be the hinge region from IgG1, the CH₂CH₃ region of immunoglobulin and portions of CD3, a portion of a TCRα polypeptide, a portion of a TCRβ polypeptide, a portion of a CD28 polypeptide, a portion of a CD8 polypeptide, or a synthetic spacer sequence. In certain embodiments, the hinge/spacer region comprises a portion of a TCRα polypeptide. In certain embodiments, the hinge/spacer region comprises a portion of the variable region (TRAV), a portion of the diversity region (TRAD), a portion of the joining region (TRAJ), a portion of the constant region (TRAC), or a combination thereof. In certain embodiments, the hinge/spacer region comprises a portion of the TRAJ region and a portion of the TRAC region of the TCRα polypeptide. In certain embodiments, the hinge/spacer region comprises or consists of the amino acid sequence set forth in SEQ ID NO: 76. In certain embodiments, the hinge/spacer region comprises or consists of amino acids 1 to 3 of the sequence set forth in SEQ ID NO: 76. An exemplary nucleic acid sequence encoding the amino acid sequence of SEQ ID NO: 76 is set forth in SEQ ID NO: 77. SEQ ID NO: 76 and 77 are provided below.

[SEQ ID NO: 76]
IPNIQNPDPA
[SEQ ID NO: 77]
ATTCCCAATATCCAGAACCCTGACCCTGCC

In certain embodiments, the hinge/spacer region comprises a portion of a TCRβ polypeptide. In certain embodiments, the hinge/spacer region comprises a portion of the variable region (TRBV), a portion of the diversity region (TRBD), a portion of the joining region (TRBJ), a portion of the constant region (TRBC), or a combination thereof. In certain embodiments, the hinge/spacer region comprises a portion of the TRBJ region and a portion of the TRAC region (C) of the TCRβ polypeptide. In certain embodiments, the hinge/spacer region comprises or consists of the amino acid sequence set forth in SEQ ID NO: 78. In certain embodiments, the hinge/spacer region comprises or consists of amino acid 1 to 2 of the sequence set forth in SEQ ID NO: 78. An exemplary nucleic acid sequence encoding the amino acid sequence of SEQ ID NO: 78 is set forth in SEQ ID NO: 79. SEQ ID NO: 78 and 79 are provided below.

[SEQ ID NO: 78]
LEDLKNVEPPE
[SEQ ID NO: 79]
CTGGAGGATCTGAAAAACGTGTTCCCTCCTGAA

In certain embodiments, the antigen binding chain does not comprise an intracellular domain. In certain embodiments, the antigen binding chain is capable of associating with a CD3ζ polypeptide. In certain embodiments, the antigen binding chain associating with the CD3ζ polypeptide via the constant domain. In certain embodiments, the CD3ζ polypeptide is endogenous. In certain embodiments, the CD3ζ polypeptide is exogenous. In certain embodiments, binding of the antigen binding chain to a target antigen is capable of activating the CD3ζ polypeptide associated to the antigen binding chain. In certain embodiments, the exogenous CD3ζ polypeptide is fused to or integrated with a costimulatory molecule disclosed herein.

In certain embodiments, the TCR-like fusion molecule comprises an antigen binding chain that comprises an intracellular domain. In certain embodiments, the intracellular domain comprises a CD3ζ polypeptide. In certain embodiments, binding of the antigen binding chain to an antigen is capable of activating the CD3ζ polypeptide of the antigen binding chain.

In certain embodiments, the CD3ζ polypeptide comprises or consists of an amino acid sequence that is at least about 85%, about 90%, about 95%, about 96%, about 97%, about 98%, about 99% or about 100% homologous to the amino acid sequence set forth in SEQ ID NO: 12 or a fragment thereof, and/or may optionally comprise up to one or up to two or up to three conservative amino acid substitutions. In certain embodiments, the CD3ζ polypeptide comprises or consists of an amino acid sequence that is a consecutive portion of SEQ ID NO: 35, which is at least about 20, or at least about 30, or at least about 40, or at least about 50, and up to about 164 amino acids in length. In certain embodiments, the CD3ζ comprises or consists of the amino acid sequence of amino acids 1 to 164, 1 to 50, 50 to 100, 52 to 164, 100 to 150, or 150 to 164 of SEQ ID NO: 35. In certain embodiments, the CD3ζ polypeptide comprises or consists of amino acids 52 to 164 of SEQ ID NO: 35.

In certain embodiments, the TCR-like fusion molecule comprises an antigen binding chain that comprises an intracellular domain, wherein the intracellular domain comprises a co-stimulatory signaling region. In certain embodiments, the intracellular domain comprises a co-stimulatory signaling region and a CD3ζ polypeptide. In certain embodiments, the intracellular domain comprises a co-stimulatory signaling region and does not comprise a CD3ζ polypeptide. In certain embodiments, the co-stimulatory signaling region comprises at least an intracellular domain of a co-stimulatory molecule disclosed herein.

In certain embodiments, the TCR-like fusion molecule is capable of associating with a CD3 complex (also known as “T-cell co-receptor”). In certain embodiments, the TCR-like fusion molecule and the CD3 complex form an antigen recognizing receptor complex similar to a native TCR/CD3 complex. In certain embodiments, the CD3 complex is endogenous. In certain embodiments, the CD3 complex is exogenous. In certain embodiments, the TCR-like fusion molecule replaces a native and/or an endogenous TCR in the CD3/TCR complex. In certain embodiments, the CD3 complex comprises a CD3γ chain, a CD3δ chain, and two CD3ε chains.

In certain embodiments, the CD3γ chain comprises or consists of an amino acid sequence that is at least about 85%, about 90%, about 95%, about 96%, about 97%, about 98%, about 99% or about 100% homologous or identical to the amino acid sequence having a NCBI reference number: NP_000064.1 (SEQ ID NO: 80) or a fragment thereof, and/or may optionally comprise up to one or up to two or up to three conservative amino acid substitutions. SEQ ID NO: 80 is provided below.

[SEQ ID NO: 80]
MEQGKGLAVLILAIILLQGTLAQSIKGNHLVKVYDYQEDGSVLLTCDAE
AKNITWFKDGKMIGFLTEDKKKWNLGSNAKDPRGMYQCKGSQNKSKPLQ
VYYRMCQNCIELNAATISGFLFAEIVSIFVLAVGVYFIAGQDGVRQSRA
SDKQTLLPNDQLYQPLKDREDDQYSHLQGNQLRRN

In certain embodiments, the CD3δ chain comprises or consists of an amino acid sequence that is at least about 85%, about 90%, about 95%, about 96%, about 97%, about 98%, about 99% or about 100% homologous or identical to the amino acid sequence having a NCBI reference numbers: NP_000723.1 (SEQ ID NO: 81) or a fragment thereof, or the amino acid sequence having a NCBI reference numbers: NP_001035741.1 (SEQ ID NO: 82) or a fragment thereof, and/or may optionally comprise up to one or up to two or up to three conservative amino acid substitutions. SEQ ID NO: 81 and 82 are provided below.

[SEQ ID NO: 81]
MEHSTFLSGLVLATLLSQVSPFKIPIEELEDRVFVNCNTSITWVEGTVGT
LLSDITRLDLGKRILDPRGIYRCNGTDIYKDKESTVQVHYRMCQSCVELD
PATVAGIIVTDVIATLLLALGVFCFAGHETGRLSGAADTQALLRNDQVYQ
PLRDRDDAQYSHLGGNWARNK
[SEQ ID NO: 82]
MEHSTFLSGLVLATLLSQVSPFKIPIEELEDRVFVNCNTSITWVEGTVGT
LLSDITRLDLGKRILDPRGIYRCNGTDIYKDKESTVQVHYRTADTQALLR
NDQVYQPLRDRDDAQYSHLGGNWARNK

In certain embodiments, the CD3ε chain comprises or consists of an amino acid sequence that is at least about 85%, about 90%, about 95%, about 96%, about 97%, about 98%, about 99% or about 100% homologous or identical to the amino acid sequence having a NCBI reference number: NP_000724.1 (SEQ ID NO: 83) or a fragment thereof, and/or may optionally comprise up to one or up to two or up to three conservative amino acid substitutions. SEQ ID NO: 83 is provided below.

[SEQ ID NO: 83]
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI

In certain embodiments, the TCR-like fusion molecule exhibits a greater antigen sensitivity than a CAR targeting the same antigen. In certain embodiments, the TCR-like fusion molecule is capable of inducing an immune response when binding to an antigen that has a low antigen density on the surface of a tumor cell. In certain embodiments, cells comprising the TCR-like fusion molecule can be used to treat a subject having tumor cells with a low expression level of a surface antigen, e.g., from a relapse of a disease, wherein the subject received treatment which leads to residual tumor cells. In certain embodiments, the tumor cells have a low antigen density of a target molecule on the surface of the tumor cells. In certain embodiments, a target molecule having a low antigen density on the cell surface has a density of less than about 5,000 molecules per cell, less than about 4,000 molecules per cell, less than about 3,000 molecules per cell, less than about 2,000 molecules per cell, less than about 1,500 molecules per cell, less than about 1,000 molecules per cell, less than about 500 molecules per cell, less than about 200 molecules per cell, or less than about 100 molecules per cell. In certain embodiments, a target molecule having a low antigen density on the cell surface has a density of less than about 2,000 molecules per cell. In certain embodiments, a target molecule having a low antigen density on the cell surface has a density of less than about 1,500 molecules per cell. In certain embodiments, a target molecule having a low antigen density on the cell surface has a density of less than about 1,000 molecules per cell. In certain embodiments, a target molecule having a low antigen density on the cell surface has a density of between about 4,000 molecules per cell and about 2,000 molecules per cell, between about 2,000 molecules per cell and about 1,000 molecules per cell, between about 1,500 molecules per cell and about 1,000 molecules per cell, between about 2,000 molecules per cell and about 500 molecules per cell, between about 1,000 molecules per cell and about 200 molecules per cell, or between about 1,000 molecules per cell and about 100 molecules per cell.

In certain embodiments, the TCR-like fusion molecule comprises a first antigen binding chain comprising a V_{H} of an antibody and a constant domain comprising a TRBC polypeptide; and a second antigen binding chain comprising a V_{L} of an antibody and a constant domain comprising a TRAC polypeptide. In certain embodiments, the first antigen binding chain is designated as “V_{H}-TRBC chain”. In certain embodiments, the second antigen binding chain is designated as “V_{L}-TRAC chain”. In certain embodiments, the first antigen binding chain comprises a hinge region between the V_{H} and the TRBC polypeptide. In certain embodiments, the hinge region comprises or consists of the amino acid sequence set forth in SEQ ID NO: 76 or SEQ ID NO: 78. In certain embodiments, the second antigen binding chain comprises a hinge region between the V_{L} and the TRAC polypeptide. In certain embodiments, the hinge region comprises or consists of the amino acid sequence set forth in SEQ ID NO: 76 or SEQ ID NO: 78.

### 3.5.1. Exemplary TCR-Like Fusion Molecules

In certain embodiments, the antigen-recognizing receptor is a TCR-like fusion molecule that binds to CD19 (e.g., human CD19) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD19. In certain embodiments, the V_{H} Comprises a CDR1 comprising the amino acid sequence set forth in SEQ ID NO: 92, a CDR2 comprising the amino acid sequence set forth in SEQ ID NO: 93, and a CDR3 comprising the amino acid sequence set forth in SEQ ID NO: 94. In certain embodiments, the V_{H} Comprises the amino acid sequence set forth in SEQ ID NO: 98. In certain embodiments, the V_{L} Comprises a CDR1 comprising the amino acid sequence set forth in SEQ ID NO: 95, a CDR2 comprising the amino acid sequence set forth in SEQ ID NO: 97, and a CDR3 comprising the amino acid sequence set forth in SEQ ID NO: 98. In certain embodiments, the V_{L} comprises the amino acid sequence set forth in SEQ ID NO: 100. In certain embodiments, the TRAC polypeptide comprises the amino acid sequence set forth in SEQ ID NO: 57 or SEQ ID NO: 59. In certain embodiments, the TRBC polypeptide is a TRBC2 polypeptide. In certain embodiments, the TRBC2 polypeptide comprises the amino acid sequence set forth in SEQ ID NO: 62 or SEQ ID NO: 64. SEQ ID NO: 92-101 are provided in Table 4 below.

In certain embodiments, the CDRs regions/sequences disclosed herein are delineated using the Kabat system (Swindells et al., J Mol Biol. 2017 Feb. 3; 429(3):356-364).

TABLE 4

| CDRS | 1 | 2 | 3 |
|---|---|---|---|
| V_{H} | SYWMN | QIYPGDGDTNYNGKFKG | KTISSVVDFYFDY |
| | [SEQ ID NO: 92] | [SEQ ID NO: 93] | [SEQ ID NO: 94] |
| V_{L} | KASQNVGTNVA | SATYRNS | QQYNRYPYT |
| | [SEQ ID NO: 95] | [SEQ ID NO: 96] | [SEQ ID NO: 97] |

| | |
|---|---|
| FULL V_{H} | MALPVTALLLPLALLLHAEVKLQQSGAELVRPGSSVKISCKASGYAFSSYWMNWVKQRPG |
| | QGLEWIGQIYPGDGDTNYNGKFKGQATLTADKSSSTAYMQLSGLTSEDSAVYFCARKTIS |
| | SVVDFYFDYWGQGTTVTV [SEQ ID NO: 98] |
| FULL V_{H}- | ATGGCTCTCCCAGTGACTGCCCTACTGCTTCCCCTAGCGCTTCTCCTGCATGCAGAGG |
| DNA | TGAAGCTGCAGCAGTCTGGGGCTGAGCTGGTGAGGCCTGGGTCCTCAGTGAAGATTTC |
| | CTGCAAGGCTTCTGGCTATGCATTCAGTAGCTACTGGATGAACTGGGTGAAGCAGAGG |
| | CCTGGACAGGGTCTTGAGTGGATTGGACAGATTTATCCTGGAGATGGTGATACTAACT |
| | ACAATGGAAAGTTCAAGGGTCAAGCCACACTGACTGCAGACAAATCCTCCAGCACAGC |
| | CTACATGCAGCTCAGCGGCCTAACATCTGAGGACTCTGCGGTCTATTTCTGTGCAAGA |
| | AAGACCATTAGTTCGGTAGTAGATTTCTACTTTGACTACTGGGGCCAAGGGACCACGG |
| | TCACCGTC [SEQ ID NO: 99] |
| FULL V_{L} | MALPVTALLLPLALLLHADIELTQSPKFMSTSVGDRVSVTCKASQNVGTNVAWYQQKPGQ |
| | SPKPLIYSATYRNSGVPDRFTGSGSGTDFTLTITNVQSKDLADYFCQQYNRYPYTSGGGT |
| | KLEI [SEQ ID NO: 100] |
| FULL V_{L}- | ATGGCACTGCCCGTGACCGCTCTGCTTCTCCCACTAGCTCTGCTTCTCCACGCAGACA |
| DNA | TTGAGCTCACCCAGTCTCCAAAATTCATGTCCACATCAGTAGGAGACAGGGTCAGCGT |
| | CACCTGCAAGGCCAGTCAGAATGTGGGTACTAATGTAGCCTGGTATCAACAGAAACCA |
| | GGACAATCTCCTAAACCACTGATTTACTCGGCAACCTACCGGAACAGTGGAGTCCCTG |
| | ATCGCTTCACAGGCAGTGGATCTGGGACAGATTTCACTCTCACCATCACTAACGTGCA |
| | GTCTAAAGACTTGGCAGACTATTTCTGTCAACAATATAACAGGTATCCGTACACGTCC |
| | GGAGGGGGGACCAAGCTGGAGATC [SEQ ID NO: 101] |

In certain embodiments, the antigen-recognizing receptor is a TCR-like fusion molecule that binds to CD70 (e.g., human CD70) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to CD70. In certain embodiments, the V_{H} comprises a CDR1 comprising the amino acid sequence set forth in SEQ ID NO: 104, a CDR2 comprising the amino acid sequence set forth in SEQ ID NO: 105, and a CDR3 comprising the amino acid sequence set forth in SEQ ID NO: 106. In certain embodiments, the V_{H} comprises the amino acid sequence set forth in SEQ ID NO: 110. In certain embodiments, the V_{L} comprises a CDR1 comprising the amino acid sequence set forth in SEQ ID NO: 106, a CDR2 comprising the amino acid sequence set forth in SEQ ID NO: 107, and a CDR3 comprising the amino acid sequence set forth in SEQ ID NO: 108. In certain embodiments, the V_{L} comprises the amino acid sequence set forth in SEQ ID NO: 111. In certain embodiments, the TRAC polypeptide comprises the amino acid sequence set forth in SEQ ID NO: 57 or SEQ ID NO: 59. In certain embodiments, the TRBC polypeptide is a TRBC2 polypeptide. In certain embodiments, the TRBC2 polypeptide comprises the amino acid sequence set forth in SEQ ID NO: 62 or SEQ ID NO: 64. SEQ ID NO: 104-111 are provided in Table 2 above.

In certain embodiments, the antigen-recognizing receptor is a TCR-like fusion molecule that binds to IL13R (e.g., human IL13R) and comprises two antigen binding chains, e.g., a first antigen binding chain that comprises a V_{H} and a TRBC polypeptide (“V_{H}-TRBC chain”) and a second antigen binding chain that comprises a V_{L} and a TRBC polypeptide (“V_{L}-TRAC chain”), which are capable of dimerizing and binding to IL13R. In certain embodiments, the V_{H} comprises a CDR1 comprising the amino acid sequence set forth in SEQ ID NO: 112, a CDR2 comprising the amino acid sequence set forth in SEQ ID NO: 113, and a CDR3 comprising the amino acid sequence set forth in SEQ ID NO: 114. In certain embodiments, the V_{H} comprises the amino acid sequence set forth in SEQ ID NO: 118. In certain embodiments, the V_{L} comprises a CDR1 comprising the amino acid sequence set forth in SEQ ID NO: 115, a CDR2 comprising the amino acid sequence set forth in SEQ ID NO: 116, and a CDR3 comprising the amino acid sequence set forth in SEQ ID NO: 117. In certain embodiments, the V_{L} comprises the amino acid sequence set forth in SEQ ID NO: 119. In certain embodiments, the TRAC polypeptide comprises the amino acid sequence set forth in SEQ ID NO: 57 or SEQ ID NO: 59. In certain embodiments, the TRBC polypeptide is a TRBC2 polypeptide. In certain embodiments, the TRBC2 polypeptide comprises the amino acid sequence set forth in SEQ ID NO: 62 or SEQ ID NO: 64. SEQ ID NO: 112-119 are provided in Table 3 above.

In certain embodiments, the CDRs regions/sequences disclosed herein are delineated using the Kabat system (Swindells et al., J Mol Biol. 2017 Feb. 3; 429(3):356-364).

Various TCR-like fusion molecules are disclosed in International Patent Application Publication No. WO2019/133969, which is incorporated by reference hereby in its entirety.

### 3.6. Delivery of the Antigen-Recognizing Receptor

In certain embodiments, the antigen-recognizing receptor is delivered to a cell by a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adeno-associated viral vectors, vaccinia viruses, bovine papilloma viruses, and herpes viruses (e.g., such as Epstein-Barr Virus).

In certain embodiments, the antigen-recognizing receptor is delivered to a cell by a non-viral method. Any targeted genome editing methods can also be used to deliver the antigen-recognizing receptor to a cell. In certain embodiments, the antigen-recognizing receptor is delivered to a cell by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

### 4. Nucleic Acids and Vectors

The presently disclosed subject matter provides nucleic acids and compositions thereof comprising a first polynucleotide encoding an immunoevasin disclosed herein (e.g., disclosed in Section 2) and a second polynucleotide encoding an antigen-recognizing receptor disclosed herein (e.g., disclosed in Section 3). Also provided are cells comprising such nucleic acids. In certain embodiments, the nucleic acids further comprise a first promoter that is operably linked to the immunoevasin. In certain embodiments, the nucleic acids further comprise a second promoter that is operably linked to the antigen-recognizing receptor.

In certain embodiments, one or both of the first and second promoters are endogenous or exogenous.

In certain embodiments, the exogenous promoter is selected from an elongation factor (EF)-1 promoter, a CMV promoter, a SV40 promoter, a PGK promoter, and a metallothionein promoter. In certain embodiments, the exogenous promoter is the elongation factor (EF)-1 promoter.

In certain embodiments, the elongation factor (EF)-1 (EF1) comprises or consists of a nucleotide sequence that is at least about 80%, at least about 85%, at least about 90%, at least about 95%, at least about 96%, at least about 97%, at least about 98%, at least about 99% or at least about 100% homologous or identical to the nucleotide sequence having SEQ ID NO: 90. SEQ ID NO: 90 is provided below. In certain embodiments, the elongation factor (EF)-1 comprises or consists of the nucleotide sequence having SEQ ID NO: 90.

[SEQ ID NO: 90]
GTGCCCGTCAGTGGGCAGAGCGCACATCGCCCACAGTCCCCGAGAAGTTGGGGGGAGGGGTCGGCAATTG
AACCGGTGCCTAGAGAAGGTGGCGCGGGGTAAACTGGGAAAGTGATGTCGTGTACTGGCTCCGCCTTTTT
CCCGAGGGTGGGGGAGAACCGTATATAAGTGCAGTAGTCGCCGTGAACGTTCTTTTTCGCAACGGGTTTG
CCGCCAGAACACAGGTAAGTGCCGTGTGTGGTTCCCGCGGGCCTGGCCTCTTTACGGGTTATGGCCCTTG
CGTGCCTTGAATTACTTCCACGCCCCTGGCTGCAGTACGTGATTCTTGATCCCGAGCTTCGGGTTGGAAG
TGGGTGGGAGAGTTCGAGGCCTTGCGCTTAAGGAGCCCCTTCGCCTCGTGCTTGAGTTGAGGCCTGGCCT
GGGCGCTGGGGCCGCCGCGTGCGAATCTGGTGGCACCTTCGCGCCTGTCTCGCTGCTTTCGATAAGTCTC
TAGCCATTTAAAATTTTTGATGACCTGCTGCGACGCTTTTTTTCTGGCAAGATAGTCTTGTAAATGCGGG
CCAAGATCTGCACACTGGTATTTCGGTTTTTGGGGCCGCGGGCGGCGACGGGGCCCGTGCGTCCCAGCGC
ACATGTTCGGCGAGGCGGGGCCTGCGAGCGCGGCCACCGAGAATCGGACGGGGGTAGTCTCAAGCTGGCC
GGCCTGCTCTGGTGCCTGGCCTCGCGCCGCCGTGTATCGCCCCGCCCTGGGCGGCAAGGCTGGCCCGGTC
GGCACCAGTTGCGTGAGCGGAAAGATGGCCGCTTCCCGGCCCTGCTGCAGGGAGCTCAAAATGGAGGACG
CGGCGCTCGGGAGAGCGGGCGGGTGAGTCACCCACACAAAGGAAAAGGGCCTTTCCGTCCTCAGCCGTCG
CTTCATGTGACTCCACGGAGTACCGGGCGCCGTCCAGGCACCTCGATTAGTTCTCGAGCTTTTGGAGTAC
GTCGTCTTTAGGTTGGGGGGAGGGGTTTTATGCGATGGAGTTTCCCCACACTGAGTGGGTGGAGACTGAA
GTTAGGCCAGCTTGGCACTTGATGTAATTCTCCTTGGAATTTGCCCTTTTTGAGTTTGGATCTTGGTTCA
TTCTCAAGCCTCAGACAGTGGTTCAAAGTTTTTTTCTTCCATTTCAGGTGTCGTGA

In certain embodiments, one or both of the first and second promoters are inducible promoters. In certain embodiment, the inducible promoter is selected from a NFAT transcriptional response element (TRE) promoter, a CD69 promoter, a CD25 promoter, and an IL-2 promoter.

In certain embodiments, the nucleic acids and composition thereof can be a vector. In certain embodiments, the vector is a retroviral vector (e.g., a gamma-retroviral vector or a lentiviral vector). In certain embodiments, the vector is viral vectors selected from the group consisting of adenoviral vectors, adena-associated viral vectors, vaccinia viruses, bovine papilloma viruses, and herpes viruses (e.g., such as Epstein-Barr Virus).

Additionally, the nucleic acids and compositions thereof can be administered to subjects or and/delivered into cells by art-known methods or as described herein. Genetic modification of a cell (e.g., a T cell or a NK cell) can be accomplished by transducing a substantially homogeneous cell composition with a recombinant DNA construct. In certain embodiments, a retroviral vector (either gamma-retroviral or lentiviral) is employed for the introduction of the nucleic acid compositions into the cell. For example, the first polynucleotide and the second polynucleotide can be cloned into a retroviral vector and expression can be driven from its endogenous promoter, from the retroviral long terminal repeat, or from a promoter specific for a target cell type of interest. Non-viral vectors may be used as well.

The first polynucleotide and the second polynucleotide can be constructed in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Natl. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J. Clin. Invest. 89:1817.

Other transducing viral vectors can be used to modify a cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adena-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

Non-viral approaches can also be employed for genetic modification of a cell. For example, a nucleic acid molecule can be delivered into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Transient expression may be obtained by RNA electroporation.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

In certain embodiments, the delivery methods include use of colloids. As used herein, the term “colloid” refers to systems in which there are two or more phases, with one phase (e.g., the dispersed phase) distributed in the other phase (e.g., the continuous phase). Moreover, at least one of the phases has small dimensions (in the range of about 10⁻⁹ to about 10⁻⁶ m). Non-limiting examples of colloids encompassed by the presently disclosed subject matter include macromolecule complexes, nanocapsules, microspheres, beads, and lipid-based systems (e.g., micelles, liposomes, and lipid nanoparticles).

In certain embodiments, the delivery methods include use of liposomes. The term “liposome,” as used herein, refers to single- or multi-layered spherical lipid bilayer structures produced from lipids dissolved in organic solvents and then dispersed in aqueous media. Experimentally and therapeutically used for delivering an active pharmaceutical ingredient (e.g., nucleic acid compositions disclosed herein) to cells, liposomes fuse with cell membranes so the contents are transferred into the cytoplasm.

In certain embodiments, the delivery methods include use of lipid nanoparticles. As used herein, the term “lipid nanoparticle” refers to a particle having at least one dimension in the order of nanometers (e.g., from about 1 nm to about 1,000 nm) and including at least one lipid. In certain embodiments, the lipid nanoparticles can include an active pharmaceutical ingredient (e.g., nucleic acid compositions disclosed herein) for delivering to cells. The morphology of the lipid nanoparticles can be different from liposomes. While liposomes are characterized by a lipid bilayer surrounding a hydrophilic core, lipid nanoparticles have an electron-dense core where cationic lipids and/or ionizable lipids are organized into inverted micelles around an active pharmaceutical ingredient (e.g., nucleic acid compositions disclosed herein). Additional information on the morphology and properties of lipid nanoparticles and liposomes can be found in Wilczewska, et al., Pharmacological reports 64, no. 5 (2012): 1020-1037; Eygeris et al., Accounts of Chemical Research 55, no. 1 (2021): 2-12; Zhang et al., Chemical Reviews 121, no. 20 (2021): 12181-12277; and Fan et al., Journal of pharmaceutical and biomedical analysis 192 (2021): 113642.

In certain embodiments, the lipid nanoparticles have a mean diameter of from about 30 nm to about 150 nm, from about 40 nm to about 150 nm, from about 50 nm to about 150 nm, from about 60 nm to about 130 nm, from about 70 nm to about 110 nm, from about 70 nm to about 100 nm, from about 80 nm to about 100 nm, from about 90 nm to about 100 nm, from about 70 to about 90 nm, from about 80 nm to about 90 nm, from about 70 nm to about 80 nm, or about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In certain embodiments, the lipid nanoparticles can include a cationic lipid or an ionizable lipid. The term “cationic lipid” refers to lipids including a head group with permanent positive charges. Non-limiting examples of cationic lipids encompassed by the presently disclosed subject matter include 1,2-di-O-octadecenyl-3-trimethylammonium-propane (DOTMA), 1,2-dioleoyl-3-trimethylammonium-propane (DOTAP), 2,3-dioleyloxy-N-[2-(sperminecarboxamido)ethyl]-N,N-dimethyl-1-propanaminium trifluoroacetate (DOSPA), and ethylphosphatidylcholine (ePC).

As used herein, the term “ionizable lipid” refers to lipids that are protonated at low pH and are neutral at physiological pH. The pH-sensitivity of ionizable lipids is particularly beneficial for delivery in vivo (e.g., delivery of nucleic acid compositions disclosed herein), because neutral lipids have less interactions with the anionic membranes of blood cells and, thus, improve the biocompatibility of the lipid nanoparticles. Once trapped in endosomes, ionizable lipids are protonated and promote membrane destabilization to allow the endosomal escape of the nanoparticles. Non-limiting example of ionizable lipids encompassed by the presently disclosed subject matter include tetrakis(8-methylnonyl) 3,3′,3″,3′″-(((methylazanediyl) bis(propane-3,1 diyl))bis (azanetriyl))tetrapropionate; decyl (2-(dioctylammonio)ethyl) phosphate; ((4-hydroxybutyl)azanediyl)bis(hexane-6,1-diyl)bis(2-hexyldecanoate); bis(2-(dodecyldisulfanyl)ethyl) 3,3′-((3-methyl-9-oxo-10-oxa-13,14-dithia-3,6-diazahexacosyl)azanediyl)dipropionate; 1,1′-((2-(4-(2-((2-(bis(2-hydroxydodecyl)amino)ethyl) (2-hydroxydodecyl)amino)ethyl) piperazin-1-yl)ethyl)azanediyl) bis(dodecan-2-ol); cKK-E12, 3,6-bis(4-(bis(2-hydroxydodecyl)amino)butyl)piperazine-2,5-dione; (6Z,9Z,28Z,31Z)-heptatriaconta-6,9,28,31-tetraen-19-yl 4-(dimethylamino) butanoate; hexa(octan-3-yl) 9,9′,9″,9′″,9″″,9′″″-((((benzene-1,3,5-tricarbonyl)yris(azanediyl)) tris (propane-3,1-diyl)) tris(azanetriyl))hexanonanoate; heptadecan-9-yl 8-((2-hydroxyethyl)(6-oxo-6-(undecyloxy)hexyl)amino) octanoate; and (((3,6-dioxopiperazine-2,5-diyl)bis(butane-4, 1-diyl))bis(azanetriyl))tetrakis(ethane-2,1-diyl) (9Z,9′Z,9″Z,9′″Z,12Z,12′Z,12″Z,12′″Z)-tetrakis (octadeca-9,12-dienoate).

Additionally, in certain embodiments, the lipid nanoparticles can include other lipids. For example, but without any limitation, the lipid nanoparticles of the presently disclosed subject matter can include phospholipids, cholesterol, polyethylene glycol (PEG)-functionalized lipids (PEG-lipids). These lipids can improve certain properties of the lipid nanoparticles (e.g., stability, biodistribution, etc.). For example, cholesterol enhances the stability of the lipid nanoparticles by modulating the integrity and rigidity. Non-limiting examples of other lipids present in lipid nanoparticles include cholesterol, DC-cholesterol, β-sitosterol, BHEM-cholesterol, ALC-0159, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoylphosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (transDOPE).

In certain embodiments, the lipid nanoparticles can include a targeting moiety that binds to a ligand. The use of the targeting moieties allows selective delivery of an active pharmaceutical ingredient (e.g., nucleic acid compositions disclosed herein) to target cells expressing the ligand (e.g., T cells). In certain embodiments, the targeting moiety can be an antibody or antigen-binding fragment thereof that binds to a cell surface receptor. For example, but without any limitation, the targeting domain is an antibody or antigen-binding fragment thereof that binds to a receptor expressed on the surface of a T cell (e.g., CD3, CD4, CD8, CD16, CD40L, CD95, FasL, CTLA-4, OX40, GITR, LAG3, ICOS, and PD-1).

In certain embodiments, the delivery methods are in vivo delivery methods. In certain embodiments, the delivery methods are ex vivo delivery methods.

### 4.1. Exemplified Nucleic Acids

In certain embodiments, the presently disclosed nucleic acid comprises a first polynucleotide. In certain embodiments, the first polynucleotide encodes an immunoevasin. In certain embodiments, the immunoevasin is a NEF polypeptide. In certain embodiments, the NEF polypeptide comprises the amino acid sequence set forth in SEQ ID NO: 1 or SEQ ID NO: 2. In certain embodiments, the nucleic acid further comprise a first promoter that is operably linked to the immunoevasin. In certain embodiments, the promoter is a EF-1 promoter. In certain embodiments, the EF-1 promoter comprises the nucleotide sequence set forth in SEQ ID NO: 90. In certain embodiments, the second polynucleotide encodes an antigen recognizing receptor. In certain embodiments, the antigen-recognizing receptor is a CAR. In certain embodiments, the CAR is 1928z1XX. CAR designated as 1928z1XX is described in Section 3.3.4.1.

In certain embodiments, the presently disclosed nucleic acid comprises a first polynucleotide. In certain embodiments, the first polynucleotide encodes an immunoevasin. In certain embodiments, the immunoevasin is a NEF polypeptide. In certain embodiments, the NEF polypeptide comprises the amino acid sequence set forth in SEQ ID NO: 1 or SEQ ID NO: 2. In certain embodiments, the nucleic acid further comprise a first promoter that is operably linked to the immunoevasin. In certain embodiments, the promoter is a EF-1 promoter. In certain embodiments, the EF-1 promoter comprises the nucleotide sequence set forth in SEQ ID NO: 90. In certain embodiments, the second polynucleotide encodes an antigen recognizing receptor. In certain embodiments, the antigen-recognizing receptor is a CAR. In certain embodiments, the CAR is IL13-28z1XX. CAR designated as IL13-28z1XX is described in Section 3.3.4.1.

In certain embodiments, the presently disclosed nucleic acid comprises a first polynucleotide encoding an immunoevasin and a second polynucleotide encoding an antigen recognizing receptor. In certain embodiments, the immunoevasin is a NEF polypeptide. In certain embodiments, the nucleic acid further comprise a first promoter that is operably linked to the immunoevasin. In certain embodiments, the promoter is the elongation factor (EF)-1 promoter. In certain embodiments, the antigen-recognizing receptor is a 1928z1XX CAR. In certain embodiments, the nucleic acid comprises or consists of a nucleotide sequence that is at least about 80%, at least about 85%, at least about 90%, at least about 95%, at least about 96%, at least about 97%, at least about 98%, at least about 99% or at least about 100% homologous or identical to the nucleotide sequence having SEQ ID NO: 102. SEQ ID NO: 102 is provided below. In certain embodiments, the nucleic acid comprises or consists of the nucleotide sequence having SEQ ID NO: 102.

[SEQ ID NO: 102]
ATGGCTCTCCCAGTGACTGCCCTACTGCTTCCCCTAGCGCTTCTCCTGCATGCAGAGGTGAAGCTGCAGC
AGTCTGGGGCTGAGCTGGTGAGGCCTGGGTCCTCAGTGAAGATTTCCTGCAAGGCTTCTGGCTATGCATT
CAGTAGCTACTGGATGAACTGGGTGAAGCAGAGGCCTGGACAGGGTCTTGAGTGGATTGGACAGATTTAT
CCTGGAGATGGTGATACTAACTACAATGGAAAGTTCAAGGGTCAAGCCACACTGACTGCAGACAAATCCT
CCAGCACAGCCTACATGCAGCTCAGCGGCCTAACATCTGAGGACTCTGCGGTCTATTTCTGTGCAAGAAA
GACCATTAGTTCGGTAGTAGATTTCTACTTTGACTACTGGGGCCAAGGGACCACGGTCACCGTCTCCTCA
GGTGGAGGTGGATCAGGTGGAGGTGGATCTGGTGGAGGTGGATCTGACATTGAGCTCACCCAGTCTCCAA
AATTCATGTCCACATCAGTAGGAGACAGGGTCAGCGTCACCTGCAAGGCCAGTCAGAATGTGGGTACTAA
TGTAGCCTGGTATCAACAGAAACCAGGACAATCTCCTAAACCACTGATTTACTCGGCAACCTACCGGAAC
AGTGGAGTCCCTGATCGCTTCACAGGCAGTGGATCTGGGACAGATTTCACTCTCACCATCACTAACGTGC
AGTCTAAAGACTTGGCAGACTATTTCTGTCAACAATATAACAGGTATCCGTACACGTCCGGAGGGGGGAC
CAAGCTGGAGATCAAACGGGCGGCCGCAATTGAAGTTATGTATCCTCCTCCTTACCTAGACAATGAGAAG
AGCAATGGAACCATTATCCATGTGAAAGGGAAACACCTTTGTCCAAGTCCCCTATTTCCCGGACCTTCTA
AGCCCTTTTGGGTGCTGGTGGTGGTTGGTGGAGTCCTGGCTTGCTATAGCTTGCTAGTAACAGTGGCCTT
TATTATTTTCTGGGTGAGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTCGCAGCCTATCGCTCCA
GAGTGAAGTTCAGCAGGAGCGCAGACGCCCCCGCGTACCAGCAGGGCCAGAACCAGCTCTATAACGAGCT
CAATCTAGGACGAAGAGAGGAGTACGATGTTTTGGACAAGAGACGTGGCCGGGACCCTGAGATGGGGGGA
AAGCCGAGAAGGAAGAACCCTCAGGAAGGCCTGTTCAATGAACTGCAGAAAGATAAGATGGCGGAGGCCT
TCAGTGAGATTGGGATGAAAGGCGAGCGCCGGAGGGGCAAGGGGCACGATGGCCTTTTCCAGGGTCTCAG
TACAGCCACCAAGGACACCTTCGACGCCCTTCACATGCAGGCCCTGCCCCCTCGCTAAATCCCTAGGCGA
CTGTGCCTTCTAGTTGCCAGCCGTCTGTTGTTTGCCCCTCCCCCGTGCCTTCCTTGACCCTGGAAGGTGC
CACTCCCACTGTCCTTTCCTAATAAAATGAGGAAATTGCATCGCATTGTCTGAGTAGGTGTCATTCTATT
CTGGGGGGTGGGGTGGGGCAGGACAGCAAGGGGGAGGATTGGGAAGACAATAGCAGGCATGCTCGAGGAA
TTCGACGTGCCCGTCAGTGGGCAGAGCGCACATCGCCCACAGTCCCCGAGAAGTTGGGGGGAGGGGTCGG
CAATTGAACCGGTGCCTAGAGAAGGTGGCGCGGGGTAAACTGGGAAAGTGATGTCGTGTACTGGCTCCGC
CTTTTTCCCGAGGGTGGGGGAGAACCGTATATAAGTGCAGTAGTCGCCGTGAACGTTCTTTTTCGCAACG
GGTTTGCCGCCAGAACACAGGTAAGTGCCGTGTGTGGTTCCCGCGGGCCTGGCCTCTTTACGGGTTATGG
CCCTTGCGTGCCTTGAATTACTTCCACGCCCCTGGCTGCAGTACGTGATTCTTGATCCCGAGCTTCGGGT
TGGAAGTGGGTGGGAGAGTTCGAGGCCTTGCGCTTAAGGAGCCCCTTCGCCTCGTGCTTGAGTTGAGGCC
TGGCCTGGGCGCTGGGGCCGCCGCGTGCGAATCTGGTGGCACCTTCGCGCCTGTCTCGCTGCTTTCGATA
AGTCTCTAGCCATTTAAAATTTTTGATGACCTGCTGCGACGCTTTTTTTCTGGCAAGATAGTCTTGTAAA
TGCGGGCCAAGATCTGCACACTGGTATTTCGGTTTTTGGGGCCGCGGGCGGCGACGGGGCCCGTGCGTCC
CAGCGCACATGTTCGGCGAGGCGGGGCCTGCGAGCGCGGCCACCGAGAATCGGACGGGGGTAGTCTCAAG
CTGGCCGGCCTGCTCTGGTGCCTGGCCTCGCGCCGCCGTGTATCGCCCCGCCCTGGGCGGCAAGGCTGGC
CCGGTCGGCACCAGTTGCGTGAGCGGAAAGATGGCCGCTTCCCGGCCCTGCTGCAGGGAGCTCAAAATGG
AGGACGCGGCGCTCGGGAGAGCGGGCGGGTGAGTCACCCACACAAAGGAAAAGGGCCTTTCCGTCCTCAG
CCGTCGCTTCATGTGACTCCACGGAGTACCGGGCGCCGTCCAGGCACCTCGATTAGTTCTCGAGCTTTTG
GAGTACGTCGTCTTTAGGTTGGGGGGAGGGGTTTTATGCGATGGAGTTTCCCCACACTGAGTGGGTGGAG
ACTGAAGTTAGGCCAGCTTGGCACTTGATGTAATTCTCCTTGGAATTTGCCCTTTTTGAGTTTGGATCTT
GGTTCATTCTCAAGCCTCAGACAGTGGTTCAAAGTTTTTTTCTTCCATTTCAGGTGTCGTGAGGAATTAG
GATCCATCGCCACCATGGGTGGCAAGTGGTCAAAACGTAGTGTGGTTGGATGGCCTGCTGTAAGGGAAAG
AATGAGACGAGCTGAGCCAGCAGCAGATGGGGTGGGAGCAGTATCTCGAGACCTGGAAAAACATGGAGCA
ATCACAAGTAGCAATACAGCAGCTAACAATGCTGATTGTGCCTGGCTAGAAGCACAAGAGGAGGAGGAGG
TGGGTTTTCCAGTCAGACCTCAGGTACCTTTAAGACCAATGACTTACAAGGCAGCTGTAGATCTTAGCCA
CTTTTTAAAAGAAAAGGGGGGACTGGAAGGGCTAATTTACTCCCAAAAAAGACAAGATATCCTTGATCTG
TGGGTCTACCACACACAAGGCTACTTCCCTGATTGGCAGAACTACACACCAGGGCCAGGGATCAGATATC
CACTGACCTTTGGATGGTGCTTCAAGCTAGTACCAGTTGAGCCAGAGAAGGTAGAAGAGGCCAATGAAGG
AGAGAACAACAGCTTGTTACACCCTATGAGCCTGCATGGGATGGATGACCCGGAGAAAGAAGTGTTAGTG
TGGAAGTTTGACAGCCGCCTAGCATTTCATCACATGGCCCGAGAGCTGCATCCGGAGTACTACAAGGACT
GCTAA

In certain embodiments, the presently disclosed nucleic acid comprises a first polynucleotide. In certain embodiments, the first polynucleotide encodes a first immunoevasin and a second immunoevasin. In certain embodiments, the first immunoevasin is a NEF polypeptide. In certain embodiments, the NEF polypeptide comprises the amino acid sequence set forth in SEQ ID NO: 1 or SEQ ID NO: 2. In certain embodiments, the second immunoevasin is a BNLF2a polypeptide. In certain embodiments, the BNLF2a polypeptide comprises the amino acid sequence set forth in SEQ ID NO: 5. In certain embodiments, the nucleic acid further comprise a first promoter that is operably linked to the first polynucleotide. In certain embodiments, the promoter is a EF-1 promoter. In certain embodiments, the EF-1 promoter comprises the nucleotide sequence set forth in SEQ ID NO: 90. In certain embodiments, the second polynucleotide encodes an antigen recognizing receptor. In certain embodiments, the antigen-recognizing receptor is a CAR. In certain embodiments, the CAR is 1928z1XX.

In certain embodiments, the presently disclosed nucleic acid comprises a first polynucleotide. In certain embodiments, the first polynucleotide encodes an immunoevasin. In certain embodiments, the immunoevasin is a NEF polypeptide. In certain embodiments, the NEF polypeptide comprises the amino acid sequence set forth in SEQ ID NO: 1 or SEQ ID NO: 2. In certain embodiments, the nucleic acid further comprise a first promoter that is operably linked to the immunoevasin. In certain embodiments, the promoter is a EF-1 promoter. In certain embodiments, the EF-1 promoter comprises the nucleotide sequence set forth in SEQ ID NO: 90. In certain embodiments, the second polynucleotide encodes an antigen recognizing receptor. In certain embodiments, the antigen-recognizing receptor is a CAR. In certain embodiments, the CAR is 70-28z1XX. CAR designated as 70-28z1XX is described in Section 3.3.4.1.

In certain embodiments, the presently disclosed nucleic acid comprises a first polynucleotide. In certain embodiments, the first polynucleotide encodes an immunoevasin. In certain embodiments, the immunoevasin is a NEF polypeptide. In certain embodiments, the NEF polypeptide comprises the amino acid sequence set forth in SEQ ID NO: 1 or SEQ ID NO: 2. In certain embodiments, the nucleic acid further comprise a first promoter that is operably linked to the immunoevasin. In certain embodiments, the promoter is a EF-1 promoter. In certain embodiments, the EF-1 promoter comprises the nucleotide sequence set forth in SEQ ID NO: 90. In certain embodiments, the second polynucleotide encodes an antigen recognizing receptor. In certain embodiments, the antigen-recognizing receptor is a CAR. In certain embodiments, the CAR is IL13-28z1XX. CAR designated as IL13-28z1XX is described in Section 3.3.4.1.

In certain embodiments, the presently disclosed nucleic acid comprises a first polynucleotide. In certain embodiments, the first polynucleotide encodes a first immunoevasin and a second immunoevasin. In certain embodiments, the first immunoevasin is a NEF polypeptide. In certain embodiments, the NEF polypeptide comprises the amino acid sequence set forth in SEQ ID NO: 1 or SEQ ID NO: 2. In certain embodiments, the second immunoevasin is a BNLF2a polypeptide. In certain embodiments, the BNLF2a polypeptide comprises the amino acid sequence set forth in SEQ ID NO: 5. In certain embodiments, the nucleic acid further comprise a first promoter that is operably linked to the first polynucleotide. In certain embodiments, the promoter is a EF-1 promoter. In certain embodiments, the EF-1 promoter comprises the nucleotide sequence set forth in SEQ ID NO: 90. In certain embodiments, the second polynucleotide encodes an antigen recognizing receptor. In certain embodiments, the antigen-recognizing receptor is a CAR. In certain embodiments, the CAR is 70-28z1XX.

In certain embodiments, the presently disclosed nucleic acid comprises a first polynucleotide. In certain embodiments, the first polynucleotide encodes a first immunoevasin and a second immunoevasin. In certain embodiments, the first immunoevasin is a NEF polypeptide. In certain embodiments, the NEF polypeptide comprises the amino acid sequence set forth in SEQ ID NO: 1 or SEQ ID NO: 2. In certain embodiments, the second immunoevasin is a BNLF2a polypeptide. In certain embodiments, the BNLF2a polypeptide comprises the amino acid sequence set forth in SEQ ID NO: 5. In certain embodiments, the nucleic acid further comprise a first promoter that is operably linked to the first polynucleotide. In certain embodiments, the promoter is a EF-1 promoter. In certain embodiments, the EF-1 promoter comprises the nucleotide sequence set forth in SEQ ID NO: 90. In certain embodiments, the second polynucleotide encodes an antigen recognizing receptor. In certain embodiments, the antigen-recognizing receptor is a CAR. In certain embodiments, the CAR is IL13-28z1XX.

### 5. Cells

The presently disclosed subject matter provides cells comprising a) an immunoevasin polypeptide, and b) a antigen-recognizing receptor that targets an antigen.

In certain embodiments, the cell is selected from the group consisting of cells of lymphoid lineage and cells of myeloid lineage. In certain embodiments, the cell is an immunoresponsive cell. In certain embodiments, the immunoresponsive cell is a cell of lymphoid lineage.

In certain embodiments, the cell is a cell of the lymphoid lineage. Cells of the lymphoid lineage can provide production of antibodies, regulation of cellular immune system, detection of foreign agents in the blood, detection of cells foreign to the host, and the like. Non-limiting examples of cells of the lymphoid lineage include T cells, Natural Killer (NK) cells, B cells, dendritic cells, stem cells from which lymphoid cells may be differentiated. In certain embodiments, the stem cell is a pluripotent stem cell (e.g., embryonic stem cell).

In certain embodiments, the cell is a T cell. T cells can be lymphocytes that mature in the thymus and are chiefly responsible for cell-mediated immunity. T cells are involved in the adaptive immune system. The T cells of the presently disclosed subject matter can be any type of T cells, including, but not limited to, helper T cells, cytotoxic T cells, memory T cells (including central memory T cells, stem-cell-like memory T cells (or stem-like memory T cells), and two types of effector memory T cells: e.g., T_{EM} cells and T_{EMRA} cells, Regulatory T cells (also known as suppressor T cells), tumor-infiltrating lymphocyte (TIL), Natural Killer T cells, Mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of an antigen-recognizing receptor, e.g., a CAR or a TCR. The T cell can be a CD4⁺ T cell or a CD8⁺ T cell. In certain embodiments, the T cell is a CD4⁺ T cell. In certain embodiments, the T cell is a CD8⁺ T cell. In certain embodiments, the CD8⁺ T cell is CD4 independent. In certain embodiments, the T cell is derived from an induced pluripotent stem cell (iPSC). In certain embodiments, the T cell is a CD8⁺ T cell that is CD4 independent, and the CD8⁺ T cell is derived from an iPSC.

In certain embodiments, the T cell is a CD62L⁺ T cell. In certain embodiments, the T cell is a CD45RA⁺ T cell. In certain embodiments, the T cell is a CD62L⁺/CD45RA⁺ T cell.

In certain embodiments, the cell is an NK cell. Natural Killer (NK) cells can be lymphocytes that are part of cell-mediated immunity and act during the innate immune response. NK cells do not require prior activation in order to perform their cytotoxic effect on target cells.

Types of human lymphocytes of the presently disclosed subject matter include, without limitation, peripheral donor lymphocytes, e.g., those disclosed in Sadelain, M., et al. 2003 Nat Rev Cancer 3:35-45 (disclosing peripheral donor lymphocytes genetically modified to express CARs), in Morgan, R. A., et al. 2006 Science 314:126-129 (disclosing peripheral donor lymphocytes genetically modified to express a full-length tumor antigen-recognizing T cell receptor complex comprising the α and β heterodimer), in Panelli, M. C., et al. 2000 J Immunol 164:495-504; Panelli, M. C., et al. 2000 J Immunol 164:4382-4392 (disclosing lymphocyte cultures derived from tumor infiltrating lymphocytes (TILs) in tumor biopsies), and in Dupont, J., et al. 2005 Cancer Res 65:5417-5427; Papanicolaou, G. A., et al. 2003 Blood 102:2498-2505 (disclosing selectively in vitro-expanded antigen-specific peripheral blood leukocytes employing artificial antigen-presenting cells (AAPCs) or pulsed dendritic cells).

In certain embodiments, the cell (e.g., T cell) is autologous. As used herein, the term “autologous” refers to a cell, a cell line, a population of cells, a tissue, or an organ that is obtained from a subject that is intended to receive the cell, cell line, population of cells, tissue, or organ.

In certain embodiments, the cell (e.g., T cell) is non-autologous. In certain embodiments, the cell (e.g., T cell) is allogeneic. As used herein, the term “allogeneic” refers to a cell, a cell line, a population of cells, a tissue, or an organ that is obtained from a subject that is different from the subject intended to receive the cell, cell line, population of cells, tissue, or organ.

In certain embodiments, the cell (e.g., T cell) is derived in vitro from an engineered progenitor or stem cell.

In certain embodiments, the cell is a cell of the myeloid lineage. Non-limiting examples of cells of the myeloid lineage include monocytes, macrophages, neutrophils, basophils, eosinophils, erythrocytes, megakaryocytes, and stem cells from which myeloid cells may be differentiated.

In certain embodiments, the stem cell is a pluripotent stem cell (e.g., an embryonic stem cell or an induced pluripotent stem cell).

### 5.1. CCRs

In certain embodiments, a presently disclosed cell comprising an immunoevasin polypeptide, and an antigen-recognizing receptor that targets an antigen further comprises a CCR. The term “chimeric co-stimulating receptor” or “CCR” refers to a chimeric receptor that binds to an antigen and provides a co-stimulatory signal, but does not provide a T-cell activation signal to a cell comprising the CCR. Various CCRs are described in U.S. Patent Publication No. 2002/0018783, the content of which is incorporated by reference in its entirety. CCRs mimic co-stimulatory signals, but unlike, CARs, do not provide a T-cell activation signal. In certain embodiments, the CCR lacks a CD3ζ polypeptide.

CCRs provide co-stimulation signal (e.g., a CD28-like signal or 4-1BB-like signal), in the absence of the natural co-stimulatory ligand on the antigen-presenting cell. A combinatorial antigen recognition, i.e., use of a CCR in combination with a CAR, can augment T-cell reactivity against the dual-antigen expressing T cells, thereby improving selective tumor targeting. Kloss et al., describe a strategy that integrates combinatorial antigen recognition, split signaling, and, critically, balanced strength of T-cell activation and co-stimulation to generate T cells that eliminate target cells that express a combination of antigens while sparing cells that express each antigen individually (Kloss et al., Nature Biotechnology (2013); 31(1):71-75, the content of which is incorporated by reference in its entirety). With this approach, T-cell activation requires CAR-mediated recognition of one antigen, whereas co-stimulation is independently mediated by a CCR specific for a second antigen. To achieve tumor selectivity, the combinatorial antigen recognition approach diminishes the efficiency of T-cell activation to a level where it is ineffective without rescue provided by simultaneous CCR recognition of the second antigen.

In certain embodiments, the CCR comprises an extracellular antigen-binding domain that binds to a second antigen and an intracellular domain that is capable of delivering a costimulatory signal to the cell but does not alone deliver an activation signal to the cell. In certain embodiments, the CCR further comprises a transmembrane domain. In certain embodiments, the intracellular domain of the CCR comprises at least an intracellular domain of a co-stimulatory molecule or a portion thereof. In certain embodiments, the co-stimulatory molecule is selected from the group consisting of CD28, 4-1BB, OX40, CD27, CD40, CD154, CD97, CD11a/CD18, ICOS, DAP-10, CD2, CD150, CD226, and NKG2D.

In certain embodiments, the CCR comprises an intracellular domain of CD28 or a portion thereof. In certain embodiments, the CCR comprises an intracellular domain of 4-1BB or a portion thereof. In certain embodiments, the CCR comprises an intracellular domain of CD28 or a portion thereof, and an intracellular domain of 4-1BB or a portion thereof.

In certain embodiments, the second antigen is selected so that expression of both of the first antigen and the second antigen is restricted to the targeted cells (e.g., cancerous tissue or cancerous cells). Similar to a CAR, the extracellular antigen-binding domain can be an scFv, a Fab, a F(ab)₂, or a fusion protein with a heterologous sequence to form the extracellular antigen-binding domain.

In certain embodiments, the cell comprising the immunoevasin polypeptide, the antigen-recognizing receptor that targets an antigen, and the CCR exhibits a greater degree of cytolytic activity against cells that are positive for both the first and the second antigen as compared to against cells that are singly positive for the first antigen. In certain embodiments, the cell comprising the first antigen-recognizing receptor, the second antigen-recognizing receptor, and the CCR exhibits substantially no or negligible cytolytic activity against cells that are singly positive for the first antigen.

In certain embodiments, the antigen recognizing receptor binds to the antigen with a low binding affinity, e.g., a dissociation constant (K_{D}) of about 1×10⁻⁸ M or more, about 5×10⁻⁸ M or more, about 1×10⁻⁷ M or more, about 5×10⁻⁷ M or more, or about 1×10⁻⁶ M or more, or from about 1×10⁻⁸ M to about 1×10⁻⁶ M. In certain embodiments, the antigen recognizing receptor (e.g., a CAR, a TCR, or a TCR-like fusion molecule) binds to the antigen with a low binding avidity. In certain embodiments, the antigen recognizing receptor (e.g., a CAR, a TCR, or a TCR-like fusion molecule) binds to the antigen at an epitope of low accessibility. In certain embodiments, the antigen recognizing receptor (e.g., a CAR, a TCR, or a TCR-like fusion molecule) binds to the antigen with a binding affinity that is lower compared to the binding affinity with which the second antigen-recognizing receptor (e.g., a CCR) binds to the second antigen. In certain embodiments, the CCR binds to the second antigen with a binding affinity K_{D} of from about 1×10⁻⁹ M to about 1×10⁻⁷ M, e.g., about 1×10⁻⁷ M or less, about 1×10⁻⁸ M or less, or about 1×10⁻⁹ M or less.

### 5.2. Co-Stimulatory Ligands

In certain embodiments, a presently disclosed cell comprising an immunoevasin polypeptide, and an antigen-recognizing receptor that targets an antigen further comprises at least one recombinant or exogenous co-stimulatory ligand. For example, a presently disclosed cell can be further transduced with at least one co-stimulatory ligand, such that the cell expresses or is induced to express the first antigen-recognizing receptor, the second antigen-recognizing receptor, and the at least one co-stimulatory ligand. The at least one co-stimulatory ligand provides a co-stimulation signal to the cell.

Non-limiting examples of co-stimulatory ligands include, but are not limited to, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Non-limiting examples of TNF superfamily members include nerve growth factor (NGF), CD40L (also known as “CD154”), 4-1BBL, TNF-α, OX40L, CD70, Fas ligand (FasL), CD30L, tumor necrosis factor beta (TNFβ)/lymphotoxin-alpha (LTα), lymphotoxin-beta (LTβ), CD257/B cell-activating factor (BAFF)/Blys/THANK/Tall-1, glucocorticoid-induced TNF Receptor ligand (GITRL), TNF-related apoptosis-inducing ligand (TRAIL), and LIGHT (TNFSF14). The immunoglobulin (Ig) superfamily is a large group of cell surface and soluble proteins that are involved in the recognition, binding, or adhesion processes of cells. These proteins share structural features with immunoglobulins—they possess an immunoglobulin domain (fold). Non-limiting examples of immunoglobulin superfamily ligands include CD80, CD86, and ICOSLG. In certain embodiments, the at least one co-stimulatory ligand is selected from the group consisting of 4-1BBL, CD80, CD86, CD70, GITRL, CD40L, OX40L, CD30L, TNFRSF14, ICOSLG, TRAIL, and combinations thereof.

In certain embodiments, the cell further comprises one exogenous co-stimulatory ligand that is 4-1BBL. In certain embodiments, the co-stimulatory ligand is human 4-1BBL. In certain embodiments, the 4-1BBL comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a Uniprot Reference No: P41273-1 (SEQ ID NO: 84) or a fragment thereof, and/or may optionally comprise up to one or up to two or up to three conservative amino acid substitutions. In certain embodiments, the 4-1BBL comprises or consists of an amino acid sequence that is a consecutive portion of the amino acid sequence of SEQ ID NO: 84. SEQ ID NO: 84 is provided below.

[SEQ ID NO: 84]
MEYASDASLDPEAPWPPAPRARACRVLPWALVAGLLLLLLLAAACAVFLACPWAVSGARASPGSAASPRL
REGPELSPDDPAGLLDLRQGMFAQLVAQNVLLIDGPLSWYSDPGLAGVSLTGGLSYKEDTKELVVAKAGV
YYVFFQLELRRVVAGEGSGSVSLALHLQPLRSAAGAAALALTVDLPPASSEARNSAFGFQGRLLHLSAGQ
RLGVHLHTEARARHAWQLTQGATVLGLFRVTPEIPAGLPSPRSE

An exemplary nucleotide sequence encoding the amino acid sequence of SEQ ID NO: 84 is set forth in SEQ ID NO: 85.

[SEQ ID NO: 85]
ATGGAATACGCCTCTGACGCTTCACTGGACCCCGAAGCCCCGTGGCCTCCCGCGCCCCGCGCTCGCGCCT
GCCGCGTACTGCCTTGGGCCCTGGTCGCGGGGCTGCTGCTGCTGCTGCTGCTCGCTGCCGCCTGCGCCGT
CTTCCTCGCCTGCCCCTGGGCCGTGTCCGGGGCTCGCGCCTCGCCCGGCTCCGCGGCCAGCCCGAGACTC
CGCGAGGGTCCCGAGCTTTCGCCCGACGATCCCGCCGGCCTCTTGGACCTGCGGCAGGGCATGTTTGCGC
AGCTGGTGGCCCAAAATGTTCTGCTGATCGATGGGCCCCTGAGCTGGTACAGTGACCCAGGCCTGGCAGG
CGTGTCCCTGACGGGGGGCCTGAGCTACAAAGAGGACACGAAGGAGCTGGTGGTGGCCAAGGCTGGAGTC
TACTATGTCTTCTTTCAACTAGAGCTGCGGCGCGTGGTGGCCGGCGAGGGCTCAGGCTCCGTTTCACTTG
CGCTGCACCTGCAGCCACTGCGCTCTGCTGCTGGGGCCGCCGCCCTGGCTTTGACCGTGGACCTGCCACC
CGCCTCCTCCGAGGCTCGGAACTCGGCCTTCGGTTTCCAGGGCCGCTTGCTGCACCTGAGTGCCGGCCAG
CGCCTGGGCGTCCATCTTCACACTGAGGCCAGGGCACGCCATGCCTGGCAGCTTACCCAGGGCGCCACAG
TCTTGGGACTCTTCCGGGTGACCCCCGAAATCCCAGCCGGACTCCCTTCACCGAGGTCGGAA

In certain embodiments, the cell further comprises one exogenous co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence having a NCBI Reference No: NP_005182 (SEQ ID NO: 86) or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 86. SEQ ID NO: 86 is provided below.

[SEQ ID NO: 86]
MGHTRRQGTSPSKCPYLNFFQLLVLAGLSHFCSGVIHVTKEVKEVATLSCGHNVSVEELAQTRIYWQKEK
KMVLTMMSGDMNIWPEYKNRTIFDITNNLSIVILALRPSDEGTYECVVLKYEKDAFKREHLAEVTLSVKA
DFPTPSISDFEIPTSNIRRIICSTSGGFPEPHLSWLENGEELNAINTTVSQDPETELYAVSSKLDFNMTT
NHSFMCLIKYGHLRVNQTFNWNTTKQEHFPDNLLPSWAITLISVNGIFVICCLTYCFAPRCRERRRNERL
RRESVRPV

An exemplary nucleotide sequence encoding the amino acid sequence of SEQ ID NO: 86 is set forth in SEQ ID NO: 87. SEQ ID NO: 87 is provided below.

[SEQ ID NO: 87]
ATGGGCCACACACGGAGGCAGGGAACATCACCATCCAAGTGTCCATACCTCAATTTCTTTCAGCTCTTGG
TGCTGGCTGGTCTTTCTCACTTCTGTTCAGGTGTTATCCACGTGACCAAGGAAGTGAAAGAAGTGGCAAC
GCTGTCCTGTGGTCACAATGTTTCTGTTGAAGAGCTGGCACAAACTCGCATCTACTGGCAAAAGGAGAAG
AAAATGGTGCTGACTATGATGTCTGGGGACATGAATATATGGCCCGAGTACAAGAACCGGACCATCTTTG
ATATCACTAATAACCTCTCCATTGTGATCCTGGCTCTGCGCCCATCTGACGAGGGCACATACGAGTGTGT
TGTTCTGAAGTATGAAAAAGACGCTTTCAAGCGGGAACACCTGGCTGAAGTGACGTTATCAGTCAAAGCT
GACTTCCCTACACCTAGTATATCTGACTTTGAAATTCCAACTTCTAATATTAGAAGGATAATTTGCTCAA
CCTCTGGAGGTTTTCCAGAGCCTCACCTCTCCTGGTTGGAAAATGGAGAAGAATTAAATGCCATCAACAC
AACAGTTTCCCAAGATCCTGAAACTGAGCTCTATGCTGTTAGCAGCAAACTGGATTTCAATATGACAACC
AACCACAGCTTCATGTGTCTCATCAAGTATGGACATTTAAGAGTGAATCAGACCTTCAACTGGAATACAA
CCAAGCAAGAGCATTTTCCTGATAACCTGCTCCCATCCTGGGCCATTACCTTAATCTCAGTAAATGGAAT
TTTTGTGATATGCTGCCTGACCTACTGCTTTGCCCCAAGATGCAGAGAGAGAAGGAGGAATGAGAGATTG
AGAAGGGAAAGTGTACGCCCTGTA

In certain embodiments, the cell further comprises two exogenous co-stimulatory ligands that are 4-1BBL and CD80. In certain embodiments, the cell further comprises two exogenous co-stimulatory ligands that are 4-1BBL and CD80, wherein the 4-1BBL comprises or consists of the amino acid sequence set forth in SEQ ID NO: 84, and the CD80 comprises or consists of the amino acid sequence set forth in SEQ ID NO: 86.

Receptor-comprising cells comprising at least one exogenous co-stimulatory ligand are described in U.S. Pat. No. 8,389,282, which is incorporated by reference in its entirety.

### 5.3. Fusion Polypeptides

In certain embodiments, a presently disclosed cell comprising an immunoevasin polypeptide, and an antigen-recognizing receptor that targets an antigen further comprises a fusion polypeptide. For example, a presently disclosed cell can be further transduced with the fusion polypeptide, such that the cell expresses or is induced to express the first antigen-recognizing receptor, the second antigen-recognizing receptor, and the fusion polypeptide. The fusion polypeptide provides a co-stimulation signal to the cell. The fusion polypeptides are capable of enhancing the activity and/or efficacy of a cell comprising the first antigen-recognizing receptor (e.g., a CAR or a TCR-like fusion molecule). In certain embodiments, the fusion polypeptide comprises a) an extracellular domain and a transmembrane domain of a co-stimulatory ligand, and b) an intracellular domain of a first co-stimulatory molecule.

Non-limiting examples of the co-stimulatory ligand include tumor necrosis factor (TNF) family members, immunoglobulin (Ig) superfamily members, and combinations thereof. The TNF family member can be selected from the group consisting of 4-1BBL, OX40L, CD70, GITRL, CD40L, and combinations thereof. The Ig superfamily member can be selected from the group consisting of CD80, CD86, ICOS ligand (ICOSLG (also known as “CD275”), and combinations thereof. In certain embodiments, the co-stimulatory ligand is selected from the group consisting of 4-1BBL, OX40L, CD70, GITRL, CD40L, CD80, CD86, ICOSLG, and combinations thereof.

In certain embodiments, the fusion polypeptide comprises an extracellular domain and a transmembrane domain of a co-stimulatory ligand that is CD80. In certain embodiments, the co-stimulatory ligand is human CD80. In certain embodiments, the CD80 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 86 or a fragment thereof, and/or may optionally comprise up to one or up to two or up to three conservative amino acid substitutions. In certain embodiments, the CD80 comprises or consists of an amino acid sequence that is a consecutive portion of the amino acid sequence of SEQ ID NO: 86.

In certain embodiments, the extracellular domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 1-242 of SEQ ID NO: 86. In certain embodiments, the extracellular domain of CD80 comprises or consists of amino acids 1-242 of SEQ ID NO: 86 or a functional fragment thereof. A functional fragment can be a consecutive portion of amino acids 1-242 of SEQ ID NO: 86, which is at least about 50, at least about 75, at least about 100, at least about 125, at least about 150, at least about 175, or at least about 200, or at least about 220 amino acids in length. In certain embodiments, the functional fragment retains at least about 80%, at least about 85%, at least about 90%, at least about 95%, or at least about 100% of the primary function of the extracellular domain of CD80. Non-limiting examples of the primary functions of the extracellular domain of CD80 include binding to/interacting with CD28, binding to/interacting with CTLA-4, binding to/interacting with PD-L1, and contributing to CD80 homodimerization. In certain embodiments, an extracellular domain of CD80 comprises or consists of amino acids 1-242 of SEQ ID NO: 86.

In certain embodiments, the transmembrane domain of CD80 comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to amino acids 243-263 of SEQ ID NO: 86. In certain embodiments, the transmembrane domain of CD80 comprises or consists of amino acids 243-263 of SEQ ID NO: 86 or a fragment thereof. Such fragment can be at least about 5, at least about 10, at least about 15, or at least about 20 amino acids in length. In certain embodiments, the transmembrane domain of CD80 comprises or consists of amino acids 243-263 of SEQ ID NO: 86.

Non-limiting examples of co-stimulatory molecules include CD28, 4-1BB, OX40, ICOS, DAP-10, CD27, CD40, NKG2D, CD2, and combinations thereof.

In certain embodiments, the fusion polypeptide comprises an extracellular domain and a transmembrane domain of a co-stimulatory molecule that is 4-1BB. In certain embodiments, the co-stimulatory molecule is human 4-1BB. In certain embodiments, the 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 55 or a fragment thereof, and/or may optionally comprise up to one or up to two or up to three conservative amino acid substitutions. In certain embodiments, the 4-1BB comprises or consists of an amino acid sequence that is a consecutive portion of the amino acid sequence of SEQ ID NO: 55. In certain embodiments, the intracellular domain of 4-1BB comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 214-255 of SEQ ID NO: 55 or a fragment thereof, and/or may optionally comprise up to one or up to two or up to three conservative amino acid substitutions. In certain embodiments, the intracellular domain of 4-1BB comprises or consists of amino acids 214-255 of SEQ ID NO: 55 or a functional fragment thereof. Such functional fragment can be a consecutive portion of amino acids 214-255 of SEQ ID NO: 55, which is at least about 20, at least about 25, at least about 30, at least about 35, or at least about 40 amino acids in length. In certain embodiments, the functional fragment of amino acids 214-255 of SEQ ID NO: 55 retains at least about 80%, at least about 85%, at least about 90%, at least about 95%, or at least about 100% of the primary functions of the intracellular domain of 4-1BB. Non-limiting examples of the primary functions of the intracellular domain of 4-1BB include providing co-stimulatory signaling for the activation and proliferation of an immunoresponsive cell (e.g., a T cell), and interacting and activating downstream adaptors (e.g., TRAFs). In certain embodiments, the intracellular domain of 4-1BB comprises or consists of amino acids 214-255 of SEQ ID NO: 55.

In certain embodiments, the co-stimulatory molecule is CD28. In certain embodiments, the co-stimulatory molecule is human CD28. In certain embodiments, the CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to the amino acid sequence set forth in SEQ ID NO: 31 or a fragment thereof, and/or may optionally comprise up to one or up to two or up to three conservative amino acid substitutions. In certain embodiments, the CD28 comprises or consists of an amino acid sequence that is a consecutive portion of the amino acid sequence of SEQ ID NO: 31. In certain embodiments, the intracellular domain of CD28 comprises or consists of an amino acid sequence that is at least about 80%, at least about 85%, at least about 90%, at least about 95%, at least about 96%, at least about 97%, at least about 98%, or at least about 99%, at least about 100% homologous or identical to amino acids 180 to 219 of SEQ ID NO: 31 or a fragment thereof, and/or may optionally comprise up to one or up to two or up to three conservative amino acid substitutions. In certain embodiments, the intracellular domain of CD28 comprises or consists of amino acids 180 to 219 of SEQ ID NO: 31 or a functional fragment thereof. A functional fragment of amino acids 180 to 219 of SEQ ID NO: 31 can be a consecutive portion of amino acids 180 to 219 of SEQ ID NO: 31, which is at least about 20, at least about 25, at least about 30, or at least about 35 amino acids in length. In certain embodiments, such functional fragment retains at least about 80%, at least about 85%, at least about 90%, at least about 95%, or at least about 100% of the primary function of the intracellular domain of CD28. Non-limiting examples of the primary functions of the intracellular domain of CD28 include providing co-stimulatory signaling for the activation and proliferation of an immunoresponsive cell (e.g., a T cell), and interacting with protein adaptors (e.g., PI3K, GRB2, and LCK). In certain embodiments, the intracellular domain of CD28 comprises or consists of amino acids 180 to 219 of SEQ ID NO: 31.

In certain embodiments, the fusion polypeptide comprises an intracellular domain of a second co-stimulatory molecule. In certain embodiments, the fusion polypeptide comprises an intracellular domain of a third co-stimulatory molecule. In certain embodiments, the fusion polypeptide comprises an intracellular domain of a fourth co-stimulatory molecule. In certain embodiments, the fusion polypeptide comprises an intracellular domain of a fifth co-stimulatory molecule. In certain embodiments, the first, second, third, fourth, and fifth co-stimulatory molecules can be the same or different from each other.

In certain embodiments, the fusion polypeptide comprises an extracellular domain and a transmembrane domain of a co-stimulatory ligand that is CD80, and an intracellular domain of a co-stimulatory molecule that is 4-1BB. In certain embodiments, the fusion polypeptide comprises or consists of an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 88. In certain embodiments, the fusion polypeptide comprises or consists of the amino acid sequence set forth in SEQ ID NO: 88. SEQ ID NO: 88 is provided below.

[SEQ ID NO: 88]
MGHTRRQGTSPSKCPYLNFFQLLVLAGLSHFCSGVIHVTKEVKEVATLSCGHNVSVEELAQTRIYWQKEK
KMVLTMMSGDMNIWPEYKNRTIFDITNNLSIVILALRPSDEGTYECVVLKYEKDAFKREHLAEVTLSVKA
DFPTPSISDFEIPTSNIRRIICSTSGGFPEPHLSWLENGEELNAINTTVSQDPETELYAVSSKLDFNMTT
NHSFMCLIKYGHLRVNQTFNWNTTKQEHFPDNLLPSWAITLISVNGIFVICCLTYCFKRGRKKLLYIFKQ
PFMRPVQTTQEEDGCSCRFPEEEEGGCEL

In certain embodiments, the fusion polypeptide comprises an extracellular domain and a transmembrane domain of a co-stimulatory ligand that is CD80, an intracellular domain of a first co-stimulatory molecule that is 4-1BB, and an intracellular domain of a second co-stimulatory molecule that is CD28.

In certain embodiments, the fusion polypeptide comprises an amino acid sequence that is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or at least about 100% homologous or identical to the amino acid sequence set forth in SEQ ID NO: 89. In certain embodiments, the fusion polypeptide comprises or consists of the amino acid sequence set forth in SEQ ID NO: 89. SEQ ID NO: 89 is provided below.

[SEQ ID NO: 89]
MGHTRRQGTSPSKCPYLNFFQLLVLAGLSHFCSGVIHVTKEVKEVATLSCGHNVSVEELAQTRIYWQKEK
KMVLTMMSGDMNIWPEYKNRTIFDITNNLSIVILALRPSDEGTYECVVLKYEKDAFKREHLAEVTLSVKA
DFPTPSISDFEIPTSNIRRIICSTSGGFPEPHLSWLENGEELNAINTTVSQDPETELYAVSSKLDFNMTT
NHSFMCLIKYGHLRVNQTFNWNTTKQEHFPDNLLPSWAITLISVNGIFVICCLTYCFRSKRSRLLHSDYM
NMTPRRPGPTRKHYQPYAPPRDFAAYRKRGRKKLLYIFKQPFMRPVQTTQEEDGCSCRFPEEEEGGCEL

Various modified fusion polypeptides are disclosed in International Patent Application No. PCT/US20/42753, which is incorporated by reference hereby in its entirety.

### 5.4. Gene Disruptions

In certain embodiments, a presently disclosed cell comprising an immunoevasin polypeptide and an antigen-recognizing receptor that targets an antigen further comprises a gene disruption of a TCR locus. Non-limiting examples of TCR loci include a TRAC locus, a TRBC locus, a TRDC locus, a TRGC locus, or a combination thereof. In certain embodiments, the gene disruption of the TCR locus results in a non-functional T cell receptor. In certain embodiments, the gene disruption of the TCR locus results in knockout of the gene expression of TCRα, TCRβ, TCRγ, TCRδ, or a combination thereof.

In certain embodiments, the gene disruption of the TCR locus can be a disruption of the coding region of the TRAC locus and/or a disruption of the non-coding region of the TRAC locus. In certain embodiments, the gene disruption of the TRAC locus comprises a disruption of the coding region of the TRAC locus. In certain embodiments, the gene disruption of the TRAC locus comprises an insertion at the coding region of the TRAC locus. Human TRAC protein comprises four exons: exon 1, exon 2, exon 3, and exon 4. In certain embodiments, the gene disruption of the TRAC locus comprises a disruption at one or more of exon 1, exon 2, exon 3, and exon 4 of the TRAC locus. In certain embodiments, the gene disruption of the TRAC locus comprises a disruption at exon 1 of the TRAC locus. In certain embodiments, the gene disruption of the TRAC locus comprises an insertion at exon 1 of the TRAC locus.

In certain embodiments, the gene disruption of the TCR locus can be a disruption of the coding region of the TRBC locus and/or a disruption of the non-coding region of the TRBC locus. In certain embodiments, the gene disruption of the TRBC locus comprises a disruption of the coding region of the TRBC locus. In certain embodiments, the gene disruption of the TRBC locus comprises an insertion at the coding region of the TRBC locus. Human TRBC protein comprises four exons: exon 1, exon 2, exon 3, and exon 4. In certain embodiments, the gene disruption of the TRBC locus comprises a disruption at one or more of exon 1, exon 2, exon 3, and exon 4 of the TRBC locus. In certain embodiments, the gene disruption of the TRBC locus comprises a disruption at exon 1 of the TRBC locus. In certain embodiments, the gene disruption of the TRBC locus comprises an insertion at exon 1 of the TRBC locus.

In certain embodiments, the gene disruption of the TCR locus can be a disruption of the coding region of the TRDC locus and/or a disruption of the non-coding region of the TRDC locus. In certain embodiments, the gene disruption of the TRDC locus comprises a disruption of the coding region of the TRDC locus. In certain embodiments, the gene disruption of the TRDC locus comprises an insertion at the coding region of the TRDC locus. Human TRDC protein comprises four exons: exon 1, exon 2, exon 3, and exon 4. In certain embodiments, the gene disruption of the TRDC locus comprises a disruption at one or more of exon 1, exon 2, exon 3, and exon 4 of the TRDC locus. In certain embodiments, the gene disruption of the TRDC locus comprises a disruption at exon 1 of the TRDC locus. In certain embodiments, the gene disruption of the TRDC locus comprises an insertion at exon 1 of the TRDC locus.

In certain embodiments, the gene disruption of the TCR locus can be a disruption of the coding region of the TRGC locus and/or a disruption of the non-coding region of the TRGC locus. In certain embodiments, the gene disruption of the TRGC locus comprises a disruption of the coding region of the TRGC locus. In certain embodiments, the gene disruption of the TRGC locus comprises an insertion at the coding region of the TRGC locus. Human TRGC protein comprises three exons: exon 1, exon 2, and exon 3. In certain embodiments, the gene disruption of the TRGC locus comprises a disruption at one or more of exon 1, exon 2, and exon 3 of the TRGC locus. In certain embodiments, the gene disruption of the TRGC locus comprises a disruption at exon 1 of the TRGC locus. In certain embodiments, the gene disruption of the TRGC locus comprises an insertion at exon 1 of the TRGC locus.

In certain embodiments, the TCR locus is a human TCR locus. The gene disruption of the TCR locus can be generated by any suitable gene editing methods. In certain embodiments, the gene disruption of the TCR locus (e.g., knockout of the TCR locus) is generated using a viral method. In certain embodiments, the viral method comprises a viral vector. In certain embodiments, the viral vector is a retroviral vector (e.g., a gamma-retroviral vector or a lentiviral vector). Other viral vectors include adenoviral vectors, adena-associated viral vectors, vaccinia viruses, bovine papilloma viruses, and herpes viruses (e.g., such as Epstein-Barr Virus).

In certain embodiments, the gene disruption of the TCR locus (e.g., knockout of the TCR locus) is generated using a non-viral method. Non-viral approaches can also be employed for genetic modification of a cell. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue or are injected systemically. Recombinant receptors can also be derived or obtained using transposases or targeted nucleases (e.g. Zinc finger nucleases, meganucleases, or TALE nucleases, CRISPR). Transient expression may be obtained by RNA electroporation.

Any targeted genome editing methods can also be used to generate the gene disruption of the TCR locus. In certain embodiments, the gene disruption of the TCR locus is generated by a method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

In certain embodiments, a CRISPR system is used to generate the gene disruption of the TCR locus.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to form a single-guide RNA (sgRNA). This sgRNA can be joined together with the Cas9 gene and made into a plasmid in order to be transfected into cells. In certain embodiments, the CRISPR system comprises base editors. In certain embodiments, the CRISPR system comprises transposases/recombinases. In certain embodiments, the CRISPR system comprises prime editors. In certain embodiments, the CRISPR system comprises an epigenetic modulator. In certain embodiments, the CRISPR system comprises is a CRISPRoff system. Additional details on the CRISPR systems of the presently disclosed subject matter can be found in Anzalone et al., Nature biotechnology 38.7 (2020): 824-844 and in Nuñez et al., Cell 184.9 (2021): 2503-2519, the contents of each of which are incorporated by reference in their entireties.

In certain embodiments, the TCR locus is disrupted using a gRNA molecule to knockout expression of TCR. The gRNA molecule can target a coding sequence of a TCR locus (e.g., a human TRAC gene) or a non-coding sequence of a TCR locus (e.g., a human TRAC gene). In certain embodiments, the gRNA molecule targets a coding sequence of a TCR locus (e.g., a human TRAC gene). In certain embodiments, the gRNA molecule targets a target sequence within a human TRAC gene.

In certain embodiments, zinc-finger nucleases are used to generate the gene disruption of the TCR locus. A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

In certain embodiments, a TALEN system is used to generate the gene disruption of the TCR locus. Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome. cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

In certain embodiments, the cell is a T cell, and the antigen-recognizing receptor (e.g., one disclosed in Section 3) is integrated at a TCR locus within the genome of the T cell. In certain embodiments, the cell is a T cell, and the antigen-recognizing receptor is integrated at a TRAC locus. Methods of targeting an antigen-recognizing receptor (e.g., a CAR) to a site within the genome of T cell are disclosed in WO2017180989 and Eyquem et al., Nature. (2017 Mar. 2); 543(7643): 113-117, both of which are incorporated by reference in their entireties. In certain embodiments, the cell is a T Cell, the antigen-recognizing receptor is a CAR, and the CAR is integrated at a TRAC locus. In certain embodiments, the cell further comprises a gene disruption of a TRBC locus. In certain embodiments, the gene disruption of a TRBC locus results in knockout of TRBC locus.

In certain embodiments, a presently disclosed cell comprising an antigen-recognizing receptor that targets an antigen further comprises a gene disruption of a NLRC5 locus. Additionally or alternatively, a presently disclosed cell comprising an immunoevasin polypeptide and an antigen-recognizing receptor that targets an antigen further comprises a gene disruption of a NLRC5 locus. In certain embodiments, the gene disruption of the NLRC5 locus results in a non-functional NLRC5. In certain embodiments, the gene disruption of the NLRC5 locus results in knockout of the gene expression of NLRC5. In certain embodiments, the gene disruption of the NLRC5 locus can be a disruption of the coding region of the NLRC5 locus and/or a disruption of the non-coding region of the NLRC5 locus. In certain embodiments, the gene disruption of the NLRC5 locus comprises a disruption of the coding region of the NLRC5 locus. In certain embodiments, the gene disruption of the NLRC5 locus comprises an insertion at the coding region of the NLRC5 locus. Human NLRC5 protein comprises 49 exons: exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, and exon 49. In certain embodiments, the gene disruption of the NLRC5 locus comprises a disruption at one or more of exon 1, exon 2, exon 3, exon 4, exon 5, exon 6, exon 7, exon 8, exon 9, exon 10, exon 11, exon 12, exon 13, exon 14, exon 15, exon 16, exon 17, exon 18, exon 19, exon 20, exon 21, exon 22, exon 23, exon 24, exon 25, exon 26, exon 27, exon 28, exon 29, exon 30, exon 31, exon 32, exon 33, exon 34, exon 35, exon 36, exon 37, exon 38, exon 39, exon 40, exon 41, exon 42, exon 43, exon 44, exon 45, exon 46, exon 47, exon 48, and exon 49 of the NLRC5 locus. In certain embodiments, the gene disruption of the NLRC5 locus comprises a disruption at exon 1 of the NLRC5 locus. In certain embodiments, the gene disruption of the NLRC5 locus comprises an insertion at exon 1 of the NLRC5 locus.

Immune rejection of stem cells is due to the expression of major histocompatibility complex (MHC) on the surface of these cells (Zhang et al., J Cell Mol Med. (2020); 24:695-710). MHC presents “non-self” antigens to CD8⁺ T cells that eliminate the transplanted cells through direct cytotoxic effect (Zhang 2020). Due to the polymorphic nature of the MHC genes, it is often difficult to identify a perfect match between donor and recipient prior to transplantation (Zhang 2020). NLRC5 is a transcription factor that regulates the classical MHC class I genes (e.g., HLA-A, HLA-B and HLA-C) and the non-classical MHC class I genes (e.g., HLA-E). Further, NLRC5 also upregulates the expression of the MHC class I accessory genes (e.g., B2M, LMP2 and TAP1) (Kobayashi and van den Elsen, Nat Rev Immunol 12, 813-820 (2012)). In certain embodiments, the gene disruption of the NLRC5 locus can reduce immune rejection, thereby making the cells suitable for allogeneic settings.

In certain embodiments, a presently disclosed cell comprising an immunoevasin polypeptide and an antigen-recognizing receptor that targets an antigen further comprises a gene disruption of a RFX5 locus. In certain embodiments, the gene disruption of the RFX5 locus results in a non-functional RFX5. In certain embodiments, the gene disruption of the RFX5 locus results in knockout of the gene expression of RFX5. In certain embodiments, the gene disruption of the RFX5 locus can be a disruption of the coding region of the RFX5 locus and/or a disruption of the non-coding region of the RFX5 locus. In certain embodiments, the gene disruption of the NLRC5 locus comprises a disruption of the coding region of the RFX5 locus. In certain embodiments, the gene disruption of the RFX5 locus comprises an insertion at the coding region of the RFX5 locus. Human RFX5 protein comprises 11 exons: exon 1, exon 2, exon 3, exon 4, exon 5, exon 6, exon 7, exon 8, exon 9, exon 10, and exon 11. In certain embodiments, the gene disruption of the RFX5 locus comprises a disruption at one or more of exon 1, exon 2, exon 3, exon 4, exon 5, exon 6, exon 7, exon 8, exon 9, exon 10, and exon 11 of the RFX5 locus. In certain embodiments, the gene disruption of the RFX5 locus comprises a disruption at exon 1 of the RFX5 locus. In certain embodiments, the gene disruption of the RFX5 locus comprises an insertion at exon 1 of the RFX5 locus.

Immune rejection of stem cells is due to the expression of major histocompatibility complex (MHC) on the surface of these cells (Zhang et al., J Cell Mol Med. (2020); 24:695-710). MHC presents “non-self” antigens to CD8⁺ T cells that eliminate the transplanted cells through direct cytotoxic effect (Zhang 2020). Due to the polymorphic nature of the MHC genes, it is often difficult to identify a perfect match between donor and recipient prior to transplantation (Zhang 2020). RFX5 is a transcription factor that activates transcription of the MHC class II genes (e.g., HLA-A, HLA-B and HLA-C) and the non-classical MHC class I genes (e.g., HLA-E). In certain embodiments, the gene disruption of the RFX5 locus can reduce immune rejection, thereby making the cells suitable for allogeneic settings.

### 5.5. Exemplified Cells

In certain embodiments, the cell comprises an immunoevasin and an antigen-recognizing receptor. In certain embodiments, the immunoevasin comprises an expression profile regulated by a TRAC promoter. In certain embodiments, the immunoevasin is a NEF polypeptide. In certain embodiments, the antigen-recognizing receptor comprises an expression profile regulated by a TRAC promoter. In certain embodiments, the antigen-recognizing receptor is a CAR. In certain embodiments, the CAR comprises a 1XX CD3ζ polypeptide. In certain embodiments, the cell further comprises a gene disruption of a TRAC locus. In certain embodiments, the gene disruption of the TRAC locus results in knockout of the TRAC locus. In certain embodiments, the cell is a T cell.

In certain embodiments, the cell comprises an immunoevasin and an antigen-recognizing receptor. In certain embodiments, the immunoevasin comprises an expression profile regulated by a EF1 promoter. In certain embodiments, the immunoevasin is a NEF polypeptide. In certain embodiments, the antigen-recognizing receptor comprises an expression profile regulated by a TRAC promoter. In certain embodiments, the antigen-recognizing receptor is a CAR. In certain embodiments, the CAR comprises a 1XX CD3ζ polypeptide. In certain embodiments, the cell further comprises a gene disruption of a TRAC locus. In certain embodiments, the gene disruption of the TRAC locus results in knockout of the TRAC locus. In certain embodiments, the cell is a T cell.

In certain embodiments, the cell comprises an immunoevasin and an antigen-recognizing receptor. In certain embodiments, the immunoevasin comprises an expression profile regulated by a TRAC promoter. In certain embodiments, the immunoevasin is a BNLF2a polypeptide. In certain embodiments, the antigen-recognizing receptor comprises an expression profile regulated by a TRAC promoter. In certain embodiments, the antigen-recognizing receptor is a CAR. In certain embodiments, the CAR comprises a 1XX CD3ζ polypeptide. In certain embodiments, the cell further comprises a gene disruption of a TRAC locus. In certain embodiments, the gene disruption of the TRAC locus results in knockout of the TRAC locus. In certain embodiments, the cell is a T cell.

In certain embodiments, the cell comprises an immunoevasin and an antigen-recognizing receptor. In certain embodiments, the immunoevasin comprises an expression profile regulated by a EF1 promoter. In certain embodiments, the immunoevasin is a BNLF2a polypeptide. In certain embodiments, the antigen-recognizing receptor comprises an expression profile regulated by a TRAC promoter. In certain embodiments, the antigen-recognizing receptor is a CAR. In certain embodiments, the CAR comprises a 1XX CD3ζ polypeptide. In certain embodiments, the cell further comprises a gene disruption of a TRAC locus. In certain embodiments, the gene disruption of the TRAC locus results in knockout of the TRAC locus. In certain embodiments, the cell is a T cell.

In certain embodiments, the cell comprises at least one immunoevasin and an antigen-recognizing receptor. In certain embodiments, the immunoevasin comprises an expression profile regulated by a TRAC promoter. In certain embodiments, the cell comprises a NEF polypeptide and a BNLF2a polypeptide. In certain embodiments, the antigen-recognizing receptor comprises an expression profile regulated by a TRAC promoter. In certain embodiments, the antigen-recognizing receptor is a CAR. In certain embodiments, the CAR comprises a 1XX CD3ζ polypeptide. In certain embodiments, the cell further comprises a gene disruption of a TRAC locus. In certain embodiments, the gene disruption of the TRAC locus results in knockout of the TRAC locus. In certain embodiments, the cell is a T cell.

In certain embodiments, the cell comprises at least one immunoevasin and an antigen-recognizing receptor. In certain embodiments, the immunoevasin comprises an expression profile regulated by a EF1 promoter. In certain embodiments, the cell comprises a NEF polypeptide and a BNLF2a polypeptide. In certain embodiments, the antigen-recognizing receptor comprises an expression profile regulated by a TRAC promoter. In certain embodiments, the antigen-recognizing receptor is a CAR. In certain embodiments, the CAR comprises a 1XX CD3ζ polypeptide. In certain embodiments, the cell further comprises a gene disruption of a TRAC locus. In certain embodiments, the gene disruption of the TRAC locus results in knockout of the TRAC locus. In certain embodiments, the cell is a T cell.

In certain embodiments, the cell comprises at least one immunoevasin and an antigen-recognizing receptor. In certain embodiments, the immunoevasin comprises an expression profile regulated by a EF1 promoter or a TRAC promoter. In certain embodiments, the cell comprises a NEF polypeptide and/or a BNLF2a polypeptide. In certain embodiments, the antigen-recognizing receptor comprises an expression profile regulated by a TRAC promoter. In certain embodiments, the antigen-recognizing receptor is a CAR. In certain embodiments, the CAR comprises a 1XX CD3ζ polypeptide. In certain embodiments, the cell further comprises a gene disruption of a TRAC locus, a NLRC5 locus, a RFX5, or a combination thereof. In certain embodiments, the gene disruption of the TRAC locus results in knockout of the TRAC locus. In certain embodiments, the gene disruption of the NLRC5 locus results in knockout of the NLRC5 locus. In certain embodiments, the gene disruption of the RFX5 locus results in knockout of the RFX5 locus. In certain embodiments, the cell is a T cell.

In certain embodiments, the cell comprises an antigen-recognizing receptor and at least one gene disruption. In certain embodiments, the antigen-recognizing receptor comprises an expression profile regulated by a TRAC promoter. In certain embodiments, the antigen-recognizing receptor is a CAR. In certain embodiments, the CAR comprises a 1XX CD3ζ polypeptide. In certain embodiments, the at least one gene disruption is a gene disruption of a TRAC locus, a NLRC5 locus, a RFX5, or a combination thereof. In certain embodiments, the gene disruption of the TRAC locus results in knockout of the TRAC locus. In certain embodiments, the gene disruption of the NLRC5 locus results in knockout of the NLRC5 locus. In certain embodiments, the gene disruption of the RFX5 locus results in knockout of the RFX5 locus. In certain embodiments, the cell is a T cell.

### 6. Formulations and Administration

The presently disclosed subject matter provides compositions comprising presently disclosed cells (e.g., disclosed in Section 5). In certain embodiments, the compositions are pharmaceutical compositions that further comprise a pharmaceutically acceptable excipient.

Compositions comprising the presently disclosed cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Compositions comprising the presently disclosed cells can be provided systemically or directly to a subject for inducing and/or enhancing an immune response to an antigen and/or treating and/or preventing a neoplasm. In certain embodiments, the presently disclosed cells or compositions comprising thereof are directly injected into an organ of interest (e.g., an organ affected by a neoplasm). Alternatively, the presently disclosed cells or compositions comprising thereof are provided indirectly to the organ of interest, for example, by administration into the circulatory system (e.g., the tumor vasculature). Expansion and differentiation agents can be provided prior to, during or after administration of the cells or compositions to increase production of cells in vitro or in vivo.

The quantity of cells to be administered can vary for the subject being treated. In certain embodiments, between about 10⁴ and about 10¹⁰, between about 10⁴ and about 10⁷, between about 10⁵ and about 10⁷, between about 10⁵ and about 10⁹, or between about 10⁶ and about 10⁸ of the presently disclosed cells are administered to a subject. In certain embodiments, between about 10⁵ and about 10⁷ of the presently disclosed cells are administered to a subject. More effective cells may be administered in even smaller numbers. Usually, at least about 1×10⁵ cells will be administered, eventually reaching about 1×10¹⁰ or more. In certain embodiments, at least about 1×10⁵, about 5×10⁵, about 1×10⁶, about 5×10⁶, about 1×10⁷, about 5×10⁷, about 1×10⁸, or about 5×10⁸ of the presently disclosed cells are administered to a subject. In certain embodiments, about 1×10⁵ of the presently disclosed cells are administered to a subject. In certain embodiments, about 5×10⁵ of the presently disclosed cells are administered to a subject. In certain embodiments, about 1×10⁶ of the presently disclosed cells are administered to a subject. The precise determination of what would be considered an effective dose can be based on factors individual to each subject, including their size, age, sex, weight, and condition of the particular subject. Dosages can be readily ascertained by those skilled in the art from this disclosure and the knowledge in the art.

The presently disclosed cells and compositions can be administered by any method known in the art including, but not limited to, intravenous administration, subcutaneous administration, intranodal administration, intratumoral administration, intrathecal administration, intrapleural administration, intraosseous administration, intraperitoneal administration, pleural administration, and direct administration to the subject. The presently disclosed cells can be administered in any physiologically acceptable vehicle, normally intravascularly, although they may also be introduced into bone or other convenient site where the cells may find an appropriate site for regeneration and differentiation (e.g., thymus). The cells can be introduced by injection, catheter, or the like.

Compositions comprising the presently disclosed cells can be provided systemically or directly to a subject for inducing and/or enhancing an immune response to an antigen and/or treating and/or preventing a neoplasm (e.g., cancer), pathogen infection, or infectious disease. In certain embodiments, the presently disclosed cells, compositions, or nucleic acid compositions are directly injected into an organ of interest (e.g., an organ affected by a neoplasm). Alternatively, the presently disclosed cells, compositions, or nucleic acid compositions are provided indirectly to the organ of interest, for example, by administration into the circulatory system (e.g., the tumor vasculature). Expansion and differentiation agents can be provided prior to, during or after administration of the cells, compositions, or nucleic acid compositions to increase production of the cells (e.g., T cells (e.g., CTL cells) or NK cells) in vitro or in vivo.

The presently disclosed compositions can be pharmaceutical compositions comprising the presently disclosed cells or their progenitors and a pharmaceutically acceptable carrier. Administration can be autologous or heterologous. For example, cells, or progenitors can be obtained from one subject, and administered to the same subject or a different, compatible subject. Peripheral blood derived cells or their progeny (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed cell), it can be formulated in a unit dosage injectable form (solution, suspension, emulsion).

### 7. Methods of Treatment

The presently disclosed subject matter provides various methods of using the presently disclosed cells or compositions comprising thereof. The presently disclosed cells and compositions comprising thereof can be used in a therapy or medicament. For example, the presently disclosed subject matter provides methods for inducing and/or increasing an immune response in a subject in need thereof. The presently disclosed cells and compositions comprising thereof can be used for reducing tumor burden in a subject. The presently disclosed cells and compositions comprising thereof can reduce the number of tumor cells, reduce tumor size, and/or eradicate the tumor in the subject. The presently disclosed cells and compositions comprising thereof can be used for treating and/or preventing a tumor (or neoplasm) in a subject. The presently disclosed cells and compositions comprising thereof can be used for prolonging the survival of a subject suffering from a tumor. In certain embodiments, the tumor is cancer. The presently disclosed cells, compositions, and nucleic acid compositions can also be used for treating and/or preventing a pathogen infection or other infectious disease in a subject, such as an immunocompromised human subject. The presently disclosed cells, compositions, and nucleic acid compositions can also be used for treating and/or preventing an autoimmune disease in a subject. In certain embodiments, each of the above-noted methods comprises administering the presently disclosed cells or a composition (e.g., a pharmaceutical composition) comprising thereof to achieve the desired effect, e.g., palliation of an existing condition or prevention of recurrence. For treatment, the amount administered is an amount effective in producing the desired effect. An effective amount can be provided in one or a series of administrations. An effective amount can be provided in a bolus or by continuous perfusion.

Non-limiting examples of tumors (or neoplasms) include blood cancers (e.g. leukemias, lymphomas, and myelomas), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, throat cancer, melanoma, neuroblastoma, adenocarcinoma, glioma, soft tissue sarcoma, and various carcinomas (including prostate and small cell lung cancer). Suitable carcinomas further include any known in the field of oncology, including, but not limited to, astrocytoma, fibrosarcoma, myxosarcoma, liposarcoma, oligodendroglioma, ependymoma, medulloblastoma, primitive neural ectodermal tumor (PNET), chondrosarcoma, osteogenic sarcoma, pancreatic ductal adenocarcinoma, small and large cell lung adenocarcinomas, chordoma, angiosarcoma, endotheliosarcoma, squamous cell carcinoma, bronchoalveolar carcinoma, epithelial adenocarcinoma, and liver metastases thereof, lymphangiosarcoma, lymphangioendotheliosarcoma, hepatoma, cholangiocarcinoma, synovioma, mesothelioma, Ewing's tumor, rhabdomyosarcoma, colon carcinoma, basal cell carcinoma, sweat gland carcinoma, papillary carcinoma, sebaceous gland carcinoma, papillary adenocarcinoma, cystadenocarcinoma, medullary carcinoma, bronchogenic carcinoma, renal cell carcinoma, bile duct carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia, and heavy chain disease, breast tumors such as ductal and lobular adenocarcinoma, squamous and adenocarcinomas of the uterine cervix, uterine and ovarian epithelial carcinomas, prostatic adenocarcinomas, transitional squamous cell carcinoma of the bladder, B and T cell lymphomas (nodular and diffuse) plasmacytoma, acute and chronic leukemias, malignant melanoma, soft tissue sarcomas and leiomyosarcomas. In certain embodiments, the neoplasm is cancer. In certain embodiments, the neoplasm is selected from the group consisting of blood cancers (e.g. leukemias, lymphomas, and myelomas), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and throat cancer. In certain embodiments, the presently disclosed cells, compositions, nucleic acid compositions can be used for treating and/or preventing blood cancers (e.g., leukemias, lymphomas, and myelomas) or ovarian cancer, which are not amenable to conventional therapeutic interventions.

In certain embodiments, the tumor and/or neoplasm is a solid tumor. Non limiting examples of solid tumor include renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

In certain embodiments, the tumor and/or neoplasm is a blood cancer. Non-limiting examples of blood cancer include multiple myeloma, leukemia, and lymphomas. Non-limiting examples of leukemia include acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, B cell prolymphocytic leukemia, B-cell precursor acute lymphoblastic leukemia, and T-cell precursor acute lymphoblastic leukemia. The lymphoma can be Hodgkin's lymphoma or non-Hodgkin's lymphoma. In certain embodiments, the lymphoma is non-Hodgkin's lymphoma, including B-cell non-Hodgkin's lymphoma and T-cell non-Hodgkin's lymphoma. In certain embodiments, the lymphoma is T-cell precursor acute lymphoblastic lymphoma.

In certain embodiments, the tumor and/or neoplasm is a B cell malignancy. Non-limiting examples of B cell malignancy include B cell non-Hodgkin lymphomas (NHL), B cell Hodgkin's lymphomas, B cell acute lymphocytic leukemia (ALL), B cell chronic lymphocytic leukemia (CLL), multiple myeloma (MM), CLL with Richter's transformation, and CNS lymphoma.

In certain embodiments, the tumor and/or neoplasm is a B cell-related neoplasm. Non-limiting examples of B cell-related neoplasm include chronic lymphocytic leukemia/small lymphocytic lymphoma (CLL/SLL), B-cell prolymphocytic leukemia, splenic marginal zone lymphoma, hairy cell leukemia, splenic B-cell lymphoma/leukemia (unclassifiable), splenic diffuse red pulp small B-cell lymphoma, lymphoplasmacytic lymphoma, Waldenström macroglobulinemia, monoclonal gammopathy of undetermined significance (MGUS, IgM), heavy-chain diseases (μ, γ, α), MGUS (IgG/A), plasma cell myeloma, solitary plasmacytoma of bone, extraosseous plasmacytoma, monoclonal immunoglobulin deposition diseases, extranodal marginal zone lymphoma of mucosa-associated lymphoid tissue (MALT lymphoma), nodal marginal zone lymphoma, pediatric nodal marginal zone lymphoma, follicular lymphoma, in situ follicular neoplasia, duodenal-type follicular lymphoma, pediatric-type follicular lymphoma, large B-cell lymphoma with IRF4 rearrangement, primary cutaneous follicle center cell lymphoma, mantle cell lymphoma, in situ mantle cell neoplasia, diffuse large B-cell lymphoma (DLBCL) (not otherwise specified (NOS)), germinal center B-cell type, activated B-cell type, T-cell/histiocyte-rich large B-cell lymphoma, primary DLBCL of the central nervous system (CNS), primary cutaneous DLBCL (leg type), Epstein-Barr virus (EBV)-positive DLBCL (NOS), EBV-positive mucocutaneous ulcer, DLBCL associated with chronic inflammation, lymphomatoid granulomatosis, primary mediastinal (thymic) large B-cell lymphoma, intravascular large B-cell lymphoma, anaplastic lymphoma kinase (ALK)-positive large B-cell lymphoma, plasmablastic lymphoma, primary effusion lymphoma, human herpesvirus 8 (HHV-8)-associated DLBCL (NOS), Burkitt lymphoma, Burkitt-like lymphoma with 11q aberration, high-grade B-cell lymphoma with MYC and BLC2 and/or BCL6 rearrangements, high-grade B-cell lymphoma (NOS), and B-cell lymphoma (unclassifiable).

In certain embodiments, the tumor and/or neoplasm is a myeloid disorder. Non-limiting examples of myeloid disorders include myelodysplastic syndromes, myeloproliferative neoplasms, chronic myelomonocytic leukemia, acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

The presently disclosed subject matter provides methods for treating and/or preventing a viral infection in a subject. The method can comprise administering an effective amount of the presently disclosed cells, a presently disclosed composition, or a presently disclosed nucleic acid composition to a subject having a viral infection. Non-limiting examples of viral infections include those caused by cytomegalovirus (CMV), Epstein-Barr virus (EBV), hepatitis A, B, C, D, E, F or G, human immunodeficiency virus (HIV), adenovirus, BK polyomavirus, coronavirus, coxsackievirus, poliovirus, herpes simplex type 1, herpes simplex type 2, human cytomegalovirus, human herpesvirus type 8, varicella-zoster virus, influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picornaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). In certain embodiments, the viral infections include human respiratory coronavirus, influenza viruses A-C, hepatitis viruses A to G, and herpes simplex viruses 1-9. In certain embodiments, the subject has an immunodeficiency.

The presently disclosed subject matter provides methods for treating and/or preventing a bacterial infection in a subject. The method can comprise administering an effective amount of the presently disclosed cells, a presently disclosed composition, or a presently disclosed nucleic acid composition to a subject having a bacterial infection. Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Escherichia coli, Bacillus anthracis, Rickettsia, Bartonella henselae, Bartonella quintana, Coxiella burnetii, chlamydia, Mycobacterium leprae, Salmonella, Shigella, Yersinia enterocolitica, Yersinia pseudotuberculosis; Legionella pneumophila; Mycobacterium tuberculosis; Listeria monocytogenes; Mycoplasma spp., Pseudomonas fluorescens, Vibrio cholerae, Haemophilus influenzae, Bacillus anthracis, Treponema pallidum, Leptospira, Borrelia, Corynebacterium diphtheriae, Francisella, Brucella melitensis, Campylobacter jejuni, Enterobacter, Proteus mirabilis, Proteus, and Klebsiella pneumoniae.

The presently disclosed subject matter provides methods for treating and/or preventing an autoimmune disease in a subject. The method can comprise administering an effective amount of the presently disclosed cells, a presently disclosed composition, or a presently disclosed nucleic acid composition to a subject having an autoimmune disease.

The presently disclosed subject matter provides methods for treating and/or preventing an inflammatory disease in a subject. The method can comprise administering an effective amount of the presently disclosed cells, a presently disclosed composition, or a presently disclosed nucleic acid composition to a subject having an infectious disease.

Non-limiting examples of autoimmune diseases and inflammatory diseases or conditions thereof include arthritis, e.g., rheumatoid arthritis (RA), Type I diabetes, systemic lupus erythematosus (SLE), inflammatory bowel disease, ulcerative colitis, psoriasis, psoriatic arthritis, scleroderma, autoimmune thyroid disease, Grave's disease, Crohn's disease, multiple sclerosis, systemic sclerosis, asthma, organ transplant rejection, a disease or condition associated with transplant, Takayasu arteritis, giant-cell arteritis, Kawasaki disease, polyarteritis nodosa, Behcet's syndrome, Wegener's granulomatosis, ANCA-vasculitides, Churg-Strauss syndrome, microscopic polyangiitis, vasculitis of connective tissue diseases, Hennoch-Schonlein purpura, cryoglobulinemic vasculitis, cutaneous leukocytoclastic angiitis, Sarcoidosis, Cogan's syndrome, Wiskott-Aldrich Syndrome, primary angiitis of the CNS, thromboangiitis obliterans, paraneoplastic arteritis, myelodysplastic syndrome, erythema elevatum diutinum, amyloidosis, autoimmune myositis, Guillain-Barre Syndrome, histiocytosis, atopic dermatitis, pulmonary fibrosis, glomerulonephritis, Whipple's disease, Still's disease, Sjogren's syndrome, osteomyelofibrosis, chronic inflammatory demyelinating polyneuropathy, Kimura's disease, systemic sclerosis, chronic periaortitis, chronic prostatitis, idiopathic pulmonary fibrosis, chronic granulomatous disease, idiopathic, bleomycin-induced lung inflammation, cytarabine-induced lung inflammation, autoimmune thrombocytopenia, autoimmune neutropenia, autoimmune hemolytic anemia, autoimmune lymphocytopenia, chronic autoimmune thyroiditis, autoimmune hepatitis, Hashimoto's thyroiditis, atopic thyroiditis, Graves disease, autoimmune polyglandular syndrome, autoimmune Addison syndrome, and/or myasthenia gravis. In accordance with the presently disclosed subject matter, the above-described various methods can comprise administering to the subject a checkpoint immune blockade agent.

The subjects can have an advanced form of disease, in which case the treatment objective can include mitigation or reversal of disease progression, and/or amelioration of side effects. The subjects can have a history of the condition, for which they have already been treated, in which case the therapeutic objective will typically include a decrease or delay in the risk of recurrence.

Further modification can be introduced to the presently disclosed cells to avert or minimize the risks of immunological complications (known as “malignant T-cell transformation”), e.g., graft versus-host disease (GvHD), or when healthy tissues express the same target antigens as the tumor cells, leading to outcomes similar to GvHD. A potential solution to this problem is engineering a suicide gene into the presently disclosed cells. Suitable suicide genes include, but are not limited to, Herpes simplex virus thymidine kinase (hsv-tk), inducible Caspase 9 Suicide gene (iCasp-9), and a truncated human epidermal growth factor receptor (EGFRt) polypeptide. In certain embodiments, the suicide gene is an EGFRt polypeptide. The EGFRt polypeptide can enable T-cell elimination by administering anti-EGFR monoclonal antibody (e.g., cetuximab). EGFRt can be covalently joined to the upstream of the antigen-recognizing receptor. The suicide gene can be included within the vector comprising nucleic acids encoding a presently disclosed antigen-recognizing receptor. In this way, administration of a prodrug designed to activate the suicide gene (e.g., a prodrug (e.g., AP1903 that can activate iCasp-9) during malignant T-cell transformation (e.g., GVHD) triggers apoptosis in the suicide gene-activated cells expressing the presently disclosed antigen-recognizing receptor. The incorporation of a suicide gene into a presently disclosed antigen-recognizing receptor gives an added level of safety with the ability to eliminate the majority of receptor-expressing cells within a very short time period. A presently disclosed cell incorporated with a suicide gene can be pre-emptively eliminated at a given timepoint post the cell infusion, or eradicated at the earliest signs of toxicity.

### 8. Kits

The presently disclosed subject matter provides kits for inducing and/or enhancing an immune response and/or treating and/or preventing a neoplasm, a pathogen infection, an autoimmune disease, or an inflammation disease in a subject. In certain embodiments, the kit comprises an effective amount of presently disclosed cells, a presently disclosed composition, or a presently disclosed nucleic acids. In certain embodiments, the kit comprises a sterile container; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In certain non-limiting embodiments, the kit includes an isolated nucleic acid molecule encoding an immunoevasin and an antigen-recognizing receptor (e.g., a CAR, a TCR, or a TCR-like fusion molecule) directed toward an antigen of interest in expressible form, which may optionally be comprised in the same or different vectors.

If desired, the cells, composition, or nucleic acids are provided together with instructions for administering the cells, composition, or nucleic acid composition to a subject having or at risk of developing a tumor (e.g., a cancer) or a pathogen infection (e.g., an infectious disease), or immune disorder (e.g., an autoimmune disease). The instructions generally include information about the use of the cell, composition or nucleic acids for the treatment and/or prevention of a neoplasm, or a pathogen infection (e.g., an infectious disease), or an immune disorder (e.g., an autoimmune disease). In certain embodiments, the instructions include at least one of the following: description of the therapeutic agent; dosage schedule and administration for treatment or prevention of a neoplasm, pathogen infection (e.g., an infectious disease), or immune disorder (e.g., an autoimmune disease) or symptoms thereof; precautions; warnings; indications; counter-indications; over-dosage information; adverse reactions; animal pharmacology; clinical studies; and/or references. The instructions may be printed directly on the container (when present), or as a label applied to the container, or as a separate sheet, pamphlet, card, or folder supplied in or with the container.

### 9. Exemplary Embodiments

Clause 1. A cell comprising an immunoevasin and an antigen recognizing receptor that targets an antigen.

Clause 2. The cell of clause 1, wherein the immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 3. The cell of clause 2, wherein the immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 4. The cell of clause 3, wherein the immunoevasin comprises the amino acid sequence set forth in SEQ ID NO: 1.

Clause 5. The cell of any one of clauses 1-3, further comprising a second immunoevasin.

Clause 6. The cell of clause 4, wherein the second immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 7. The cell of clause 5, wherein the second immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 8. The cell of clause 7, wherein the immunoevasin comprises the amino acid sequence set forth in SEQ ID NO: 5.

Clause 9. The cell of any one of clauses 1-8 further comprising a gene disruption of a NLRC5 locus, a RFX5 locus, or a combination thereof.

Clause 10. A cell comprising an antigen recognizing receptor that targets an antigen and a gene disruption of a NLRC5 locus, a RFX5 locus, or a combination thereof.

Clause 11. The cell of any one of clauses 1-10, wherein the antigen-recognizing receptor is a T cell receptor (TCR), a chimeric antigen receptor (CAR), or a TCR like fusion molecule.

Clause 12. The cell of clause 11, wherein the antigen-recognizing receptor is a chimeric antigen receptor (CAR).

Clause 13. The cell of clause 11 or 12, wherein the CAR comprises an extracellular antigen-binding domain that binds to the antigen, and an intracellular signaling domain that is capable of delivering an activation signal to the cell.

Clause 14. The cell of clause 13, wherein the intracellular signaling domain of the CAR comprises a CD3ζ polypeptide.

Clause 15. The cell of clause 14, wherein the CD3ζ polypeptide is a native CD3ζ polypeptide or a modified CD3ζ polypeptide.

Clause 16. The cell of clause 15, wherein the modified CD3ζ polypeptide comprises a native ITAM1, an ITAM2 variant consisting of two loss-of-function mutations, and an ITAM3 variant consisting of two loss-of-function mutations.

Clause 17. The cell of clause 16, wherein the modified CD3ζ polypeptide comprises the amino acid sequence set forth in SEQ ID NO: 52.

Clause 18. The cell of any one of clauses 13-17, wherein the intracellular signaling domain of the CAR further comprises at least one costimulatory signaling region.

Clause 19. The cell of clause 18, wherein the at least one costimulatory signaling region comprises at least an intracellular domain of a co-stimulatory molecule or a portion thereof.

Clause 20. The cell of clause 19, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

Clause 21. The cell of any one of clauses 13-20, wherein the CAR comprises a transmembrane domain.

Clause 22. The cell of clause 21, wherein the antigen-recognizing receptor is a TCR-like fusion molecule comprising i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the antigen, wherein the TCR-like fusion molecule binds to the antigen in an HLA-independent manner.

Clause 23. The cell of clause 22, wherein at least one of the TRAC polypeptide and the TRBC polypeptide is endogenous.

Clause 24. The cell of clause 22 or 23, wherein the first and the second antigen-binding chains bind to the antigen with a dissociation constant (KD) of about 1×10-8 M or less.

Clause 25. The cell of any one of clauses 22-24, wherein the first and the second antigen-binding chains bind to the antigen with a dissociation constant (KD) of about 5×10-9 M or less.

Clause 26. The cell of any one of clauses 22-25, wherein the first antigen-binding chain comprises an antigen-binding fragment of a VH of an antibody and a TRBC polypeptide, and the second antigen-binding chain comprises an antigen-binding fragment of a VL of the antibody and a TRAC polypeptide.

Clause 27. The cell of any one of clauses 22-26, wherein the first antigen-binding chain comprises an antigen-binding fragment of a VH of an antibody and a TRAC polypeptide, and the second antigen-binding chain comprises an antigen-binding fragment of a VL of the antibody and a TRBC polypeptide.

Clause 28. The cell of any one of clauses 22-27, wherein the first and second antigen binding chains are capable of associating with a CD3ζ polypeptide.

Clause 29. The cell of clause 28, wherein the first and second antigen binding chains, upon binding to the antigen, are capable of activating the CD3ζ polypeptide.

Clause 30. The cell of clause 29, wherein the activation of the CD3ζ polypeptide is capable of activating the cell.

Clause 31. The cell of any one of clauses 1-30, wherein the cell further comprises a gene disruption of a TCR locus.

Clause 32. The cell of clause 31, wherein the TCR locus is a TRAC locus.

Clause 33. The cell of any one of clauses 1-32, wherein the cell is a cell of the lymphoid lineage or a cell of the myeloid lineage.

Clause 34. The cell of clause 33, wherein the cell of the lymphoid lineage is selected from the group consisting of a T cell, a B cell, a Natural Killer (NK) cell, and a dendritic cell.

Clause 35. The cell of any one of clauses 1-34, wherein the cell is a T cell.

Clause 36. The cell of clause 35, wherein the T cell is derived from an induced pluripotent stem cell.

Clause 37. The cell of clause 35 or 36, wherein the T cell is a CD8+ T cell.

Clause 38. The cell of clause 37, wherein the CD8+ T cell is CD4 independent.

Clause 39. The cell of any one of clauses 34-38, wherein the T cell is selected from the group consisting of a cytotoxic T lymphocyte (CTL), a γδ T cell, a tumor-infiltrating lymphocyte (TIL), a regulatory T cell, and a Natural Killer T (NKT) cell.

Clause 40. The cell of any one of clauses 34-39, wherein the T cell is CD62L+, CD45RA+, or CD45RA+ and CD62L+.

Clause 41. The cell of any one of clauses 1-9 and 11-41, wherein the immunoevasin is encoded by a polynucleotide integrated at a locus within the genome of the T cell.

Clause 42. The cell of clause 41, wherein the locus is selected from the group consisting of a TRAC locus, a TRBC locus, a TRDC locus, and a TRGC locus.

Clause 43. The cell of clause 41 or 42, wherein the locus is a TRAC locus or a TRBC locus.

Clause 44. The cell of clause 43, wherein the locus is a TRAC locus.

Clause 45. The cell of any one of clauses 41-44, wherein the polynucleotide comprises an EF1 promoter.

Clause 46. The cell of clause 45, wherein the EF1 promoter comprises the nucleotide sequence set forth in SEQ ID NO: 90.

Clause 47. The cell of any one of clauses 1-46, wherein the antigen recognizing receptor is encoded by a polynucleotide integrated at a locus within the genome of the T cell.

Clause 48. The cell of clause 47, wherein the locus is selected from the group consisting of a TRAC locus, a TRBC locus, a TRDC locus, and a TRGC locus.

Clause 49. The cell of clause 47 or 48, wherein the locus is a TRAC locus or a TRBC locus.

Clause 50. The cell of clause 49, wherein the locus is a TRAC locus.

Clause 51. The cell of any one of clauses 1-50, wherein the antigen is a tumor antigen or a pathogen antigen.

Clause 52. The cell of clause 51, wherein the tumor antigen is selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLDN18.2, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GPRC5d, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TROP2, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

Clause 53. The cell of any one of clauses 1-52, further comprising a chimeric co-stimulating receptor (CCR).

Clause 54. The cell of clause 53, wherein the CCR comprises an extracellular antigen-binding domain that binds to a second antigen and an intracellular domain that is capable of delivering a costimulatory signal to the cell but does not alone deliver an activation signal to the cell.

Clause 55. The cell of clause 55, wherein the intracellular domain of the CCR comprises at least an intracellular domain of a co-stimulatory molecule or a portion thereof.

Clause 56. The cell of clause 54, wherein the costimulatory molecule is selected from the group consisting of CD28, 4-1BB, OX40, CD27, CD40, CD154, CD97, CD11a/CD18, ICOS, DAP-10, CD2, CD150, CD226, and NKG2D.

Clause 57. The cell of any one of clauses 1-53, wherein the cell further comprises at least one exogenous costimulatory ligand.

Clause 58. The cell of clause 57, wherein the at least one exogenous co-stimulatory ligand is selected from the group consisting of a tumor necrosis factor (TNF) family member, an immunoglobulin (Ig) superfamily member, and combinations thereof.

Clause 59. The cell of clause 58, wherein the TNF family member is selected from the group consisting of 4-1BBL, OX40L, CD70, FasL, GITRL, TNF-related apoptosis-inducing ligand (TRAIL), CD30L, LIGHT (TNFSF14), CD40L.

Clause 60. The cell of clause 59, wherein the Ig superfamily member is selected from the group consisting of CD80, CD86, ICOSLG, and combinations thereof.

Clause 61. The cell of any one of clauses 57-60, wherein the at least one exogenous costimulatory ligand comprises CD80.

Clause 62. The cell of any one of clauses 57-60, wherein the at least one exogenous a costimulatory ligand comprises 4-1BBL.

Clause 63. The cell of any one of clauses 57-60, wherein the cell comprises two exogenous costimulatory ligands.

Clause 64. The cell of clause 63, wherein the at least two exogenous costimulatory ligands comprise CD80 and 4-1BBL.

Clause 65. The cell of any one of clauses 1-64, wherein the cell further comprises a fusion polypeptide comprising: a) an extracellular domain and a transmembrane domain of a co-stimulatory ligand, and b) an intracellular domain of a first co-stimulatory molecule.

Clause 66. The cell of clause 65, wherein the co-stimulatory ligand is selected from the group consisting of a tumor necrosis factor (TNF) family member, an immunoglobulin (Ig) superfamily member, and combinations thereof.

Clause 67. The cell of clause 66, wherein the TNF family member is selected from the group consisting of 4-1BBL, OX40L, CD70, GITRL, CD40L, and combinations thereof.

Clause 68. The cell of clause 66, wherein the Ig superfamily member is selected from the group consisting of CD80, CD86, ICOSLG, and combinations thereof.

Clause 69. The cell of any one of clauses 65-68, wherein the co-stimulatory ligand is CD80.

Clause 70. The cell of any one of clauses 65-69, wherein the first co-stimulatory molecule is selected from the group consisting of CD28, 4-1BB, OX40, ICOS, DAP-10, CD27, CD40, NKG2D, CD2, and combinations thereof.

Clause 71. The cell of clause 70, wherein the first co-stimulatory molecule is 4-1BB.

Clause 72. The cell of any one of clauses 65-71, wherein the co-stimulatory ligand is CD80 and the first co-stimulatory molecule is 4-1BB.

Clause 73. The cell of any one of clauses 65-72, wherein the fusion polypeptide further comprises an intracellular domain of a second co-stimulatory molecule.

Clause 74. The cell of clause 73, wherein the second co-stimulatory molecule is selected from the group consisting of CD28, 4-1BB, OX40, ICOS, DAP-10, CD27, CD40, NKG2D, CD2, and combinations thereof.

Clause 75. The cell of clause 73 or 74, wherein the second co-stimulatory molecule is CD28.

Clause 76. The cell of any one of clauses 73-75, wherein the co-stimulatory ligand is CD80, the first co-stimulatory molecule is 4-1BB, and the second co-stimulatory molecule is CD28.

Clause 77. The cell of any one of clauses 1-76, wherein the immunoevasin reduces the expression level of the major histocompatibility complex I (MHCI) from between about 60% to about 90% compared to a cell non expressing the immunoevasin.

Clause 78. The cell of any one of clauses 1-77, wherein the cell is autologous.

Clause 79. The cell of any one of clauses 1-77, wherein the cell is allogeneic.

Clause 80. A composition comprising the cell of any one of clauses 1-79.

Clause 81. The composition of clause 80, which is a pharmaceutical composition further comprising a pharmaceutically acceptable excipient.

Clause 82. A nucleic acid comprising a first polynucleotide encoding an immunoevasin and a second polynucleotide encoding an antigen recognizing receptor that targets an antigen.

Clause 83. The nucleic acid of clause 82, wherein the immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 84. The nucleic acid of clause 83, wherein the immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 85. The nucleic acid of any one of clauses 82-84 further comprising a third polynucleotide encoding a second immunoevasin.

Clause 86. The nucleic acid of clause 85, wherein the second immunoevasin comprising or consisting of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 87. The nucleic acid of clause 86, wherein the second immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 88. The nucleic acid of any one of clauses 82-87, wherein one or more of the first, second, and third polynucleotide is operably linked to a promoter element.

Clause 89. The nucleic acid of clause 88, wherein the promoter element is an endogenous promoter or an exogenous promoter.

Clause 90. The nucleic acid of clause 89, wherein the endogenous promoter is a TRAC promoter.

Clause 91. The nucleic acid of clause 89, wherein the exogenous promoter is a EF1 promoter.

Clause 92. The nucleic acid of clause 91, wherein the EF1 promoter comprises or consists of the nucleotide sequence set forth in SEQ ID NO: 90.

Clause 93. A nucleic acid composition comprising a first polynucleotide encoding an immunoevasin and a second polynucleotide encoding an antigen recognizing receptor that targets an antigen.

Clause 94. The nucleic acid composition of clause 93, wherein the immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 95. The nucleic acid composition of clause 94, wherein the immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

Clause 96. The nucleic acid composition of any one of clauses 93-95 further comprising a third polynucleotide encoding a second immunoevasin.

Clause 97. The nucleic acid composition of clause 96, wherein the second immunoevasin comprising or consisting of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, SEQ ID NO: 91.

Clause 98. The nucleic acid composition of clause 97, wherein the second immunoevasin comprises the amino acid sequence set forth in SEQ ID NO: 1, SEQ ID NO: 2, SEQ ID NO: 3, SEQ ID NO: 4, SEQ ID NO: 5, SEQ ID NO: 91.

Clause 99. The nucleic acid composition of any one of clauses 93-98, wherein one or more of the first, second, and third polynucleotide is operably linked to a promoter element.

Clause 100. The nucleic acid composition of clause 99, wherein the promoter element is a EF1 promoter.

Clause 101. The nucleic acid composition of clause 100, wherein the EF1 promoter comprises or consists of the nucleotide sequence set forth in SEQ ID NO: 90.

Clause 102. A vector comprising the nucleic acid of any one of clauses 82-92 or the nucleic acid composition of any one of clauses 93-101.

Clause 103. The vector of clause 102, wherein the vector is a lentiviral vector.

Clause 104. The vector of clause 102, wherein the vector is a γ-retroviral vector.

Clause 105. A lipid nanoparticle comprising the nucleic acid of any one of clauses 82-92 or the nucleic acid composition of any one of clauses 93-101.

Clause 106. A composition comprising the nucleic acid of any one of clauses 82-92, the vector of any one of clauses 102-104, or the lipid nanoparticle of clause 105.

Clause 107. The composition of clause 102, which is a pharmaceutical composition further comprising a pharmaceutically acceptable excipient.

Clause 108. A method for producing a cell of any one of clauses 1-79, the method comprising introducing into the cell the nucleic acid of any one of clauses 82-92, the nucleic acid composition of any one of clauses 93-101, the vector of any one of clauses 102-104, the lipid nanoparticle of clause 105, or a composition of clause 106 or 107.

Clause 109. The method of clause 108, further comprising generating a gene disruption of a TRAC locus, a NLRC5 locus, and a RFX5 locus, or a combination thereof.

Clause 110. The method of clause 109, wherein generating the gene disruption of comprises a gene editing method comprising homologous recombination, a Zinc finger nuclease, a meganuclease, a Transcription activator-like effector nuclease (TALEN), a Clustered regularly-interspaced short palindromic repeats (CRISPR) system, or a combination thereof.

Clause 111. A cell produced by the method of any one of clauses 108-110.

Clause 112. A method of reducing tumor burden in a subject, the method comprising administering to the subject an effective amount of the cells of any one of clauses 1-79 or 111, or the composition of any one of clauses 80, 81, 106, or 107.

Clause 113. The method of clause 112, wherein the method reduces the number of tumor cells, reduces tumor size, and/or eradicates the tumor in the subject.

Clause 114. A method of preventing and/or treating a neoplasm or a tumor in the subject, administering to the subject an effective amount of the cells of any one of clauses 1-79 or 111, or the composition of any one of clauses 80, 81, 106, or 107.

Clause 115. The method of any one of clauses 112-114, wherein the neoplasm or tumor is cancer.

Clause 116. The method of any one of clauses 112-115, wherein the neoplasm or tumor is a solid tumor.

Clause 117. The method of clause 116, wherein the solid tumor is selected from the group consisting of renal cell carcinoma, non-small-cell lung cancer, lung adenocarcinoma, lung squamous cell carcinoma, lung neuroendocrine carcinoma, small-cell lung cancer, pancreatic cancer, breast cancer, astrocytoma, glioblastoma, laryngeal/pharyngeal carcinoma, EBV-associated nasopharyngeal carcinoma, and ovarian carcinoma.

Clause 118. The method of any one of clauses 112-115, wherein the neoplasm or tumor is a blood cancer.

Clause 119. The method of clause 118, wherein the neoplasm or tumor is a myeloid disorder.

Clause 120. The method of clause 119, wherein the myeloid disorder is selected from the group consisting of myelodysplastic syndromes, myeloproliferative neoplasms, chronic myelomonocytic leukemia, or acute myeloid leukemia (AML), blastic plasmacytoid dendritic cell neoplasm, acute myeloblastic leukemia, acute promyelocytic leukemia, acute myelomonocytic leukemia, chronic myelocytic leukemia, and polycythemia vera.

Clause 121. The method of clause 120, wherein the myeloid disorder is acute myeloid leukemia (AML).

Clause 122. The method of clause 118, wherein the neoplasm or tumor is a B-cell malignancy.

Clause 123. The method of clause 122, wherein the B-cell malignancy is selected from the group consisting of B cell non-Hodgkin lymphoma (NHL), B cell Hodgkin's lymphoma, B cell acute lymphocytic leukemia (ALL), B cell chronic lymphocytic leukemia (CLL), multiple myeloma (MM), CLL with Richter's transformation, and CNS lymphoma.

Clause 124. The method of clause 118, wherein the neoplasm or tumor is a leukemia.

Clause 125. The method of clause 124, wherein the leukemia is selected from the group consisting of acute myeloid leukemia (AML), chronic myeloid leukemia (CML), acute lymphocytic leukemia (ALL), chronic lymphocytic leukemia (CLL), acute promyelocytic leukemia (APL), mixed-phenotype acute leukemia (MLL), hairy cell leukemia, B cell prolymphocytic leukemia, B-cell precursor acute lymphoblastic leukemia, and T-cell precursor acute lymphoblastic leukemia.

Clause 126. The method of clause 118, wherein the neoplasm or tumor is a lymphoma.

Clause 127. The method of clause 126, wherein the lymphoma is selected from the group consisting of Hodgkin's lymphoma, non-Hodgkin's lymphoma, B-cell non-Hodgkin's lymphoma, T-cell non-Hodgkin's lymphoma, and T-cell precursor acute lymphoblastic lymphoma.

Clause 128. A method of preventing and/or treating a pathogen infection in a subject, the method comprising administering to the subject an effective amount of the cells of any one of clauses 1-79, or the composition of any one of clauses 80, 81, 106, or 107.

Clause 129. A method of preventing and/or treating an autoimmune disease in a subject, the method comprising administering to the subject an effective amount of the cells of any one of clauses 1-79 or 111, or the composition of any one of clauses 80, 81, 106, or 107.

Clause 130. A method of preventing and/or treating an infectious disease in a subject, the method comprising administering to the subject an effective amount of the cells of any one of clauses 1-79 or 111, or the composition of any one of clauses 80, 81, 106, or 107.

Clause 131. The cells of any one of clauses 1-79 or 111, or the composition of any one of clauses 80, 81, 106, or 107 for use in reducing tumor burden, treating and/or preventing a neoplasm or a tumor, preventing and/or treating a pathogen infection, preventing and/or treating an autoimmune disease, and/or preventing and/or treating an infectious disease, in a subject.

Clause 132. The cells of any one of clauses 1-79 or 111, or the composition of any one of clauses 80, 81, 106, or 107 for reducing tumor burden, treating and/or preventing a neoplasm or a tumor, preventing and/or treating a pathogen infection, preventing and/or treating an autoimmune disease, and/or preventing and/or treating an infectious disease, in a subject.

Clause 133. Use of the cells of any one of clauses 1-79 or 111, or the composition of any one of clauses 80, 81, 106, or 107 for the manufacture of a medicament for reducing tumor burden, treating and/or preventing a neoplasm or a tumor, preventing and/or treating a pathogen infection, preventing and/or treating an autoimmune disease, and/or preventing and/or treating an infectious disease, in a subject.

Clause 134. A kit comprising the cells of any one of clauses 1-79 or 111, or the composition of any one of clauses 80, 81, 106, or 107.

Clause 135. The kit of clause 134, wherein the kit further comprises written instructions for reducing tumor burden, treating and/or preventing a neoplasm or a tumor, preventing and/or treating a pathogen infection, preventing and/or treating an autoimmune disease, and/or preventing and/or treating an infectious disease.

### EXAMPLES

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides disclosed herein, and, as such, may be considered in making and practicing the presently disclosed subject matter. Particularly useful techniques for particular embodiments will be discussed in the sections that follow.

The following examples are put forth so as to provide those of ordinary skill in the art with a complete disclosure and description of how to make and use the presently disclosed cells and compositions, and are not intended to limit the scope of what the inventors regard as their invention.

### Example 1

Allogeneic cell therapies must overcome immune rejection between donor and host cells. Currently, techniques to eliminate T cell receptors (TCR) by CRISPR-Cas9 editing to avoid Graft-versus-host immunity are available. Conversely, host immune cells such as T and NK cells can reject allogeneic CAR T cells through Host-Versus-Graft Immunity (HVGI), impairing allogeneic CAR T cell expansion and function. Existing approaches to avoid HVGI include immune suppression and immune evasion, both of which have limitations. For example, immune suppression raises the risk of developing infectious diseases. Immune evasion is achieved by knockout of the immune molecule MHC-I to avoid rejection by host CD8 T cells. However, while deletion of MHC-I avoids CD8 T cell rejection, it sensitizes cells to NK cell rejection.

The presently disclosed subject matter identified an intermediate ‘Goldilocks’ level of MHC-I expression that avoids T cell rejection without triggering NK cells. As detailed below, this level is achieved by modifying allogeneic CAR T cells with the viral evasion proteins such as BNLF2a and NEF.

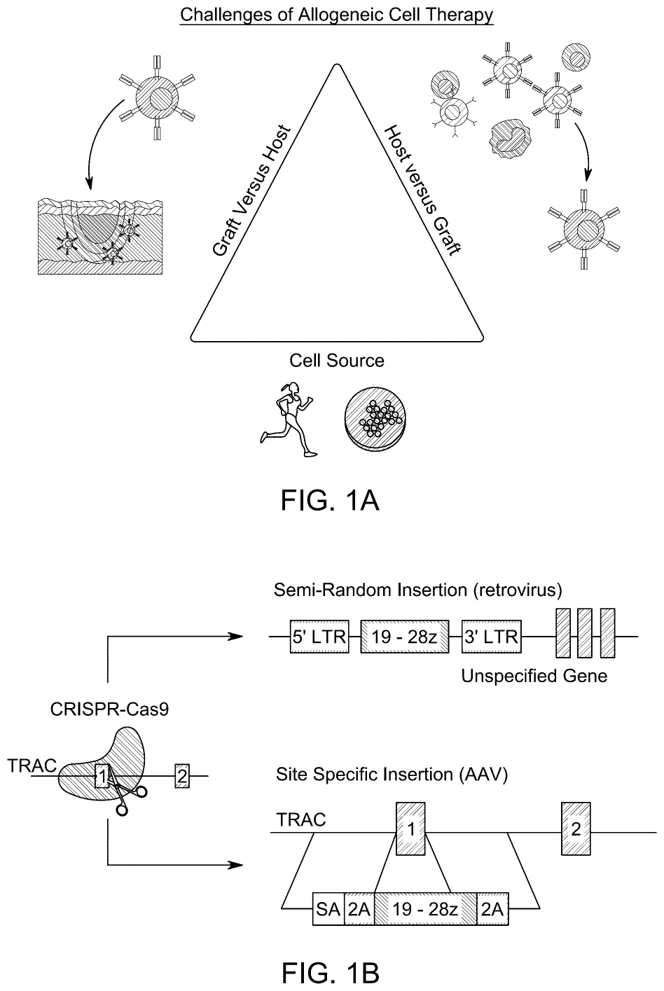

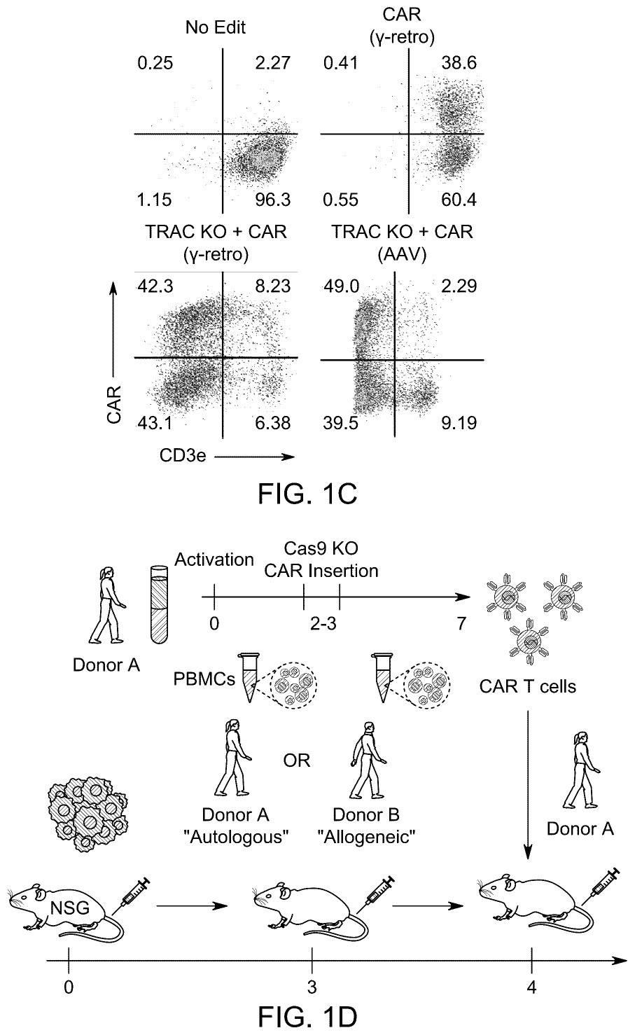

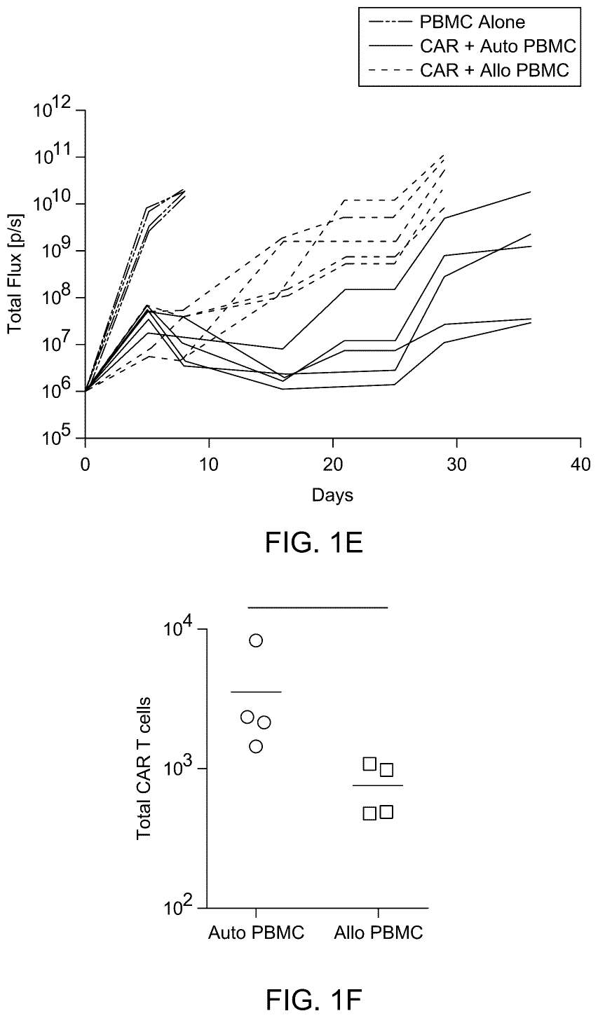

Allogeneic CAR T cells must avoid graft-versus-host and host-versus-graft immune rejection. To eliminate GVHD, T cell receptor is eliminated via CRISPR-Cas9 editing. To model HVG immune rejection, the presently disclosed subject matter developed an animal model that showed impaired activity of allogeneic compared to autologous CAR T cells due to immune rejection (FIG. 1A-1F). In order to eliminate GVHD, T cell receptor knockout CAR T cells were generated by CRISPR-Cas9 targeting of TRAC locus. CAR insertion occurred via either semi-random gamma-retrovirus mediated transduction, or homology-directed repair driven insertion of CAR into the TRAC locus using adeno-associated virus (AAV). CARs edited with Cas9 and a TRAC-directed guide RNA had loss of expression of the TCRα-associated membrane protein CD3ε, while CARs generated with either γ-retrovirus or AAV showed robust CAR expression. Next, as seen in FIG. 1D, NALM6 leukemia cell line transduced with GFP and firefly luciferase for bioluminescent imaging were injected in animal models and PBMCs from either the same donor (autologous) or a different donor (allogeneic) as CAR T cells were injected intravenously on Day 3, followed by CAR T cells the next day. CARs infused with autologous PBMCs had superior tumor control to either a PBMC-alone control or CARs infused with allogeneic PBMCs (FIG. 1E). Further, fewer CAR T cells were found in bone marrow ten days after CAR injection in mice carrying allogeneic compared to autologous PBMCs, consistent with immune rejection (FIG. 1F).

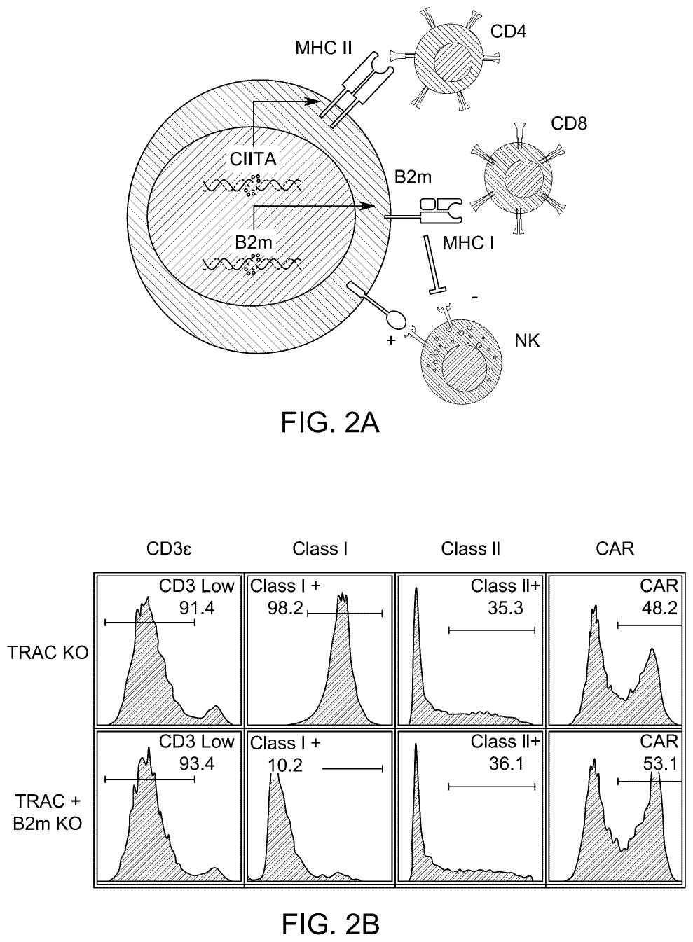

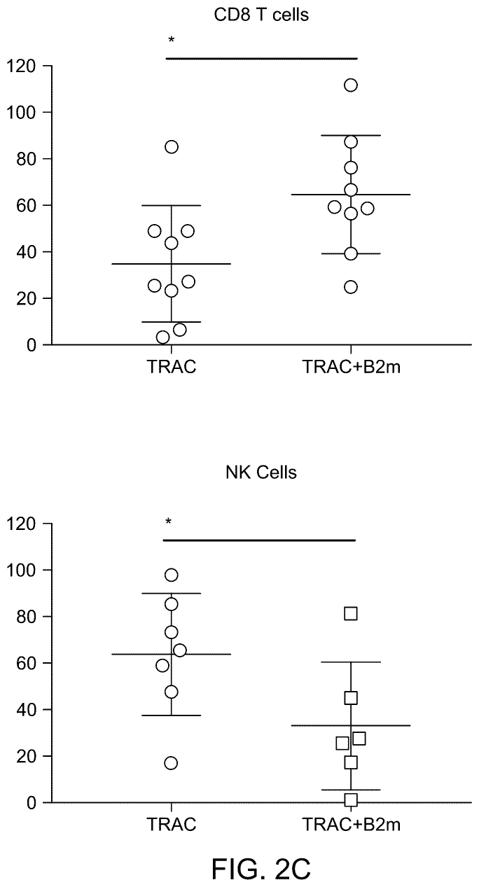

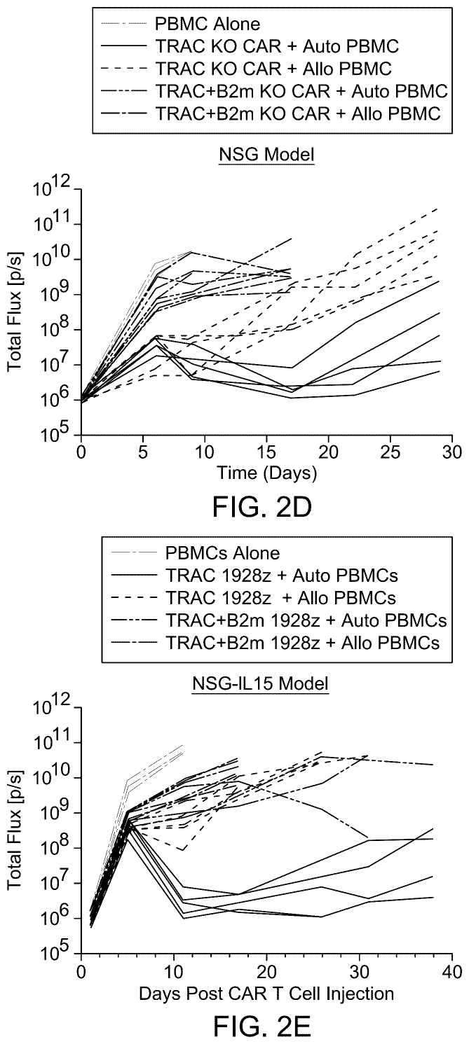

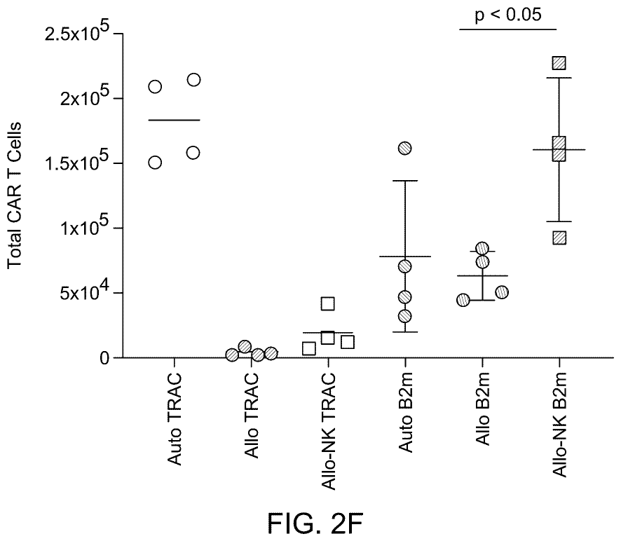

A common approach to evading rejection uses beta-2-macroglobulin knockout to eliminate MHC expression and thus CD8 T cell rejection. However, B2m KO cells were sensitive to NK cell rejection (FIGS. 2A-2F).

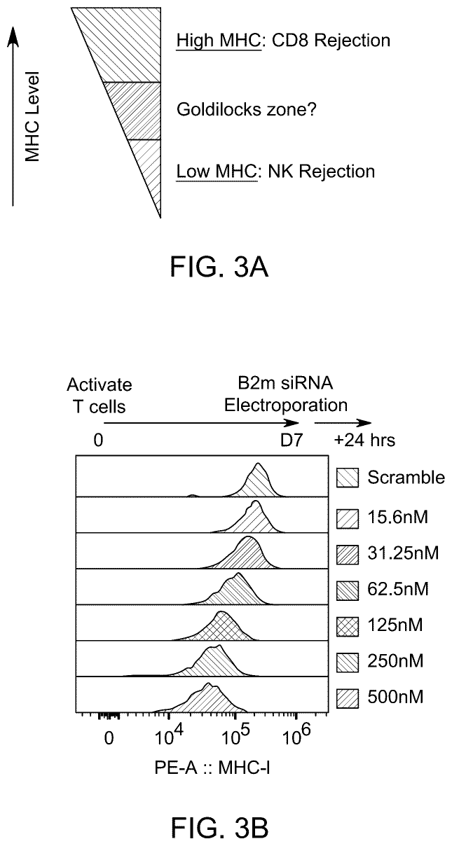

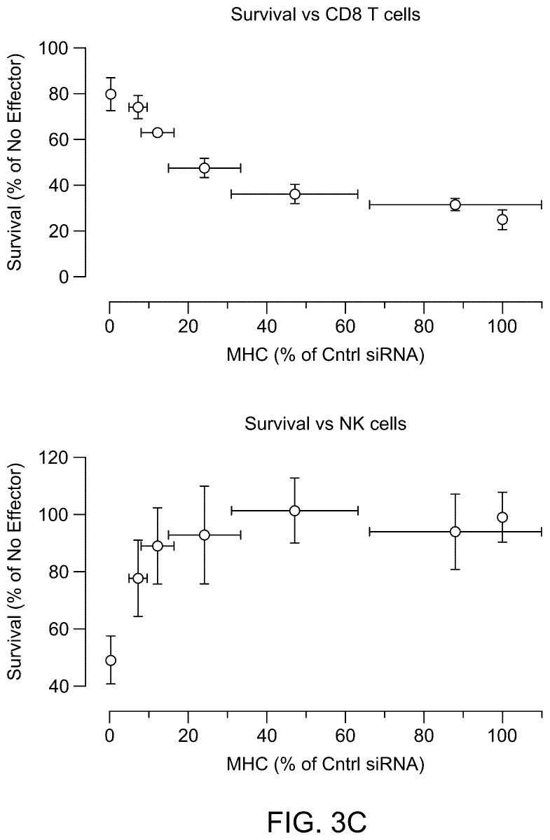

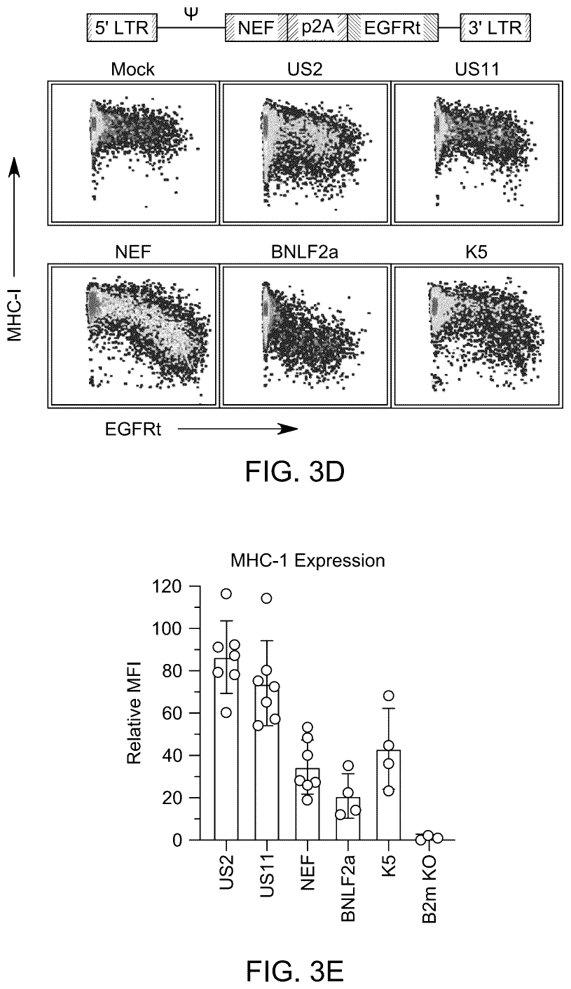

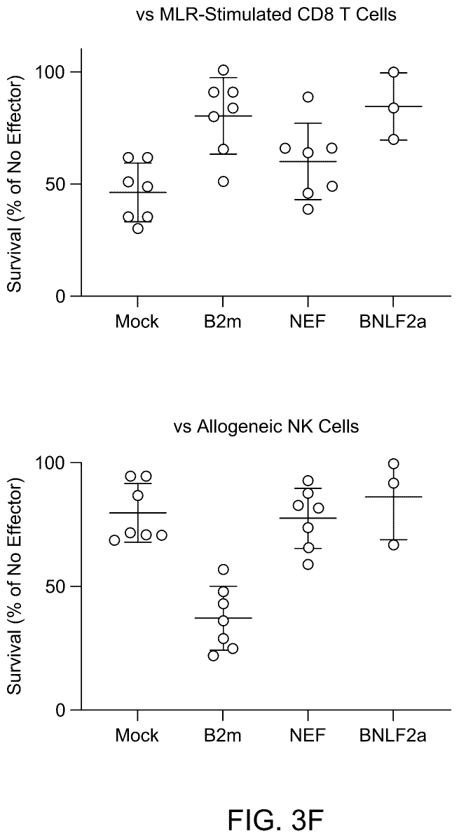

Thus, it was hypothesized that there is an intermediate (‘goldilocks’) level of MHC that balance CD8 T cell and NK cell rejection. Using siRNA knockdown experiments, it was found that this level was approximately 10-40% of the native level. Intermediate levels of MHC were achieved by overexpressing viral evasion proteins that reduce but do not eliminate surface MHC expression, leading to protection from CD8 T cell killing without triggering NK cells. The proteins NEF (from HIV-1) and BNLF2a (from EBV) were selected as potential candidates (FIGS. 3A-3F).

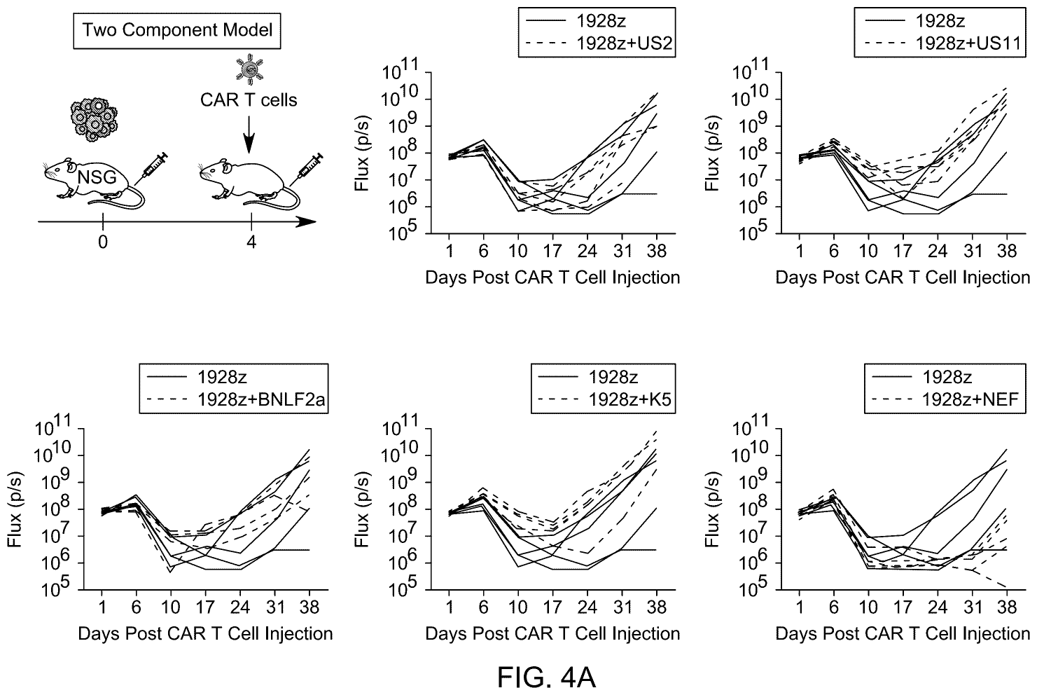

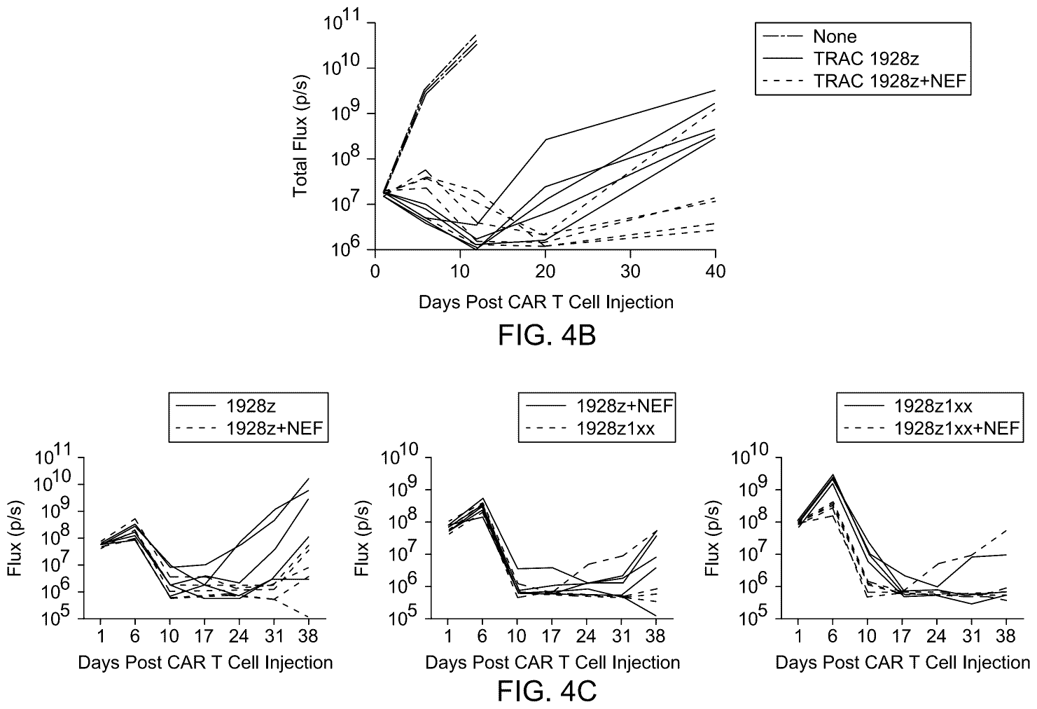

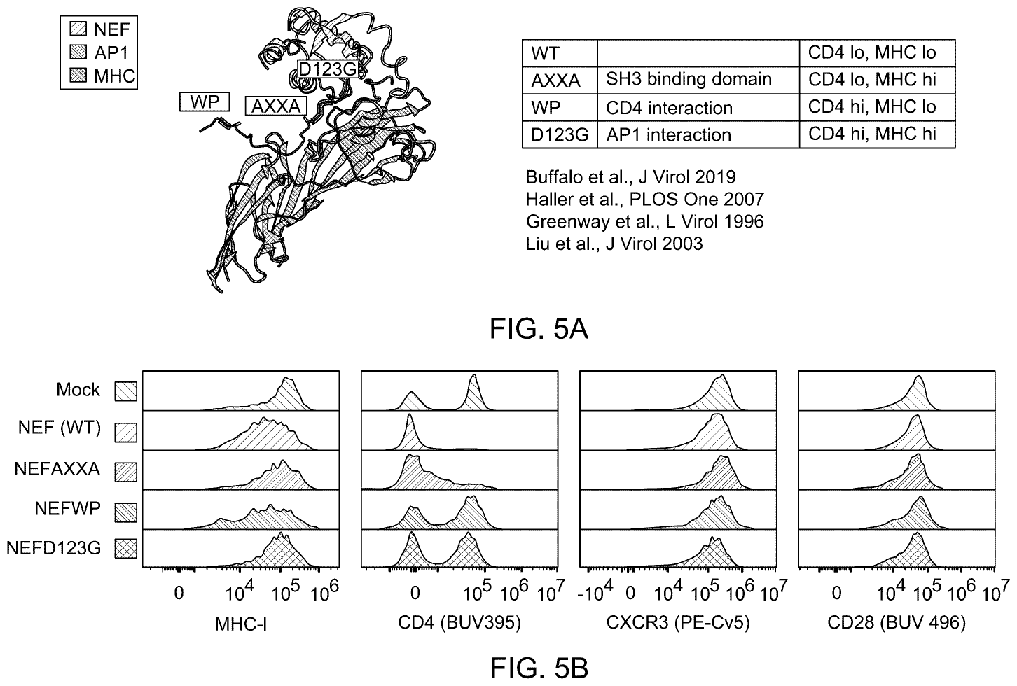

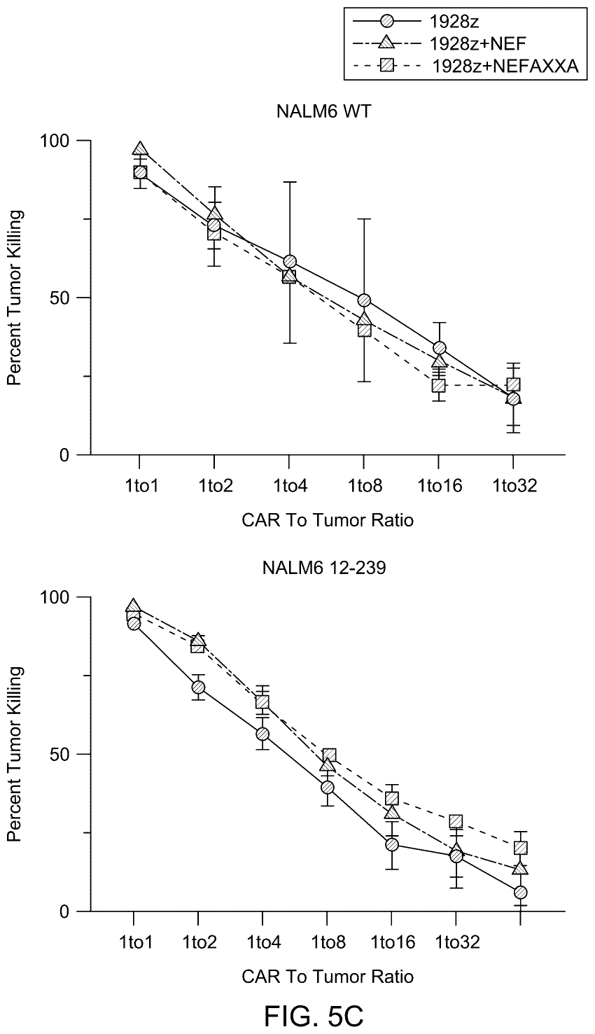

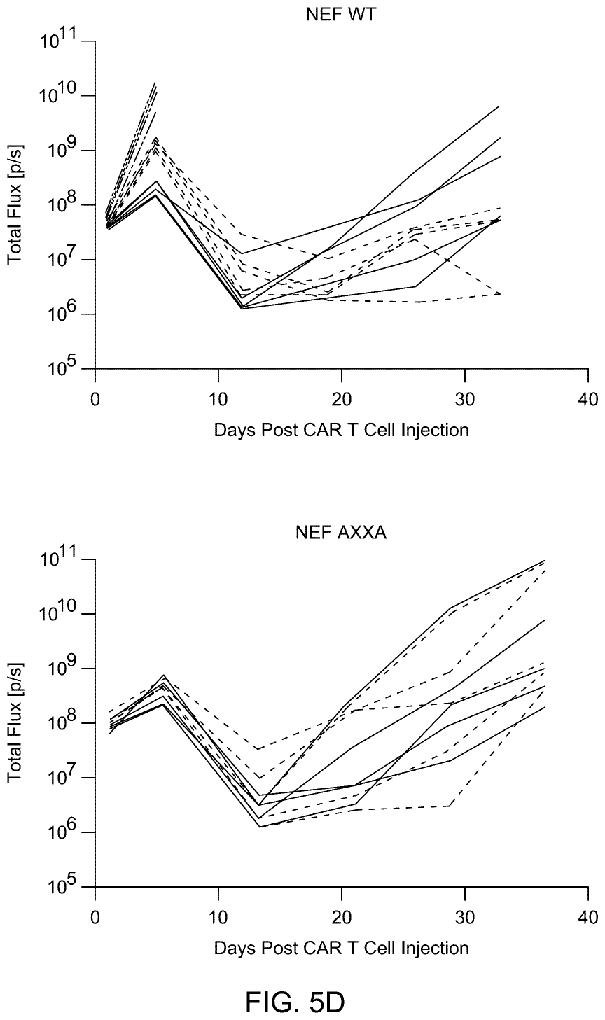

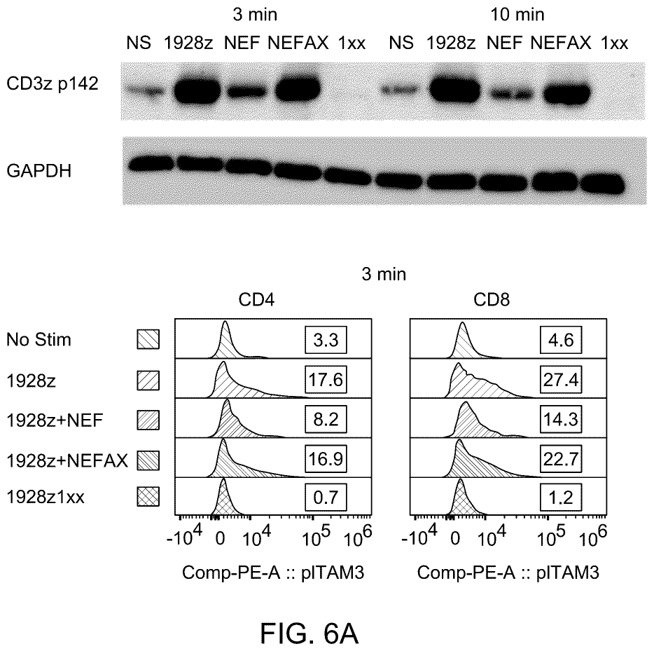

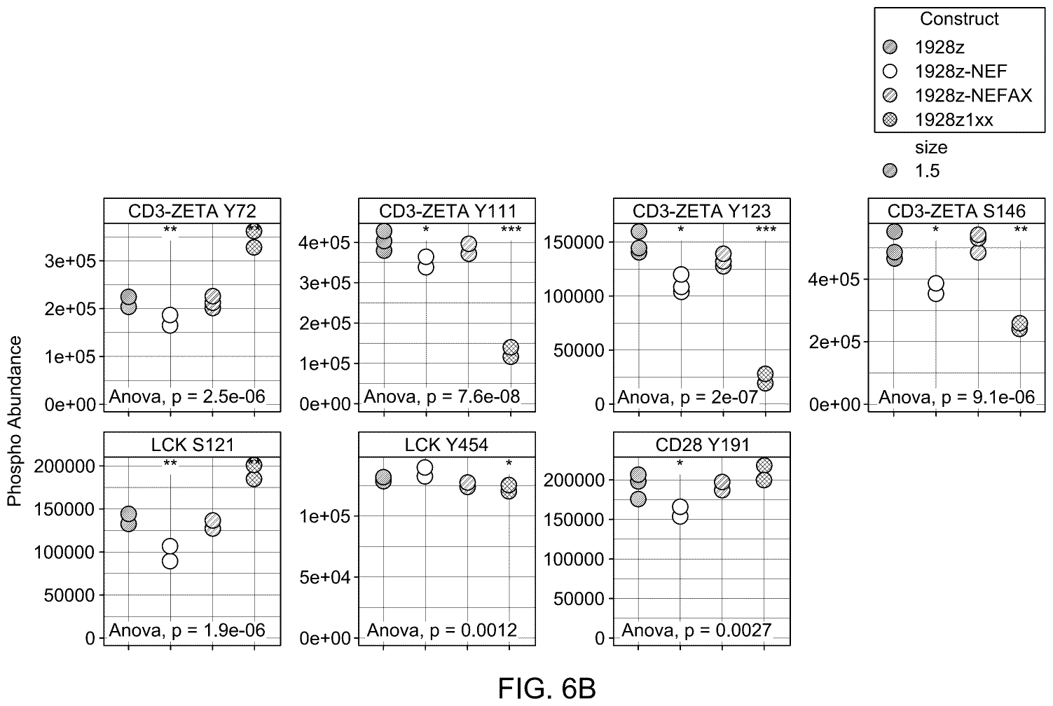

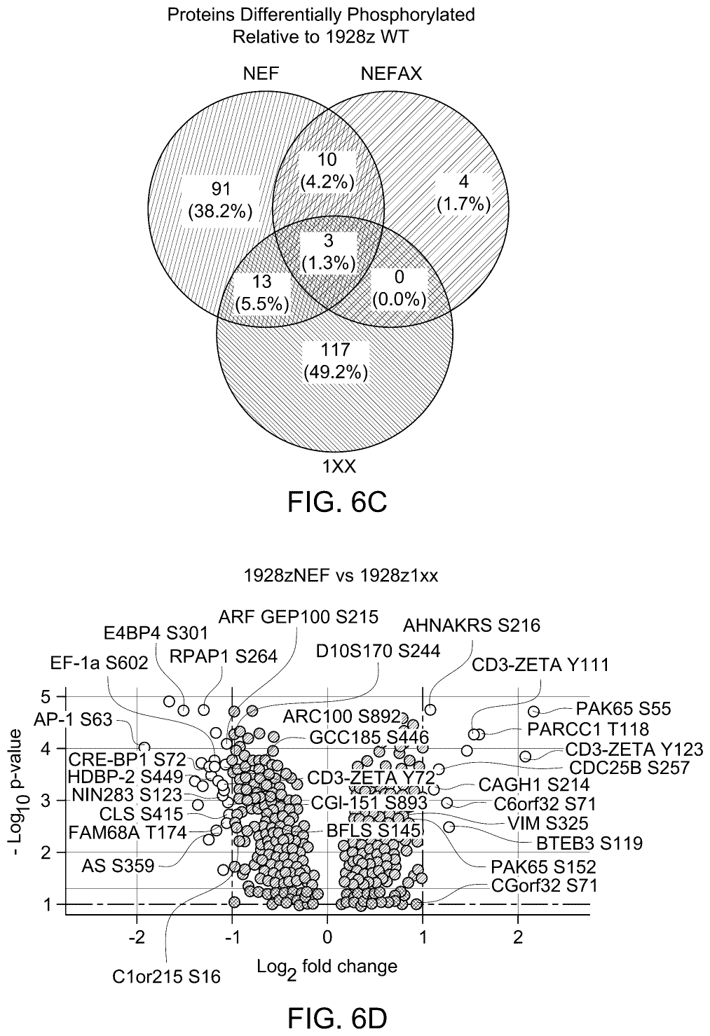

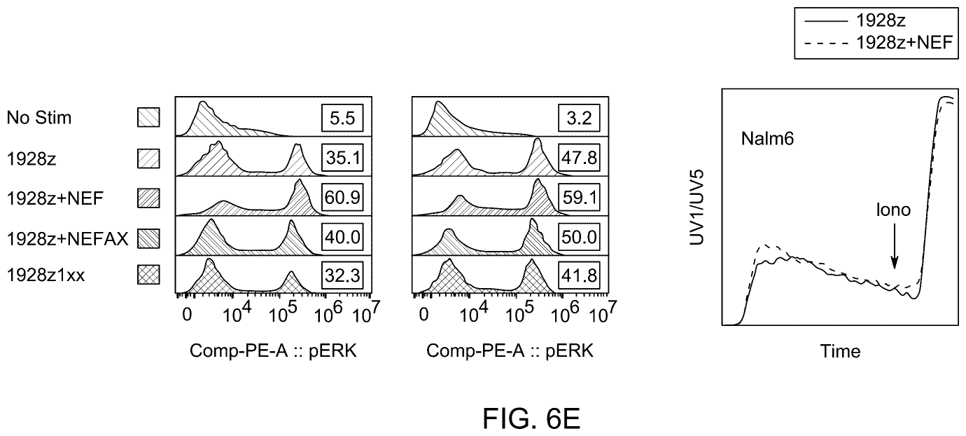

NEF is a multifunctional protein. Here, it is demonstrated that, in vivo, NEF not only reduced MHC expression, but actually improved CAR T cell tumor rejection (FIGS. 4A-4C). Next, variants of the NEF protein (e.g., NEFWP, NEFAXXA, NEFD123G) were used to study the mechanism of NEF enhancement (FIGS. 5A-5D). NEF improved CAR T cell signaling in a distinct manner than the previously described 1928z1xx variant of the typical 1928z CAR construct, and thus these two interventions were combined in a single strategy (FIGS. 6A-6E).

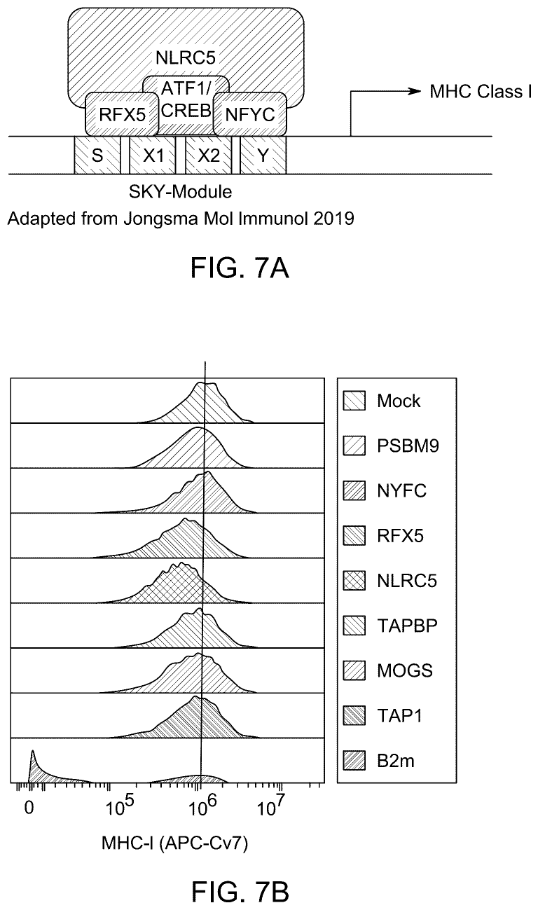

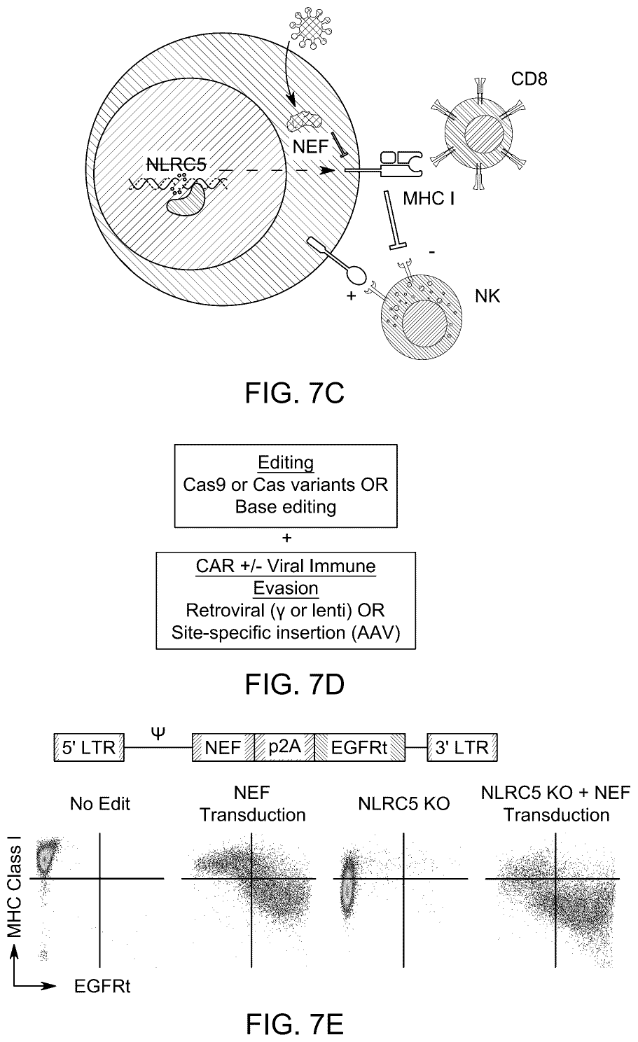

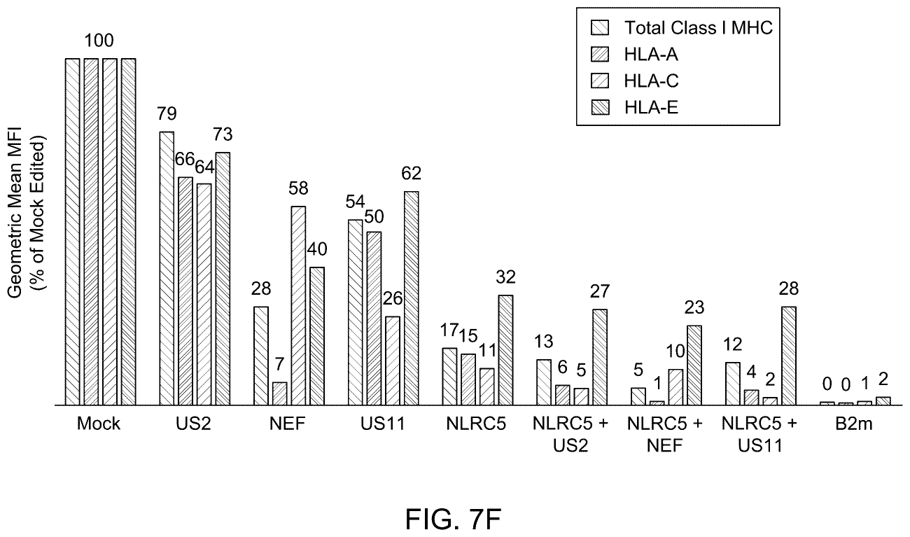

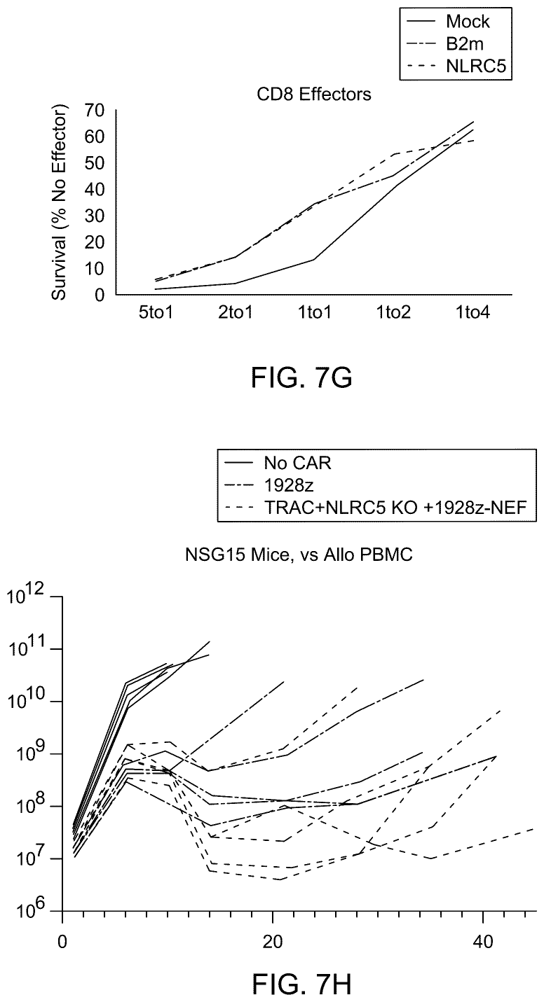

Additionally or alternatively, downmodulation of MHC expression involved knockout or knockdown of MHC regulating transcription factors such as NLRC5 and RFX5. These strategies were used independently or combined with viral immunoevasins such as NEF (FIGS. 7A-7H).

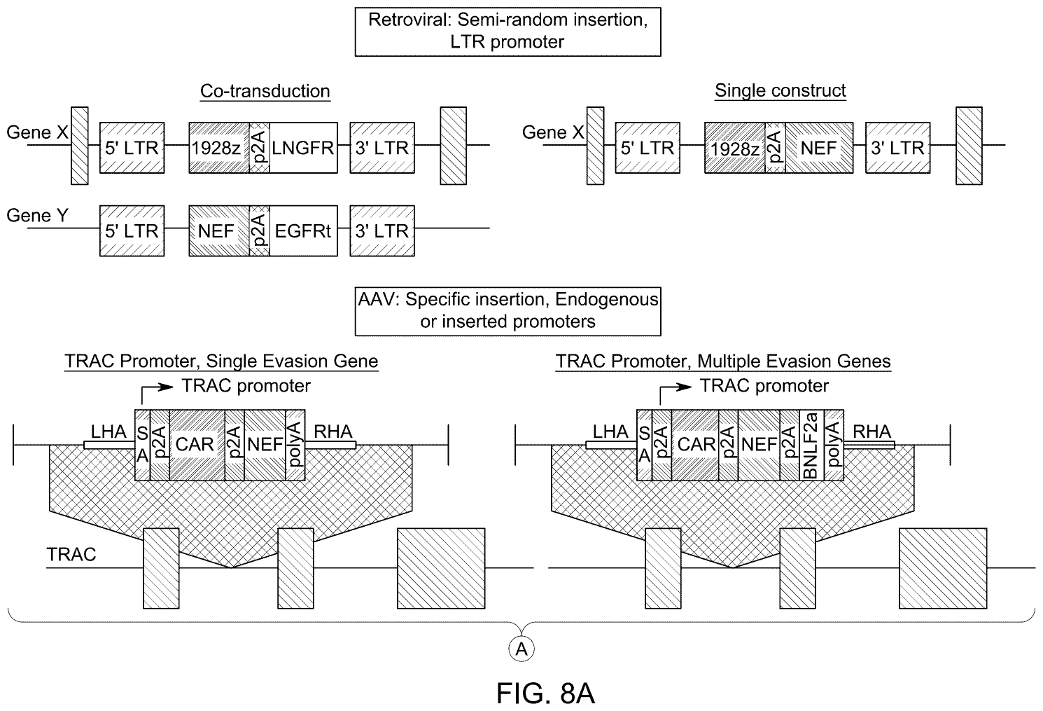

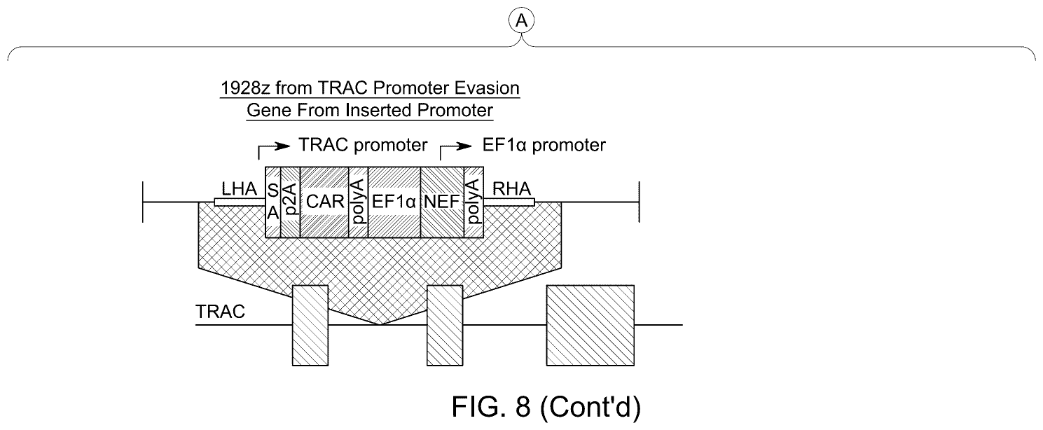

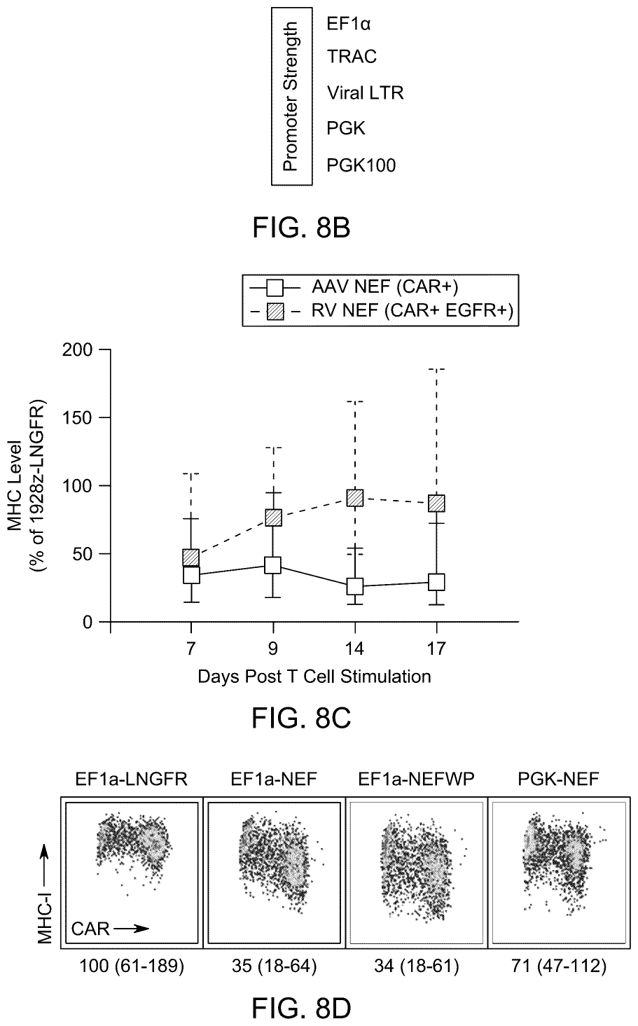

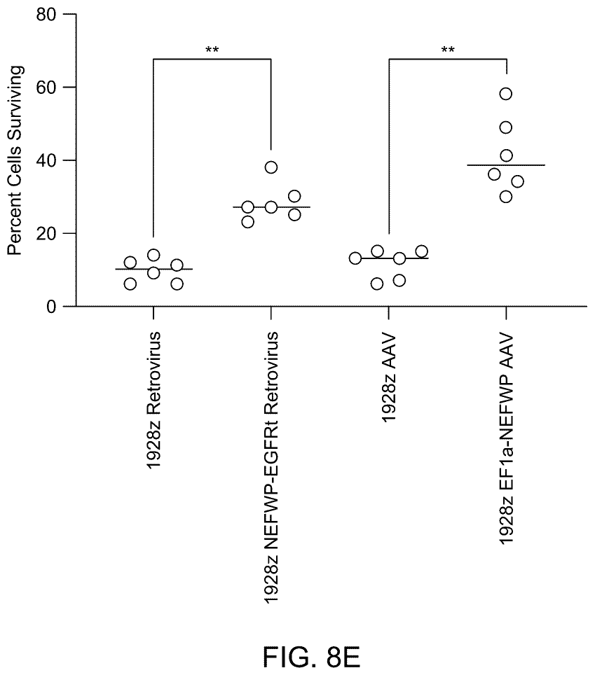

Previously, it was shown that CAR expressed from the TRAC promoter and locus, achieved via AAV and Cas9 driven site-specific insertion, led to a more effective CAR T cell in vivo. The presently disclosed subject matter shows that expression of viral evasion proteins from the TRAC locus led to more stable reduction in MHC. This was achieved by the TRAC promoter or by introduction of exogenous promoters such as EF1a (FIGS. 8A-8E).

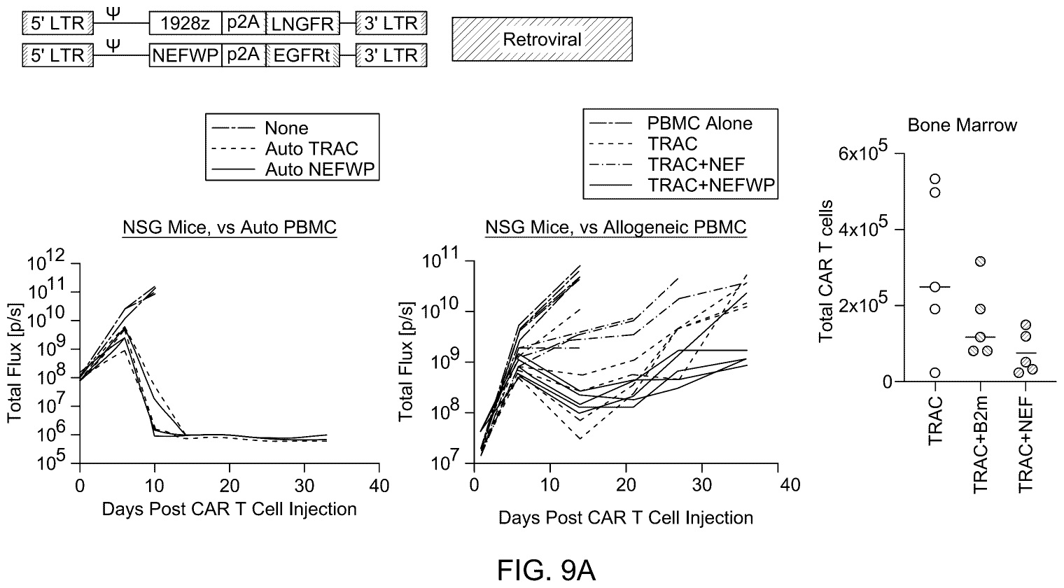

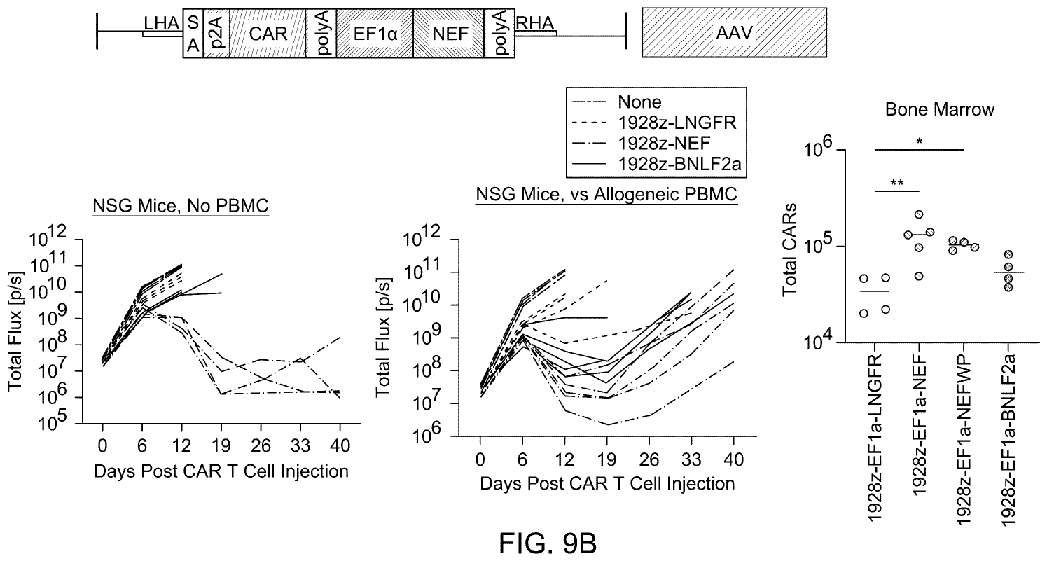

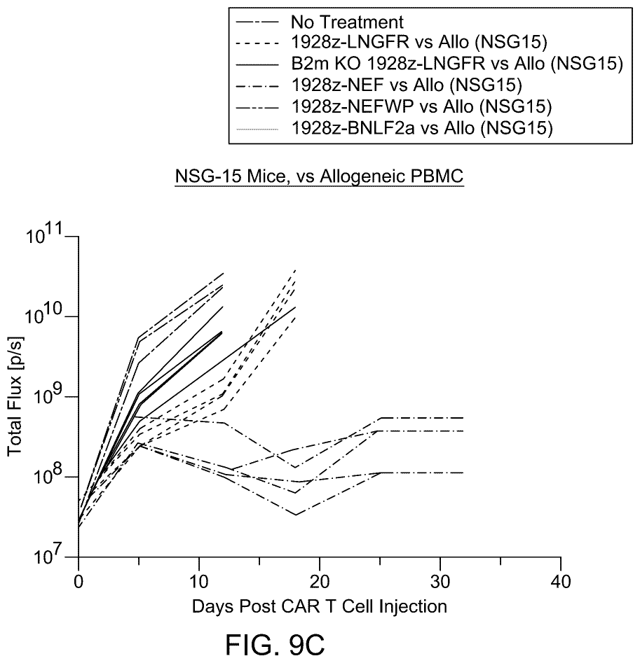

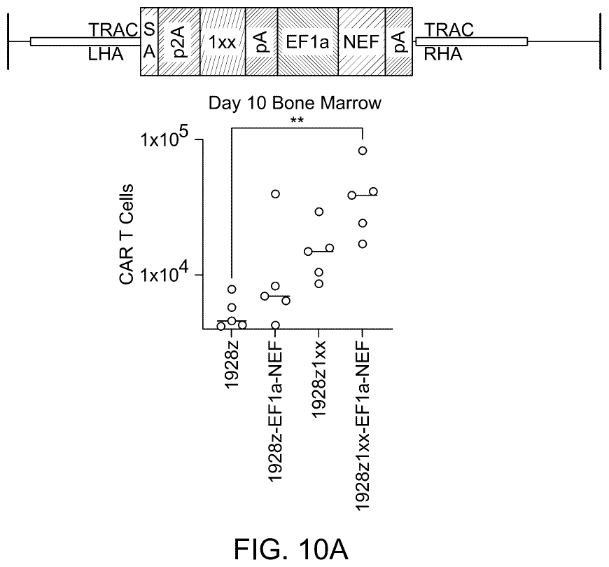

Viral evasion proteins expressed from a retroviral vector did not improve in vivo allogeneic CAR T cell efficacy. However, NEF expressed from EF1a promoter in the TRAC locus led to resistance to rejection and enhanced activity in an allogeneic model in vivo (FIGS. 9A-9C).

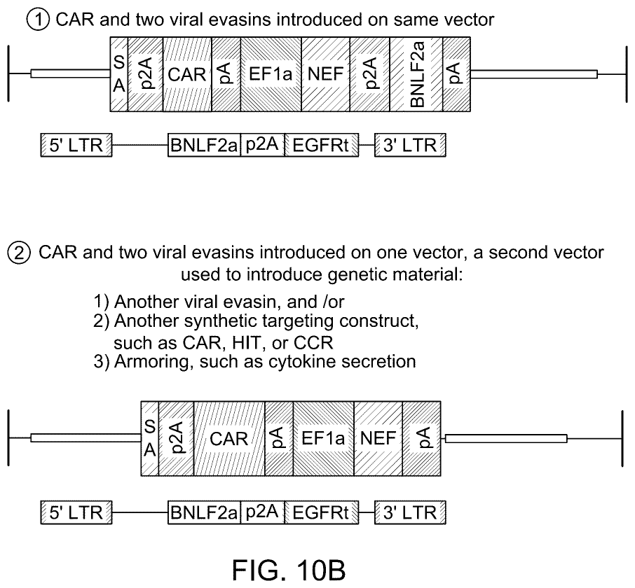

Finally, additional construct were developed to generate allogeneic CAR T cells. As illustrated in FIGS. 10A and 10B, upon insertion in the TRAC locus driven by homologous arms (e.g., LHA and RHA), the cell expresses a CAR under the control of the TRAC promoter and a polypeptide NEF (e.g., one disclosed in Section 2) under the control of the EF1 promoter. Alternatively, multiple immunoevasins can be expressed under the control of the EF1 promoter by using the 2A polypeptides.

Overall, the presently disclosed subject matter provides novel CAR T cell modified with CRISPR-CAS9 editing of the TRAC locus, and insertion of CAR and viral evasion proteins. The cell source can be healthy donor cells, or renewable stem cell sources such as induced pluripotent stem cells. The CAR can be 1928z as well as other versions of CD19 targeted CARs including 1xx and bbz. Additionally or alternatively, the CAR can be any CAR targeting other tumor antigens such as BCMA, GPRC5D, CD70, etc. Further, the viral evasion protein can be expressed from the TRAC promoter, or from exogenous promoters.

Moreover, the presently disclosed subject matter provides novel constructs that can contain one viral evasion protein (e.g., NEF) or multiple proteins (e.g., NEF and BNLF2a).

The presently disclosed subject matter provides novel strategies that involves knockout of the TRAC locus to avoid GVHD and to allow site-specific insertion of construct. In certain embodiments, the strategy can be combined with NLRC5 or RFX5 knockout.

### Example 2

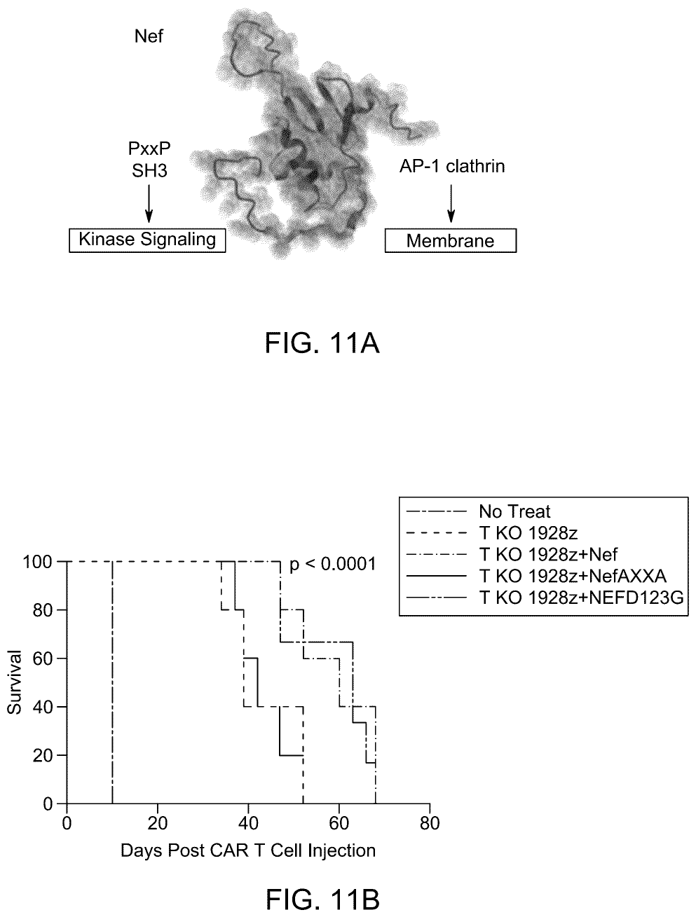

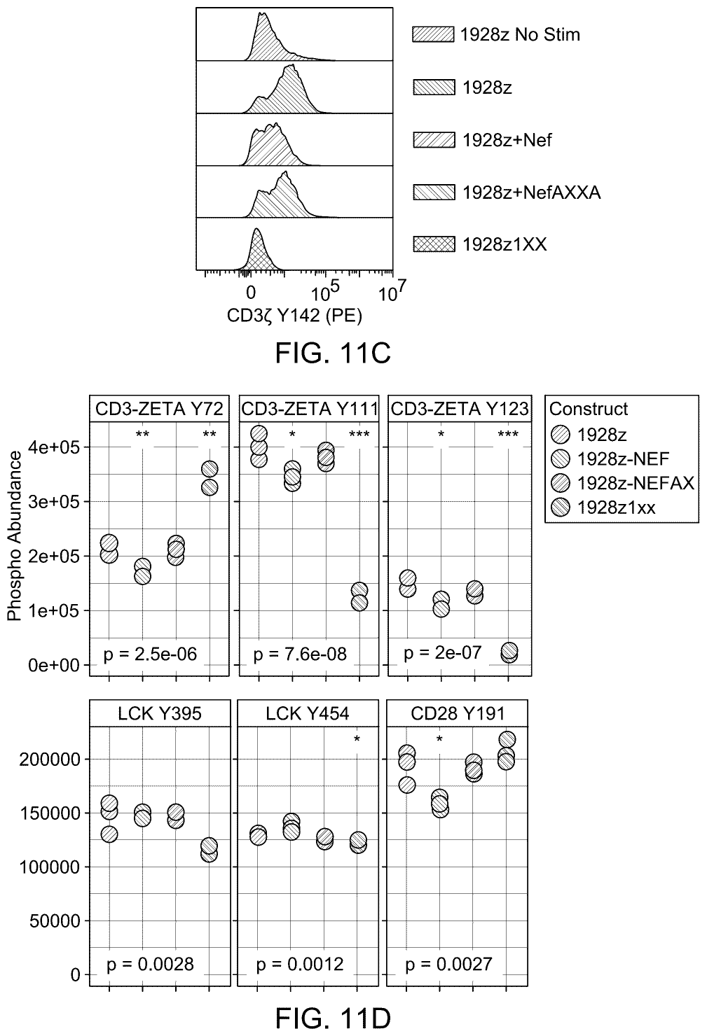

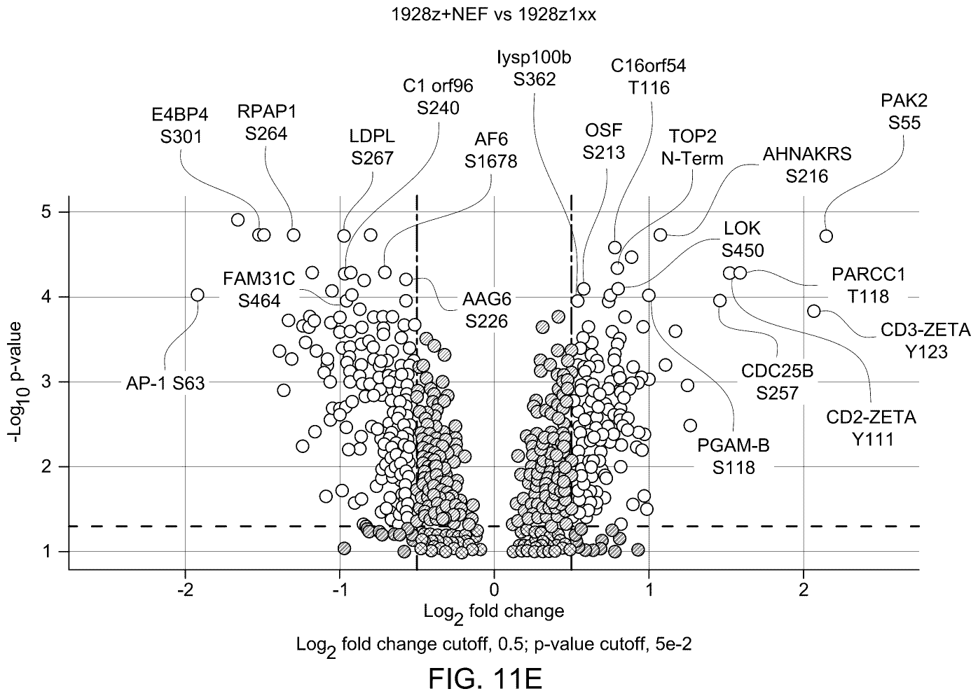

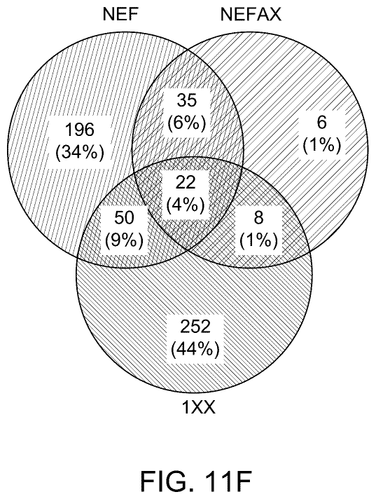

Nef is a multifunctional protein that modulates the expression of extracellular receptors and T cell signaling through two distinct functional domains (FIG. 11A). Surface receptor expression is modulated through an AP-1/clathrin associated domain, while T cell signaling is modulated through the PxxP domain. It was hypothesized that Nef signaling may be critical for its enhancement of T cell function. Here it is demonstrated that a mutation in the PxxP domain (NefAXXA mutant) but not the AP-1/clathrin associated domain (NEFD123G) abrogated the ability of Nef to enhance tumor control and survival when expressed in T cells with 1928z CAR in vivo (FIG. 11B). Thus, the presently disclosed subject matter demonstrates that Nef signaling through the PxxP domain is critical for its enhancement of T cell function.

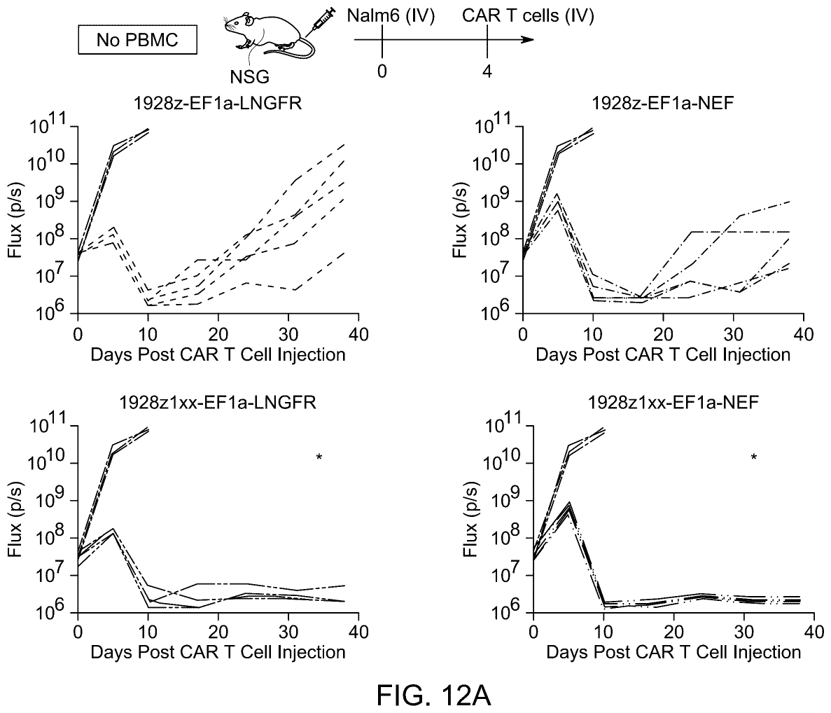

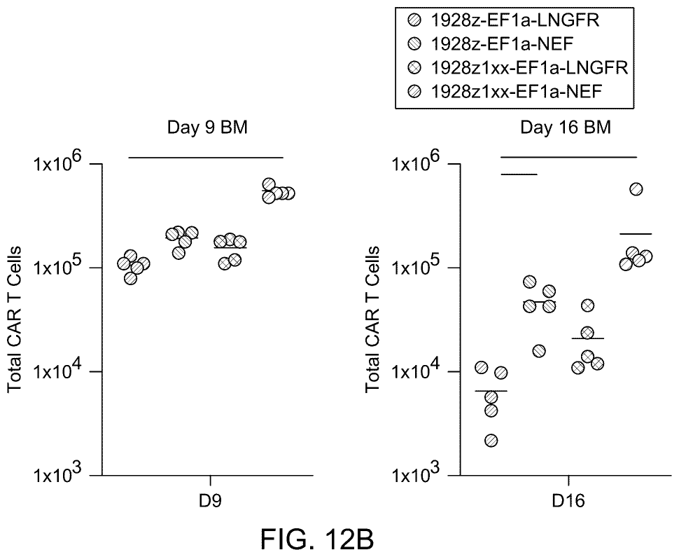

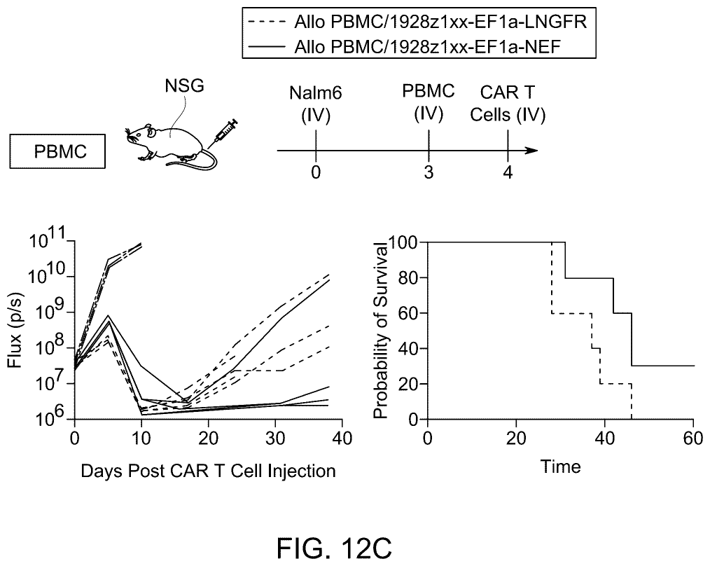

The PxxP domain interacts with and attenuates early T cell signaling kinases including Lck and Zap70. Thus, the presently disclosed subject matter assessed the impact of Nef on early T cell signaling by stimulating Nef-expressing CAR T cells with NALM6 tumor and evaluating CD3 zeta ITAM3 phosphorylation by flow cytometry. Here it was demonstrated that early T cell signaling at the CD3 zeta ITAM3 domain was attenuated with wild-type Nef, but not the NefAXXA mutant (FIG. 11C). A modified version of the 1928z CAR which contains mutations that abrogate activity of the ITAM2 and ITAM3 signaling domains of CD3zeta (termed ‘1928z1xx’ or ‘1xx’) demonstrates superior tumor control (FIG. 12A) in a NALM6 tumor xenograft model by modulating T cell signaling. In the presently disclosed flow cytometry phosphorylation assay, 1928z1xx demonstrated absent ITMA3 phosphorylation as expected (FIG. 11C).

Since both Nef and 1928z1xx demonstrate reduction of ITAM3 signaling, it was hypothesized that they function through a similar mechanism. Surprisingly, the presently disclosed subject matter found that the signaling cascades of 1928x1xx and 1928z+Nef were distinct. CAR T cells containing 1928z, 1928z+Nef, 1928z+NefAXXA, or 1928z1xx were stimulated with NALM6 cells for 10 minutes, and phosphoproteomic analysis of T cell signaling cascades was performed. While both 1xx and Nef were associated with ITAM2 (Y11) and ITAM3 (Y123) attenuation, Nef also attenuated ITAM1 and CD28 Y191, whereas signaling of both early activators was enhanced by 1xx (FIG. 11D). Likewise, differential phosphorylation of Nef and 1xx demonstrated highly distinct phosphorylation patterns (FIG. 12E), and Nef and 1xx had a high frequency of phosphosites with distinct changes relative to 1928z (FIG. 12F). Thus, 1xx and Nef impact the T cell signaling cascade in a distinct fashion.

After having determined that Nef and 1xx function through distinct molecular pathways, it was hypothesized that the two modification could be combined to enhance CAR T function. Next, AAV constructs containing both 1928z1xx and expressing Nef under a EF1α promoter were generated and transduced into T cells. The presently disclosed CAR T cells were first injected into a NALM6 xenograft model lacking PBMCs (no allogeneic rejection). In this tumor only model, both 1928z1xx and 1928z1xx-Nef demonstrated superior tumor control to 1928z-Nef and 1928z (FIG. 12A). However, the combination of 1928z1xx and Nef led to a significantly greater numbers of CAR T cells at Days 9 and 16 post-injection (FIG. 12B), indicating that the combination of 1xx and Nef enhance both CAR activity and the persistence of CAR T cell. These results indicate that Nef and 1xx function through non-overlapping pathways and are additive in enhancing the CAR function.

Next, the presently disclosed CAR T cells were tested in an allogeneic rejection model (PBMC). In a PBMC engrafted model, 1928z1xx-Nef outperformed 1928z1xx, with superior tumor control and prolonged survival (FIG. 12C). Thus, while both Nef and 1xx enhance intrinsic CAR function, only the combination of Nef and 1xx leads to optimal function in an allogeneic setting.

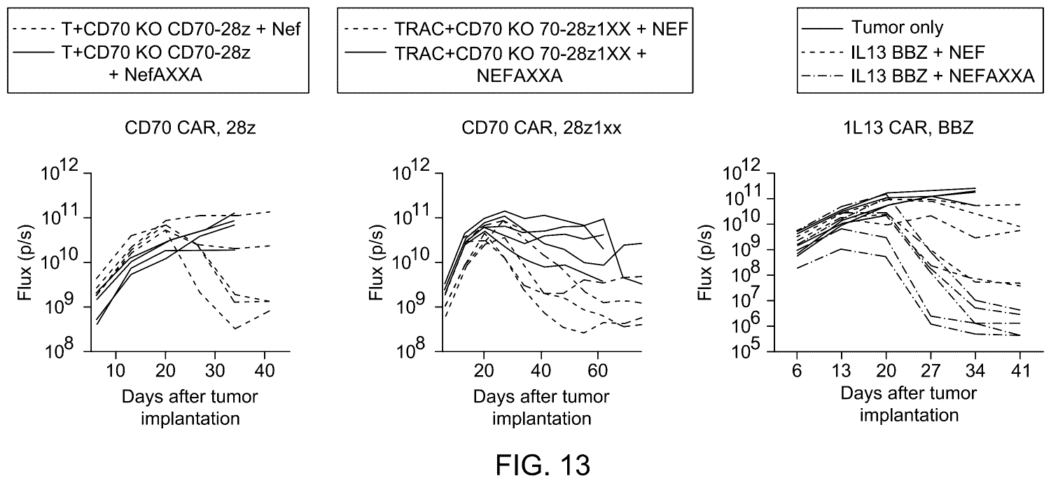

Finally, the presently disclosed subject matter demonstrated that Nef cannot enhance the function of all CAR variants and architectures. In a GBM xenograft flank model of the U251 cell line, which expresses CD70 and IL13, a CD70 directed CAR expressing either the wild type 28z or the 28z1xx CAR construct had enhanced tumor control when Nef was expressed. However, an IL13 directed CAR with the 41bb costimulatory domain (BBz) was impeded by expression of the wild-type Nef (FIG. 13). This data illustrates that Nef cannot be combined with all CAR variants, and the combination of NEF with the 28z and 28z1xx CAR is a unique combination.

### Example 3

Chimeric antigen receptor (CAR) T cells are a genetically engineered T cell-based therapy that is highly effective against B cell malignancies and multiple myeloma. Most CAR T cells are currently manufactured individually for each patient from their own leukapheresis product. The length and cost of autologous manufacturing limit access and leave some patients vulnerable to disease progression and complications while waiting for CAR T cell production; in some instances, the patient's own T cells are defective or fail manufacturing. In contrast, CAR T cells derived from healthy donors or renewable stem cells would be immediately available for infusion, could be manufactured in bulk at reduced cost, and display more proliferative and functional capacity than cells derived from patients with cancer. However, allogeneic cell therapies are limited by immune interactions between donor and host. Graft-versus-host responses occur when ap-T cell receptors (TCR) recognize host tissue. Allo-recognition can be mitigated by the use of T cells lacking an alloreactive TCR or by altogether eliminating TCR expression. Conversely, Host-Versus-Graft (HVG) responses, in which host immune cells such as T and NK cells reject donor cells, could impair allogeneic CAR T cell expansion and persistence, thereby curtailing their efficacy. Initial clinical experience with allogeneic CAR T cells has demonstrated limited CAR T cell persistence in the absence of deep immune suppression establishing that HVG responses are a major barrier to allogeneic CAR T cell efficacy. To devise a strategy to protect CAR T cells from immune rejection, the presently disclosed subject matter turned to lymphotropic viruses that have evolved integrated mechanisms to enable immune escape and persistence of virus-infected lymphocytes. A common strategy shared by HIV, EBV and CMV17-19 is to reduce the expression of Human Leukocyte Antigen class I (HLA-I), thus mitigating CD8 T cell recognition. Distinct pathways are utilized by viral evasins: CMV proteins US2 and US11 cause retrograde translocation of HLA-I for proteasomal destruction, while the HIV-1 Nef protein functions via clathrin-mediated endocytosis of a broad range of surface proteins, including HLA-I. The complete loss of HLA expression in human cells leads to recognition and rejection by NK cells. Here, it is demonstrated that HLA-I expression below 40% of baseline provides relative protection from allogeneic CD8 T cell killing, but that HLA-I expression below approximately 20% of baseline renders CAR T cells vulnerable to NK cell-mediated rejection. Reduction to the 20-40% range can be achieved through controlled expression of the HIV-1 Nef protein. Compact viral genomes often encode polyfunctional proteins that co-opt multiple cellular pathways to promote viral replication. Indeed, it was found that Nef deploys additional mechanisms that enhance CAR T cell survival and tumor control in vivo. Here, a key activity of Nef on the serine kinase Pak2 was identified, which promotes anti-apoptotic BCL-2 signaling and protects CAR T cells from activation induced cell death, further extending their longevity and function. Thus, Nef acts via multiple pathways to extend CAR T cell survival, leading to highly effective allogeneic CAR T cells.

### Results

### Allogeneic CAR T Cells are Eliminated by Host CD8 T Cells

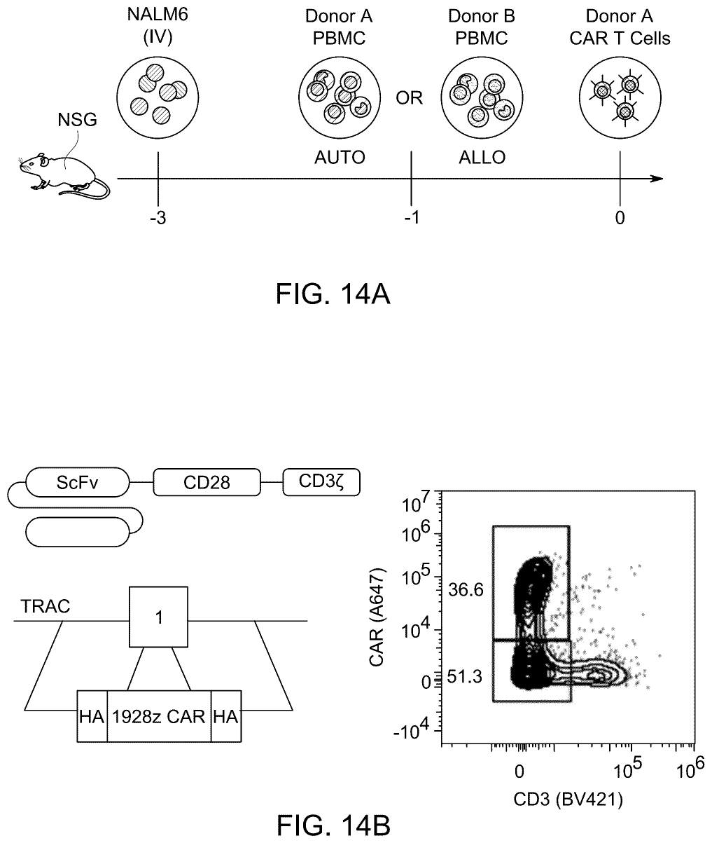

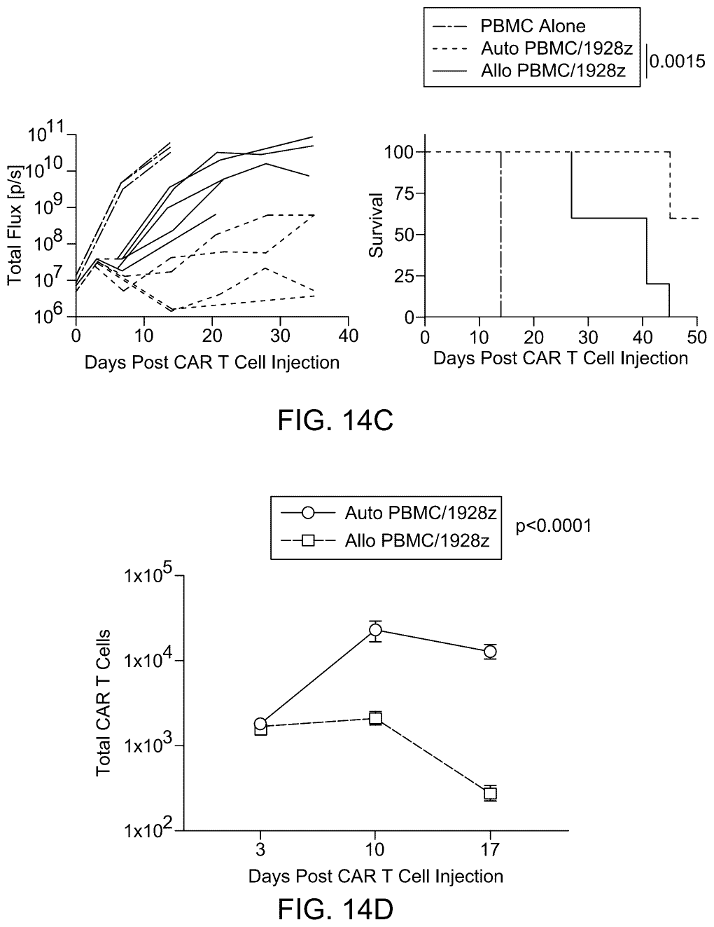

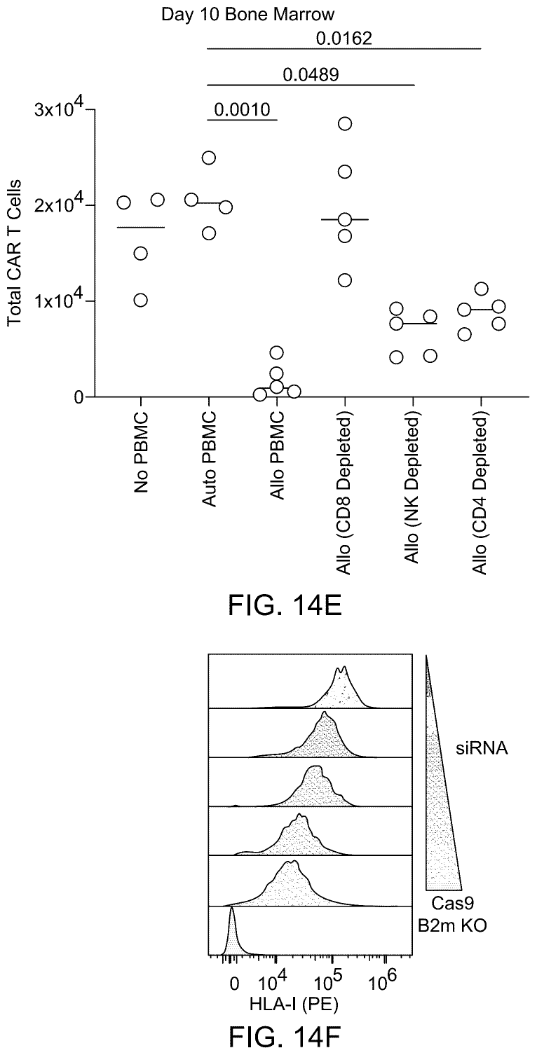

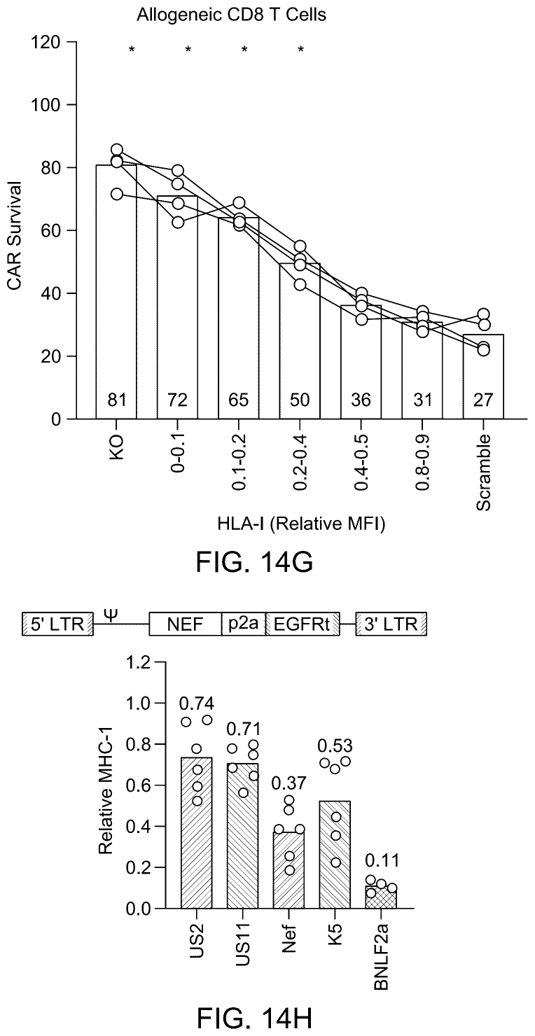

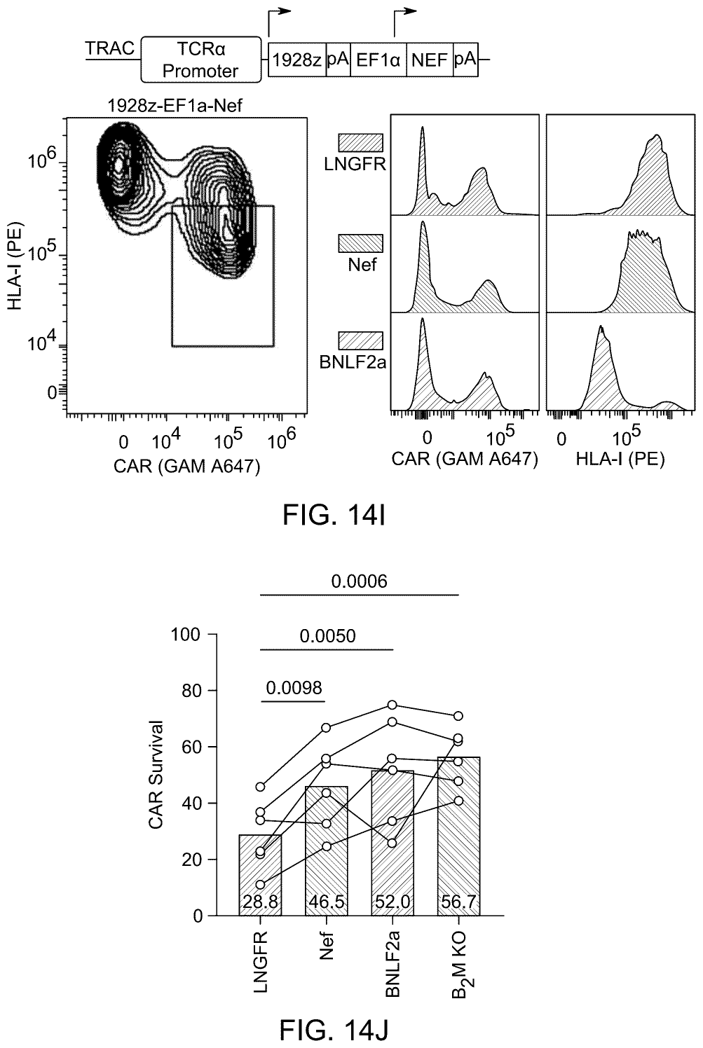

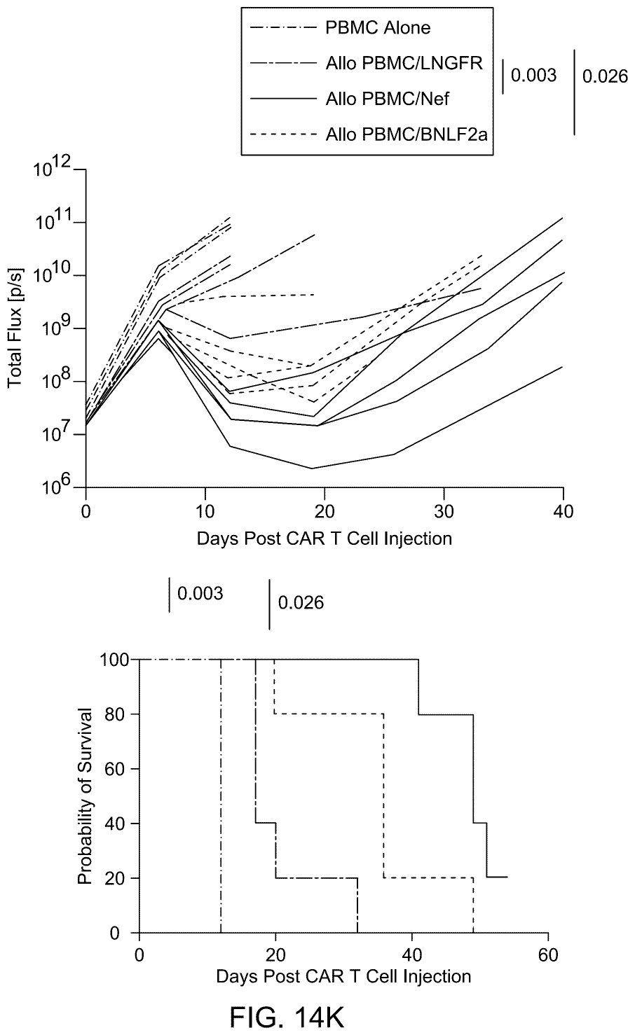

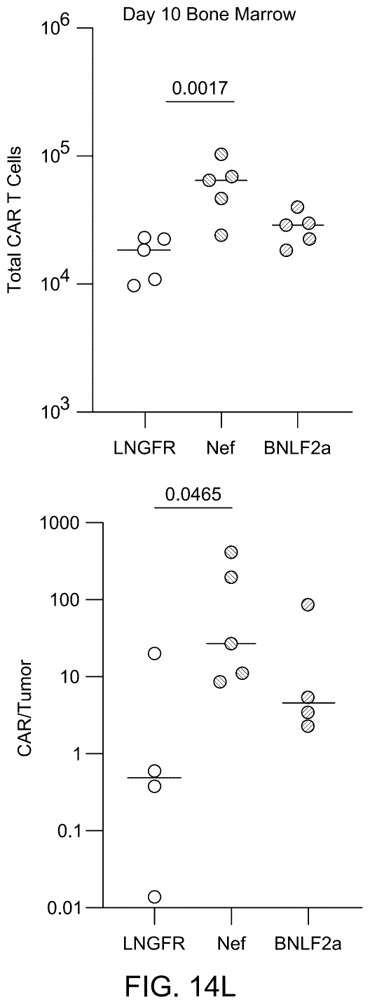

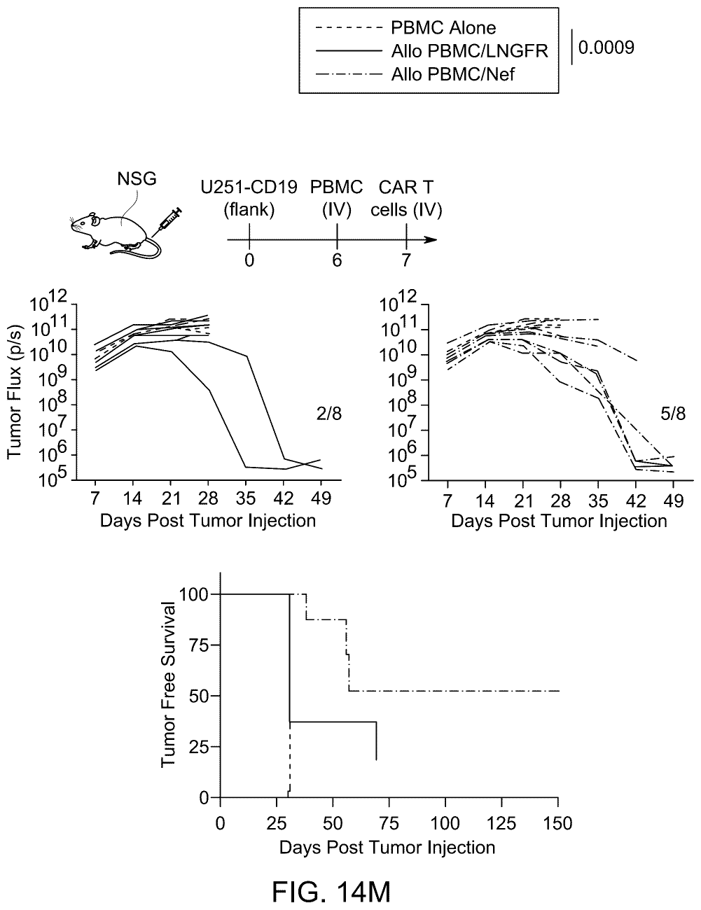

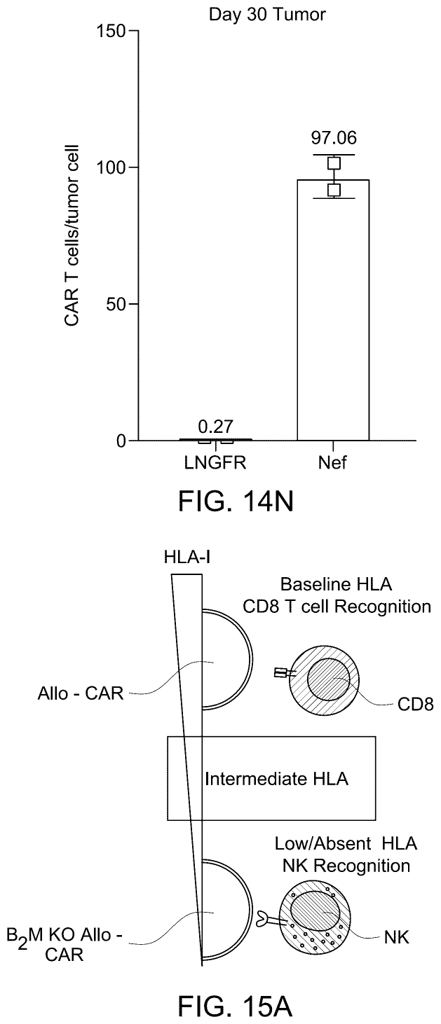

To investigate host-versus-graft (HVG) allogeneic rejection of CAR T cells and its impact on tumor control, the CD19+ NALM6 acute lymphoblastic leukemia model was adapted. Tumor-bearing NSG mice were engrafted with peripheral blood mononuclear cells (PBMC) from either the CAR T cell donor or an unrelated donor to model autologous and allogeneic cell therapy settings, respectively (FIG. 14A). The activity of the adoptively transferred CAR T cells was monitored by measuring tumor responses using bioluminescence imaging. CAR T cells were generated by targeting the CAR cDNA via Cas9-editing to the TCR alpha constant (TRAC) gene locus (FIG. 14A) to provide consistent and effective CAR expression. 1928z is an extensively reported CD19-specific CAR comprising the CD28 costimulatory domain and the CD3ζ signaling domain (FIG. 14B). In removing endogenous TCR expression, TRAC integration prevents graft-versus-host disease (GVHD) while yielding potent CAR T cells owing to regulated CAR expression. Tumor control by CD19 CAR T cells was more effective in mice bearing autologous PBMC compared to allogeneic PBMCs (FIGS. 1E and 14C), with superior tumor control and survival (median survival NR vs 41 days, p=0.0015). This pattern was consistently observed across allogeneic donor pairings (FIG. 1E). CAR T cells were approximately 10-fold more abundant in bone marrow ten days after their infusion in autologous mice compared to their allogeneic counterparts (FIG. 14D), consistent with host-versus-graft (HVG) CAR T cell rejection occurring in the presence of allogeneic PBMC. Depletion of CD8+ but not CD4+ T cells or NK cells from allogeneic PBMC prior to their infusion restored CAR T cell levels to those seen in mice without PBMC or mice reconstituted with autologous PBMC (FIG. 14E). Thus, CAR T cell accumulation and tumor control was limited by immune rejection primarily mediated by host CD8 T cells in NSG mice.

### Reduction of HLA Expression Protects Against CD8 T Cell Killing

A common mechanism used by viral evasins to escape immune recognition is down-regulation of HLA-I. Thus, it was sought to first determine the degree of reduction sufficient to thwart CD8 T cell-mediated elimination. Transient HLA-I knockdown resulting in median expression 10-80% of endogenous levels was achieved by electroporation of serially diluted siRNA targeting the HLA-I structural component beta-2 microglobulin (B2M) (FIG. 14F). Complete and stable HLA-I knockout was achieved by Cas9 editing of the B2M locus (B2MKO). B2MKO provided the greatest protection from allogeneic CD8 T cell killing (FIG. 14G). However, siRNA-modified T cells with median HLA levels below 40% of control were partially protected from CD8 T cell killing, with increasing protection as HLA levels approached complete knockout (FIG. 14G). To partially but stably reduce HLA-I expression, five candidate immune evasion proteins known to modulate HLA levels were selected: Nef from HIV-1 Clade B, BNLF2a from EBV, US2 and US11 from hCMV, and K5 from KSV17. Each was individually expressed in bicistronic retroviral vectors containing a truncated EGFR reporter gene. EGFR+ cells had decreased HLA-I expression, but the magnitude of HLA reduction varied: US2 and US11 expression generated cells with HLA-I at 80% of baseline levels, while K5 achieved 40-50%, Nef achieved 30-40%, and BNLF2a achieved 10-20% (FIG. 14H). HIV-1 Nef was noted to downregulate HLA-A and HLA-B alleles relative to HLA-C and E.

### Nef and BNLF2a Confer Allogeneic Resistance to CD8 T Cells

As Nef and BNLF2a regulated HLA levels in the <40% range that provided CD8 T cell protection in siRNA experiments (FIG. 14F), AAV homology vectors for site-specific integration to achieve consistent levels of HLA downregulation were generated. The TRAC locus was used to co-express the CAR and either Nef, BNLF2a, or LNGFR, a reporter gene used as a control (FIG. 14I). Different vectors to express viral evasins under the EF1α promoter were evaluated, which led to consistent and reproducible HLA-I downregulation that was maintained in the desired range. CAR T cells that expressed either Nef or BNLF2a showed decreased HLA-I expression and acquired resistance to CD8 T cell killing in a 16-hour in vitro survival assay (FIG. 14J). To test whether expression of Nef and BNLF2a protected cells against immune rejection in vivo, CAR T cells were infused at a low “stress test” dose (10⁵ CAR T cells) in mice engrafted with allogeneic PBMC. BNLF2a and Nef expression improved allogeneic CAR T cell tumor control in vivo (FIG. 14K; median survival 36 days and 49 days, respectively, compared to 17 days for the LNGFR control group). Nef provided the most durable tumor control and resulted in the largest number of CAR+ cells in bone marrow (FIG. 14L), approximately 4-fold more than control (median 6.5e4 vs 1.7e4, p<0.001). Nef likewise enhanced allogeneic CAR T cell function in a solid tumor model of CD19-expressing U251 glioblastoma cells implanted in the flank of PBMC-bearing mice. Nef-expressing CAR T cells (hereafter referred to as Nef-CARs) again significantly extended survival (FIG. 14M), with 5/8 mice as compared to 2/8 mice achieving long-term tumor control, and led to robust CAR T cell expansion and survival (FIG. 14N). Thus, Nef expression extended CAR T cell survival and improved tumor rejection in two in vivo allo-rejection models.

### Intermediate HLA-I Levels Prevent NK Cell Rejection

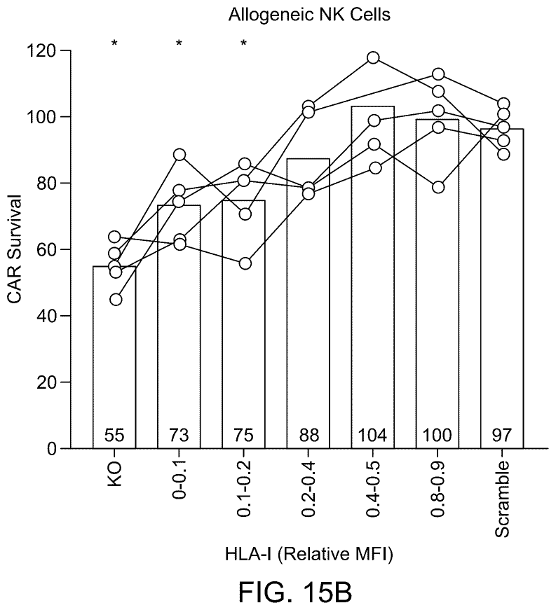

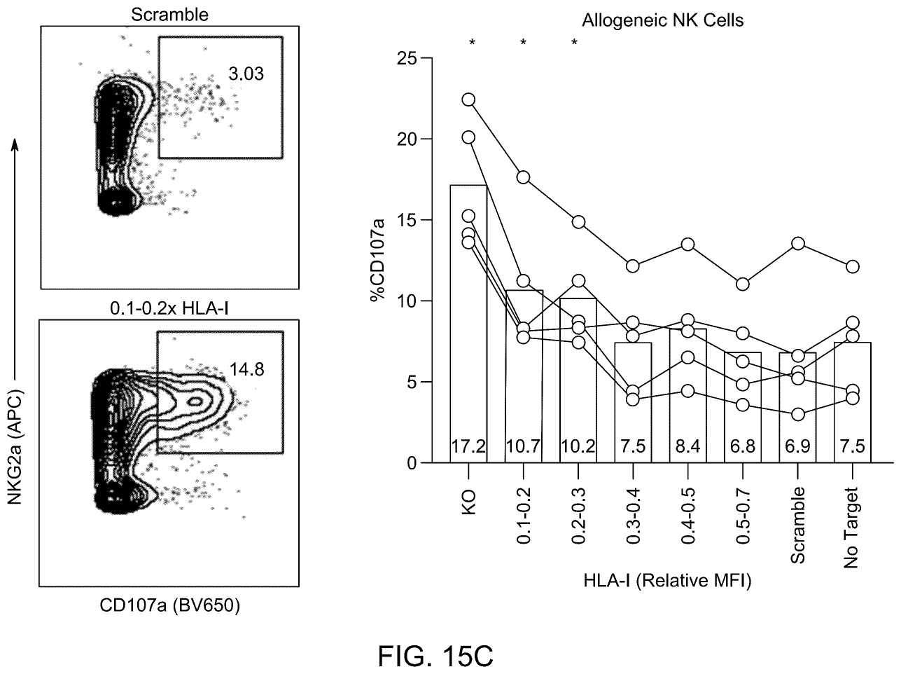

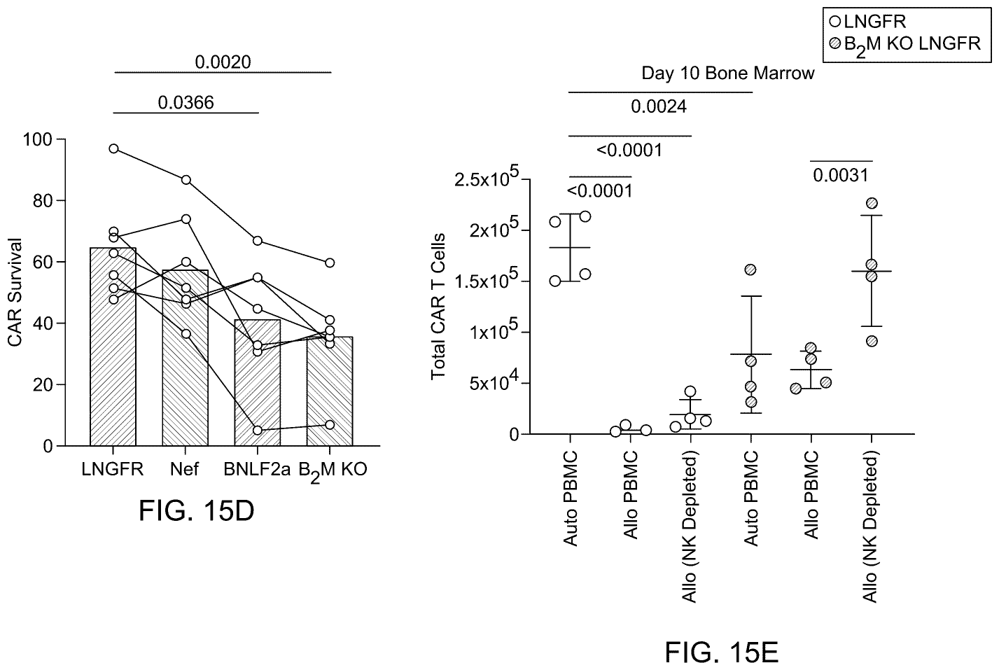

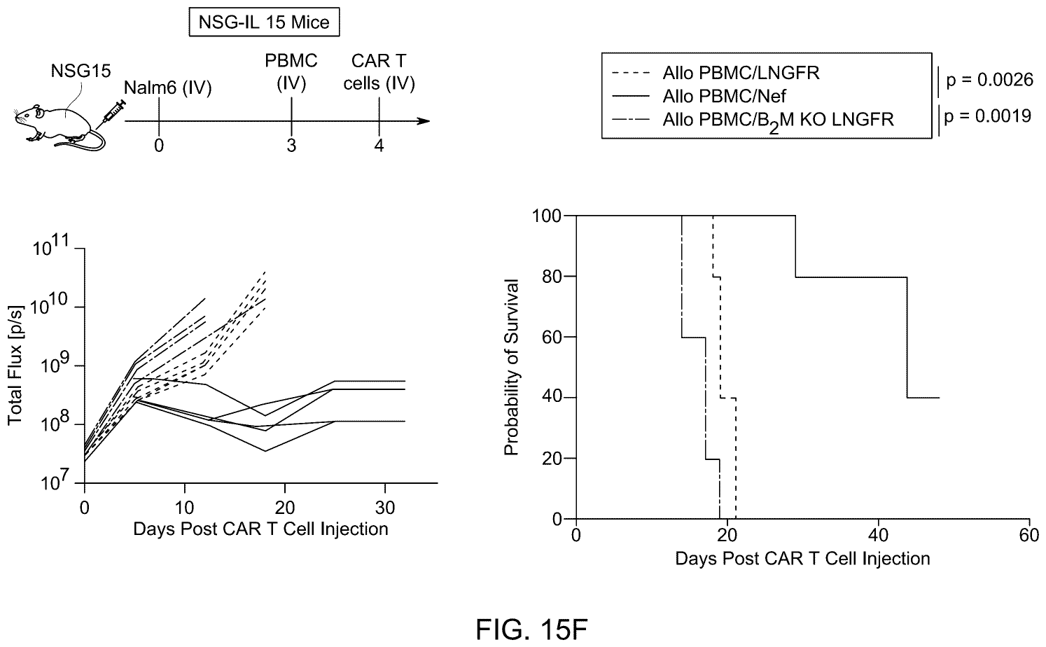

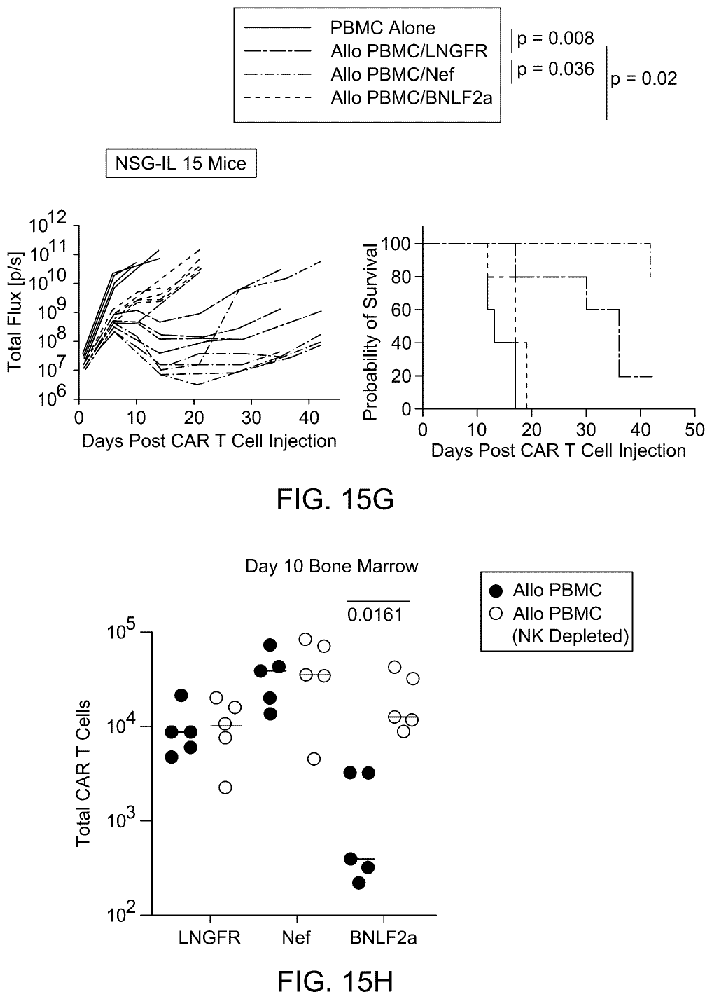

Compared to BNLF2a, Nef expression achieved superior tumor control and cell survival in vivo despite lesser HLA-I reduction. As NK cells can be activated by low or absent HLA-I expression, it was hypothesized that low HLA-I levels achieved by BNLF2a may lead to NK cell rejection (FIG. 15A). B2M siRNA-treated T cells with varying levels of HLA-I were studied and it was found that treated cells were sensitive to in vitro NK cell killing at HLA-I levels less than 20% of baseline (FIG. 15B). Degranulation in NK cells was observed when exposed to CAR T cells expressing 20% or less of endogenous HLA-I (FIG. 15C). Degranulation was prominent in NK cells expressing NKG2A, an inhibitory receptor that broadly binds HLA-I alleles. Consistent with this range predicted by siRNA knockdown, both B2MKO and BNLF2a-expressing (10-20% expression) CAR T cells were sensitive to NK cell killing in vitro compared to Nef and LNGFR expressing cells (FIG. 15D). To establish whether low levels of HLA-I led to NK rejection in vivo, PBMC-engrafted model was further adapted to NSG15 mice. Whereas NK cells are rapidly lost in NSG mice, NSG15 mice secrete human IL-15 and promote durable human NK cell (as well as T cell) activation and survival. To validate in vivo NK cell rejection of HLA-I deficient T cells in the NSG15 model, B2MKO LNGFR CAR T cells (TRACKOB2MKO 1928z-EF1α-LNGFR) were compared to HLA intact LNGFR CAR T cells (TRACKO 1928z-EF1α-LNGFR). Magnetic depletion of NK cells prior to PBMC infusion led to recovery of B2MKO LNGFR cells but not control LNGFR, confirming that NK cells were responsible for limiting B2MKO cell expansion (FIG. 15E). In allogeneic PBMC treated NSG15 mice, Nef-CARs showed superior tumor control compared to control LNGFR or B2MKO LNGFR CAR T cells (FIG. 15F). Strikingly, B2MKO CAR T cells showed particularly poor tumor control in NSG15 mice despite normal tumor killing in vitro, further supporting that HLA-I-deficient, NK-sensitive CAR T cells display reduced ability to control tumor. Similarly, BNLF2a-CARs with very low HLA expression were less effective than control LNGFR-CARs in NSG15 mice (FIG. 15G). LNGFR- and Nef-CAR T cell accumulation were not affected by the presence or absence of NK cells, in contrast to BNLF2a-CAR T cells, which were significantly higher in NSG15 mice reconstituted with NK-depleted PBMC (FIG. 15H). Thus, both in vitro and in vivo modeling demonstrate that complete HLA-I knockout or reduction of HLA-I to levels 10-20% of baseline is sufficient to render CAR T cells susceptible to robust NK cell rejection, whereas Nef-CAR T cells mitigated CD8 T cell rejection without triggering NK cells.

### Nef Enhances CAR T Cell Tumor Control Via its SH3-Binding Domain

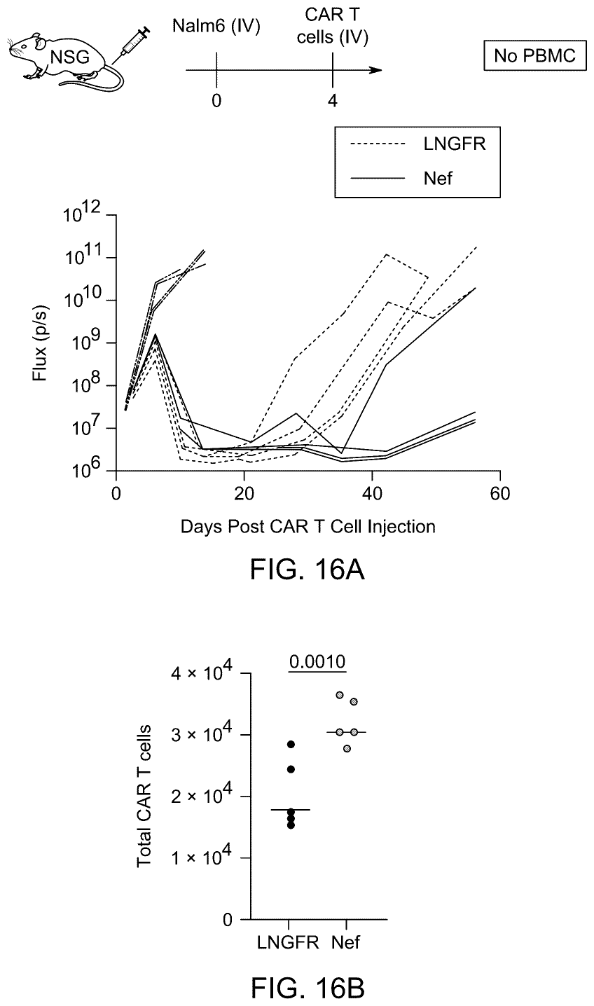

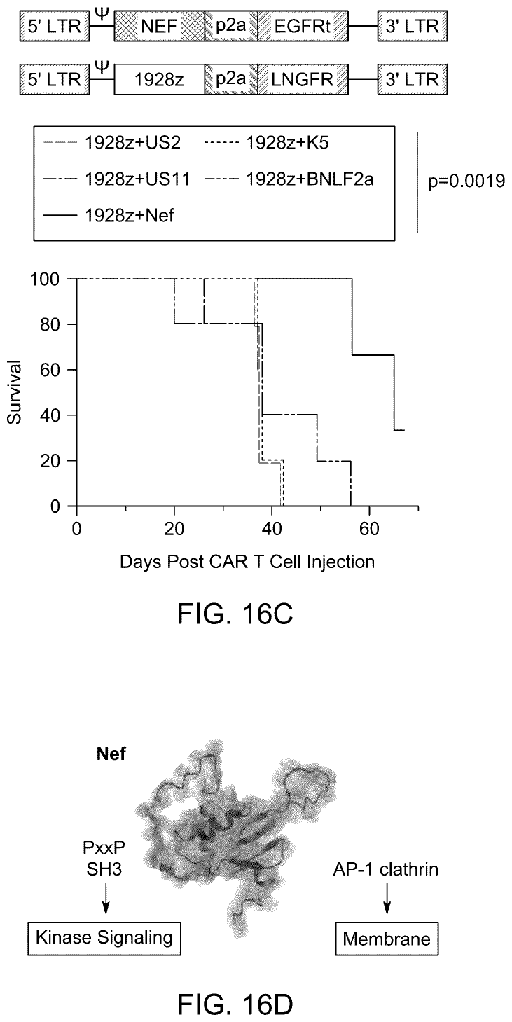

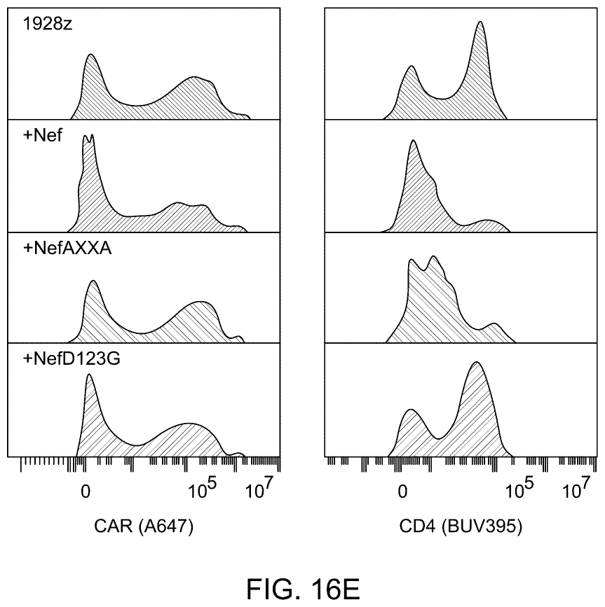

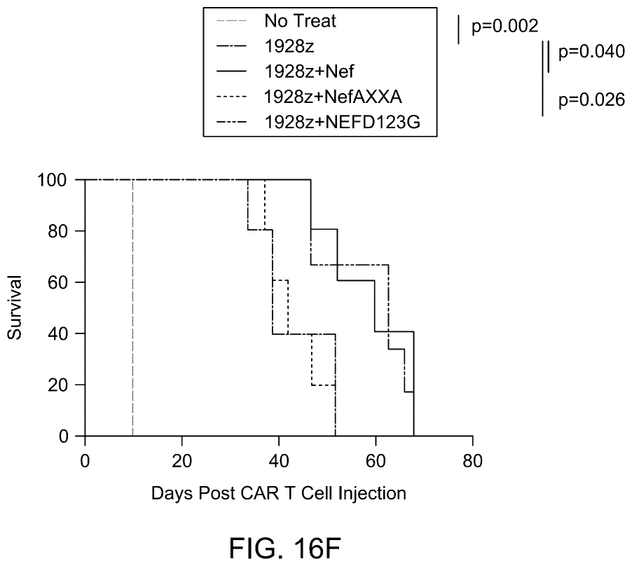

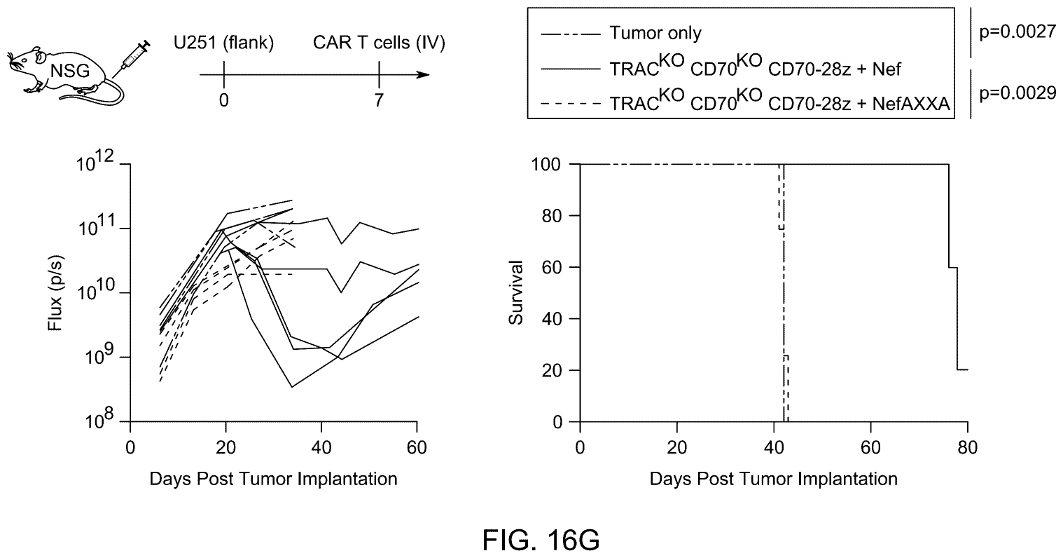

Nef-CARs also outperformed BNLF2a constructs in NSG mice where NK engraftment is not durable (FIG. 14K); therefore it was hypothesized that Nef provides an additional benefit to CAR T cells, distinct from its effect on HLA down-regulation. To test this possibility, the presently disclosed subject matter returned to the classic NALM6 xenograft model without PBMC reconstitution and assessed the intrinsic CAR T cell activity afforded by Nef expression. Nef-CARs showed enhanced tumor control (FIG. 16A) and greater CAR T cell expansion than LNGFR control cells (FIG. 16B). In contrast, expression of BNLF2a and other viral evasins did not augment tumor control by CAR T cells (FIG. 16C). Thus, Nef uniquely increased CAR T cell frequency and improved tumor control, even in the absence of immune rejection. Nef is a polyfunctional protein, with the ability to downregulate surface receptors including HLA-I, CD4, SERINC5, CD28, CD80/86 through interactions with the AP-1 clathirin adapter. It further contains an SH3-binding domain that mediates interactions with a wide variety of proteins, including Src-family tyrosine kinases (FIG. 16D). Therefore it was sought to determine whether SH3-domain mediated interactions or downregulation of additional surface proteins were responsible for promoting CAR T cell anti-tumor activity. To address this question, well-established Nef mutants known to abrogate specific Nef functions were studied. Wild-type Nef was compared to Nef-D123G, which impairs interaction with the AP-1 clathrin adapter, and thus interferes with both HLA-I and CD4 downregulation (FIG. 16E), and NefAxxA, which abrogates function of the SH3 binding domain. Expression of WT Nef and NefD123G but not NefAxxA enhanced tumor control by 1928z CAR T cells (FIG. 16F), suggesting the SH3 domain was critical for Nef-enhanced tumor rejection. Nef but not the NefAxxA variant also enhanced the performance of a CD70-targeted CAR T cell in a glioblastoma flank tumor model (FIG. 16G). The NefD123G variant as well as the NefWP variant, which both lose the ability to downregulate CD4, were further studied. Both variants outperformed control LNGFR CAR T cells similarly to WT Nef CAR T cells, establishing that CD4 downregulation was dispensable for the Nef enhancement of in vivo CAR T cell survival. Furthermore, no loss of proteins known to be affected by Nef in other contexts including macrophage infection (CD80, CD86, CD28, CXCR3, or CXCR4) by wild-type Nef or any variant was detected. Thus, the Nef SH3 binding domain rather than downregulation of additional surface proteins is required to enhance intrinsic CAR T cell function.

Nef Induces Pak2 Phosphorylation and Protects from Activation-Induced Cell Death

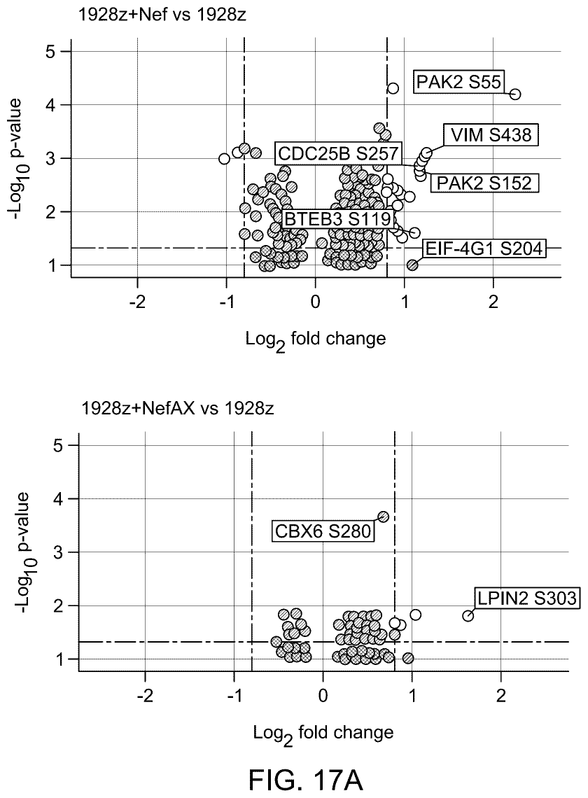

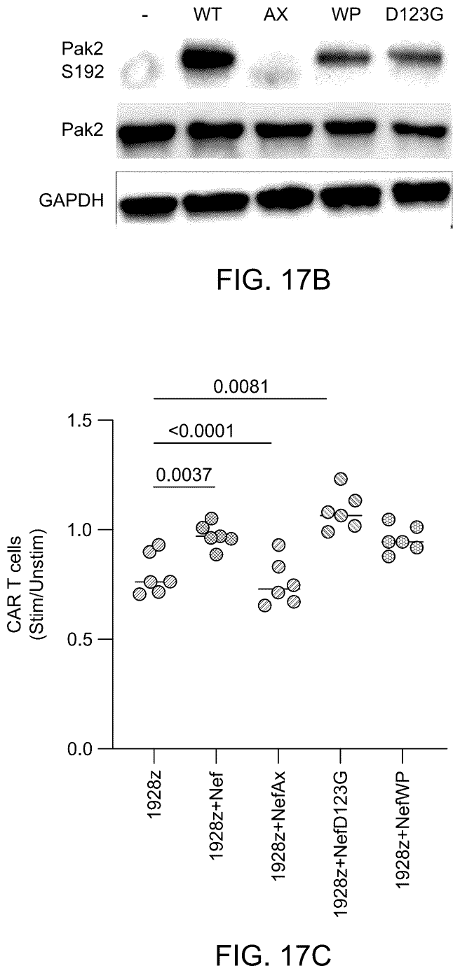

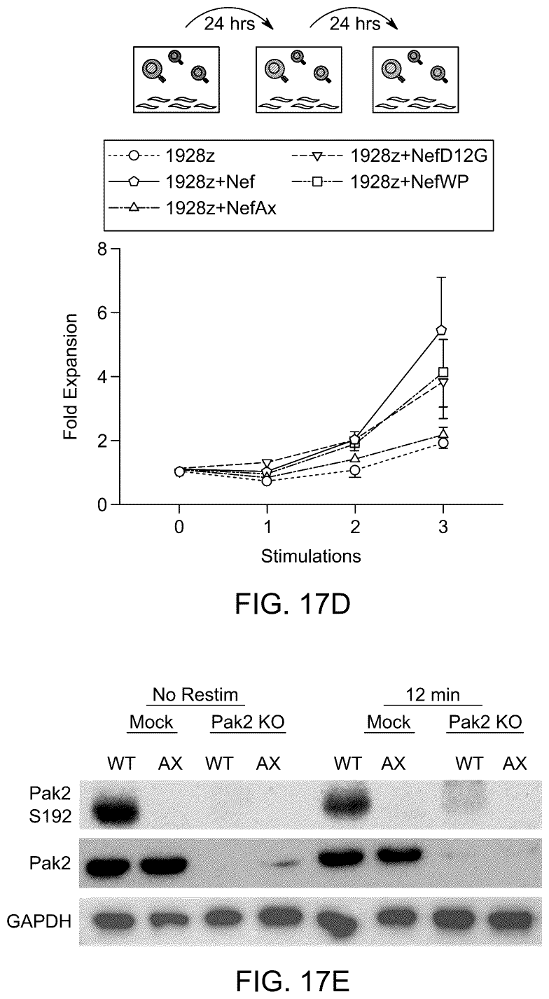

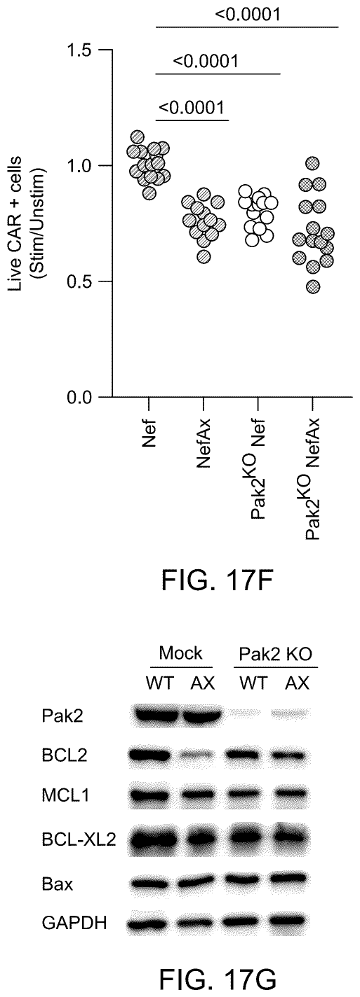

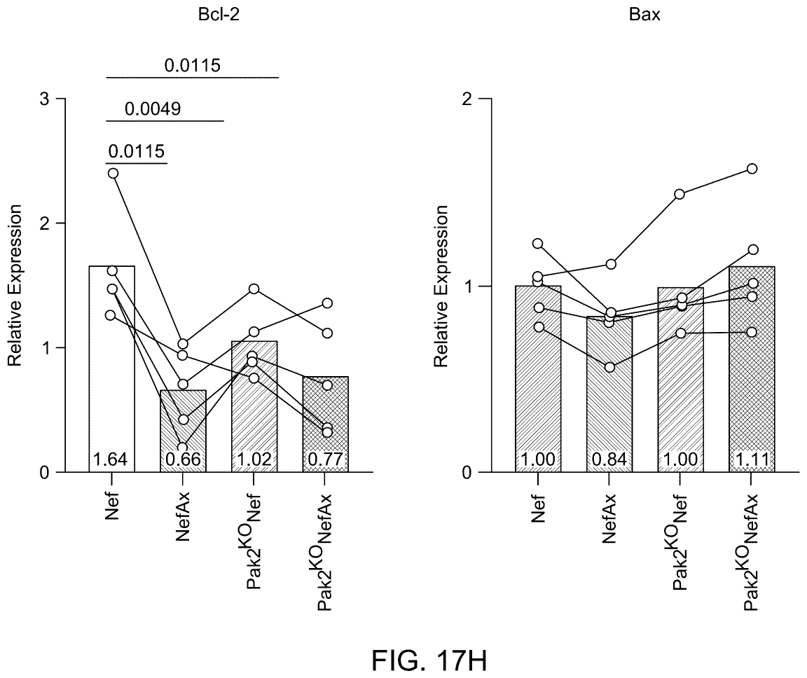

To investigate the mechanism by which the Nef SH3-binding domain enhances CAR T cells, quantitative phosphoproteomic analysis of CAR T cells stimulated by SILAC-labeled NALM6 cells was performed. Expression of Nef-WT compared to NefAxxA CAR T cells led to major alterations in cellular phosphorylation state, with particularly prominent phosphorylation of the Pak2 kinase at multiple sites, including S55 (log 2Fc 2.15, p<1e-5) and S152 (log 2Fc 1.17, p<1e-3) (FIG. 17A). Nef is known to interact with Pak2, and Pak2 activation triggers autophosphorylation at eight regulatory sites including S55, S142, S152, and S192. It was confirmed by gel electrophoresis that Nef variants that promote CAR T cell activity in vivo (Nef WT, NefWP and NefD123G) but not NefAxxA expressing CARs also promoted Pak2 phosphorylation (FIG. 17B). Pak2 is a serine threonine kinase that has been implicated in actin signaling, mechanotransduction, and apoptosis, including both pro- and anti-apoptotic effects. As CAR T cells may undergo activation-induced cell death (AICD), particularly in the presence of high tumor burden and repeated stimulation, it was evaluated the impact of Nef-mediated Pak2 signaling on apoptosis and AICD. Five hours after stimulation with either NALM6 tumor cells, 1928z CAR T cells demonstrated characteristic signatures of apoptosis: phosphatidylserine exposure by Annexin V staining, loss of membrane integrity by Sytox viability dye uptake, and loss of mitochondrial membrane potential by TMRM staining. Sixteen hours after stimulation with Nalm6 (FIG. 17C) or anti-CAR or anti-CD3 antibodies, fewer 1928z CAR T cells were present in stimulated compared to unstimulated conditions, reflecting activation-induced cell death. CAR T cells expressing wild-type Nef and CAR-enhancing variants (D123G, and WP) but not NefAxxA were protected from AICD (FIG. 17C). Furthermore, only enhancing Nef variants showed robust expansion when stressed with repeated stimulation every 24 hours (FIG. 17D). Dilution of a cell viability dye was identical 1 and 3 days after stimulation, confirming that reduced apoptosis rather than increased proliferation was responsible for higher cell frequencies. Remaining viable cells subsequently underwent robust proliferation approximately 3-4 days after stimulation.

### Nef Promotes Survival Signaling Via Pak2 and Increases T Cell Accumulation Independently of CAR Activation Strength

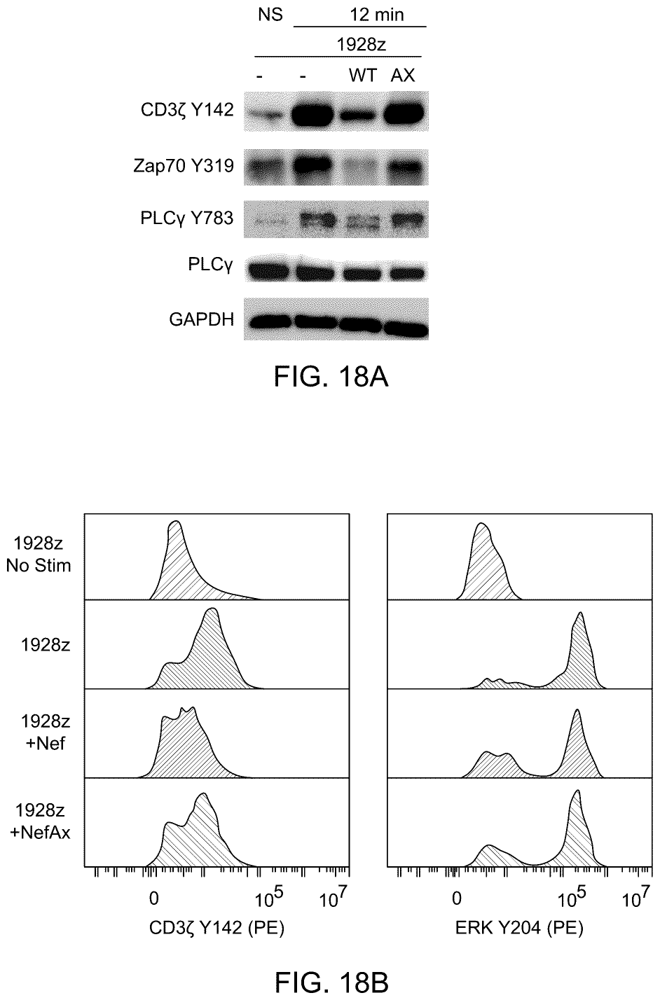

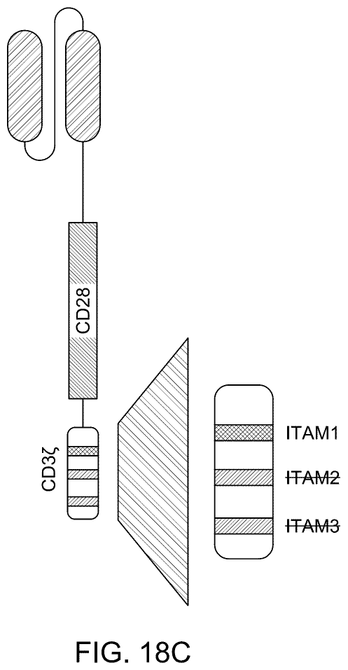

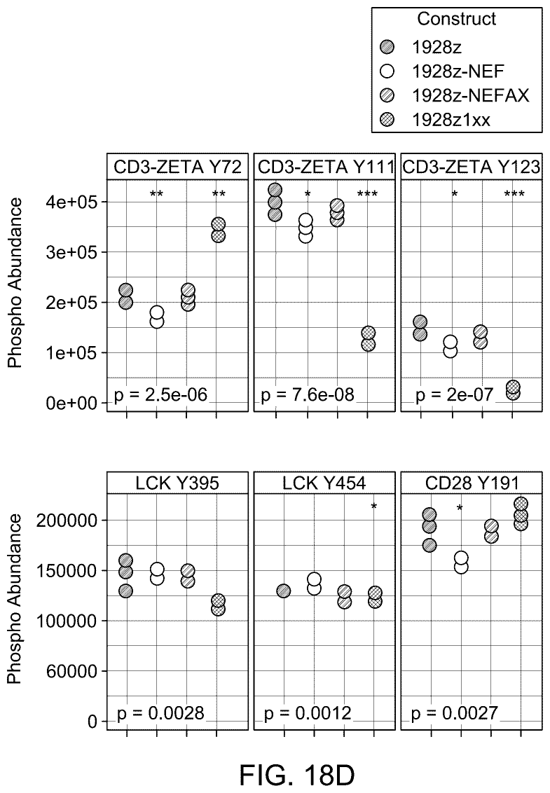

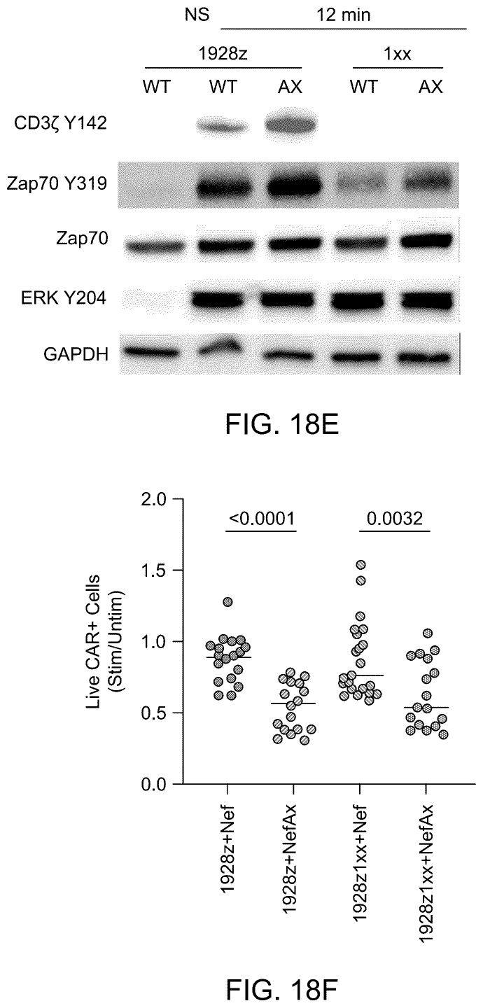

To confirm the requirement for Pak2 in protection from AICD, Cas9 editing was used to generate Pak2KO Nef- and NefAxxA-CARs (FIG. 17E). Pak2 KO did not impair ERK phosphorylation downstream of CAR signaling, or the ability of CAR T cells to kill tumor cells. However, only Nef-CARs with intact Pak2 resisted AICD after activation; Pak2KO Nef CARs did not display greater survival than NefAxxA CARs (FIG. 17F). It was hypothesized that Nef-expressing cells are protected from AICD by modulating Bcl-2 family apoptotic regulatory proteins. Indeed, Nef-CARs showed strikingly increased expression of the anti-apoptotic Bcl-2 without a concomitant increase in pro-apoptotic Bax expression (FIG. 17G). This effect was abrogated by ablating Pak2. Quantitative measurement of Bcl-2 expression in Nef-expressing CAR T cells from 5 independent donors showed a 1.6-fold increase in Bcl-2 in Nef compared to Pak2KO Nef cells (FIG. 17H). Expression of surface death receptors (Fas, DR5, TNFR1) was unchanged with Nef expression. Thus, Nef expression leads to phosphorylation of Pak2, upregulation of anti-apoptotic Bcl-2, and protects against AICD in a Pak2-dependent manner. In addition to its effect on Pak2, the Nef SH3 binding domain is known to interact with and attenuate early T cell signaling proteins, including LCK and ZAP70. It was confirmed that expression of wild-type Nef in CD19-bead stimulated CAR T cells led to diminished early signaling events including decreased phosphorylation of CD3ζ Y142, ZAP70 Y319, and PLCγ Y783, relative to mock transduced and NefAxxA transduced CAR T cells (FIG. 18A). Phospho-flow cytometry analysis of CAR T cells stimulated against NALM6 tumor cells further confirmed that Nef had attenuated CD3ζ (Y142) phosphorylation relative to 1928z and 1928z+NefAxxA CAR T cells (FIG. 18B). This was seen in both CD4 and CD8 CAR T cells. Despite this early signaling attenuation, downstream ERK phosphorylation was preserved (FIG. 18B), confirming that T cell activation could still be triggered in response to antigen. Since the Nef SH3 domain interacts with both Pak2 and T cell signaling machinery, Nef mutants alone could not be used to determine whether signaling attenuation contributed to protection from AICD. However, it was investigated whether Nef limited AICD following activation of an attenuated CAR. Thus, Nef activities in T cells expressing either the 1928z CAR or its attenuated version retaining only one of CD3ζ's three activation motifs (termed 1928z1XX or 1XX; FIG. 18C) were compared. By quantitative phosphoproteomics, Nef-expressing 1928z CARs showed reduced phosphorylation at CD3ζ ITAMs 2 and 3, as did 1XX CARs, compared to mock-transduced 1928z CAR (FIG. 18D). Expressing Nef in 1XX-CARs led to further reduction of Zap70 phosphorylation downstream of CAR signaling, with ERK phosphorylation preserved (FIG. 18E) as was observed with 1928z (FIG. 18B). Nonetheless, expressing Nef conferred protection from AICD to both constructs (FIG. 18F). Thus, Nef provided a comparable anti-apoptotic effect in the presence of either strong or attenuated T cell activation.

### 1XX and Nef Promote In Vivo Survival and Function

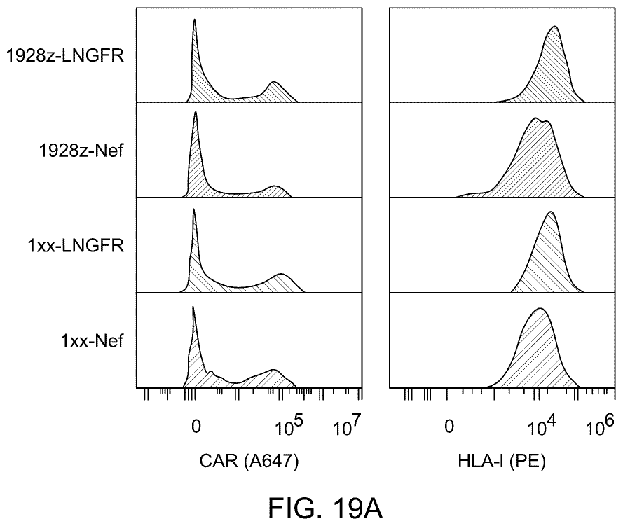

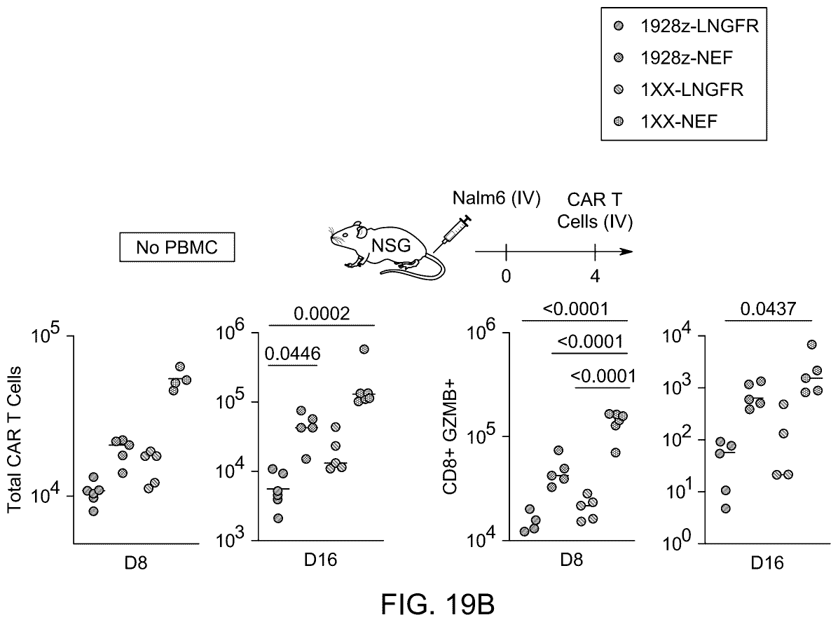

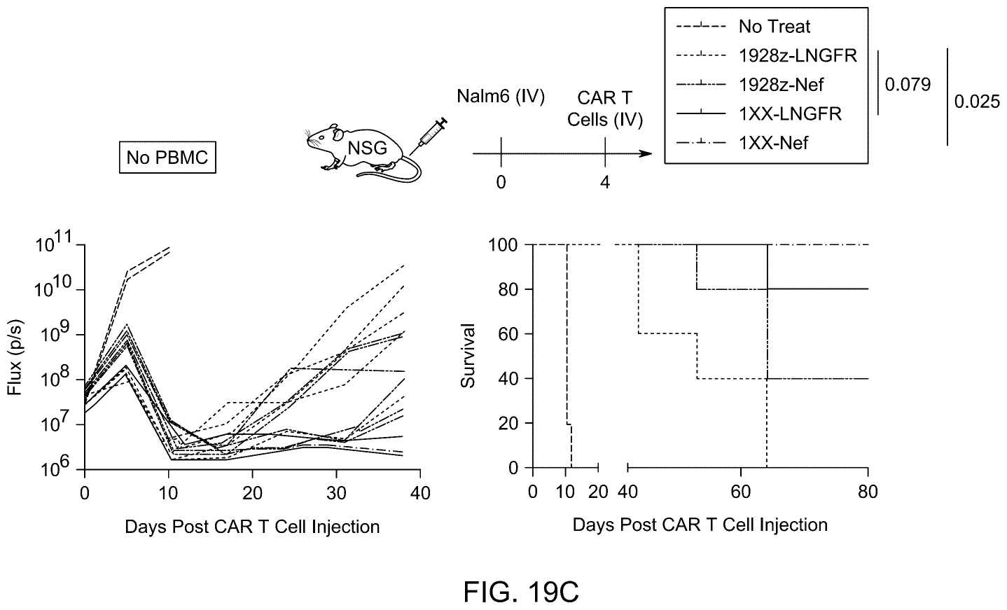

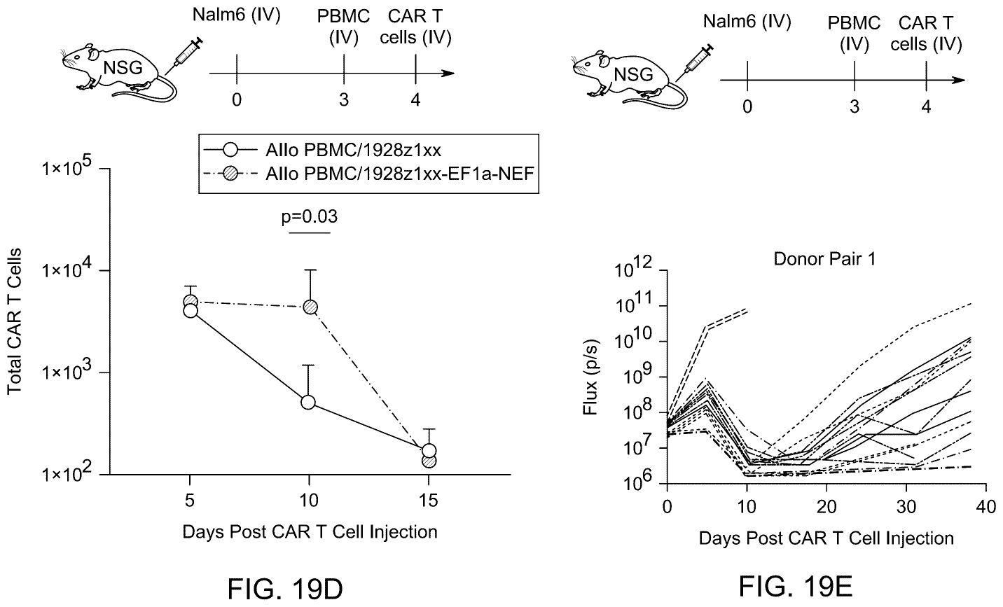

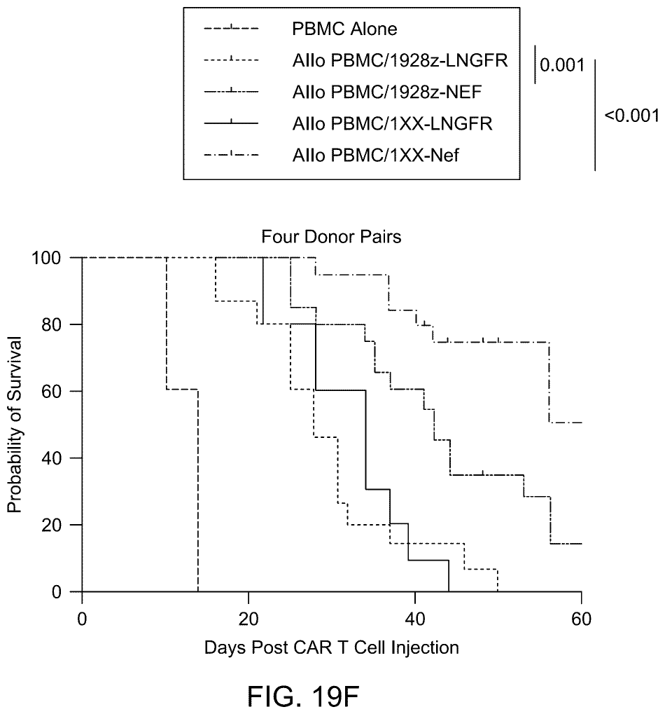

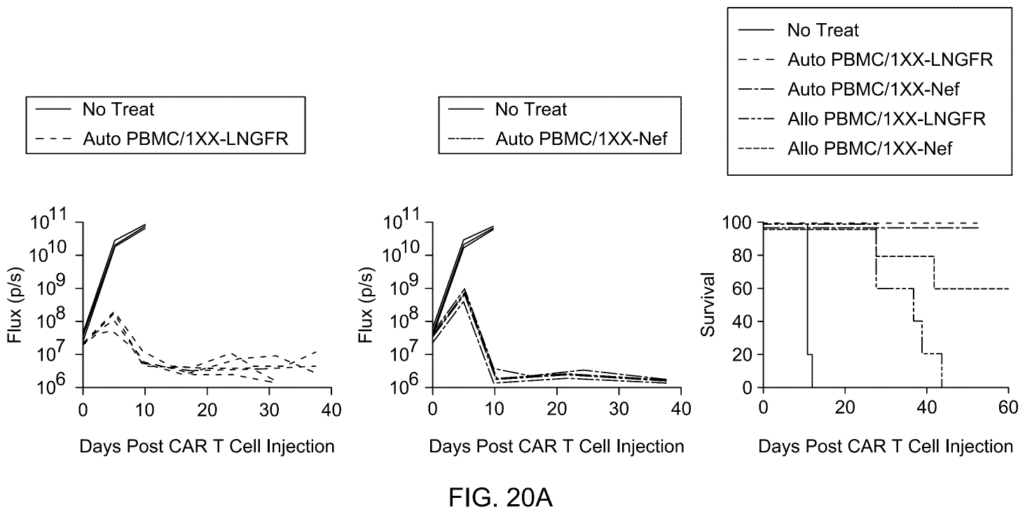

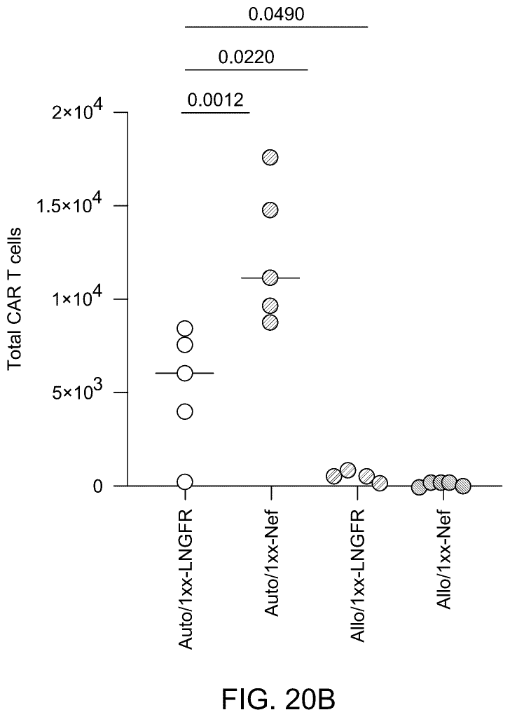

1XX CAR T cells demonstrate calibrated signaling, enhanced memory potential, and durable persistence compared to traditional second-generation 1928z, with encouraging safety and efficacy in clinical trials. Since Nef protected 1XX cells from AICD, it was hypothesized that the combination of 1XX and Nef would increase the potency of 1XX CAR T cells in vivo. AAV vectors encoding either 1928z or 191XX together with either control LNGFR or wild-type Nef (1928z-LNGFR, 1928z-Nef, 1XX-LNGFR, and 1XX-Nef) were generated. CAR expression was identical with site-specific insertion (FIG. 19A), and Nef consistently downregulated HLA-I as expected. CAR T cell accumulation was measured in the described Nalm6 model in the absence of immune rejection. 1928z-Nef CAR T cells showed significantly greater accumulation at the peak of effector activity (day 8; 1.9E5±0.15E5 for 1928z-Nef vs 1.0E5±0.08E5 for 1928z-LNGFR, p<0.001), although there was significant contraction of both subsets by day 16 (0.4E5±0.09E5 for 1928z-Nef, 6E3±1E3 for 1928z-LNGFR, p<0.001) (FIG. 19B). However, the combined 1XX-NEF construct had much greater expansion as compared to Nef or 1XX modifications alone, both at day 8 peak (5.4E5±0.26E5 for 1XX-Nef vs 1.5E5±0.16E5 for 1XX-LNGFR, p<0.001) and during the day 16 contraction phase (2.1E5±0.9E5 for 1XX-Nef vs 2.1E5±0.06E5 for 1XX-LNGFR, p=0.03) (FIG. 19B). Both 1XX-LNGFR and 1XX-Nef cleared tumor even at low doses of CAR T cells (FIG. 19C), outperforming 1928z-LNGFR and 1928z-Nef. 1XX-LNGFR and 1XX-Nef similarly achieved complete tumor eradication in the presence of autologous PBMC (FIG. 20A) and robust tumor control in the GBM flank model.

### 1XX-Nef CAR T Cells are Potent Effectors Under Allogeneic Conditions

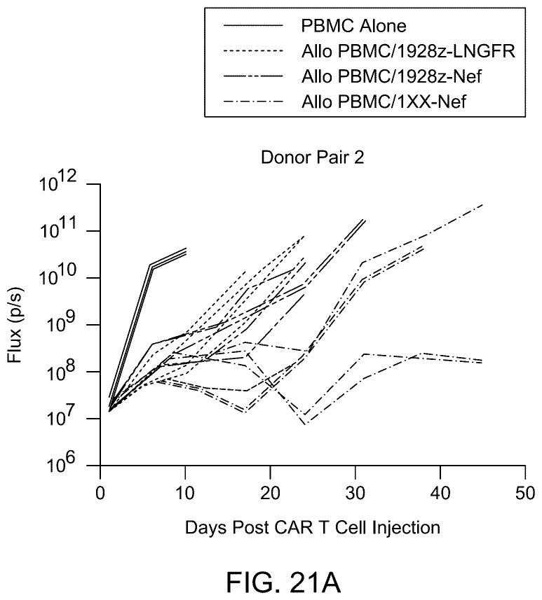

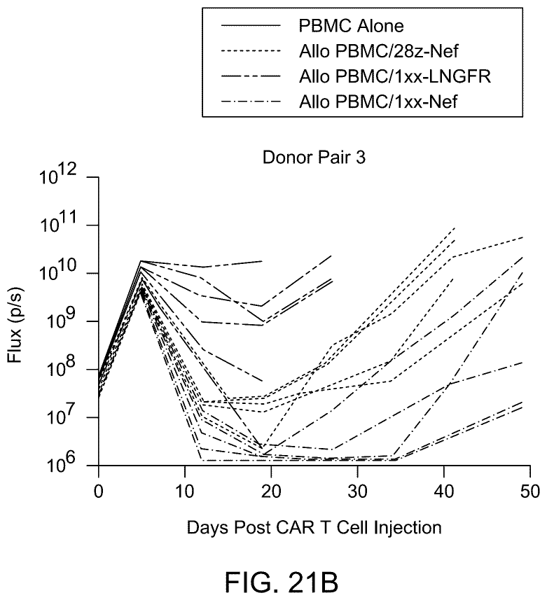

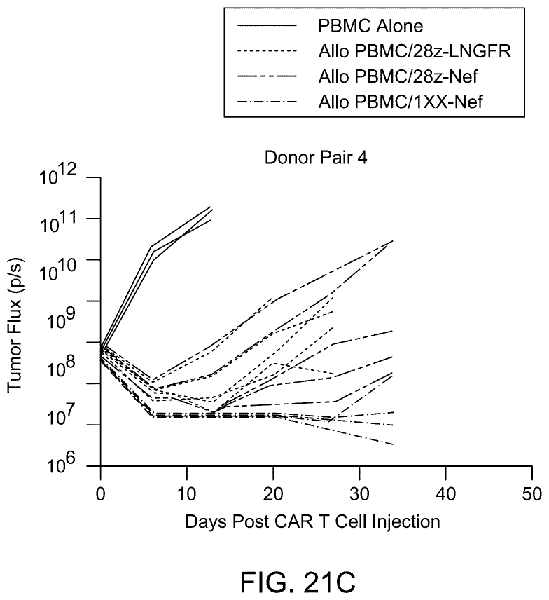

Relying upon the above-described allogeneic rejection model, it was hypothesized that 1XX-Nef would survive longer in an allogeneic setting than 1XX CAR T cells alone. Compared to 1XX CAR T cells, 1XX-Nef CAR T cells had similar early expansion kinetics at day 5, but demonstrated nearly 10-fold higher frequencies at day 10 (4599.4+/−2589.9 for 1XX-Nef vs 502.4+/−340.7 1XX-LNGFR CAR T cells, p=0.03 by Mann-Whitney), followed by contraction by day 16 (FIG. 19D). Both 1XX and 1XX-Nef cells were persistent in the setting of autologous as compared to allogeneic PBMC at day 18 (FIGS. 20A and 20B), suggesting that despite the log-fold increased survival at effector peak, 1XX-Nef-CARs were ultimately rejected. In the setting of allogeneic PBMCs, 1XX-Nef CARs T cells provided the most durable tumor control (FIG. 19E) and survival (FIG. 19F). We compared tumor control by 1XX and Nef combinations in 3 additional experiments with different mismatched donor pairs; tumor control varied by donor pair (FIGS. 21A-21C). Pooled survival results of the combined four experiments demonstrate that 1XX-Nef promoted superior survival (mOS 56 days, p<0.0001), outperforming 1XX-LNGFR (34 days), 1928z-Nef (44 days), or 1928z-LNGFR (28 days) (FIG. 19F).

### Discussion

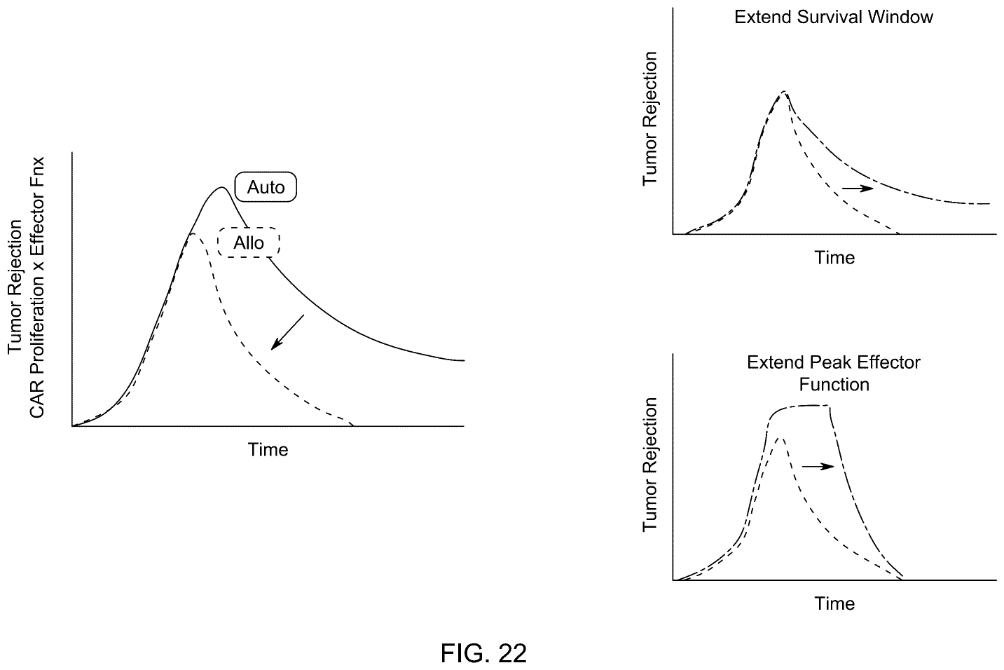

The present example sets out to explore and emulate immune evasions strategies adopted by lymphotropic viruses to protect their host cells and themselves. Virus-infected cells are threatened by extrinsic immune rejection from T and NK cells, as well as intrinsic mechanisms leading the death of their host. It was found that HIV Nef promotes the immune evasion and survival of allogeneic CAR T cells, both by reducing their allorecognition and limiting their risk of undergoing apoptotic AICD. Reduction of HLA-I levels to less than 40% of baseline attenuates CD8 T cell rejection, but levels below 20% of baseline lead to rapid clearance by NK cells. Stable HLA-I expression in the optimal range of 20-40% can be achieved by regulated expression of viral evasins including Nef. In addition, Nef uniquely opposes CAR T cell AICD. Here, it was showed that this anti-apoptotic effect is dependent on the Nef SH3 domain and the kinase Pak2. Combined with calibrated CAR activation strength, Nef expression affords markedly improved anti-tumor activity by allogeneic CAR T cells. The presently disclosed subject matter confirmed the benefit of B2M knockout as a method to evade CD8 T cells as well as the risk of rendering cells vulnerable to NK cell-mediated rejection. In the described NSG15 murine model, which supports NK cell engraftment, B2MKO allogeneic CAR T cells do not provide improved tumor control compared to unmodified allogeneic CAR T cells due to rapid clearance by NK cells. In contrast to full ablation, controlled Nef expression down-regulates HLA-I expression to a range where CD8-mediated rejection is abated without eliciting strong NK cell sensitivity. Nef preferentially down-regulates HLA-A expression relative to HLA-C and HLA-E, which it was observed here in CAR T cells. In the autologous context, HLA-C alleles can provide inhibitory signals to Killer Immunoglobulin Receptors (KIR); this benefit may not apply to the allogeneic context if donors are not matched for HLA-C allele and/or KIR ligand status. On the other hand, maintenance of invariant HLA-E alleles that provide inhibitory signals to NK cells via NKG2A60 provides further protection from NK cell killing regardless of donor HLA background. In addition to its effect on HLA-I expression, Nef promoted intrinsic T cell survival via Pak2, attenuating AICD and augmenting CAR T cell expansion upon repeated antigen stimulation. The exact pathway by which Pak2 modulates AICD, and the detailed description of its respective role in TCR and CAR signaling, warrant future studies. The effect of Nef on T cell survival herein reported may not be identical to its function in HIV-infected T cells, as the context of HIV infection differs with regards to TCR vs CAR signaling, the presence of additional HIV virulence factors such as Tat and VpR, and restriction to CD4+ T cells. Thus, it is demonstrated that Nef CD4 downregulation, which promotes viral replication by preventing reinfection, does not affect CAR T cell activity. Nef has previously been shown to protect against apoptosis in the context of HIV replication and serum deprivation. In the context of T cell activation and CAR signaling, Nef expression mitigated activation induced cell death. Immune evasion extends the window during which allogeneic cells are active, even though they are eventually eliminated. Here, in vivo modeling showed that it is critical to have highly effective CAR T cells that can achieve maximal tumor eradication within their survival window. Under these conditions, which made use of relatively low allogeneic CAR T cell dosing (FIG. 22), it was found that 1XX-Nef CARs achieve 10-fold higher frequencies in the allogeneic setting during the peak effector period owing to Nef expression. Here, the best tumor rejection was achieved using a 1XX CAR expressed from the TRAC locus and took advantage of this targeted gene delivery to achieve regulated Nef expression within an effective range. Ongoing study of autologous TRAC-inserted 1XX CAR T cells (NCT05757700) for B-cell malignancies demonstrated the feasibility of such site-specific CAR manufacturing. An attractive feature of allogeneic Nef-CARs is that expression of a single, small protein has a multitude of potentially beneficial effects that in aggregate increase the survival and therapeutic efficacy of allogeneic effector cells.

### REFERENCES

Abraham, L., and Fackler, O. T. (2012). HIV-1 Nef: a multifaceted modulator of T cell receptor signaling.
Amini, L., Silbert, S. K., Maude, S. L., Nastoupil, L. J., Ramos, C. A., Brentjens, R. J., Sauter, C. S., Shah, N. N., and Abou-El-Enein, M. (2022). Preparing for CAR T cell therapy: patient selection, bridging therapies and lymphodepletion. Nat Rev Clin Oncol 0123456789. 10.1038/s41571-022-00607-3.
Arora, V. K., Molina, R. P., Foster, J. L., Blakemore, J. L., Chernoff, J., Fredericksen, B. L., and Garcia, A. J. V. (2000). Lentivirus Nef Specifically Activates Pak2. J Virol 74, 11081-11087.
Barrett, A. J., Prockop, S., and Bollard, C. M. (2018). Virus-Specific T Cells: Broadening Applicability. Biol Blood Marrow Transplant 24, 13-18. 10.1016/j.bbmt.2017.10.004.
Benjamin, R., Jain, N., Maus, M. V., Boissel, N., Graham, C., Jozwik, A., Yallop, D., Konopleva, M., Frigault, M. J., Teshima, T., et al. (2022). UCART19, a first-in-class allogeneic anti-CD19 chimeric antigen receptor T-cell therapy for adults with relapsed or refractory B-cell acute lymphoblastic leukaemia (CALM): a phase 1, dose-escalation trial. Lancet Haematol 9, e833-e843. 10.1016/S2352-3026(22)00245-9.
Bern, M. D., Parikh, B. A., Yang, L., Beckman, D. L., Poursine-Laurent, J., and Yokoyama, W. M. (2018). Inducible down-regulation of MHC class I results in natural killer cell tolerance. Journal of Experimental Medicine, jem.20181076. 10.1084/jem.20181076.
Braud, V. M., Allan, D. S. J., O'Callaghan, C. A., Söderström, K., D'Andrea, A., Ogg, G. S., Lazetic, S., Young, N. T., Bell, J. I., Phillips, J. H., et al. (1998). HLA-E binds to natural killer cell receptors CD94/NKG2A, B and C. Nature 391, 795-799. 10.1038/35869.
Brenner, D., Krammer, P. H., and Arnold, R. (2008). Concepts of activated T cell death. Crit Rev Oncol Hematol 66, 52-64. 10.1016/j.critrevonc.2008.01.002.
Brentjens, R., Latouche, J.-B., Santos, E., Marti, F., Gong, M., Lydanne, C., King, P. D., Larson, S., Weiss, M., Riviere, I., et al. (2003). Eradication of systemic B-cell tumors by genetically targeted human T lymphocytes co-stimulated by CD80 and interleukin-15. Nat Med 9, 279. 10.1038/nm.
Brentjens, R. J., Santos, E., Nikhamin, Y., Yeh, R., Matsushita, M., La Perle, K., Quintias-Cardama, A., Larson, S. M., and Sadelain, M. (2007). Genetically targeted T cells eradicate systemic acute lymphoblastic leukemia xenografts. Clinical Cancer Research 13, 5426-5435. 10.1158/1078-0432.CCR-07-0674.
Buffalo, C. Z., Iwamoto, Y., Hurley, J. H., and Ren, X. (2019). How HIV Nef Proteins Hijack Membrane Traffic To Promote Infection. J Virol, 1-18.
Campbell, H. K., Salvi, A. M., O'Brien, T., Superfine, R., and DeMali, K. A. (2019). PAK2 links cell survival to mechanotransduction and metabolism. Journal of Cell Biology 218, 1958-1971. 10.1083/JCB.201807152.
Chong, C., Tan, L., Lim, L., and Manser, E. (2001). The mechanism of PAK activation. Autophosphorylation events in both regulatory and kinase domains control activity. Journal of Biological Chemistry 276, 17347-17353. 10.1074/jbc.M009316200.
Cohen, G. B., Gandhi, R. T., Davis, D. M., Mandelboim, O., Chen, B. K., Strominger, J. L., and Baltimore, D. (1999). The Selective Downregulation of Class I Major Histocompatibility Complex Proteins by HIV-1 Protects HIV-Infected Cells from NK Cells. 10, 661-671.
Collette, Y., Dutartre, H., Benziane, A., Ramos-Morales, F., Benarous, R., Harris, M., and Olive, D. (1996). Physical and functional interaction of Nef with Lck: HIV-1 Nef-induced T-cell signaling defects. Journal of Biological Chemistry 271, 6333-6341. 10.1074/jbc.271.11.6333.
Creelan, B. C., and Antonia, S. J. (2019). The NKG2A immune checkpoint a new direction in cancer immunotherapy. Preprint at Nature Publishing Group, 10.1038/s41571-019-0182-8 10.1038/s41571-019-0182-8.
Depil, S., Duchateau, P., Grupp, S. A., Mufti, G., and Poirot, L. (2020). ‘Off-the-shelf’ allogeneic CAR T cells: development and challenges. Nat Rev Drug Discov. 10.1038/s41573-019-0051-2.
Deuse, T., Hu, X., Gravina, A., Wang, D., Tediashvili, G., De, C., Thayer, W. O., Wahl, A., Garcia, J. V., Reichenspurner, H., et al. Hypoimmunogenic derivatives of induced pluripotent stem cells evade immune rejection in fully immunocompetent allogeneic recipients. Nat Biotechnol. 10.1038/s41587-019-0016-3.
Eron, S. J., Raghupathi, K., and Hardy, J. A. (2017). Dual Site Phosphorylation of Caspase-7 by PAK2 Blocks Apoptotic Activity by Two Distinct Mechanisms. Structure 25, 27-39. 10.1016/j.str.2016.11.001.
Eyquem, J., Mansilla-Soto, J., Giavridis, T., Van Der Stegen, S. J. C., Hamieh, M., Cunanan, K. M., Odak, A., Gönen, M., and Sadelain, M. (2017). Targeting a CAR to the TRAC locus with CRISPR/Cas9 enhances tumour rejection. Nature 543, 113-117. 10.1038/nature21405.
Faust, T. B., Binning, J. M., Gross, J. D., and Frankel, A. D. (2017). Making Sense of Multifunctional Proteins: Human Immunodeficiency Virus Type 1 Accessory and Regulatory Proteins and Connections to Transcription.
Feucht, J., Sun, J., Eyquem, J., Ho, Y. J., Zhao, Z., Leibold, J., Dobrin, A., Cabriolu, A., Hamieh, M., and Sadelain, M. (2019). Calibration of CAR activation potential directs alternative T cell fates and therapeutic potency. Nat Med 25, 82-88. 10.1038/s41591-018-0290-5.
Fraietta, J. A., Lacey, S. F., Orlando, E. J., Pruteanu-Malinici, I., Gohil, M., Lundh, S., Boesteanu, A. C., Wang, Y., O'connor, R. S., Hwang, W. T., et al. (2018). Determinants of response and resistance to CD19 chimeric antigen receptor (CAR) T cell therapy of chronic lymphocytic leukemia. Nat Med 24, 563-571. 10.1038/s41591-018-0010-1.
Gargett, T., Yu, W., Dotti, G., Yvon, E. S., Christo, S. N., Hayball, J. D., Lewis, I. D., Brenner, M. K., and Brown, M. P. (2016). GD2-specific CAR T Cells Undergo Potent Activation and Deletion Following Antigen Encounter but can be Protected from Activation-induced Cell Death by PD-1 Blockade. Molecular Therapy 24, 1135-1149. 10.1038/mt.2016.63.
Geyer, M., Fackler, O. T., and Peterlin, B. M. (2001). Structure-function relationships in HIV-1 Nef. EMBO Rep 2, 580-585. 10.1093/embo-reports/kve141.
Gornalusse, G. G., Hirata, R. K., Funk, S. E., Riolobos, L., Lopes, V. S., Manske, G., Prunkard, D., Colunga, A. G., Hanafi, L.-A., Clegg, D. O., et al. (2017). HLA-E-expressing pluripotent stem cells escape allogeneic responses and lysis by NK cells. Nat Biotechnol 35, 765-772. 10.1038/nbt.3860.
Gougeon, M. L. (2003). Apoptosis as an HIV strategy to escape immune attack. Nat Rev Immunol 3, 392-404. 10.1038/nri1087.
Greenway, A., Azad, A., Mills, J., and McPhee, D. (1996). Human immunodeficiency virus type 1 Nef binds directly to Lck and mitogen-activated protein kinase, inhibiting kinase activity. J Virol 70, 6701-6708. 10.1128/jvi.70.10.6701-6708.1996.
Guil-Luna, S., Sedlik, C., and Piaggio, E. (2020). Humanized Mouse Models to Evaluate Cancer Immunotherapeutics. Annu Rev Cancer Biol 5, 119-136. 10.1146/annurev-cancerbio-050520-100526.
Haller, C., Rauch, S., and Fackler, O. T. (2007). HIV-1 Nef employs two distinct mechanisms to modulate Lck subcellular localization and TCR induced actin remodeling. PLoS One 2. 10.1371/journal.pone.0001212.
Hansen, S. G., Powers, C. J., Richards, R., Ventura, A. B., Ford, J. C., Siess, D., Axthelm, M. K., Nelson, J. A., Jarvis, M. A., Picker, L. J., et al. (2010). Evasion of CD8+ T cells is critical for superinfection by cytomegalovirus. Science 328, 102-106. 10.1126/science.1185350.
Hansen, T. H., and Bouvier, M. (2009). MHC class i antigen presentation: Learning from viral evasion strategies. Nat Rev Immunol 9, 503-513. 10.1038/nri2575.
Huang, J., Huang, A., Poplawski, A., DiPino, F., Traugh, J. A., and Ling, J. (2020). PAK2 activated by Cdc42 and caspase 3 mediates different cellular responses to oxidative stress-induced apoptosis. Biochim Biophys Acta Mol Cell Res 1867, 118645. 10.1016/j.bbamcr.2020.118645.
Jacobson, C. A., Jayawardena, T., Yu, L., Hallman, J., Schmittling, R., Cuneo, E., Belle, I., Sampey, G., Cabral, D., Derrick, A., et al. (2022). Effective Cell Dose and Functional Attributes of Azercabtagene Zapreleucel (azer-cel; PBCAR0191) Associate with Allogeneic CAR T-Cell Safety and Efficacy in Patients with Relapsed/Refractory B-Cell Lymphoma. Blood 140, 4588-4589. 10.1182/BLOOD-2022-165311.
June, C. H., and Sadelain, M. (2018). Chimeric Antigen Receptor Therapy. New England Journal of Medicine 379, 64-73. 10.1056/NEJMra1706169.
Jung, J. H., and Traugh, J. A. (2005). Regulation of the interaction of Pak2 with Cdc42 via autophosphorylation of serine 141. Journal of Biological Chemistry 280, 40025-40031. 10.1074/jbc.M509075200.
Künkele, A., Johnson, A. J., Rolczynski, L. S., Chang, C. A., Hoglund, V., Kelly-Spratt, K. S., and Jensen, M. C. (2015). Functional Tuning of CARs Reveals Signaling Threshold above Which CD8+ CTL Antitumor Potency Is Attenuated due to Cell Fas-FasL-Dependent AICD. Cancer Immunol Res 3, 368-379. 10.1158/2326-6066.CIR-14-0200.
Levine, B. L., Miskin, J., Wonnacott, K., and Keir, C. (2017). Global Manufacturing of CAR T Cell Therapy. Mol Ther Methods Clin Dev 4, 92-101. 10.1016/j.omtm.2016.12.006.
Liu, H., Liu, K., and Dong, Z. (2021). The Role of p21-Activated Kinases in Cancer and Beyond: Where Are We Heading? Front Cell Dev Biol 9, 1-12. 10.3389/fcell.2021.641381.
Liu, L. X., Heveker, N., Fackler, O. T., Arold, S., Le Gall, S., Janvier, K., Peterlin, B. M., Dumas, C., Schwartz, O., Benichou, S., et al. (2000). Mutation of a Conserved Residue (D123) Required for Oligomerization of Human Immunodeficiency Virus Type 1 Nef Protein Abolishes Interaction with Human Thioesterase and Results in Impairment of Nef Biological Functions. J Virol 74, 5310-5319. 10.1128/jvi.74.11.5310-5319.2000.
Maher, J., Brentjens, R. J., Gunset, G., Riviere, I., and Sadelain, M. (2002). Human T-lymphocyte cytotoxicity and proliferation directed by a single chimeric TCRzeta/CD28 receptor. Nat Biotechnol 20, 70-75. 10.1038/nbt0102-70.
Malmberg, K. J., Carlsten, M., Björklund, A., Sohlberg, E., Bryceson, Y. T., and Ljunggren, H. G. (2017). Natural killer cell-mediated immunosurveillance of human cancer. Semin Immunol 31, 20-29. 10.1016/j.smim.2017.08.002.
Mancini, M., and Vidal, S. M. (2020). Mechanisms of Natural Killer Cell Evasion Through Viral Adaptation. Annu Rev Immunol 38, 511-539. 10.1146/annurev-immunol-082619-124440.
Mansilla-Soto, J., Eyquem, J., Haubner, S., Hamieh, M., Feucht, J., Paillon, N., Zucchetti, A. E., Li, Z., Sjöstrand, M., Lindenbergh, P. L., et al. (2022). HLA-independent T cell receptors for targeting tumors with low antigen density. Nat Med 28, 345-352. 10.1038/s41591-021-01621-1.
Marino, J., Paster, J., and Benichou, G. (2016). Allorecognition by T Lymphocytes and Allograft Rejection. Front Immunol 7, 1-9. 10.3389/fimmu.2016.00582.
Markle, T. J., Mwimanzi, P., and Brockman, M. A. (2013). HIV-1 Nef and T-cell activation: A history of contradictions. Future Virol 8, 391-404. 10.2217/fvl.13.20.
O'Reilly, R. J., Prockop, S., Hasan, A. N., Koehne, G., and Doubrovina, E. (2016). Virus-specific T-cell banks for “off the shelf” adoptive therapy of refractory infections. Bone Marrow Transplant 51, 1163-1172. 10.1038/bmt.2016.17.
Paim, A. C., Badley, A. D., and Cummins, N. W. (2020). Mechanisms of Human Immunodeficiency Virus-Associated Lymphocyte Regulated Cell Death. AIDS Res Hum Retroviruses 36, 101-115. 10.1089/aid.2019.0213.
Park, J. H., Palomba, M. L., Devlin, S. M., Valtis, Y. K., Sikder, D. S., Senechal, B., Wang, X., Cathcart, E., Liotta, K., Yu, A., et al. (2023). A Phase I Study of CD19-Targeted 19(T2)28z1xx CAR T Cells in Adult Patients with Relapsed or Refractory Diffuse Large B-Cell Lymphoma. Blood 142, 892. 10.1182/BLOOD-2023-187623.
Perica, K., Flynn, J., Curran, K. J., Wang, X., Halton, E., Diamonte, C., Pineda, J., Bernal, Y., Gonen, M., Riviere, I., et al. (2021). Impact of bridging chemotherapy on clinical outcome of CD19 CAR T therapy in adult ALL. Leukemia Published. 10.1200/jco.2019.37.15_suppl.2520.
Phee, H., Au-yeung, B. B., Pryshchep, O., Hagan, K. L. O., Fairbairn, S. G., Radu, M., Kosoff, R., Mollenauer, M., Cheng, D., Chernoff, J., et al. (2014). Pak2 is required for actin cytoskeleton remodeling, TCR signaling, and normal thymocyte development and maturation. 1-23. 10.7554/eLife.02270.
Philip, L. P. B., Schiffer-Mannioui, C., Le Clerre, D., Chion-Sotinel, I., Derniame, S., Potrel, P., Bas, C., Lemaire, L., Galetto, R., Lebuhotel, C., et al. (2015). Multiplex genome-edited T-cell manufacturing platform for “off-the-shelf” adoptive T-cell immunotherapies. Cancer Res 75, 3853-3864. 10.1158/0008-5472.CAN-14-3321.
Ren, J., Liu, X., Fang, C., Jiang, S., June, C. H., and Zhao, Y. (2017). Multiplex Genome Editing to Generate Universal CAR T Cells Resistant to PD1 Inhibition. Clin Cancer Res 23, 2255-2266. 10.1158/1078-0432.CCR-16-1300.
Sanber, K., Savani, B., and Jain, T. (2021). Graft-versus-host disease risk after chimeric antigen receptor T-cell therapy: the diametric opposition of T cells. Br J Haematol 195, 660-668. 10.1111/bjh.17544.
Singh, N., Perazzelli, J., Grupp, S. A., and Barrett, D. M. (2016). Early memory phenotypes drive T cell proliferation in patients with pediatric malignancies. Sci Transl Med 8, 1-10. 10.1126/scitranslmed.aad5222.
Themeli, M., Riviiere, I., and Sadelain, M. (2015). New cell sources for T cell engineering and adoptive immunotherapy. Cell Stem Cell 16, 357-366. 10.1016/j.stem.2015.03.011.
Torikai, H., Mi, T., Gragert, L., Maiers, M., Najjar, A., Ang, S., Maiti, S., Dai, J., Switzer, K. C., Huls, H., et al. (2016). Genetic editing of HLA expression in hematopoietic stem cells to broaden their human application. Sci Rep 6, 21757. 10.1038/srep21757.
Torikai, H., Reik, A., Soldner, F., Warren, E. H., Yuen, C., Zhou, Y., Crossland, D. L., Huls, H., Littman, N., Zhang, Z., et al. (2013). Toward eliminating HLA class I expression to generate universal cells from allogeneic donors. Blood 122, 1341-1349. 10.1182/blood-2013-03-478255.
Tschumi, B. O., Dumauthioz, N., Marti, B., Zhang, L., Schneider, P., Mach, J.-P., Romero, P., and Donda, A. (2018). CART cells are prone to Fas- and DR5-mediated cell death. J Immunother Cancer 6, 71. 10.1186/s40425-018-0385-z.
Wang, B., Iriguchi, S., Waseda, M., Ueda, N., Ueda, T., Xu, H., Minagawa, A., Ishikawa, A., Yano, H., Ishi, T., et al. (2021). Generation of hypoimmunogenic T cells from genetically engineered allogeneic human induced pluripotent stem cells. Nat Biomed Eng 5, 429-440. 10.1038/s41551-021-00730-z.
Wang, X., and Rivière, I. (2016). Clinical manufacturing of CAR T cells: foundation of a promising therapy. Mol Ther Oncolytics 3, 1-7. 10.1038/mto.2016.15.
Wiebking, V., Lee, C. M., Mostrel, N., Lahiri, P., Bak, R., Bao, G., Roncarolo, M. G., Bertaina, A., and Porteus, M. H. (2021). Genome editing of donor-derived T cells to generate allogeneic chimeric antigen receptor-modified T cells: optimizing aβ T-cell-depleted haploidentical hematopoietic stem cell transplantation. Haematologica 106, 847-858. 10.3324/haematol.2019.233882.
Wolf, D., Witte, V., Laffert, B., Blume, K., Stromer, E., Trapp, S., D'aloja, P., Schurmann, A., and Baur, A. S. (2001). HIV-1 Nef associated PAK and PI3-kinases stimulate Akt-independent bad-phosphorylation to induce anti-apoptotic signals. Nat Med 7, 1217-1224. 10.1038/nm1101-1217.

### Embodiments of the Presently Disclosed Subject Matter

From the foregoing description, it will be apparent that variations and modifications may be made to the presently disclosed subject matter to adopt it to various usages and conditions. Such embodiments are also within the scope of the following claims.

The recitation of a listing of elements in any definition of a variable herein includes definitions of that variable as any single element or combination (or sub-combination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A cell comprising an immunoevasin and an antigen recognizing receptor that targets an antigen, wherein the immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91.

2. The cell of claim 1, wherein the immunoevasin comprises the amino acid sequence set forth in SEQ ID NO: 1, SEQ ID NO: 2, SEQ ID NO: 3, SEQ ID NO: 4, SEQ ID NO: 5, or SEQ ID NO: 91.

3. The cell of claim 1, wherein the immunoevasin comprises the amino acid sequence set forth in SEQ ID NO: 1.

4. The cell of claim 1, further comprising a second immunoevasin.

5. The cell of claim 1, wherein the immunoevasin comprises the amino acid sequence set forth in SEQ ID NO: 5.

6. The cell of claim 1 further comprising a gene disruption of a NLRC5 locus, a RFX5 locus, a TCR locus, or a combination thereof.

7. A cell comprising an antigen recognizing receptor that targets an antigen and a gene disruption of a NLRC5 locus, a RFX5 locus, or a combination thereof.

8. The cell of claim 1, wherein the antigen-recognizing receptor is a T cell receptor (TCR), a chimeric antigen receptor (CAR), or a TCR like fusion molecule.

9. The cell of claim 8, wherein the antigen-recognizing receptor is a chimeric antigen receptor (CAR) comprising an extracellular antigen-binding domain that binds to the antigen, and an intracellular signaling domain comprising a native CD3ζ polypeptide or a modified CD3ζ polypeptide.

10. The cell of claim 9, wherein the modified CD3ζ polypeptide comprises a native ITAM1, an ITAM2 variant consisting of two loss-of-function mutations, and an ITAM3 variant consisting of two loss-of-function mutations.

11. The cell of claim 8, wherein the antigen-recognizing receptor is a TCR-like fusion molecule comprising i) a first antigen-binding chain comprising an antigen-binding fragment of a heavy chain variable region (VH) of an antibody; and ii) a second antigen-binding chain comprising an antigen-binding fragment of a light chain variable region (VL) of the antibody; wherein the first and second antigen-binding chains a) each comprise the TRAC polypeptide or the TRBC polypeptide, and b) bind to the antigen, wherein the TCR-like fusion molecule binds to the antigen in an HLA-independent manner.

12. The cell of claim 1, wherein the cell of the lymphoid lineage is selected from the group consisting of a T cell, a B cell, a Natural Killer (NK) cell, and a dendritic cell.

13. The cell of claim 1, wherein the cell is a T cell.

14. The cell of claim 13, wherein the T cell is CD62L⁺, CD45RA⁺, or CD45RA⁺ and CD62L⁺.

15. The cell of claim 1, wherein the immunoevasin is encoded by a first polynucleotide integrated at a locus within the genome of the T cell, and/or the antigen recognizing receptor is encoded by a second polynucleotide integrated at a locus within the genome of the T cell.

16. The cell of claim 15, wherein the first polynucleotide comprises an EF1 promoter comprising the nucleotide sequence set forth in SEQ ID NO: 90.

17. The cell of claim 1, wherein the antigen is a tumor antigen selected from the group consisting of CD19, CD70, IL1RAP, ABCG2, AChR, ACKR6, ADAMTS13, ADGRE2, ADGRE2 (EMR2), ADORA3, ADRA1D, AGER, ALS2, an antigen of a cytomegalovirus (CMV) infected cell (e.g. a cell surface antigen), ANO9, AQP2, ASIC3, ASPRV1, ATP6V0A4, B3GNT4, B7-H3, BCMA, BEST4, C3orf35, CADM3, CAIX, CAPN3, CCDC155, CCR1, CD10, CD117, CD123, CD133, CD135 (FLT3), CD138, CD20, CD22, CD244 (2B4), CD25, CD26, CD30, CD300LF, CD312, CD32, CD321, CD33, CD34, CD36, CD38, CD41, CD44, CD44V6, CD47, CD49f, CD56, CD7, CD71, CD74, CD8, CD82, CD96, CD98, CD99, CDH13, CDHR1, CEA, CEACAM6, CHST3, CLDN18.2, CLEC12A, CLEC1A, CLL1, CNIH2, COL15A1, COLEC12, CPM, CR1, CX3CR1, CXCR4, CYP4F11, DAGLB, DARC, DFNB31, DGKI, EGF1R, EGFR-VIII, EGP-2, EGP-40, ELOVL6, EMB, EMC10, EMR2, ENG, EpCAM, EphA2, EPHA4, ERBB, ERBB2, Erb-B3, Erb-B4, E-selectin, EXOC3L4, EXTL3, FAM186B, FBP, FCGR1A, FKBP1B, FLRT1, folate receptor-α, FOLR2, FRMD5, GABRB2, GAS2, GD2, GD3, GDPD3, GNA14, GNAZ, GPR153, GPR56, GPRC5d, GYPA, HEPHL1, HER-2, hERT, HILPDA, HLA-DR, HOOK1, hTERT, HTR2A, ICAM1, IGFBP3, IL10RB, IL20RB, IL23R, ILDR1, Interleukin-13 receptor subunit alpha-2 (IL-13Rα2), ITFG3, ITGA4, ITGA5, ITGA8, ITGAX, ITGB5, ITGB8, JAM3, KCND1, KCNJ5, KCNK13, KCNN4, KCNV2, KDR, KIF19, KIF26B, κ-light chain, L1CAM, LAX1, LEPR, Lewis Y (CD174), Lewis Y (LeY), LILRA2, LILRA6, LILRB2, LILRB3, LILRB4, LOXL4, LPAR2, LRRC37A3, LRRC8E, LRRN2, LRRTM2, LTB4R, MAGE-A1, MAGEA3, MANSC1, MART1, GP100, MBOAT1, MBOAT7, melanoma antigen family A, Mesothelin (MSLN), MFAP3L, MMP25, MRP1, MT-ND1, Mucin 1 (MUC1), Mucin 16 (MUC16), MYADM, MYADML2, NGFR, NKCS1, NKG2D ligands, NLGN3, NPAS2, NY-ESO-1, oncofetal antigen (h5T4), OTOA, P2RY13, p53, PDE3A, PEAR1, PIEZO1, PLXNA4, PLXNC1, PNPLA3, PPFIA4, PPP2R5B, PRAME, PRAME, prostate stem cell antigen (PSCA), prostate-specific membrane antigen (PSMA), Proteinase3 (PR1), PSD2, PTPRJ, RDH16, receptor tyrosine-protein kinase Erb-B2, RHBDL3, RNF173, RNF183, ROR1, RYR2, SCIN, SCN11A, SCN2A, SCNN1D, SEC31B, SEMA4A, SH3PXD2A, SIGLEC11, SIRPB1, SLC16A6, SLC19A1, SLC22A5, SLC25A36, SLC25A41, SLC30A1, SLC34A3, SLC43A3, SLC44A1, SLC44A3, SLC45A3, SLC6A16, SLC6A6, SLC8A3, SLC9A1, SLCO2B1, SPAG17, STC1, STON2, SUN3, Survivin, SUSD2, SYNC, TACSTD2, TAS1R3, TEX29, TFR2, TIM-3 (HAVCR2), TLR2, TMEFF2, TMEM145, TMEM27, TMEM40, TMEM59L, TMEM89, TMPRSS5, TNFRSF14, TNFRSF1B, TRIM55, TROP2, TSPEAR, TTYH3, tumor-associated glycoprotein 72 (TAG-72), Tyrosinase, vascular endothelial growth factor R2 (VEGF-R2), VLA-4, Wilms tumor protein (WT-1), WNT4, WT1, and ZDHHC11.

18. The cell of claim 1, wherein the immunoevasin reduces the expression level of the major histocompatibility complex I (MHCI) from between about 60% to about 90% compared to a cell non expressing the immunoevasin.

19. The cell of claim 1, wherein the cell is autologous or allogeneic.

20. A composition comprising the cell of claim 1.

21. A nucleic acid comprising a first polynucleotide encoding an immunoevasin and a second polynucleotide encoding an antigen recognizing receptor that targets an antigen, wherein the immunoevasin comprises or consists of an amino acid sequence that is at least about 80% identical to the amino acid sequence set forth in SEQ ID NO: 1, SEQ ID NO: 2, SEQ ID NO: 3, SEQ ID NO: 4, SEQ ID NO: 5, or SEQ ID NO: 91.

22. A vector comprising the nucleic acid of claim 21.

23. A lipid nanoparticle comprising the nucleic acid of claim 21.

24. A method for producing a modified cell, the method comprising introducing into a cell the nucleic acid of claim 21.

25. A method of reducing tumor burden, treating and/or preventing a neoplasm or a tumor, preventing and/or treating a pathogen infection, preventing and/or treating an autoimmune disease, and/or preventing and/or treating an infectious disease, the method comprising administering to the subject an effective amount of the cell of claim 1.

26. A kit comprising the cell of claim 1.


SEQUENCE LISTING (120 sequences)

SEQ ID NO: 1 - protein, 206 aa, synthetic construct
MGGKWSKRSVVGWPAVRERMRRAEPAADGVGAVSRDLEKHGAITSSNTAANNADCAWLEAQEEEEVGFPVRPQVPLRPMTYKAAVDLSHFLKEKGGLEGLIYSQKRQDILDLWVYHTQGYFPDWQNYTPGPGIRYPLTFGWCFKLVPVEPEKVEEANEGENNSLLHPMSLHGMDDPEKEVLVWKFDSRLAFHHMARELHPEYYKDC

SEQ ID NO: 2 - protein, 85 aa, synthetic construct
MGGKWLKSSMVRWPAVREKMKQAEPAAEGVGAISRDLGKHGAIPSSNTTTNNANCAWLEAQEEEEVGFPVKPQVPLRPMTYKATF

SEQ ID NO: 3 - protein, 252 aa, synthetic construct
SKKLSKHSRGLRERLLRARGDGYGKQRDASGGEYSQFQEESGREQNSPSCEGQQYQQGEYMNSPWRNPATERQKDLYRQQNMDDVDSDDDDLIGVPVTPRVPRREMTYKLAIDMSHFIKEKGGLQGMFYSRRRHRILDIYLEKEEGIIPDWQNYTHGPGVRYPMYFGWLWKLVSVELSQEAEEDEANCLVHPAQTSRHDDEHGETLVWQFDSMLAYNYKAFTLYPEEFGHKSGLPEKEWKAKLKARGIPYSE

SEQ ID NO: 4 - protein, 229 aa, synthetic construct
MGASGSKKLSKSQFQEESGREQNSPSCEGQQYQQGEYMNSPWRNPATERQKDLYRQQNMDDVDSDDDDLIGVPVTPRVPRREMTYKLAIDMSHFIKEKGGLQGMFYSRRRHRILDIYLEKEEGIIPDWQNYTHGPGVRYPMYFGWLWKLVSVELSQEAEEDEANCLVHPAQTSRHDDEHGETLVWQFDSMLAYNYKAFTLYPEEFGHKSGLPEKEWKAKLKARGIPYSE

SEQ ID NO: 5 - protein, 60 aa, synthetic construct
MVHVLERALLEQQSSACGLPGSSTETRPSHPCPEDPDVSRLRLLLVVLCVLFGLLCLLLI

SEQ ID NO: 6 - protein, 15 aa, synthetic construct
GGGGSGGGGSGGGGS

SEQ ID NO: 7 - protein, 19 aa, synthetic construct
GGGGSGGGGSGGGSGGGGS

SEQ ID NO: 8 - protein, 24 aa, synthetic construct
GGGGSGGGGSGGGGSGGGSGGGGS

SEQ ID NO: 9 - protein, 29 aa, synthetic construct
GGGGSGGGGSGGGGSGGGGSGGGSGGGGS

SEQ ID NO: 10 - protein, 5 aa, synthetic construct
GGGGS

SEQ ID NO: 11 - protein, 10 aa, synthetic construct
GGGGSGGGGS

SEQ ID NO: 12 - protein, 20 aa, synthetic construct
MYRMQLLSCIALSLALVTNS

SEQ ID NO: 13 - protein, 20 aa, synthetic construct
MYSMQLASCVTLTLVLLVNS

SEQ ID NO: 14 - protein, 20 aa, synthetic construct
METPAQLLFLLLLWLPDTTG

SEQ ID NO: 15 - protein, 20 aa, synthetic construct
METDTLLLWVLLLWVPGSTG

SEQ ID NO: 16 - protein, 21 aa, synthetic construct
MALPVTALLLPLALLLHAARP

SEQ ID NO: 17 - protein, 18 aa, synthetic construct
MALPVTALLLPLALLLHA

SEQ ID NO: 18 - protein, 16 aa, synthetic construct
MKWVTFISLLFSSAYS

SEQ ID NO: 19 - protein, 30 aa, synthetic construct
MDSKGSSQKGSRLLLLLVVSNLLLCQGVVS

SEQ ID NO: 20 - protein, 6 aa, synthetic construct
GYAFSS

SEQ ID NO: 21 - protein, 6 aa, synthetic construct
YPGDGD

SEQ ID NO: 22 - protein, 9 aa, synthetic construct
KTISSVVDF

SEQ ID NO: 23 - protein, 11 aa, synthetic construct
KASQNVGTNVA

SEQ ID NO: 24 - protein, 6 aa, synthetic construct
SATYRN

SEQ ID NO: 25 - protein, 9 aa, synthetic construct
QQYNRYPYT

SEQ ID NO: 26 - protein, 122 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSS

SEQ ID NO: 27 - protein, 120 aa, synthetic construct
EVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTV

SEQ ID NO: 28 - protein, 108 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 29 - protein, 106 aa, synthetic construct
DIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 30 - protein, 263 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTVSSGGGGSGGGGSGGGGSDIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEIKR

SEQ ID NO: 31 - protein, 220 aa, synthetic construct
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 32 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 33 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 34 - DNA, 117 bp, synthetic construct
  source 1..117: mol_type = other DNA
attgaagttatgtatcctcctccttacctagacaatgagaagagcaatggaaccattatccatgtgaaagggaaacacctttgtccaagtcccctatttcccggaccttctaagccc

SEQ ID NO: 35 - protein, 164 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 36 - protein, 339 aa, synthetic construct
AGAGTGAAGTTCAGCAGGAGCGCAGACGCCCCCGCGTACCAGCAGGGCCAGAACCAGCTCTATAACGAGCTCAATCTAGGACGAAGAGAGGAGTACGATGTTTTGGACAAGAGACGTGGCCGGGACCCTGAGATGGGGGGAAAGCCGAGAAGGAAGAACCCTCAGGAAGGCCTGTACAATGAACTGCAGAAAGATAAGATGGCGGAGGCCTACAGTGAGATTGGGATGAAAGGCGAGCGCCGGAGGGGCAAGGGGCACGATGGCCTTTACCAGGGTCTCAGTACAGCCACCAAGGACACCTACGACGCCCTTCACATGCAGGCCCTGCCCCCTCGCTAA

SEQ ID NO: 37 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 38 - protein, 112 aa, synthetic construct
RVKFSRSADAPAYQQGQNQLYNELNLGRREEYDVLDKRRGRDPEMGGKPRRKNPQEGLFNELQKDKMAEAFSEIGMKGERRRGKGHDGLFQGLSTATKDTFDALHMQALPPR

SEQ ID NO: 39 - DNA, 336 bp, synthetic construct
  source 1..336: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccaggggctcagtacagccaccaaggacaccttcgacgcccttcacatgcaggccctgccccctcgc

SEQ ID NO: 40 - protein, 22 aa, synthetic construct
QNQLYNELNLGRREEYDVLDKR

SEQ ID NO: 41 - DNA, 66 bp, synthetic construct
  source 1..66: mol_type = other DNA
cagaaccagctctataacgagctcaatctaggacgaagagaggagtacgatgttttggacaagaga

SEQ ID NO: 42 - protein, 22 aa, synthetic construct
QNQLFNELNLGRREEFDVLDKR

SEQ ID NO: 43 - DNA, 66 bp, synthetic construct
  source 1..66: mol_type = other DNA
cagaaccagctctttaacgagctcaatctaggacgaagagaggagttcgatgttttggacaagaga

SEQ ID NO: 44 - protein, 23 aa, synthetic construct
QEGLYNELQKDKMAEAYSEIGMK

SEQ ID NO: 45 - DNA, 69 bp, synthetic construct
  source 1..69: mol_type = other DNA
caggaaggcctgtacaatgaactgcagaaagataagatggcggaggcctacagtgagattgggatgaaa

SEQ ID NO: 46 - protein, 23 aa, synthetic construct
QEGLFNELQKDKMAEAFSEIGMK

SEQ ID NO: 47 - DNA, 69 bp, synthetic construct
  source 1..69: mol_type = other DNA
caggaaggcctgttcaatgaactgcagaaagataagatggcggaggccttcagtgagattgggatgaaa

SEQ ID NO: 48 - protein, 22 aa, synthetic construct
HDGLYQGLSTATKDTYDALHMQ

SEQ ID NO: 49 - DNA, 66 bp, synthetic construct
  source 1..66: mol_type = other DNA
cacgatggcctttaccagggtctcagtacagccaccaaggacacctacgacgcccttcacatgcag

SEQ ID NO: 50 - protein, 22 aa, synthetic construct
HDGLFQGLSTATKDTFDALHMQ

SEQ ID NO: 51 - DNA, 66 bp, synthetic construct
  source 1..66: mol_type = other DNA
cacgatggccttttccaggggctcagtacagccaccaaggacaccttcgacgcccttcacatgcag

SEQ ID NO: 52 - protein, 112 aa, synthetic construct; identical to SEQ ID NO: 38

SEQ ID NO: 53 - DNA, 336 bp, synthetic construct; identical to SEQ ID NO: 39
  source 1..336: mol_type = other DNA

SEQ ID NO: 54 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 55 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 56 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 57 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - DNA, 420 bp, synthetic construct
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 59 - protein, 143 aa, synthetic construct
IPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 60 - DNA, 429 bp, synthetic construct
  source 1..429: mol_type = other DNA
attcccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 61 - DNA, 4627 bp, synthetic construct
  source 1..4627: mol_type = other DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 62 - protein, 178 aa, synthetic construct
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 63 - DNA, 534 bp, synthetic construct
  source 1..534: mol_type = other DNA
gatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 64 - protein, 180 aa, synthetic construct
LEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 65 - DNA, 540 bp, synthetic construct
  source 1..540: mol_type = other DNA
ctggaggatctgaaaaacgtgttccctcctgaagtggctgtctttgaaccatccgaggccgagatttcccatacccagaaagcaactctggtctgtctggccactggattctaccccgatcacgtggaactgtcttggtgggtgaacggcaaggaagtccattccggagtctctaccgaccctcagcccctcaaggagcagcctgctctcaacgattctcggtactgcctgtcatctcgactgagagtgtctgccaccttctggcagaaccctagaaaccactttcggtgtcaggtccagttttacggcctgagcgagaacgatgagtggacacaggatagagccaaacctgtgacacagattgtgagcgccgaggcttggggacgagccgattgtggcttcacatccgagtcttaccagcagggagtgctgtctgctacaatcctctacgaaattctcctggggaaggccaccctgtacgctgtcctcgtgtctgctctggtgctcatggctatggtcaaacgaaaggactctagaggc

SEQ ID NO: 66 - protein, 175 aa, synthetic construct
LNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 67 - protein, 176 aa, synthetic construct
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 68 - DNA, 528 bp, synthetic construct
  source 1..528: mol_type = other DNA
gacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 69 - DNA, 1448 bp, synthetic construct
  source 1..1448: mol_type = other DNA
aggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcagacaagactagatccaaaaagaaaggaaccagcgcacaccatgaaggagaattgggcacctgtggttcattcttctcccagattctcagcccaacagagccaagcagctgggtcccctttctatgtggcctgtgtaactctcatctgggtggtgccccccatccccctcagtgctgccacatgccatggattgcaaggacaatgtggctgacatctgcatggcagaagaaaggaggtgctgggctgtcagaggaagctggtctgggcctgggagtctgtgccaactgcaaatctgactttacttttaattgcctatgaaaataaggtctctcatttattttcctctccctgctttctttcagactgtggctttacctcgggtaagtaagcccttccttttcctctccctctctcatggttcttgacctagaaccaaggcatgaagaactcacagacactggagggtggagggtgggagagaccagagctacctgtgcacaggtacccacctgtccttcctccgtgccaacagtgtcctaccagcaaggggtcctgtctgccaccatcctctatgagatcctgctagggaaggccaccctgtatgctgtgctggtcagcgcccttgtgttgatggccatggtaagcaggagggcaggatggggccagcaggctggaggtgacacactgacaccaagcacccagaagtatagagtccctgccaggattggagctgggcagtagggagggaagagatttcattcaggtgcctcagaagataacttgcacctctgtaggatcacagtggaagggtcatgctgggaaggagaagctggagtcaccagaaaacccaatggatgttgtgatgagccttactatttgtgtggtcaatgggccctactactttctctcaatcctcacaactcctggctcttaataacccccaaaactttctcttctgcaggtcaagagaaaggatttctga

SEQ ID NO: 70 - DNA, 1489 bp, synthetic construct
  source 1..1489: mol_type = other DNA
aggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcaggtgagtggggcctggggagatgcctggaggagattaggtgagaccagctaccagggaaaatggaaagatccaggtagcggacaagactagatccagaagaaagccagagtggacaaggtgggatgatcaaggttcacagggtcagcaaagcacggtgtgcacttcccccaccaagaagcatagaggctgaatggagcacctcaagctcattcttccttcagatcctgacaccttagagctaagctttcaagtctccctgaggaccagccatacagctcagcatctgagtggtgtgcatcccattctcttctggggtcctggtttcctaagatcatagtgaccacttcgctggcactggagcagcatgagggagacagaaccagggctatcaaaggaggctgactttgtactatctgatatgcatgtgtttgtggcctgtgagtctgtgatgtaaggctcaatgtccttacaaagcagcattctctcatccatttttcttcccctgttttctttcagactgtggcttcacctccggtaagtgagtctctcctttttctctctatctttcgccgtctctgctctcgaaccagggcatggagaatccacggacacaggggcgtgagggaggccagagccacctgtgcacaggtgcctacatgctctgttcttgtcaacagagtcttaccagcaaggggtcctgtctgccaccatcctctatgagatcttgctagggaaggccaccttgtatgccgtgctggtcagtgccctcgtgctgatggccatggtaaggaggagggtgggatagggcagatgatgggggcaggggatggaacatcacacatgggcataaaggaatctcagagccagagcacagcctaatatatcctatcacctcaatgaaaccataatgaagccagactggggagaaaatgcagggaatatcacagaatgcatcatgggaggatggagacaaccagcgagccctactcaaattaggcctcagagcccgcctcccctgccctactcctgctgtgccatagcccctgaaaccctgaaaatgttctctcttccacaggtcaagagaaaggattccagaggctag

SEQ ID NO: 71 - protein, 173 aa, synthetic construct
DKQLDADVSPKPTIFLPSIAETKLQKAGTYLCLLEKFFPDVIKIHWQEKKSNTILGSQEGNTMKTNDTYMKFSWLTVPEKSLDKEHRCIVRHENNKNGVDQEIIFPPIKTDVITMDPKDNCSKDANDTLLLQLTNTSAYYMYLLLLLKSVVYFAIITCCLLRRTAFCCNGEKS

SEQ ID NO: 72 - protein, 189 aa, synthetic construct
DKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKS

SEQ ID NO: 73 - DNA, 5591 bp, synthetic construct
  source 1..5591: mol_type = other DNA
ataaacaacttgatgcagatgtttcccccaagcccactatttttcttccttcaattgctgaaacaaagctccagaaggctggaacatacctttgtcttcttgagaaatttttccctgatgttattaagatacattggcaagaaaagaagagcaacacgattctgggatcccaggaggggaacaccatgaagactaacgacacatacatgaaatttagctggttaacggtgccagaaaagtcactggacaaagaacacagatgtatcgtcagacatgagaataataaaaacggagttgatcaagaaattatctttcctccaataaagacaggtatgtgtttacgcatatcatctgtcagaacacttctttgaaagtgaatgctgcattttttcctttcagtattaatgaaaaacaaacataaatctttcttaaatattgttacatttaatggtagcataaatgccctgctacttttctatagaattaaaatggtataggttttggagaaaacaaaattgaaaaagttactgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaatgatgtttatctgttgttgacagaatgatgtttatctaaaaacagttccaattttctatttcctttgctgagacacaaaggggaggcaaatgtgcaaagcttgagggtagtcttaccactgtgcttaagtgttctgatttttctagtgatcagggcaaaataaaaagtatagtaagttccaaggcagtgaatattatacaggagagaagttacagttttataatgtgttttcctttacactaaattctaaaagtaaaaagtctttttttttttttgacagagtttcactcttgttgcccaagcaggtgtgctatggtatgatctcagctcactgcaacctccacctcccgggttcaagtgattctcttacttcagcctcccgacaggctgggattgcaggcgcctgccaccacacctggctaatttttgtgtttttagtagagatggggtttcaccatgttggccaggctggtctcaaattcctgacctcaagtgatccatccacctcggcctccaagtgctgggattatgggcgtcagccactgtgcccagcctaaaagtaaaatgtctttcatgagcttcccaaggcagctacgttaaggaggacacttctcttaatgtcattctacagtagatttctaatgctctttcttggaagtttgtttttctgagaaaagctaaaaatataacatggaagtgatcatattatataatcaatgaagtgcttttcaaggagataaaactaatctggtccacacttgcaaccaaccttgattgagagagagagagaactcaggatacacttgaagattttattatggggaacagttactttattctttttacctcaatcaatgcatggaaataagtgatagtcattttcatttatcttttaataaatgaagtcaccatgaggaaaataaaaagacattgaaaacccattaaagtcagcccttaaagatatttggacatgcagacttgataactaacgtttgcattcttgagacttacccaaaacccatacctcaagtccaagtttttagaattcatgaaataaagatctcagtgagtgcataaaattgcgcaccagaatcatatccgtatagacaagaacacatctactagaaaaataataaaccaacacaccaatgcaactgtgttttcttctgttttaaagtatgttgtctttgtatgcatgtttgcttcttcctttttttttttaacatcacagataaattcaactctcacctcaggttttattgagagaactgtcaatgtgacttggcctctgtctttctagtcccagaaagaattgcactgaaatctgagctcctgtaataaaaacaaccatttgctgagagtaattaacatactgaaagagattttcttagagtacacaatggtgacattatattgcctctttataaataactttctatctatttctgtggattattcctacaaagtacttttcatatgtccaatttcttttcttcccctacaactactgtctgaatactggctctgctatttgctgatatgattctcggcaagttgcctgcactttttaaactttatttcctcattcagaacatggggccatacataatacaactcacttcagtgttattggggaattaaacaaaaaatgcatgggaagcatttaacatagtgcctgacacaataatgagtactcagtagatgttagcttttattaatattgttgttgttatgtccagaaacactatacctccagaaaatcatgggtacttgctggggacattggggatatgcatgatttggaaaagaatgactgctttttttgcttagatgagaaatttttctaagccagactccttcaaatatgtaagattctgttgtggattcaaggactgaaagaattcttggccgagtgtggtggcttatccctgtaatcccagcattttgtgaggacaaggcaggaagattgcttgagtccaggagtttgaaaccagcctgcgcaacatggcgaaaccctgtctctacaaaaaatacaaacattagctcggagtgagtgctgacatgtgcctgtactcccagctactcagaaggctgagatgggaggatctcatgagcctggggagtttgaggcttcagtgagccgtgatgacaccgtactatactccactccagcctgggtgacagtgagaccctgcctcaaaaaacaaacaaacaaacaaacaaaacaaaattaatctttttgctgatgtcatgtcagcagtgtgtgttgaaggctgtaaagcagccatttgttcagtttatttttccattgaacaagtatttatcaaaaacatactttgtggcagtcactatgctaggagctatgaatacagaaggaaaagtaaatgctcttggatactacactccagttgtgataaaaaagaaaaaatgtattcttcaccaacttcaacatcttgatgtgcaaaaacataatacatgaattagatctacctaattacacagaattagaccaattgtttctggaattgtgggctcatatttttaataactgtcctcctgcctctctgtcgacaggttttataaatattcatttaattacacacacacacacgaacaattgactagtacttgctctcattcttctagatgtcatcacaatggatcccaaagacaattgttcaaaagatgcaaatggtaagcttttgtgtttttcccttcctcctgatcattttgttttgaacttctctggcttgaaaaatcagggaatggattttgctaggttggatgctgcagaatggacctagtgatattttaaattagtccctcattttctaggagttgtattaacaaacctaactactgctttggggtatgagatgactgtaaattagagagggtacagtggtatagtgatatgcttttaattatttcaaaaaaaagattttattcattcatgtgtcttttttctttttcttttcttttttttttttttttggacagagtcttgctctgtcacccaggctggagtgcggtggcagtatctcagctcaccacaacctccgcctcccggcttcaagtgattctcctgcctcagcttctcgagtagctgggactacaggcgcgtgccaccatgcccggctaatttttgtatttttagtagagttggggtttcaccatgttggccaggatggcctcgaatttgtgacctcgtgatctgccccctcgccctcccgaactgttgggattacaggcgtgagtcactgtgcccggcctcctgtcctgtcttttgtttaatgactgggaaaaacatgataccatgttgcttctcgagttgttttgttttagtctttggtctttgctagtagctaataacacgaactagtgtttatcaagtgctttttacacagaagggcttgggctgtgttctgcattttcttgtttaaccctcttaaaactcctataaaatggtacatatttttctcccaatttacagtccctttaaagcaaataattataaaaatccctatacatgtcacacagctagatctgggatttcaaatcaggccatcaaacaaagagtttatgtacttagtaagttttctgttctttttctacaatagagtcagatagcaagaaattaccaagccaggaacctgaaacaaaacggacatcatgtggggctgggtgggtgcatgggctttgcagactggactttcactccagctcttttaatgattaggtgtaagtgacctacattttgtgagcaacagttttctcatcagccaacaaagaataattacaccagattcacagttattgaagagataaaggcatgaatgtgagatgtctggcatagggcatctcatttagcagacacagaatgagtacttgtttctggctttttctctctacatatgcacaaagaatgcgactagaagcatgggctctagccctgctcaactttcctctatttccaataccaaggggctctgacttaggctgccacaccaggcaaggagggcagtaccacctcacttgaccaagggcagggagtcacggacacatcacttcttgagatccttttccacaccaaggactgatgtttctggaattctcactttatgaagacaaaacatataaatggaaattttctcaggtagagactcactcttgtagctcattgagtaggcactagtggtccacccccactgtctttacttattccttgacatcacatatctcttgcaaaacctcaaataatattaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatgtatcttattctgtaaaaagaacatgtgcaatttctcttagatacactactgctgcagctcacaaacacctctgcatattacatgtacctcctcctgctcctcaagagtgtggtctattttgccatcatcacctgctgtctgcttagaagaacggctttctgctgcaatggagagaaatcataa

SEQ ID NO: 74 - DNA, 9549 bp, synthetic construct
  source 1..9549: mol_type = other DNA
ataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacaggtatgtgtttacacatatcatctgtcagaacacttctttgaaagtgaatgctgcattttttcctttcagtattaatgaaaaacataaatctttcttaaaaattgttacatttaatggtagcgtaaatgccctgctacttttctatagaattaaaatggtataggttttggagaaaacaaaattgaaaaagttgctgaaggtttgtcagcctcagctccattatccaaaataagaaagtcacgtgctggtttttagggttgttagatggattaaagaaacaacatacacagaagcatctagcaacgtgacacgtggtaaacgctcaaaaagtgttctcccttcttttgatgactttacttgatcaggaaataacatatatatgtctttcaggaatgttctgcccaagcaggagagtcactcacctcaatcttgctacccacaaagtttaacctaaaaacaacgggttcattgttgacaaaataatgtttatctgaagataactgtagatcatatttatctgtagataatgtttatctgtggagtgtggctctacaaaacatagaatagtcttggtcactgcagttttatagaggccttgggtttttcagagtttcattttatatatcaccataaagtaacatttcataattacaggttggtaaggcttacatgtacaaacattcttccattttccataataaatgcatttcctgccattggtgaatgcagctcaataaacatttattgtacaattatgacacgccaggcttagtggaaatgtggatgaacagacaaggatgagttactgtcctaaggatgatgcatgacagtgcagagaatatactctcttcctgatcactcagggtcactcatgattcatgcgcgaggtcccaaaacagtgcctttgatgcagattctgtacatctctagacgattggtccaagggctgaatgtgctctggcccagtggtccagtctgtcactatatgtcaacatcctgaatatgaacataacagtccaacatctcaagagtgggcatgaaaaggactcattttgtgctttttcctgtggttaacaagtcctttttagcctgggggaacaagcattaacaaaatgtttgaagatctttgccacgtaccattccaaatttctagggtaagtctttagcttttcagatcctgagtttctgcaatgatcaaatgtgatttggacagttgcgttgactttctcctggggctataatggagtgcaaaggaaacaatggcagggaaaatgcttgctttcaaaatggtagcatggatgtgttcattcgtgtagttactgtattaggtatagcctttcctgaaactaactgaagtggggttataaaaacagtcccaattttctatttcctttgctgagacacaaagaggagacaaaagagcaaagcttgagggtagttttaccactgtgcttaagtgttctgatttttccagtgatcagggtgaaataaaaagcatagtaagttccagggcagtgaataccatacaggagacaagttacagttttataatgtgttttactttacactaaattctaaaagtaaaatgtctttttttttttccgagacagagtttcactcttgtagcccaggcaggagtgctatggtgtgatctcggctcacagcaacctccacctcccagtttcaagcgattcttctgcctcagcctcccgagaagttgaaattacaggtgcctggcaccatatctcgctaattattctatttttagtagagatcgggttttaccatgttggccaggctggtctcgaactcctgacttcaagtgatccacccgcctcagcctcccaaagtgctgggattacaggtgtgagtcactgtgccggacctaacagtaaaatgtctttcatgtgcttctcaaggcaactacattaaggaggacacatctcttaatgtcattctacagtagatttctaatgctctttcttggaagtttgtttttctgagaagagctaaaaatataataacatggaagtgatcatattatataatcaatgaagtgctttcaaaggagataaaactaacctggtctgcatttgcaaccagccttgattgagagagagagaactcaggatacacttagagattttattatggggaatagttactttattcattttacctcaatcaatgcatggaaataagtgacagtcattttcatttatcttttaataaataaagtcaccatgaggaaaatgaaaacccattaaagtcagtccttaaagatatttggacatgcagacatgataactaacatttccattcgtgagacttacccaaaacctatacctcaagtccatttcttagaatacatgaaataaagatctcagtgagtgtataaaactgcacaccagaatcatatccgtatagacaagaatacatctactagaaaaatataaaccaaaacaccaaggtgactctgtttttttctgttttaaaatatgttgtctttgtatgcatgtttgcttcttcctttttttttttaaacatcgcagataaattcaactctcacctcagttgagagagaactgtcaatgtgacttggcctctctctttctagtcccagaaagaattgcactgaaatgctgagctcctgtaataaaaatgaccatttgctgagagtaattaacatactgaaagagattttcttagaatagtgcacaatggcccaatggtgacattatattgtctctttataaattattttctatctatttctgtggattatttctacaaagcacttttcatatgtccaattccttttattcccctacaagtactgactgactactggctctgctgttcactgatatgactttcggcaagttgcctgcactttttaaacgttatttcctcattcagaacatggggccatacaaaatacaactcacttcagtgttattggggaattaaacaaataaatgcatgggaagcatttaacatagtgcctgacacaataatgagcactcagtagatgttagcttttattaatattgttgttgctatgtccagaaacactatacctccagaaaatcatgggtacttgctggggacgttggggatatgcatgattttgaaaggagtgactgctctttactgctcagatgagaaatttttctaagccagactccttcaaacatgtaagattctgttgtggattctaggactgaaagaattcttggccgagtgtggtggcttatcctggtaatctcatcatttgggaggacaaggcaggaagattgcttgagcccaggagttggaaacaagcctggacaacatggcgaaaccctgtctctacaaaaaatacaaacattagctggtcatgggagtgagtgcctgtactcccagctactcaggaggctaagataggaggatcacctgagcctgggcagtttgaggtttcagtgagccgtgatgacaccatactatactccactccagcctgggtgacagtgacatcctgcctcaaaaaaacccccaaaattattctttttgctgatttcatgtcagcagtgtgtgctgaaggctgtaaagtagccacttgttctgtttatttttccattgaacaagtatttatcaaaaacgtactttgtggaaggcactgtgctaggaactatgcatacagaaggaaaaccaaatgttcttggatactacactccagttgtgataaaaaagaaaaaagtattcttcacaaacttcaacattttgatgtgcaaaaacataatatatgaattagatctacctaactacacagaattagaccaattatttctgggattatgggctcatatttttaataactgtcctcctacctctctgttgacaggttttataaatattcatttaattacacacagtcacagacacactcagacacacacacatacacacacacacacaccttgacaaataatgggcatgaacaattgactggtacttgctctcattcttctagatgtcaccacagtggatcccaaatacaattattcaaaggatgcaaatggtaagtttttgtgttttttatttcctcctgatcattttaagttttgaacttctctggcttgaaaaatcagggaatggattttgctaggttggatgctgcagaatggacctaatcatattttaaattagtccctctttttctaggagttgtattaacaaacctaactactgcttcatgtaagagatgactgtaaattgaagggtacagtgatatgctttcagttatttcaaaaaacagactttactcatccatgtgtcttttttcttttcttttttttcttttttgagacggagtctcgctctgttgaacaggctggattgcagtgacgcgatctcacctcactacaacctccgcctctggagttcaagcgattctccagcctcagcttctcaagtagctgggactacaggcacatgccaccatgtccgggtcatctttgtatttttagcagagaccgggtttcactatgttggccaggctggtctagaattcctgacttcgtgatctgccccctcagccctccgaagtgctgggattacagacgtgagtcactgtgcccggcctaacagtaaaatgtctttcatgcgcttctcaaggcaactacgttaaggaggacacttctcttaatgtcattctacagtagatttctaatgctctttcttggaagtttgtttttctgagaaaagctaaaaatataacatggaagtgatcatattgtataatcaatgaagtgcttttcaaggagataaaactaatctggtccacgtttgcaaccaaccttgattgagagagagagagaactcaggatacacttggagattttattatggggaatagttactttattcttttttcctcaatcaattcatggaaataagtgatagtcatattcatttatcttttaataaatgaagtcaccatgaggaaaataaaaagacattgaaaacccattaaagttagcccttaaagatatttggacatgcagacttgataactaacgtttgcattcttgagacttacccaaaacccatacctcaagtccatgtttttagaattcatgaaataaagatctcagtgagtgcataaaattgcgcaccagaatcatatccgtatagacaagaacacatctactagaaaaataataaaccaacacaccaatgcaactgtgttttcttctgttttaaaatatgttgtctttgtatgcatgtttgcttcttccttttttttttttaacatcacagataaattcaactctcacctcaggttttattgagagaactgtcaatgtgacttggcctctgtctttctagtcccagaaagaatcgcactgaaatgctgagctcctgtaataaaaatgaccatttgctgagagtaattaacatactgaaagagattttcttagagtacacaatggtgacattatattgtctctttataaataactttctatctatttctgtggattattcctacaaagtacttttcatatgtccagtttcttttcttcccctacaactaccgtctgaatactggctctgctatttgctgatatgattctcggcaagttgcctgcactttttaaactttatttcctcattcagaacatggggccatgtaatactcatgtacgtgagtattacgtaataatgctcacttaagtgttactggggaattaaacaaaaaaatgcatggcaagcatttaacatagtgcctgacacaataatgagcactcagtagatgttagattttattaatattgttgttgttatgtccggaaacactatacctccagaaaatcatgggtacttgcttgggatgttggggatatgcatgatttggaaaggtatgactgcttttttctgcttagatgagaaatttttctaagccagactccttcaaatatgtaagattctgttgtggattctaggacggaaagaattcttggtcaggtgtggtttcttatccctgtaatcccagaattttgggaggacaaggcaggaagattgcttgagcccaggagtttgaaaccagcctgggcaacaagacgaaaccctgtctctacaaaagtacataaattagcttggcttggtggtgtgtgcctgtattaccagctattcgggagactgagatgggaggatctcctgaacctgtgaagtttgaggcttcagtgagccgtgatgacaccatactatactcgactccagcctgtgcgacagtgagactctgcgtcaaaaaaaaaaccccaaaattattgtttttgctgatttcaggtcagcagtgtgtgctgaagggtgtaaagtagccacttgatcagtttatttttccactgaacaagtatttatcaaaaacatactttgtggtctgtttttgataaataaaaaggcactgtgctaggagccatgaatacagaaggaaaaccaaatgttcttggatactacactccagttgtgataaaaaagaaaaatgtattcttcacgaacttcaacattttgatatgcaaaaacatagtatataaattagatctacctgattacgtagaatcagaccaattatttctggaattgagggctcatatttttaataactgtcctcctgcctctctgttgacaggttttataaatattcatttaattacacacacacacacacacaccttgacaaataatggacatgaacaattgactagtacttgctctcattcttctagatgtcatcacaatggatcccaaagacaattggtcaaaagatgcaaatggtaagcttttgtgtttttcctttcctcctgatcattttaagttttgaacttctctggcttgaaaaatcagggaatgggccgggtgcggtggctcacgcctgtaatcccagcactttgggaggccgaggcgggcggatcacgaggtcaggagatcgagaccatcccggctaaaacggtgaaaccccgtctctactaaaaatacaaaaaattagccgggcttagtggcgggcgcctgtagtcccagctacttgggaggctgaggcaggagaatggcgtgaacccgggaggcggagcttgcagtgagccgagattgcgccactgcactccactccagcctgggcgacagagcgagactccgtctcaaaaaaaaaaaaaaaaaaaaaaaaagaaaaatcagggaatggattttgctaggttggatgctgcagaatggacctagtgatattttaaattagtccctctttttctaggagttgtattaacaaacctaactactgcttcgggtatgagatgactgtaaattagagggtacagtgatatgctttcagttatttcaaaaaacagactttattcatccgtctgtcttttttttttttttttttttttttttttgagacggaggagtctcactctatcacccaggctggagtgcagtggcgcgatctcggctcaccataacctccgccttactggttcaagcgattctccagcctcagcttctcaagtagctgggactacaggtgcacaccaccatacctggctaatttttgtatttttaatagagatggggtttcaccacgctggccaggatggtcttgaattcttgacctcgtgatctgccccctcgggctcccaaacttctgggattataggcgtgagccactgtgcccggccttctgtcttttgttataatgactggggaaaacatgataccatgttgcttcttgagttgttttgttttagtctttggtctttgctagtagctaataacacgaactagtgtttatcaagtgctttttacacagaagggcttgttctgcattttctagtttaatcatcttaatactcctataaagtagtacaatatattttctcccattttacagtccctttaaagtaaataactataaaaatcccttatacatgtcacacagctaggtctggcatttcaaatcaggacatcaaacaaagaattcgtgcagttactaagtcctctattttttctacaatagaaaaaatagcaagaattacagatagcaagacattacaaggcaggaatctgaaacgaaagggacataatgtggggctgggtgggtgcatgagctttgcagactagactttcattccagctcttttaatgattaggtgtaagtgacctacattttgtgagtaacagttttctcatcagccaactaagaataattacaccagattcacagttattgaagagataagggcatgaatgtgagatgtctggcgtagggtatctcatttagcagacacagaatgaatacttgtttctggctttttctctctacatatgcacaaagaatgtgactagaagcattggctctagccctgctcaactttcctctatttccaataccaaggggctctgacttaggctgccacaccaggcaaggaggggcagtaccacctcacttgaccaagggcagggagtcacggacacatcacttcctgagatccttttccacaccaaggactgatgtttctggaattctcactttatgaagacaaaacatataaatggaaatttctgcaggaagagactcactcttgtagctcattgagtaggcactagtggtccacccccactgtctttacttattccttgacatcacatatctcttgtaaaacctcaaataatgttaaatgcaatcacccaataatagcatagccataattagaggcatttaggaaagacaggtgagtgtgccacaactacctaacacatcagcaaatctggattaaccactttctttgattttccacaatgcaaccttactttttaatagttgggaatgttctaagtgaatttagcagaggttgttaatcaacttgaaagctgaattctgacttgtctgactcttggtggtgctggtagcagtagatgtttacttttaggttttggtggtggtggaatatcacttcaacgtaaatcatcagaaataagtatttgtgaacccctctcgcattaatatatcttattctgtaaaaagaacatgtgcaatttctcttagatacactactgctgcagctcacaaacacctctgcatattacacgtacctcctcctgctcctcaagagtgtggtctattttgccatcatcacctgctgtctgcttagaagaacggctttctgctgcaatggagagaaatcataa

SEQ ID NO: 75 - protein, 153 aa, synthetic construct
SQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 76 - protein, 10 aa, synthetic construct
IPNIQNPDPA

SEQ ID NO: 77 - DNA, 30 bp, synthetic construct
  source 1..30: mol_type = other DNA
attcccaatatccagaaccctgaccctgcc

SEQ ID NO: 78 - protein, 11 aa, synthetic construct
LEDLKNVFPPE

SEQ ID NO: 79 - DNA, 33 bp, synthetic construct
  source 1..33: mol_type = other DNA
ctggaggatctgaaaaacgtgttccctcctgaa

SEQ ID NO: 80 - protein, 182 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 81 - protein, 171 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 82 - protein, 127 aa, synthetic construct
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 83 - protein, 207 aa, synthetic construct
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 84 - protein, 254 aa, synthetic construct
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 85 - DNA, 762 bp, synthetic construct
  source 1..762: mol_type = other DNA
atggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaa

SEQ ID NO: 86 - protein, 288 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 87 - DNA, 864 bp, synthetic construct
  source 1..864: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgta

SEQ ID NO: 88 - protein, 309 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFKRGRKKLLYIFKQPFMRPVQTTQEEDGCSCRFPEEEEGGCEL

SEQ ID NO: 89 - protein, 349 aa, synthetic construct
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFRSKRSRLLHSDYMNMTPRRPGPTRKHYQPYAPPRDFAAYRKRGRKKLLYIFKQPFMRPVQTTQEEDGCSCRFPEEEEGGCEL

SEQ ID NO: 90 - DNA, 1176 bp, synthetic construct
  source 1..1176: mol_type = other DNA
gtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 91 - protein, 257 aa, synthetic construct
MGASGSKKLSKHSRGLRERLLRARGDGYGKQRDASGGEYSQFQEESGREQNSPSCEGQQYQQGEYMNSPWRNPATERQKDLYRQQNMDDVDSDDDDLIGVPVTPRVPRREMTYKLAIDMSHFIKEKGGLQGMFYSRRRHRILDIYLEKEEGIIPDWQNYTHGPGVRYPMYFGWLWKLVSVELSQEAEEDEANCLVHPAQTSRHDDEHGETLVWQFDSMLAYNYKAFTLYPEEFGHKSGLPEKEWKAKLKARGIPYSE

SEQ ID NO: 92 - protein, 5 aa, synthetic construct
SYWMN

SEQ ID NO: 93 - protein, 17 aa, synthetic construct
QIYPGDGDTNYNGKFKG

SEQ ID NO: 94 - protein, 13 aa, synthetic construct
KTISSVVDFYFDY

SEQ ID NO: 95 - protein, 11 aa, synthetic construct
KASQNVGTNVA

SEQ ID NO: 96 - protein, 7 aa, synthetic construct
SATYRNS

SEQ ID NO: 97 - protein, 9 aa, synthetic construct
QQYNRYPYT

SEQ ID NO: 98 - protein, 138 aa, synthetic construct
MALPVTALLLPLALLLHAEVKLQQSGAELVRPGSSVKISCKASGYAFSSYWMNWVKQRPGQGLEWIGQIYPGDGDTNYNGKFKGQATLTADKSSSTAYMQLSGLTSEDSAVYFCARKTISSVVDFYFDYWGQGTTVTV

SEQ ID NO: 99 - DNA, 414 bp, synthetic construct
  source 1..414: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtc

SEQ ID NO: 100 - protein, 124 aa, synthetic construct
MALPVTALLLPLALLLHADIELTQSPKFMSTSVGDRVSVTCKASQNVGTNVAWYQQKPGQSPKPLIYSATYRNSGVPDRFTGSGSGTDFTLTITNVQSKDLADYFCQQYNRYPYTSGGGTKLEI

SEQ ID NO: 101 - DNA, 372 bp, synthetic construct
  source 1..372: mol_type = other DNA
atggcactgcccgtgaccgctctgcttctcccactagctctgcttctccacgcagacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatc

SEQ ID NO: 102 - DNA, 3505 bp, synthetic construct
  source 1..3505: mol_type = other DNA
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgttcaatgaactgcagaaagataagatggcggaggccttcagtgagattgggatgaaaggcgagcgccggaggggcaaggggcacgatggccttttccagggtctcagtacagccaccaaggacaccttcgacgcccttcacatgcaggccctgccccctcgctaaatccctaggcgactgtgccttctagttgccagccgtctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctcgaggaattcgacgtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattaggatccatcgccaccatgggtggcaagtggtcaaaacgtagtgtggttggatggcctgctgtaagggaaagaatgagacgagctgagccagcagcagatggggtgggagcagtatctcgagacctggaaaaacatggagcaatcacaagtagcaatacagcagctaacaatgctgattgtgcctggctagaagcacaagaggaggaggaggtgggttttccagtcagacctcaggtacctttaagaccaatgacttacaaggcagctgtagatcttagccactttttaaaagaaaaggggggactggaagggctaatttactcccaaaaaagacaagatatccttgatctgtgggtctaccacacacaaggctacttccctgattggcagaactacacaccagggccagggatcagatatccactgacctttggatggtgcttcaagctagtaccagttgagccagagaaggtagaagaggccaatgaaggagagaacaacagcttgttacaccctatgagcctgcatgggatggatgacccggagaaagaagtgttagtgtggaagtttgacagccgcctagcatttcatcacatggcccgagagctgcatccggagtactacaaggactgctaa

SEQ ID NO: 103 - DNA, 366 bp, synthetic construct
  source 1..366: mol_type = other DNA
caggtccaactgcagcagcctggggctgagctggtgaggcctggggcttcagtgaagctgtcctgcaaggcttctggctacacgttctccaactacttgatgaactgggttaagcagaggcctgagcaagaccttgactggattggaaggattgatccttacgatggtgacattgactacaatcaaaacttcaaggacaaggccatattgactgtagacaaatcctccagcacagcctacatgcaactcagcagcctgacatctgaggactctgcggtctattactgtgcaagaggttatggcacggcctatggtgtggactactggggtcaaggaacctcagtcaccgtctcctcagcggccgca

SEQ ID NO: 104 - protein, 5 aa, synthetic construct
SYIMH

SEQ ID NO: 105 - protein, 17 aa, synthetic construct
VISYDGRNKYYADSVKG

SEQ ID NO: 106 - protein, 9 aa, synthetic construct
DTDGYDFDY

SEQ ID NO: 107 - protein, 11 aa, synthetic construct
RASQSVSSYLA

SEQ ID NO: 108 - protein, 7 aa, synthetic construct
DASNRAT

SEQ ID NO: 109 - protein, 9 aa, synthetic construct
QQRTNWPLT

SEQ ID NO: 110 - protein, 135 aa, synthetic construct
MELGLSWIFLLAILKGVQCQVQLVESGGGVVQPGRSLRLSCAASGFTFSSYIMHWVRQAPGKGLEWVAVISYDGRNKYYADSVKGRFTISRDNSKNTLYLQMNSLRAEDTAVYYCARDTDGYDFDYWGQGTLVTV

SEQ ID NO: 111 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRTNWPLTFGGGTKVEIK

SEQ ID NO: 112 - protein, 5 aa, synthetic construct
NYLMN

SEQ ID NO: 113 - protein, 17 aa, synthetic construct
RIDPYDGDIDYNQNFKD

SEQ ID NO: 114 - protein, 10 aa, synthetic construct
GYGTAYGVDY

SEQ ID NO: 115 - protein, 15 aa, synthetic construct
RASESVDNYGISFMN

SEQ ID NO: 116 - protein, 7 aa, synthetic construct
AASRQGS

SEQ ID NO: 117 - protein, 9 aa, synthetic construct
QQSKEVPWT

SEQ ID NO: 118 - protein, 122 aa, synthetic construct
QVQLQQPGAELVRPGASVKLSCKASGYTFSNYLMNWVKQRPEQDLDWIGRIDPYDGDIDYNQNFKDKAILTVDKSSSTAYMQLSSLTSEDSAVYYCARGYGTAYGVDYWGQGTSVTVSSAAA

SEQ ID NO: 119 - protein, 111 aa, synthetic construct
DIVLTQSPASLAVSLGQRATISCRASESVDNYGISFMNWFQQKPGQPPKLLIYAASRQGSGVPARFSGSGSGTDFSLNIHPMEEDDTAMYFCQQSKEVPWTFGGGTKLEIK

SEQ ID NO: 120 - protein, 366 aa, synthetic construct
CAGGTCCAACTGCAGCAGCCTGGGGCTGAGCTGGTGAGGCCTGGGGCTTCAGTGAAGCTGTCCTGCAAGGCTTCTGGCTACACGTTCTCCAACTACTTGATGAACTGGGTTAAGCAGAGGCCTGAGCAAGACCTTGACTGGATTGGAAGGATTGATCCTTACGATGGTGACATTGACTACAATCAAAACTTCAAGGACAAGGCCATATTGACTGTAGACAAATCCTCCAGCACAGCCTACATGCAACTCAGCAGCCTGACATCTGAGGACTCTGCGGTCTATTACTGTGCAAGAGGTTATGGCACGGCCTATGGTGTGGACTACTGGGGTCAAGGAACCTCAGTCACCGTCTCCTCAGCGGCCGCA